SEQUENCE LISTING (280 sequences)

SEQ ID NO: 1 - protein, 8 aa, synthetic construct
  REGION 1..8: note = BGA-7207 HCDR1 (IMGT)
GFRLDTTE

SEQ ID NO: 2 - protein, 8 aa, synthetic construct
  REGION 1..8: note = BGA-7207 HCDR2 (IMGT)
IMGISGST

SEQ ID NO: 3 - protein, 16 aa, synthetic construct
  REGION 1..16: note = BGA-7207 HCDR3 (IMGT)
ARVVDSLFDDSAVFDY

SEQ ID NO: 4 - protein, 5 aa, synthetic construct
  REGION 1..5: note = BGA-7207 HCDR1 (Kabat)
TTEVG

SEQ ID NO: 5 - protein, 17 aa, synthetic construct
  REGION 1..17: note = BGA-7207 HCDR2 (Kabat)
TIMGISGSTYYADSVKG

SEQ ID NO: 6 - protein, 14 aa, synthetic construct
  REGION 1..14: note = BGA-7207 HCDR3 (Kabat) or BGA-7207 HCDR3 (Chothia)
VVDSLFDDSAVFDY

SEQ ID NO: 7 - protein, 7 aa, synthetic construct
  REGION 1..7: note = BGA-7207 HCDR1 (Chothia)
GFRLDTT

SEQ ID NO: 8 - protein, 6 aa, synthetic construct
  REGION 1..6: note = BGA-7207 HCDR2 (Chothia)
MGISGS

SEQ ID NO: 9 - protein, 123 aa, synthetic construct
  REGION 1..123: note = BGA-7207 VH
EVQLLESGGGLVQPGGSLRLSCAASGFRLDTTEVGWVRQAPGKGLEWVSTIMGISGSTYYADSVKGRFTISRDNSKNTLYLQMNSLRAEDTAVYYCARVVDSLFDDSAVFDYWGQGTLVTVSS

SEQ ID NO: 10 - DNA, 369 bp, synthetic construct
  misc_feature 1..369: note = BGA-7207 VH DNA
  source 1..369: mol_type = other DNA
gaggtccagttacttgagagtggtggaggtctggtccaaccaggaggttcgctgcgtttatcctgcgccgcgtctggattcagattggacaccaccgaagtaggttgggtgcgtcaagcgccggggaaaggactggaatgggtctccaccatcatgggtattagtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtcgattccctctttgatgacagcgccgtttttgattactggggacagggcaccttagttacagtctcatcg

SEQ ID NO: 11 - protein, 8 aa, synthetic construct
  REGION 1..8: note = BGA-4712 HCDR1 (IMGT)
GFMLSAED

SEQ ID NO: 12 - protein, 8 aa, synthetic construct
  REGION 1..8: note = BGA-4712 HCDR2 (IMGT) or BGA-4712-M3 HCDR2 (IMGT)
ILDFGGST

SEQ ID NO: 13 - protein, 15 aa, synthetic construct
  REGION 1..15: note = BGA-4712 HCDR3 (IMGT) or BGA-4712-M3 HCDR3 (IMGT)
ARVVYHAGGGVTFDY

SEQ ID NO: 14 - protein, 5 aa, synthetic construct
  REGION 1..5: note = BGA-4712 HCDR1 (Kabat) or BGA-4712-M3 HCDR1 (Kabat) or BGA-5623 HCDR1 (Kabat)
AEDVG

SEQ ID NO: 15 - protein, 17 aa, synthetic construct
  REGION 1..17: note = BGA-4712 HCDR2 (Kabat)
AILDFGGSTYYADSVKG

SEQ ID NO: 16 - protein, 13 aa, synthetic construct
  REGION 1..13: note = BGA-4712 HCDR3 (Kabat) or BGA-4712 HCDR3 (Chothia) or BGA-4712-M3 HCDR3 (Chothia)
VVYHAGGGVTFDY

SEQ ID NO: 17 - protein, 7 aa, synthetic construct
  REGION 1..7: note = BGA-4712 HCDR1 (Chothia)
GFMLSAE

SEQ ID NO: 18 - protein, 6 aa, synthetic construct
  REGION 1..6: note = BGA-4712 HCDR2 (Chothia) or BGA-4712-M3 HCDR2 (Chothia)
LDFGGS

SEQ ID NO: 19 - protein, 122 aa, synthetic construct
  REGION 1..122: note = BGA-4712 VH
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSS

SEQ ID NO: 20 - DNA, 366 bp, synthetic construct
  misc_feature 1..366: note = BGA-4712 VH DNA
  source 1..366: mol_type = other DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcg

SEQ ID NO: 21 - protein, 8 aa, synthetic construct
  REGION 1..8: note = BGA-4712-M3 HCDR1 (IMGT)
GFTLSAED

SEQ ID NO: 22 - protein, 17 aa, synthetic construct
  REGION 1..17: note = BGA-4712-M3 HCDR2 (Kabat)
AILDFGGSTYYAESVKG

SEQ ID NO: 23 - protein, 7 aa, synthetic construct
  REGION 1..7: note = BGA-4712-M3 HCDR1 (Chothia)
GFTLSAE

SEQ ID NO: 24 - protein, 122 aa, synthetic construct
  REGION 1..122: note = BGA-4712-M3 VH
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 25 - DNA, 366 bp, synthetic construct
  misc_feature 1..366: note = BGA-4712-M3 VH DNA
  source 1..366: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcg

SEQ ID NO: 26 - protein, 8 aa, synthetic construct
  REGION 1..8: note = BGA-5623 HCDR1 (IMGT)
GFTVSAED

SEQ ID NO: 27 - protein, 8 aa, synthetic construct
  REGION 1..8: note = BGA-5623 HCDR2 (IMGT)
ILDKGGST

SEQ ID NO: 28 - protein, 15 aa, synthetic construct
  REGION 1..15: note = BGA-5623 HCDR3 (IMGT)
ARIVYHAGGGVTFDT

SEQ ID NO: 29 - protein, 17 aa, synthetic construct
  REGION 1..17: note = BGA-5623 HCDR2 (Kabat)
AILDKGGSTYYAESVKG

SEQ ID NO: 30 - protein, 13 aa, synthetic construct
  REGION 1..13: note = BGA-5623 HCDR3 (Kabat)
IVYHAGGGVTFDT

SEQ ID NO: 31 - protein, 7 aa, synthetic construct
  REGION 1..7: note = BGA-5623 HCDR1 (Chothia)
GFTVSAE

SEQ ID NO: 32 - protein, 6 aa, synthetic construct
  REGION 1..6: note = BGA-5623 HCDR2 (Chothia)
LDKGGS

SEQ ID NO: 33 - protein, 122 aa, synthetic construct
  REGION 1..122: note = BGA-5623 VH
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 34 - DNA, 366 bp, synthetic construct
  misc_feature 1..366: note = BGA-5623 VH DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 35 - DNA, 41 bp, synthetic construct
  misc_feature 1..41: note = HCDR1a
  misc_feature 19..20: note = Unsure - Inserted by amino acid sequence ABNTTBNMCNMCVAH
  misc_feature 22..23: note = Unsure - Inserted by amino acid sequence DTR
  source 1..41: mol_type = other DNA
ctgcgccgcgtctggattcgacggttgggtgcgtcaagcgc

SEQ ID NO: 36 - DNA, 41 bp, synthetic construct; identical to SEQ ID NO: 35
  misc_feature 1..41: note = HCDR1b
  misc_feature 19..20: note = Unsure - Inserted by amino acid sequence ABNTTB
  misc_feature 22..23: note = Unsure - Inserted by amino acid sequence NMCNMCVAHDTR
  source 1..41: mol_type = other DNA

SEQ ID NO: 37 - DNA, 40 bp, synthetic construct
  misc_feature 1..40: note = HCDR2
  misc_feature 18..19: note = Unsure - Inserted by amino acid sequence RSC
  misc_feature 21..22: note = Unsure - Inserted by amino acid sequence WBKRRHWBKRRH
  source 1..40: mol_type = other DNA
ggactggaatgggtctccatcggttcgacatactatgcgg

SEQ ID NO: 38 - DNA, 34 bp, synthetic construct
  misc_feature 1..34: note = HCDR3-8
  misc_feature 19..20: note = Unsure - Inserted by amino acid sequence RNCNNKNNKNNKNNKNTCGABWW
  source 1..34: mol_type = other DNA
ggtgtactactgcgcccgctggggacagggcacc

SEQ ID NO: 39 - DNA, 32 bp, synthetic construct
  misc_feature 1..32: note = HCDR3-9
  misc_feature 18..19: note = Unsure - Inserted by amino acid sequence RNCNNKNNKNNKNNKNNKNTCGABWWC
  source 1..32: mol_type = other DNA
gtgtactactgcgcccgctggggacagggcac

SEQ ID NO: 40 - DNA, 35 bp, synthetic construct
  misc_feature 1..35: note = HCDR3-10
  misc_feature 18..19: note = Unsure - Inserted by amino acid sequence RNCNNKNNKNNKNNKNNKNNKTTB
  source 1..35: mol_type = other DNA
gtgtactactgcgcccgcgattactggggacaggg

SEQ ID NO: 41 - DNA, 34 bp, synthetic construct
  misc_feature 1..34: note = HCDR3-11
  misc_feature 18..19: note = Unsure - Inserted by amino acid sequence RNCNNKNNKNNKNNKNNKNNKNNY
  source 1..34: mol_type = other DNA
gtgtactactgcgcccgctttgattactggggac

SEQ ID NO: 42 - DNA, 32 bp, synthetic construct
  misc_feature 1..32: note = HCDR3-12
  misc_feature 16..17: note = Unsure - Inserted by amino acid sequence RNCNNKNNKNNKNNKNNKNNKNNKNNY
  source 1..32: mol_type = other DNA
gtactactgcgcccgctttgattactggggac

SEQ ID NO: 43 - DNA, 29 bp, synthetic construct
  misc_feature 1..29: note = HCDR3-13
  misc_feature 16..17: note = Unsure - Inserted by amino acid sequence RNCNNYNNYNNYNNYNNYNNYNNYNNYNNY
  source 1..29: mol_type = other DNA
gtactactgcgcccgctttgattactggg

SEQ ID NO: 44 - DNA, 32 bp, synthetic construct; identical to SEQ ID NO: 42
  misc_feature 1..32: note = HCDR3-14
  misc_feature 16..17: note = Unsure - Inserted by amino acid sequence RNCNNYNNYNNYNNYNNYNNYNNYNNYNNYNNY
  source 1..32: mol_type = other DNA

SEQ ID NO: 45 - protein, 98 aa, synthetic construct
  REGION 1..98: note = 3-23 germline AA
EVQLLESGGGLVQPGGSLRLSCAASGFTFSSYAISWVRQAPGKGLEWVSAISGSGGSTYYADSVKGRFTISRDNSKNTLYLQMNSLRAEDTAVYYCAR

SEQ ID NO: 46 - DNA, 294 bp, synthetic construct
  misc_feature 1..294: note = 3-23 germline DNA
  source 1..294: mol_type = other DNA
gaagtgcagctgctggaaagcggcggcggcctggtgcagccgggcggcagcctgcgcctgagctgcgcggcgagcggctttacctttagcagctatgcgattagctgggtgcgccaggcgccgggcaaaggcctggaatgggtgagcgcgattagcggcagcggcggtagcacctattatgcggatagcgtgaaaggccgctttaccattagccgcgataacagcaaaaacaccctgtatctgcagatgaacagcctgcgcgcggaagataccgccgtgtattattgcgcgcgt

SEQ ID NO: 47 - protein, 255 aa, synthetic construct
  REGION 1..255: note = human CD137 FL AA
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 48 - DNA, 765 bp, synthetic construct
  misc_feature 1..765: note = human CD137 FL DNA
  source 1..765: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 49 - protein, 160 aa, synthetic construct
  REGION 1..160: note = human CD137 AA 24-183
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGH

SEQ ID NO: 50 - DNA, 480 bp, synthetic construct
  misc_feature 1..480: note = human CD137 24-183 DNA
  source 1..480: mol_type = other DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacac

SEQ ID NO: 51 - protein, 183 aa, synthetic construct
  REGION 1..183: note = human CD137 -his
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHHHHHHHGGGLNDIFEAQKIEWHE

SEQ ID NO: 52 - DNA, 549 bp, synthetic construct
  misc_feature 1..549: note = human CD137 -his DNA
  source 1..549: mol_type = other DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacaccatcaccatcaccatcacggaggcggtctgaacgacatcttcgaggctcagaaaatcgaatggcacgaa

SEQ ID NO: 53 - protein, 393 aa, synthetic construct
  REGION 1..393: note = human CD137 ECD-mIgG2a AA
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 54 - DNA, 1179 bp, synthetic construct
  misc_feature 1..1179: note = human CD137 ECD-mIgG2a DNA
  source 1..1179: mol_type = other DNA
ctgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacacgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 55 - protein, 233 aa, synthetic construct
  REGION 1..233: note = mIgG2a AA
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 56 - DNA, 699 bp, synthetic construct
  misc_feature 1..699: note = mIgG2a DNA
  source 1..699: mol_type = other DNA
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 57 - protein, 254 aa, synthetic construct
  REGION 1..254: note = human CD137 ligand AA
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 58 - DNA, 765 bp, synthetic construct
  misc_feature 1..765: note = human CD137 ligand cDNA
  source 1..765: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 59 - protein, 184 aa, synthetic construct
  REGION 1..184: note = human CD137 ligand 71-254 AA
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 60 - DNA, 552 bp, synthetic construct
  misc_feature 1..552: note = human CD137 ligand 71-254 DNA
  source 1..552: mol_type = other DNA
cgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 61 - protein, 422 aa, synthetic construct
  REGION 1..422: note = human CD137 ligand-mIgG2a AA
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 62 - DNA, 1266 bp, synthetic construct
  misc_feature 1..1266: note = human CD137 ligand-mIgG2a DNA
  source 1..1266: mol_type = other DNA
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggtggaggcggttcacgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 63 - protein, 254 aa, synthetic construct
  REGION 1..254: note = cyno CD137 FL AA
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 64 - DNA, 765 bp, synthetic construct
  misc_feature 1..765: note = cyno CD137 FL DNA
  source 1..765: mol_type = other DNA
atgggaaacagctgttacaacatagtagccactctgttgctggtcctgaactttgagaggacaagatccttgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcagatcatcttctttcttgcgctgacttcgactgtggtactcttcctgctgttcttcctcgtgctccgtttctctgttgttaaacggagcagaaagaaactcctgtatatattcaaacaaccatttatgagaccggtacaaaccactcaagaggaagatggatgtagctgccgatttccagaagaagaagaaggaggatgtgaactgtga

SEQ ID NO: 65 - protein, 163 aa, synthetic construct
  REGION 1..163: note = cyno CD137 ECD 24-186 AA
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQ

SEQ ID NO: 66 - DNA, 489 bp, synthetic construct
  misc_feature 1..489: note = cyno CD137 ECD 24-186 DNA
  source 1..489: mol_type = other DNA
ctgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcag

SEQ ID NO: 67 - protein, 396 aa, synthetic construct
  REGION 1..396: note = cyno CD137 ECD-mIgG2a AA
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 68 - DNA, 1188 bp, synthetic construct
  misc_feature 1..1188: note = cyno CD137 ECD-mIgG2a DNA
  source 1..1188: mol_type = other DNA
ctgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcaggatatcactgcctgggggcagaatgcagcatgtgtgaacaggattgtaaacaaggtcaagaattgacaaaaaaaggttgtaaagactgttgctttgggacatttaatgaccagaaacgtggcatctgtcgaccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcaggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 69 - protein, 277 aa, synthetic construct
  REGION 1..277: note = human CD40 AA
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 70 - DNA, 834 bp, synthetic construct
  misc_feature 1..834: note = human CD40 DNA
  source 1..834: mol_type = other DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 71 - protein, 194 aa, synthetic construct
  REGION 1..194: note = huCD40 ECD AA (1-194)
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRA

SEQ ID NO: 72 - DNA, 582 bp, synthetic construct
  misc_feature 1..582: note = huCD40 ECD DNA (1-194)
  source 1..582: mol_type = other DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagcc

SEQ ID NO: 73 - protein, 428 aa, synthetic construct
  REGION 1..428: note = human CD40 ECD-mIgG2a AA
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRASEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 74 - DNA, 1284 bp, synthetic construct
  misc_feature 1..1284: note = human CD40 ECD-mIgG2a DNA
  source 1..1284: mol_type = other DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgagttcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagctagcgagcccagagggcccacaatcaagccctgtcctccatgcaaatgcccagcacctaacctcttgggtggaccatccgtcttcatcttccctccaaagatcaaggatgtactcatgatctccctgagccccatggtcacatgtgtggtggtggatgtgagcgaggatgacccagatgtccagatcagctggttcgtgaacaacgtggaagtactcacagctcagacacaaacccatagagaggattacaacagtactctccgggtggtcagtgccctccccatccagcaccaggactggatgagtggcaaggagttcaaatgcaaggtcaacaacaaagccctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgctcagtggtccacgagggtctgcacaatcaccacacgactaagagcttctcccggactccgggtaaa

SEQ ID NO: 75 - protein, 277 aa, synthetic construct
  REGION 1..277: note = human OX40 AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 76 - DNA, 834 bp, synthetic construct
  misc_feature 1..834: note = human OX40 DNA
  source 1..834: mol_type = other DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 77 - protein, 216 aa, synthetic construct
  REGION 1..216: note = human OX40 ECD (1-216) AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVA

SEQ ID NO: 78 - DNA, 648 bp, synthetic construct
  misc_feature 1..648: note = human OX40 ECD (1-216) DNA
  source 1..648: mol_type = other DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgcc

SEQ ID NO: 79 - protein, 222 aa, synthetic construct
  REGION 1..222: note = human OX40 ECD-His AA
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAHHHHHH

SEQ ID NO: 80 - DNA, 666 bp, synthetic construct
  misc_feature 1..666: note = human OX40 ECD-His DNA
  source 1..666: mol_type = other DNA
atgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgcccatcaccatcaccatcac

SEQ ID NO: 81 - protein, 355 aa, synthetic construct
  REGION 1..355: note = BGA-7207 VH-Fc
EVQLLESGGGLVQPGGSLRLSCAASGFRLDTTEVGWVRQAPGKGLEWVSTIMGISGSTYYADSVKGRFTISRDNSKNTLYLQMNSLRAEDTAVYYCARVVDSLFDDSAVFDYWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - DNA, 1065 bp, synthetic construct
  misc_feature 1..1065: note = BGA-7207 VH-Fc DNA
  source 1..1065: mol_type = other DNA
gaggtccagttacttgagagtggtggaggtctggtccaaccaggaggttcgctgcgtttatcctgcgccgcgtctggattcagattggacaccaccgaagtaggttgggtgcgtcaagcgccggggaaaggactggaatgggtctccaccatcatgggtattagtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtcgattccctctttgatgacagcgccgtttttgattactggggacagggcaccttagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 83 - protein, 354 aa, synthetic construct
  REGION 1..354: note = BGA-4712 VH-Fc
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1062 bp, synthetic construct
  misc_feature 1..1062: note = BGA-4712 VH-Fc DNA
  source 1..1062: mol_type = other DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 85 - protein, 227 aa, synthetic construct
  REGION 1..227: note = Inert Fc: A Fc-null version IgG1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 681 bp, synthetic construct
  misc_feature 1..681: note = Inert Fc: A Fc-null version IgG1 DNA
  source 1..681: mol_type = other DNA
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - protein, 449 aa, synthetic construct
  REGION 1..449: note = B-CD137xCEA anti-CEA Ab heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 1347 bp, synthetic construct
  misc_feature 1..1347: note = B-CD137xCEA anti-CEA Ab heavy chain DNA
  source 1..1347: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 89 - protein, 214 aa, synthetic construct
  REGION 1..214: note = A-CD137xCEA or C-CD137xCEA or D-CD137xCEA anti-CEA Ab light chain AA
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - DNA, 642 bp, synthetic construct
  misc_feature 1..642: note = A-CD137xCEA or C-CD137xCEA or D-CD137xCEA anti-CEA Ab light chain DNA
  source 1..642: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgc

SEQ ID NO: 91 - protein, 576 aa, synthetic construct
  REGION 1..576: note = A-CD137xCEA A-BGA-4712/CEA heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSS

SEQ ID NO: 92 - DNA, 1728 bp, synthetic construct
  misc_feature 1..1728: note = A-BGA-4712/CEA heavy chain DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcg

SEQ ID NO: 93 - protein, 341 aa, synthetic construct
  REGION 1..341: note = B-CD137xCEA B-BGA-4712/CEA light chain AA
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSS

SEQ ID NO: 94 - DNA, 1023 bp, synthetic construct
  misc_feature 1..1023: note = B-CD137xCEA B-BGA-4712/CEA light chain DNA
  source 1..1023: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgcgggggaggcgggtccgaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcg

SEQ ID NO: 95 - protein, 588 aa, synthetic construct
  REGION 1..588: note = C-CD137xCEA C-BGA-4712/CEA heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSEVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSSSGGGGSEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - DNA, 1764 bp, synthetic construct
  misc_feature 1..1764: note = C-CD137xCEA C-BGA-4712/CEA heavy chain DNA
  source 1..1764: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacgggggaggcgggtccgaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcgtctggtggaggcggttcagagcccaaatcttgtgacaaaactcacacatgcccaccgtgcccagcacctcctgctgccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcgcagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 97 - protein, 577 aa, synthetic construct
  REGION 1..577: note = D-CD137xCEA D-BGA-4712/CEA heavy chain AA
EVQLLESGGGLVQPGGSLRLSCAASGFMLSAEDVGWVRQAPGKGLEWVSAILDFGGSTYYADSVKGRFTISRDNSKNTLYLQMNSLRAEDTAVYYCARVVYHAGGGVTFDYWGQGTLVTVSSSGGGGSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - DNA, 1731 bp, synthetic construct
  misc_feature 1..1731: note = D-CD137xCEA D-BGA-4712/CEA heavy chain DNA
  source 1..1731: mol_type = other DNA
gaggtccagttacttgagagtggtggaggtctggtccaaccaggaggttcgctgcgtttatcctgcgccgcgtctggattcatgttgtccgccgaagacgtgggttgggtgcgtcaagcgccggggaaaggactggaatgggtctccgccatcttggattttggtggttcgacatactatgcggacagtgtcaaagggcgctttacgatctcgcgcgataactcaaaaaatactctttaccttcaaatgaatagccttcgtgctgaggacactgcggtgtactactgcgcccgcgtcgtctaccatgctggtggtggcgtcacctttgattactggggacagggcaccttagttacagtctcatcgtccggtggaggcggttcacaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 99 - protein, 354 aa, synthetic construct
  REGION 1..354: note = BGA-4712-M3-mutFc AA
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 1062 bp, synthetic construct
  misc_feature 1..1062: note = BGA-4712-M3-mutFc DNA
  source 1..1062: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 101 - protein, 602 aa, synthetic construct
  REGION 1..602: note = A-CD137xCEA-M3
GSATMDMRVPAQLLGLLLLWFPGSRSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 102 - DNA, 1821 bp, synthetic construct
  misc_feature 1..1821: note = A-CD137xCEA-M3 DNA
  source 1..1821: mol_type = other DNA
ggatccgccaccatggatatgagagttcctgctcaattgctggggttgctcttgctctggtttcctggctcgagaagccaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcgtgaaagcttcgtcag

SEQ ID NO: 103 - protein, 122 aa, synthetic construct
  REGION 1..122: note = BGA-7556 VH
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSS

SEQ ID NO: 104 - DNA, 366 bp, synthetic construct
  misc_feature 1..366: note = BGA-7556 VH DNA
  source 1..366: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcg

SEQ ID NO: 105 - protein, 354 aa, synthetic construct
  REGION 1..354: note = BGA-7556-mutFc AA
EVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - DNA, 1062 bp, synthetic construct
  misc_feature 1..1062: note = BGA-7556-mutFc DNA
  source 1..1062: mol_type = other DNA
gaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 107 - protein, 576 aa, synthetic construct
  REGION 1..576: note = Format A- BGA-7556 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYRGQGTQVTVSS

SEQ ID NO: 108 - DNA, 1728 bp, synthetic construct
  misc_feature 1..1728: note = Format A- BGA-7556 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattaccggggacagggcacccaagttacagtctcatcg

SEQ ID NO: 109 - protein, 107 aa, synthetic construct
  REGION 1..107: note = CH3 AA
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 321 bp, synthetic construct
  misc_feature 1..321: note = CH3 DNA
  source 1..321: mol_type = other DNA
ggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaa

SEQ ID NO: 111 - protein, 234 aa, synthetic construct
  REGION 1..234: note = CH3- BGA-4712-M3 AA
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDFGGSTYYAESVKGRFTISRDNAKNTLYLQMSSLRAEDTAVYYCARVVYHAGGGVTFDYWGQGTLVTVSS

SEQ ID NO: 112 - DNA, 702 bp, synthetic construct
  misc_feature 1..702: note = CH3- BGA-4712-M3
  source 1..702: mol_type = other DNA
ggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgttgtccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgattactggggacagggcaccttagttacagtctcatcg

SEQ ID NO: 113 - protein, 122 aa, synthetic construct
  REGION 1..122: note = BGA-2690 AA
EVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSS

SEQ ID NO: 114 - DNA, 366 bp, synthetic construct
  misc_feature 1..366: note = BGA-2690 DNA
  source 1..366: mol_type = other DNA
gaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 115 - protein, 122 aa, synthetic construct
  REGION 1..122: note = BGA-3849 AA
EVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSS

SEQ ID NO: 116 - DNA, 366 bp, synthetic construct
  misc_feature 1..366: note = BGA-3849 DNA
  source 1..366: mol_type = other DNA
gaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcg

SEQ ID NO: 117 - protein, 122 aa, synthetic construct
  REGION 1..122: note = BGA-7916 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 118 - DNA, 366 bp, synthetic construct
  misc_feature 1..366: note = BGA-7916 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 119 - protein, 122 aa, synthetic construct
  REGION 1..122: note = BGA-4988 AA
EVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDYRGQGTQVTVSS

SEQ ID NO: 120 - DNA, 366 bp, synthetic construct
  misc_feature 1..366: note = BGA-4988 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 121 - protein, 122 aa, synthetic construct
  REGION 1..122: note = BGA-5164 AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSS

SEQ ID NO: 122 - DNA, 366 bp, synthetic construct
  misc_feature 1..366: note = BGA-5164 DNA
  source 1..366: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 123 - protein, 122 aa, synthetic construct
  REGION 1..122: note = BGA-3953 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSS

SEQ ID NO: 124 - DNA, 366 bp, synthetic construct
  misc_feature 1..366: note = BGA-5385 AA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 125 - protein, 122 aa, synthetic construct
  REGION 1..122: note = BGA-5385 AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTFDTRGQGTQVTVSS

SEQ ID NO: 126 - DNA, 366 bp, synthetic construct
  misc_feature 1..366: note = BGA-5385 DNA
  source 1..366: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctttgatactcggggacagggcacccaagttacagtctcatcg

SEQ ID NO: 127 - protein, 122 aa, synthetic construct
  REGION 1..122: note = BGA-9468 AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSS

SEQ ID NO: 128 - DNA, 366 bp, synthetic construct
  misc_feature 1..366: note = BGA-9468 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcg

SEQ ID NO: 129 - protein, 122 aa, synthetic construct
  REGION 1..122: note = BGA-3285 AA
EVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 130 - DNA, 366 bp, synthetic construct
  misc_feature 1..366: note = BGA-3285 DNA
  source 1..366: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcg

SEQ ID NO: 131 - protein, 122 aa, synthetic construct
  REGION 1..122: note = BGA-9442 AA
EVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSS

SEQ ID NO: 132 - DNA, 366 bp, synthetic construct
  misc_feature 1..366: note = BGA-9442 DNA
  source 1..366: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcg

SEQ ID NO: 133 - protein, 122 aa, synthetic construct
  REGION 1..122: note = BGA-7746 AA
EVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 134 - DNA, 366 bp, synthetic construct
  misc_feature 1..366: note = BGA-7746 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcg

SEQ ID NO: 135 - protein, 122 aa, synthetic construct
  REGION 1..122: note = BGA-8425 AA
EVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 136 - DNA, 366 bp, synthetic construct
  misc_feature 1..366: note = BGA-8425 DNA
  source 1..366: mol_type = other DNA
gaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcg

SEQ ID NO: 137 - protein, 122 aa, synthetic construct
  REGION 1..122: note = BGA-3386 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSS

SEQ ID NO: 138 - DNA, 366 bp, synthetic construct
  misc_feature 1..366: note = BGA-3386 DNA
  source 1..366: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcg

SEQ ID NO: 139 - protein, 354 aa, synthetic construct
  REGION 1..354: note = BGA-5623-mutFc-AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - DNA, 1062 bp, synthetic construct
  misc_feature 1..1062: note = BGA-5623-mutFc DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 141 - protein, 354 aa, synthetic construct
  REGION 1..354: note = BGA-2690- mutFc AA
EVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - DNA, 1062 bp, synthetic construct
  misc_feature 1..1062: note = BGA-2690 mutFc DNA
  source 1..1062: mol_type = other DNA
gaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 143 - protein, 354 aa, synthetic construct
  REGION 1..354: note = BGA-3849-mutFc AA
EVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - DNA, 1062 bp, synthetic construct
  misc_feature 1..1062: note = BGA-3849 mutFc DNA
  source 1..1062: mol_type = other DNA
gaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 145 - protein, 354 aa, synthetic construct
  REGION 1..354: note = BGA-7916-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDTRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - DNA, 1062 bp, synthetic construct
  misc_feature 1..1062: note = BGA-7916-mutFc DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 147 - protein, 354 aa, synthetic construct
  REGION 1..354: note = BGA-4988-mutFc AA
EVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDTRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - DNA, 1062 bp, synthetic construct
  misc_feature 1..1062: note = BGA-4988-mutFc DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 149 - protein, 354 aa, synthetic construct
  REGION 1..354: note = BGA-5164-mutFc AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - DNA, 1062 bp, synthetic construct
  misc_feature 1..1062: note = BGA-5164-mutFc DNA
  source 1..1062: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 151 - protein, 354 aa, synthetic construct
  REGION 1..354: note = BGA-3953-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - DNA, 1062 bp, synthetic construct
  misc_feature 1..1062: note = BGA-3953-mutFc DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 153 - protein, 354 aa, synthetic construct
  REGION 1..354: note = BGA-5385-mutFc AA
EVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTYDYRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - DNA, 1062 bp, synthetic construct
  misc_feature 1..1062: note = BGA-5385-mutFc DNA
  source 1..1062: mol_type = other DNA
gaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctatgattatcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 155 - protein, 354 aa, synthetic construct
  REGION 1..354: note = BGA-9468-mutFc AA
EVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - DNA, 1062 bp, synthetic construct
  misc_feature 1..1062: note = BGA-9468-mutFc DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 157 - protein, 354 aa, synthetic construct
  REGION 1..354: note = BGA-3285-mutFc AA
EVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - DNA, 1062 bp, synthetic construct
  misc_feature 1..1062: note = BGA-3285-mutFc DNA
  source 1..1062: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 159 - protein, 354 aa, synthetic construct
  REGION 1..354: note = BGA-9442-mutFc AA
EVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - DNA, 1062 bp, synthetic construct
  misc_feature 1..1062: note = BGA-9442-mutFc DNA
  source 1..1062: mol_type = other DNA
gaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 161 - protein, 354 aa, synthetic construct
  REGION 1..354: note = BGA-7746-mutFc AA
EVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - DNA, 1062 bp, synthetic construct
  misc_feature 1..1062: note = BGA-7746-mutFc DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 163 - protein, 354 aa, synthetic construct
  REGION 1..354: note = BGA-8425-mutFc AA
EVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - DNA, 1062 bp, synthetic construct
  misc_feature 1..1062: note = BGA-8425-mutFc DNA
  source 1..1062: mol_type = other DNA
gaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 165 - protein, 354 aa, synthetic construct
  REGION 1..354: note = BGA-3386-mutFc AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - DNA, 1062 bp, synthetic construct
  misc_feature 1..1062: note = BGA-3386-mutFc DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcgggcggcggagggtctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 167 - protein, 576 aa, synthetic construct
  REGION 1..576: note = Format A BGA-5623AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 168 - DNA, 1728 bp, synthetic construct
  misc_feature 1..1728: note = Format A BGA-5623 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 169 - protein, 576 aa, synthetic construct
  REGION 1..576: note = Format A BGA-2690 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSREDVGWVRQAPGKGLEWVSAILDFGSSTYYAESVKGRFTISRDNAKNTLYLQMSSLRAEDTAVYYCARVVYHAGGGVTYDYRGQGTQVTVSS

SEQ ID NO: 170 - DNA, 1728 bp, synthetic construct
  misc_feature 1..1728: note = Format A BGA-2690 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttccagagaagacgtgggttgggtgcgtcaagcgccggggaaaggactggaatgggtctccgccatcttggatttcggttcttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 171 - protein, 576 aa, synthetic construct
  REGION 1..576: note = Format A BGA-3849 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSHEDVGWVRQAPGKGLEWVSAILDSGGSTYYAESVKGRFTISRDNAKNTLYLQMSSLRAEDTAVYYCARYVYHAGGGVTFDTRGQGTQVTVSS

SEQ ID NO: 172 - DNA, 1728 bp, synthetic construct
  misc_feature 1..1728: note = Format A BGA-3849 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttcccatgaagacgtgggttgggtgcgtcaagcgccggggaaaggactggaatgggtctccgccatcttggattccggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatactcggggacagggcacccaagttacagtctcatcg

SEQ ID NO: 173 - protein, 576 aa, synthetic construct
  REGION 1..576: note = Format A BGA-7916 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 174 - DNA, 1728 bp, synthetic construct
  misc_feature 1..1728: note = Format A BGA-7916 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 175 - protein, 576 aa, synthetic construct
  REGION 1..576: note = Format A BGA-4988 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDRGGSTYYAESVKGRFTISRDNAKNTLYLQMSSLRAEDTAVYYCARVVYHAGGGVTYDYRGQGTQVTVSS

SEQ ID NO: 176 - DNA, 1728 bp, synthetic construct
  misc_feature 1..1728: note = Format A BGA-4988 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggatcggggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgatactcggggacagggcacccaagttacagtctcatcg

SEQ ID NO: 177 - protein, 576 aa, synthetic construct
  REGION 1..576: note = Format A BGA-5164 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVVYHAGGGVTYDYRGQGTQVTVSS

SEQ ID NO: 178 - DNA, 1728 bp, synthetic construct
  misc_feature 1..1728: note = Format A BGA-5164 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctatgattatcggggacagggcacccaagttacagtctcatcg

SEQ ID NO: 179 - protein, 576 aa, synthetic construct
  REGION 1..576: note = Format A BGA-3953 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARIVYHAGGGVTFDTRGQGTQVTVSS

SEQ ID NO: 180 - DNA, 1728 bp, synthetic construct
  misc_feature 1..1728: note = Format A BGA-3953 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 181 - protein, 576 aa, synthetic construct
  REGION 1..576: note = Format A BGA-5385 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDHGGSTYYAESVKGRFTISRDNAKNTLYLQMSSLRAEDTAVYYCARVIYHAGGGVTFDTRGQGTQVTVSS

SEQ ID NO: 182 - DNA, 1728 bp, synthetic construct
  misc_feature 1..1728: note = Format A BGA-5385 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctttccgccgaagacgtgggttgggtgcgtcaagcgccggggaaaggactggaatgggtctccgccatcttggatcatggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttatctaccatgctggtggtggcgtcacctttgatactcggggacagggcacccaagttacagtctcatcg

SEQ ID NO: 183 - protein, 576 aa, synthetic construct
  REGION 1..576: note = Format A BGA-9468 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDSGGSTYYAESVKGRFTISRDNAKNTLYLQMSSLRAEDTAVYYCARVVYHSGGGVTFDLRGQGTQVTVSS

SEQ ID NO: 184 - DNA, 1728 bp, synthetic construct
  misc_feature 1..1728: note = Format A BGA-9468 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccattctggtggtggcgtcacctttgatcttcggggacagggcacccaagttacagtctcatcg

SEQ ID NO: 185 - protein, 576 aa, synthetic construct
  REGION 1..576: note = Format A BGA-3285 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTISAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 186 - DNA, 1728 bp, synthetic construct
  misc_feature 1..1728: note = Format A BGA-3285 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattttggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgttgtctaccatgctggtggtggcgtcacctttgatactcggggacagggcacccaagttacagtctcatcg

SEQ ID NO: 187 - protein, 576 aa, synthetic construct
  REGION 1..576: note = Format A BGA-9442 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTISAEDVGWVRQAPGKGLEWVSAILDSGGSTYYAESVKGRFTISRDNAKNTLYLQMSSLRAEDTAVYYCARYVYHAGGGVTFDLRGQGTQVTVSS

SEQ ID NO: 188 - DNA, 1728 bp, synthetic construct
  misc_feature 1..1728: note = Format A BGA-9442 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgatttccgccgaagacgtgggttgggtgcgtcaagcgccggggaaaggactggaatgggtctccgccatcttggattctggtggttcgacatactatgcggaaagtgtcaaagggcgctttacgatctcgcgcgataacgcaaaaaatactctttaccttcaaatgtctagccttcgtgctgaggacactgcggtgtactactgcgcccgctatgtctaccatgctggtggtggcgtcacctttgatcttcggggacagggcacccaagttacagtctcatcg

SEQ ID NO: 189 - protein, 576 aa, synthetic construct
  REGION 1..576: note = Format A BGA-7746 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDFGGSTYYAESVKGRFTISRDNAKNTLYLQMSSLRAEDTAVYYCARVVYHAGGGVTFDTRGQGTQVTVSS

SEQ ID NO: 190 - DNA, 1728 bp, synthetic construct
  misc_feature 1..1728: note = Format A BGA-7746 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggattttggtggttcgacatactatgcagaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctttgatacgcggggacagggcacccaagttacagtctcatcg

SEQ ID NO: 191 - protein, 576 aa, synthetic construct
  REGION 1..576: note = Format A BGA-8425 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTLSAEDVGWVRQAPGKGLEWVSAILDSGGSTYYAESVKGRFTISRDNAKNTLYLQMSSLRAEDTAVYYCARVVYHAGGGVTYDYRGQGTQVTVSS

SEQ ID NO: 192 - DNA, 1728 bp, synthetic construct
  misc_feature 1..1728: note = Format A BGA-8425 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacgctgtccgccgaagacgtgggttgggtgcgtcaagcgccggggaaaggactggaatgggtctccgccatcttggattcgggtggttcgacatactatgcggaaagtgtcaaagggcgctttacgatctcgcgcgataacgcaaaaaatactctttaccttcaaatgtctagccttcgtgctgaggacactgcggtgtactactgcgcccgcgtcgtctaccatgctggtggtggcgtcacctatgattaccggggacagggcacccaagttacagtctcatcg

SEQ ID NO: 193 - protein, 576 aa, synthetic construct
  REGION 1..576: note = Format A BGA-3386 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTWGQGTLVTVSS

SEQ ID NO: 194 - DNA, 1728 bp, synthetic construct
  misc_feature 1..1728: note = Format A BGA-3386 DNA
  source 1..1728: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatacttggggacagggcaccttagttacagtctcatcg

SEQ ID NO: 195 - protein, 354 aa, synthetic construct
  REGION 1..354: note = BGA-5623 IgG1 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - DNA, 1062 bp, synthetic construct
  misc_feature 1..1062: note = BGA-5623 IgG1 DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 197 - protein, 350 aa, synthetic construct
  REGION 1..350: note = BGA-5623 IgG4 AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 198 - DNA, 1050 bp, synthetic construct
  misc_feature 1..1050: note = BGA-5623 IgG4 DNA
  source 1..1050: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggaatccaagtatggtccgccatgtccaccgtgcccggcacccgagtttttgggagggcctagtgtgtttttgtttcctcctaagccaaaggacacgcttatgattagtcgcacacccgaggttacctgtgtagtggtggacgtatcacaagaggatccggaagtccaatttaattggtacgtcgacggagtggaagtccacaatgcaaagaccaagcctagggaagaacagttcaattctacctatcgctggtctgtactcactgtactccaccaggattggctcaatggaaaggaatacaaatgcaaggtttccaacaaagggctcccctcttccattgagaaaactattagcaaggctaaaggccaacccagagaaccacaggtttatacccttccaccttcccaggaggagatgacgaagaatcaggttagtcttacgtgcctcgttaaaggattctacccttcagatatcgcagttgaatgggagtctaacgggcaacccgagaacaactacaagaccaccccgccggtgctggactcagatggtagtttctttctttacagtcggcttaccgtcgacaagtcacgctggcaggagggaaatgtattctcctgcagcgtaatgcacgaagctctgcacaatcactatacccagaagtcactgagcctttctctgggtaag

SEQ ID NO: 199 - protein, 447 aa, synthetic construct
  REGION 1..447: note = Urelumab analog Heavy chain AA
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 200 - DNA, 1341 bp, synthetic construct
  misc_feature 1..1341: note = Urelumab analog Heavy chain DNA
  source 1..1341: mol_type = other DNA
caagttcaacttcagcagtggggagcgggtctcttgaagcctagtgagacgctgagcctgacctgtgcagtctacgggggttcattttctgggtactactggtcctggatacgccaaagcccagagaaaggcctggaatggattggggagataaatcacgggggttatgtgacatataatccttctcttgagagccgggtcacgatatctgttgacacaagtaaaaatcagttctccctcaaactgagctccgttaccgcagcagacactgccgtttactactgtgcgcgggactatgggccaggtaattacgattggtacttcgacctgtggggtagaggaacgctggtcactgtaagcagtgcatctacaaagggccccagcgtgtttccccttgcaccatgttcacggagcactagtgaaagcactgccgcgctcgggtgtctcgttaaggattattttccggagcctgtcactgtctcttggaactctggtgcactgacatcaggcgttcacacgttccctgcagtactccagagctcaggattgtacagcctgtccagtgtagttacagtgccttcctcctcccttggaacaaagacctacacctgcaacgtggaccataagcccagcaatacgaaagtagacaaacgagtcgaatccaagtatggtccgccatgtccaccgtgcccggcacccgagtttttgggagggcctagtgtgtttttgtttcctcctaagccaaaggacacgcttatgattagtcgcacacccgaggttacctgtgtagtggtggacgtatcacaagaggatccggaagtccaatttaattggtacgtcgacggagtggaagtccacaatgcaaagaccaagcctagggaagaacagttcaattctacctatcgctggtctgtactcactgtactccaccaggattggctcaatggaaaggaatacaaatgcaaggtttccaacaaagggctcccctcttccattgagaaaactattagcaaggctaaaggccaacccagagaaccacaggtttatacccttccaccttcccaggaggagatgacgaagaatcaggttagtcttacgtgcctcgttaaaggattctacccttcagatatcgcagttgaatgggagtctaacgggcaacccgagaacaactacaagaccaccccgccggtgctggactcagatggtagtttctttctttacagtcggcttaccgtcgacaagtcacgctggcaggagggaaatgtattctcctgcagcgtaatgcacgaagctctgcacaatcactatacccagaagtcactgagcctttctctgggtaag

SEQ ID NO: 201 - protein, 216 aa, synthetic construct
  REGION 1..216: note = Urelumab analog Light chain AA
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - DNA, 648 bp, synthetic construct
  misc_feature 1..648: note = Urelumab analog Light chain DNA
  source 1..648: mol_type = other DNA
gaaatcgtgctgacccaatcacctgcgaccctctcccttagccctggagaacgagcaactctcagttgcagagcaagccagagcgtcagctcatatctcgcatggtatcaacaaaaaccaggccaagctcctagactgttgatttatgatgcctctaaccgggcgactggcataccagcccgctttagcgggtctgggagtggcactgattttacgctcacgatttcatctttggagccagaagatttcgctgtatactactgtcaacaaaggtcaaactggccgcctgcacttacgttcggcgggggtaccaaggtggagattaagaggacagttgcggcgccatcagtttttatattcccaccctccgatgagcagttgaaaagtggcacggcatcagttgtctgtctgctgaacaatttctatccacgggaggcgaaagtccaatggaaagtggacaatgcgcttcagagcgggaactctcaggagagtgtcactgaacaggattcaaaggactccacgtactcactctcatccactctcaccctttcaaaggcggattacgaaaagcataaggtctacgcttgtgaggtaacacatcaggggctttccagtcctgtaaccaagagcttcaatcgcggcgaatgc

SEQ ID NO: 203 - protein, 442 aa, synthetic construct
  REGION 1..442: note = Utomilumab analog Heavy chain AA
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 204 - DNA, 1326 bp, synthetic construct
  misc_feature 1..1326: note = Utomilumab analog Heavy chain DNA
  source 1..1326: mol_type = other DNA
gaagttcaactggtacagtccggtgctgaagttaaaaaacccggagagagtctgagaatctcttgcaaggggtctgggtactcttttagtacatattggatatcttgggtgagacaaatgccggggaaaggcctcgaatggatgggcaagatttatcccggagacagctatacaaattactcacctagttttcaaggtcaggtaactatcagcgcggacaaatccataagtacggcctatctgcaatggtcttccttgaaggcatccgacacggcgatgtattactgcgcaagagggtatggtatattcgattactgggggcagggcactctcgttaccgtgagcagcgcttcaacaaaaggcccatctgtcttccctctcgcgccttgctcccgctctacttcagagtcaacagctgctctggggtgcttggtgaaggattactttcctgaaccggttactgtcagctggaattctggggctttgacgagcggagttcacactttcccagctgtactgcaaagttctggactttacagcttgagctccgtggtgacagtgccgagctcaaacttcggtacgcagacatatacatgcaatgtggatcataaacccagcaatacgaaagtagataagacagtcgaaaggaagtgttgtgtagagtgcccaccgtgccccgcccctccggttgcaggcccatcagtgtttctcttcccgcccaaacctaaggacacgctgatgatttcacgaacgcccgaggtcacctgtgtcgtagtcgatgtgtcccatgaggacccagaagttcagttcaattggtatgtagacggcgtggaggtacacaatgcaaaaacgaaaccacgcgaggaacagttcaatagtacgttccgggtcgtgagtgtgctcacggtggttcaccaagactggctgaacgggaaagaatataagtgcaaagtatccaataaaggcctgcccgcacccatcgaaaaaactataagtaaaactaaagggcaaccgcgagagcctcaggtctatactttgccaccgtcacgcgaagagatgaccaagaaccaagtaagtcttacttgcttggtcaaggggttctacccctcagatatagcggtggagtgggaatccaatggccaacccgagaacaattacaagacaacaccaccaatgctggactctgacgggagtttctttctgtatagtaaacttaccgtcgataaatcacgctggcagcaaggcaacgtattctcatgctcagtaatgcatgaagccttgcataatcactacactcagaaaagcctgtccctctctccaggtaag

SEQ ID NO: 205 - protein, 214 aa, synthetic construct
  REGION 1..214: note = Utomilumab analog Light chain AA
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 206 - DNA, 642 bp, synthetic construct
  misc_feature 1..642: note = Utomilumab analog Light chain DNA
  source 1..642: mol_type = other DNA
agctacgagttgactcagccaccatccgtcagtgttagccccggacagacggcgtcaattacttgtagtggagacaacattggcgatcagtacgcccattggtatcagcaaaaaccgggtcaatcaccagttctggtgatatatcaggataagaatcgaccatcagggattccagagaggttctcaggtagtaactcaggtaacacagctactctgaccataagtggtacccaggctatggacgaagcagactactactgcgccacctacacaggcttcggatcacttgcggttttcgggggcggaaccaaactaaccgttcttgggcaacctaaggctgccccgtccgtgactctttttcctccgtcttccgaagagcttcaagccaataaagcaaccttggtatgtttgatttccgatttttaccctggagcggtgacggtggcatggaaagctgattcaagccctgtgaaagctggggtggaaactactacgccgtccaaacagagcaataataagtacgcggcctccagctatttgtcacttaccccggagcaatggaagtcacaccgaagttacagttgtcaagttactcacgaaggcagcactgttgaaaagaccgtagcacccaccgaatgctca

SEQ ID NO: 207 - protein, 703 aa, synthetic construct
  REGION 1..703: note = BE718 A-BGA-5623- BGA-5623 heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 208 - DNA, 2109 bp, synthetic construct
  misc_feature 1..2109: note = BE718 A-BGA-5623- BGA-5623 heavy chain DNA
  source 1..2109: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggaggatcggaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 209 - protein, 577 aa, synthetic construct
  REGION 1..577: note = BE-740 AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 210 - DNA, 1731 bp, synthetic construct
  misc_feature 1..1731: note = BE-740 DNA
  source 1..1731: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 211 - protein, 450 aa, synthetic construct
  REGION 1..450: note = BE-942 (ZW1+1) anti-CEA heavy chain DNA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 212 - DNA, 1350 bp, synthetic construct
  misc_feature 1..1350: note = BE-942 (ZW1+1) anti-CEA heavy chain DNA
  source 1..1350: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaagggaccaagcgtgttcccactggcacccagctccaagagcacctccggaggaacagcatggctgggatgcgaggtgaccgactacttccccgagcctgtgacagtgtcttggaacagcggcgccctgaccagcggagtgcacacatttcctgccgtgctggagtctagcggcctgtactccctgtcctctgtggtgaccgtgcctagctcctctctgggcacccagacatatatctgtaacgtgaatcacaagccatctaatacaaaggtggataagaaggtggagccaaagtcctgcgacaagacccacacatgcccaccttgtccagcaccagaggcagcaggaggaccatccgtgttcctgtttccacccaagcccaaggataccctgatgatctctcggacccccgaggtgacatgcgtggtggtgagcgtgtcccacgaggaccctgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtctaataaggccctgcctgccccaatcgagaagaccatcagcaaggcaaagggacagcccagggagcctcaggtgtacgtgctgcctccaagccgcgacgagctgacaaagaaccaggtgtccctgctgtgcctggtgaagggcttctatccttccgatatcgccgtggagtgggagtctaatggccagccagagaacaattacctgacctggccccctgtgctggactccgatggctctttctttctgtattctaagctgacagtggataagagcaggtggcagcagggcaacgtgttttcttgcagcgtgatgcacgaggccctgcacaatcactacacccagaagtccctgtctctgagccctggcaag

SEQ ID NO: 213 - protein, 214 aa, synthetic construct
  REGION 1..214: note = BE-942 (ZW1+1) anti-CEA light chain AA
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIKRTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 214 - DNA, 642 bp, synthetic construct
  misc_feature 1..642: note = BE-942 (ZW1+1) anti-CEA light chain DNA
  source 1..642: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaaaggacagtggcagcaccaagcgtggcaatcttcccaccttccgacgagagactgaagtccggcaccgctagcgtggtgtgcgtgctgaacaacttctacccccgggaggccaaggtgcagtggaaggtggataacgccctgcagtccggcaattctcaggagagcgtgaccgagcaggactccaaggattctacatatagcctgagctccaggctgaccctgtctaaggccgactacgagaagcacaaggtgtatgcctgcgaggtgacacaccagggcctgtctagccccgtgaccaagtccttcaaccgcggcgagtgt

SEQ ID NO: 215 - protein, 354 aa, synthetic construct
  REGION 1..354: note = BE-942 (ZW1+1) anti-CD137 heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 216 - DNA, 1062 bp, synthetic construct
  misc_feature 1..1062: note = BE-942 (ZW1+1) anti-CD137 heavy chain DNA
  source 1..1062: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagccaaagtcctccgacaagacccacacatgcccaccttgtccagcaccagaggcagcaggaggaccatccgtgttcctgtttccacccaagcccaaggataccctgatgatctctcggacccccgaggtgacatgcgtggtggtgagcgtgtcccacgaggaccctgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtctaataaggccctgcctgccccaatcgagaagaccatcagcaaggcaaagggacagcccagggagcctcaggtgtacgtgtatcctccaagccgcgacgagctgaccaagaaccaggtgtccctgacatgtctggtgaagggcttttacccttccgatatcgccgtggagtgggagtctaatggccagccagagaacaattataagaccacaccccctgtgctggactccgatggctctttcgccctggtgtctaagctgaccgtggataagagcaggtggcagcagggcaacgtgttttcttgcagcgtgatgcacgaggccctgcacaatcactacacacagaagtccctgtctctgagccctggcaag

SEQ ID NO: 217 - protein, 481 aa, synthetic construct
  REGION 1..481: note = BE-755 (ZW1+2) anti-CD137 heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 218 - DNA, 1443 bp, synthetic construct
  misc_feature 1..1443: note = BE-755 (ZW1+2) anti-CD137 heavy chain DNA
  source 1..1443: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcgggcggcggagggagcgaggtgcagctgcttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagtgaccgtgtccagcgagccaaagtcctccgacaagacccacacatgcccaccttgtccagcaccagaggcagcaggaggaccatccgtgttcctgtttccacccaagcccaaggataccctgatgatctctcggacccccgaggtgacatgcgtggtggtgagcgtgtcccacgaggaccctgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtctaataaggccctgcctgccccaatcgagaagaccatcagcaaggcaaagggacagcccagggagcctcaggtgtacgtgtatcctccaagccgcgacgagctgaccaagaaccaggtgtccctgacatgtctggtgaagggcttttacccttccgatatcgccgtggagtgggagtctaatggccagccagagaacaattataagaccacaccccctgtgctggactccgatggctctttcgccctggtgtctaagctgaccgtggataagagcaggtggcagcagggcaacgtgttttcttgcagcgtgatgcacgaggccctgcacaatcactacacacagaagtccctgtctctgagccctggcaag

SEQ ID NO: 219 - protein, 582 aa, synthetic construct
  REGION 1..582: note = BE-562 (E-mutFc) heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 220 - DNA, 1746 bp, synthetic construct
  misc_feature 1..1746: note = BE-562 (E-mutFc) heavy chain DNA
  source 1..1746: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagccaaagtcctctgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcacaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgac

SEQ ID NO: 221 - protein, 583 aa, synthetic construct
  REGION 1..583: note = BE-375 (E-wtIgG1 Fc) heavy chain AA
EVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 222 - DNA, 1749 bp, synthetic construct
  misc_feature 1..1749: note = BE-375 (E-wtIgG1 Fc) heavy chain DNA
  source 1..1749: mol_type = other DNA
gaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggcggttcacaggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgac

SEQ ID NO: 223 - protein, 586 aa, synthetic construct
  REGION 1..586: note = BE-244 (A-(G4S)3) heavy chain AA
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 224 - DNA, 1758 bp, synthetic construct
  misc_feature 1..1758: note = BE-244 (A-(G4S)3) heavy chain DNA
  source 1..1758: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcaggcggcggaggatctggtggcggtgggtccgaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 225 - protein, 576 aa, synthetic construct; identical to SEQ ID NO: 167
  REGION 1..576: note = BE-146 heavy chain AA

SEQ ID NO: 226 - DNA, 1728 bp, synthetic construct; identical to SEQ ID NO: 168
  misc_feature 1..1728: note = BE-146 heavy chain DNA
  source 1..1728: mol_type = other DNA

SEQ ID NO: 227 - protein, 573 aa, synthetic construct
  REGION 1..573: note = BE-146 light chain AA
EVQLQQSGPELVKPGASMKISCKASGYSFTGYTMNWVKQSHGKNLEWIGLINPYNGGTSYNQKFKGKATLTIDKSSSTAYMELLSLTSEDSAVYYCARDYGYVLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 228 - DNA, 1719 bp, synthetic construct
  misc_feature 1..1719: note = BE-268 heavy chain DNA
  source 1..1719: mol_type = other DNA
gaagtgcaacttcaacaatcaggacctgaacttgtgaaacctggagcatcaatgaagatttcgtgcaaagcatcaggatactcatttacaggatacacaatgaactgggtgaaacaatcacacggaaagaatttggaatggatcggacttatcaacccttacaacggaggaacatcatacaaccagaagttcaagggaaaggctactcttacaatcgataaatcatcatcaacagcatacatggaacttctttcacttacatcagaagattcagcagtgtactactgcgcaagagattacggatacgtgcttgattactgggggcaggggacaacacttacagtgtcatcagctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccaaagtcctgtgacaagacccacacatgccccccttgtcctgctccaccagctgcaggaccaagcgtgttcctgtttccacccaagcccaaggataccctgatgatctctcggaccccagaggtgacatgcgtggtggtggatgtgagccacgaggaccccgaggtgaagttcaactggtatgtggacggcgtggaggtgcacaatgctaagaccaagcccagggaggagcagtacaactccacctatagagtggtgtctgtgctgacagtgctgcaccaggattggctgaacggcaaggagtataagtgcaaggtgtccaataaggccctggccgctcctatcgagaagaccatctctaaggccaagggccagcccagagagcctcaggtgtacacactgcctccatcccgggaagagatgaccaagaaccaggtgtctctgacatgtctggtcaagggcttctatccctctgacatcgccgtggagtgggagagcaatggccagcctgagaacaattacaagaccacaccccctgtgctggattccgacggctctttctttctgtatagcaagctgaccgtggacaagtcccggtggcagcagggcaacgtgttcagctgttccgtgatgcacgaagctctgcataatcactatactcagaaatccctgtcactgtcacctggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 229 - protein, 214 aa, synthetic construct
  REGION 1..214: note = BE-268 light chain AA
QIVLTQSPAIMSASPGEKVTITCSASSSVSYLHWFQQKPGTSPKLWVYSTSNLPSGVPARFGGSGSGTSYSLTISRMEAEDAATYYCQQRSIYPPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - DNA, 642 bp, synthetic construct
  misc_feature 1..642: note = BE-268 light chain DNA
  source 1..642: mol_type = other DNA
caaatcgtgcttacacaatcacctgcaatcatgtcagcatcacctggagagaaggttactatcacatgctcagcatcatcatcagtgtcataccttcactggtttcaacagaagcccgggacatcacctaaactttgggtgtactcaacatcaaaccttccttcaggagtgcctgcaagatttggaggatcaggatcaggaacatcatactcacttacaatctcaagaatggaagcagaagatgcagcaacatactactgccaacaaagatcaatctaccctccttggacatttggaggaggaacaaagttggagatcaaacgaacagtggcagccccttccgtcttcatttttcccccttctgacgaacagctgaaatcaggaactgctagcgtggtctgtctgctgaacaatttctaccccagagaggccaaggtgcagtggaaagtcgataacgctctgcagtccggcaattctcaggagagtgtgaccgaacaggactcaaaggatagcacatattccctgtctagtactctgaccctgagcaaagcagactacgagaagcacaaagtgtatgcctgtgaagtcacacaccaggggctgagttcaccagtcaccaagagtttcaacagaggggaatgc

SEQ ID NO: 231 - protein, 577 aa, synthetic construct
  REGION 1..577: note = BE-907 heavy chain AA
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 232 - DNA, 1731 bp, synthetic construct
  misc_feature 1..1731: note = BE-907 heavy chain DNA
  source 1..1731: mol_type = other DNA
caagtgcaactgcaacaatctggctctgagctgaagaagcctggggcctctgtgaaggtgagctgcaaggcctctggctacaccttcaccaactatggcatgaactgggtgaagcaagcccctggccaaggcctgaagtggatgggctggatcaacacctacactggggagcccacctacacagatgacttcaaaggtagatttgccttcagcctggacacctctgtgagcacagcctacctgcagatcagcagcctgaaggctgatgacacagctgtgtatttctgtgctagagggggctttggcagcagctactggtactttgatgtgtggggccaaggcagcctggtgactgtgagctctgctagcaccaaaggcccttctgtgttccccctggccccctcatccaagagcacctctgggggcacagctgccttaggctgtctggtgaaggactatttccctgagcctgtgacagtgagctggaattctggggccctgacctctggggtgcataccttccctgctgtgctgcagagctctggcctgtacagcctgagctctgtggtgacagtgcctagcagcagcctgggcacacagacctacatctgcaatgtgaaccacaagcctagcaacaccaaggtggacaagaaggtggagcccaagagctgtgacaaaacccacacctgtcccccctgccctgccccccctgctgctggcccctctgtgttccttttcccccccaagcccaaggacaccctgatgatcagcagaacccctgaggtgacctgtgtggtggtggatgtgagccatgaggaccctgaggtgaagttcaactggtatgtggatggggtggaggtgcacaatgccaagaccaagcctagagaggagcagtacaacagcacctacagagtggtgtctgtgctgacagtgctgcaccaagactggctgaatggcaaggagtacaagtgcaaggtgagcaacaaggccctggctgcccccattgagaagaccatcagcaaggccaaggggcagcctagagagccccaagtgtacaccctgccccctagcagagaggagatgaccaagaaccaagtgagcctgtggtgcctggtgaaaggcttctacccctctgacattgctgtggagtgggagagcaatgggcagcctgagaacaactacaagaccaccccccctgtgctggactctgatggcagcttcttcctgtacagcaagctgacagtggacaagagcagatggcagcaaggcaatgtgttcagctgctctgtgatgcatgaggccctgcacaaccactacacacagaagagcctgagcctgtcccctggtaaggggggtgggggctctgaggtgcagctgttggagtctggggggggcctggtgcagcctgggggcagtctaagactgagctgtgctgcctctggctttacagtgtctgctgaggatgtgggctgggtaagacaagcccctggcaaaggactggagtgggtgtctgccatcctggacaaggggggcagcacctactatgctgagtctgtgaagggcagatttaccattagcagagataatgccaagaataccctgtacctgcagatgagcagcctgagagctgaggacacagctgtgtattactgtgctagaattgtgtaccatgctggggggggggtgacttttgacacaagaggccaaggcacccaagtgacagtgagcagc

SEQ ID NO: 233 - protein, 214 aa, synthetic construct
  REGION 1..214: note = BE-907 light chain AA
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - DNA, 642 bp, synthetic construct
  misc_feature 1..642: note = BE-907 light chain DNA
  source 1..642: mol_type = other DNA
gatatccaacttacacaatcaccttcatcactttcggcctcggtaggcgatagggtgtcaatcacatgcaaagcatcacaagatgtgtcaatcgcagtggcatggtaccaacagaagcccggtaaggctcccaaacttcttatctactcagcatcatacagatacacaggagtgcctgatagattcagtggctcaggatcaggaacagatttcactttgacaatctcatcacttcaacctgaagatttcgctgtctactactgccaacaacactacatcacacctcttacatttggagcaggaacaaaggtagagatcaagcgaaccgtggcagcaccttcagtgtttatctttcctccttcagatgaacaacttaaatcaggaactgcgtcggtagtttgtcttcttaacaacttctatcccagagaagcaaaggtccagtggaaagtggataacgcacttcaatcaggaaactcacaagaatcagtgacagaacaagattcaaaggactccacatactcactttcatcaacacttacactttcaaaggccgactacgagaagcataaggtgtacgcatgcgaagtgacacaccaaggactttcatcacctgtgacaaagtcgttcaacagaggagaatgc

SEQ ID NO: 235 - protein, 572 aa, synthetic construct
  REGION 1..572: note = BE-830 heavy chain AA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTVSAEDVGWVRQAPGKGLEWVSAILDKGGSTYYAESVKGRFTISRDNAKNTLYLQMSSLRAEDTAVYYCARIVYHAGGGVTFDTRGQGTQVTVSS

SEQ ID NO: 236 - DNA, 1716 bp, synthetic construct
  misc_feature 1..1716: note = BE-830 heavy chain DNA
  source 1..1716: mol_type = other DNA
caggtgcagctggtgcagtctggagcagaggtgaagaagccaggagccagcgtgaaggtgtcctgcaaggcctctggctataccttcacagactacgagatgcactgggtgcggcaggcaccaggacagggactggagtggatgggcgccctggaccctaagaccggcgatacagcctatagccagaagtttaagggcagagtgaccctgacagccgacaagtccacctctacagcctacatggagctgagctccctgaccagcgaggatacagccgtgtactattgtacccggttctactcctatacatactggggccagggcaccctggtgacagtgtctagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggcggttcagaggtccagttacttgagagtggtggaggtctggtccaaccaggaggttcgctgcgtttatcctgcgccgcgtctggattcacggtttccgccgaagacgtgggttgggtgcgtcaagcgccggggaaaggactggaatgggtctccgccatcttggataagggtggttcgacatactatgcggaaagtgtcaaagggcgctttacgatctcgcgcgataacgcaaaaaatactctttaccttcaaatgtctagccttcgtgctgaggacactgcggtgtactactgcgcccgcattgtctaccatgctggtggtggcgtcacctttgatactcggggacagggcacccaagttacagtctcatcg

SEQ ID NO: 237 - protein, 219 aa, synthetic construct
  REGION 1..219: note = BE-830 light chain AA
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - DNA, 657 bp, synthetic construct
  misc_feature 1..657: note = BE-830 light chain DNA
  source 1..657: mol_type = other DNA
gacgtggtcatgacacagtcccctctgtctctgccagtgaccccaggagagcctgcctctatcagctgcaggagctcccagagcctggtgcactccaaccgcaatacatacctgcactggtatctgcagaagccaggccagtccccccagctgctgatctacaaggtgtctaaccggttcagcggcgtgcccgacagattttccggctctggcagcggcaccgatttcacactgaagatctcccgggtggaggcagaggatgtgggcgtgtactattgttctcagaatacccacgtgccccctacatttggccagggcaccaagctggagatcaagcgtacggtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 239-243 - skipped (5 entries)

SEQ ID NO: 244 - protein, 4 aa, synthetic construct
  REGION 1..4: note = linker
GGGS

SEQ ID NO: 245 - protein, 4 aa, synthetic construct
  REGION 1..4: note = SGGG
SGGG

SEQ ID NO: 246 - protein, 5 aa, synthetic construct
  REGION 1..5: note = linker
GGGGS

SEQ ID NO: 247 - protein, 7 aa, synthetic construct
  REGION 1..7: note = linker
GGGGSGS

SEQ ID NO: 248 - protein, 7 aa, synthetic construct
  REGION 1..7: note = linker
GGGGSGS

SEQ ID NO: 249 - protein, 8 aa, synthetic construct
  REGION 1..8: note = linker
GGGGSGGS

SEQ ID NO: 250 - protein, 10 aa, synthetic construct
  REGION 1..10: note = linker
GGGGSGGGGS

SEQ ID NO: 251 - protein, 15 aa, synthetic construct
  REGION 1..15: note = linker
GGGGSGGGGSGGGGS

SEQ ID NO: 252 - protein, 16 aa, synthetic construct
  REGION 1..16: note = linker
AKTTPKLEEGEFSEAR

SEQ ID NO: 253 - protein, 17 aa, synthetic construct
  REGION 1..17: note = AKTTPKLEEGEFSEARV
AKTTPKLEEGEFSEARV

SEQ ID NO: 254 - protein, 9 aa, synthetic construct
  REGION 1..9: note = linker
AKTTPKLGG

SEQ ID NO: 255 - protein, 10 aa, synthetic construct
  REGION 1..10: note = linker
SAKTTPKLGG

SEQ ID NO: 256 - protein, 17 aa, synthetic construct
  REGION 1..17: note = linker
AKTTPKLEEGEFSEARV

SEQ ID NO: 257 - protein, 6 aa, synthetic construct
  REGION 1..6: note = linker
SAKTTP

SEQ ID NO: 258 - protein, 10 aa, synthetic construct
  REGION 1..10: note = linker
SAKTTPKLGG

SEQ ID NO: 259 - protein, 6 aa, synthetic construct
  REGION 1..6: note = linker
RADAAP

SEQ ID NO: 260 - protein, 9 aa, synthetic construct
  REGION 1..9: note = RADAAPTVS
RADAAPTVS

SEQ ID NO: 261 - protein, 12 aa, synthetic construct
  REGION 1..12: note = linker
RADAAAAGGPGS

SEQ ID NO: 262 - protein, 27 aa, synthetic construct
  REGION 1..27: note = linker
RADAAAAGGGGSGGGGSGGGGSGGGGS

SEQ ID NO: 263 - protein, 6 aa, synthetic construct
  REGION 1..6: note = SAKTTP
SAKTTP

SEQ ID NO: 264 - protein, 10 aa, synthetic construct
  REGION 1..10: note = SAKTTPKLGG
SAKTTPKLGG

SEQ ID NO: 265 - protein, 18 aa, synthetic construct
  REGION 1..18: note = SAKTTPKLEEGEFSEARV
SAKTTPKLEEGEFSEARV

SEQ ID NO: 266 - protein, 5 aa, synthetic construct
  REGION 1..5: note = linker
ADAAP

SEQ ID NO: 267 - protein, 12 aa, synthetic construct
  REGION 1..12: note = linker
ADAAPTVSIFPP

SEQ ID NO: 268 - protein, 5 aa, synthetic construct
  REGION 1..5: note = linker
TVAAP

SEQ ID NO: 269 - protein, 12 aa, synthetic construct
  REGION 1..12: note = linker
TVAAPSVFIFPP

SEQ ID NO: 270 - protein, 6 aa, synthetic construct
  REGION 1..6: note = linker
QPKAAP

SEQ ID NO: 271 - protein, 13 aa, synthetic construct
  REGION 1..13: note = linker
QPKAAPSVTLFPP

SEQ ID NO: 272 - protein, 6 aa, synthetic construct
  REGION 1..6: note = liner
AKTTPP

SEQ ID NO: 273 - protein, 13 aa, synthetic construct
  REGION 1..13: note = linker
AKTTPPSVTPLAP

SEQ ID NO: 274 - protein, 6 aa, synthetic construct
  REGION 1..6: note = linker
AKTTAP

SEQ ID NO: 275 - protein, 13 aa, synthetic construct
  REGION 1..13: note = linker
AKTTAPSVYPLAP

SEQ ID NO: 276 - protein, 6 aa, synthetic construct
  REGION 1..6: note = linker
ASTKGP

SEQ ID NO: 277 - protein, 13 aa, synthetic construct
  REGION 1..13: note = linker
ASTKGPSVFPLAP

SEQ ID NO: 278 - protein, 15 aa, synthetic construct
  REGION 1..15: note = linker
GENKVEYAPALMALS

SEQ ID NO: 279 - protein, 15 aa, synthetic construct
  REGION 1..15: note = linker
GPAKELTPLKEAKVS

SEQ ID NO: 280 - protein, 15 aa, synthetic construct
  REGION 1..15: note = linker
GHEAAAVMQVQYPAS

(19) United States
(12) Patent Application Publication
(10) Pub. No.: US 2024/0209106 A1
(43) Pub. Date: Jun. 27, 2024

(54) ANTI-CD137 ANTIBODIES AND METHODS OF USE

(71) Applicant: BeiGene Switzerland GmbH, Basel (CH)

(72) Inventors: Liang QU, Beijing (CN); Tong ZHANG, Beijing (CN); Zhuo LI, Beijing (CN); Xin CHEN, Beijing (CN); Lin ZHU, Beijing (CN); Penghao WANG, Beijing (CN); Xiaosui Zhou, Beijing (CN); Yuanyuan XIE, Beijing (CN); Jie LI, Beijing (CN); Jian SUN, Beijing (CN); Jing SONG, Beijing (CN); Xuehui LI, Beijing (CN)

(73) Assignee: BeiGene Switzerland GmbH, Basel (CH)

(21) Appl. No.: 18/513,169

(22) Filed: Nov. 17, 2023

Related U.S. Application Data
(63) Continuation of application No. PCT/CN2022/093564, filed on May 18, 2022.

(30) Foreign Application Priority Data
May 21, 2021 (WO) PCT/CN2021/095111
Apr. 7, 2022 (WO) PCT/CN2022/085622

(51) Int. Cl. C07K 16/28; A61K 39/00; A61P 35/00

(52) U.S. Cl. CPC C07K 16/2878; A61P 35/00; A61K 2039/505; C07K 2317/31; C07K 2317/52; C07K 2317/565; C07K 2317/732

(57) ABSTRACT

Provided are antigen-binding fragments thereof that bind to human CD137, multispecific antibodies that recognize CD137 as one antigen and at least one other antigen, a pharmaceutical composition comprising CD137 antibodies, and use of the antibody, multispecific antibody or the composition for treating a disease, such as cancer.

### CROSS-REFERENCE TO RELATED APPLICATIONS

This application is a continuation of International Patent Application No. PCT/CN2022/093564, filed May 18, 2022, which claims priority from International Patent Application No. PCT/CN2021/095111, filed May 21, 2021, and International Patent Application No. PCT/CN2022/085622, filed Apr. 7, 2022. The contents of these applications are incorporated herein by reference in their entirety.

### SEQUENCE LISTING

The instant application contains a Sequence Listing which has been submitted electronically in XML format, and is hereby incorporated by reference in its entirety. Said XML file was created on Jan. 3, 2024, is named 138881_0297_Sequence_Listing.xml, and is 480 bytes in size.

### FIELD OF THE DISCLOSURE

Disclosed herein are antibodies that specifically bind to both human and Macaca fascicularis CD137 (TNF receptor superfamily member 9 (TNFRSF9)) without cross-reactivity with other human TNF receptor members, as well as isolated nucleic acids, vectors and host cells. These antibodies can be used to construct multispecific antibodies with other modalities such as tumor associated antigens, immune checkpoints or immune stimulators. Lastly, the anti-CD137 antibodies disclosed herein can be used in the treatment of various cancers.

### BACKGROUND

CD137 also known as TNF receptor superfamily member 9 (TNFRSF9), ILA or 4-1BB, is a member of the TNF-receptor superfamily, which plays important roles in clonal expansion, survival, and development of T cells. CD137 is a 30 kDa type I membrane glycoprotein with an extracellular domain containing four cysteine-rich pseudo repeats (CRDs), a short helical transmembrane domain and a cytoplasmic signaling domain (Kwon et al., (1989) Proc Natl Acad Sci USA, 86, 1963-7).

CD137 is expressed on various cell populations including activated CD4⁺ and CD8⁺ T cells, regulatory T cells (Treg), dendritic cells (DC), monocytes, mast cells, eosinophils and tumor endothelial cells. CD137 activation plays an important role in CD8+ T-cell activation and survival (Lee et al., (2002) J Immunol, 169, 4882-8; Pulle et al., (2006) J Immunol, 176, 2739-48). It sustains and augments effector functions and induces Th1 cytokine production (Bartkowiak et al., (2015) Front Oncol, 5, 117; Shuford et al., (1997) J Exp Med, 186, 47-55). Upon binding to its sole ligand, CD137 ligand (CD137L, 4-1BBL or TNFSF9), CD137 signaling results in increased expression of pro-survival molecules via NF-κB pathway activation (Wang et al. (2009) Immunol Rev, 229, 192-215).

Several studies have demonstrated that engagement of CD137, either by CD137L or agonistic antibodies, can inhibit tumor growth by promoting T cell activity (Dubrot et al., (2010) Cancer Immunol Immunother, 59, 1223-33; Gauttier et al., (2014) Int J Cancer, 135, 2857-67; Sallin et al., (2014) Cancer Immunol Immunother, 63, 947-58; McMillin et al., (2006) Hum Gene Ther, 17, 798-806). The effects of CD137 activation on the inhibition of activation-induced cell death (AICD) have been demonstrated both in vitro (Hurtado et al., (1997) J Immunol, 158, 2600-9) and in vivo (Takahashi et al., (1999) J Immunol, 162, 5037-40).

Both CD4+ and CD8+ T cells have been shown to respond to CD137 stimulation, however, it appears that enhancement of T-cell function is greater in CD8+ cells Shuford et al., (1997) J Exp Med, 186, 47-55; Gramaglia et al., (2000) Eur J Immunol, 30, 392-402).

In addition, CD137 agonists can synergize with several immunomodulators, including CpG, TRAIL, CD40, OX40, DR5, PD-1/PD-L1, CTLA4, Tim3, IL-2 and IL-12 (Taraban et al., (2002) Eur J Immunol, 32, 3617-27; Curran et al., (2011) PLOS One, 6, e19499; Gray et al., (2008) Eur J Immunol, 38, 2499-511; Wei et al., (2013) PLOS One, 8, e84927; Guo et al., (2013) J Transl Med, 11, 215; Kwong et al., (2013) Cancer Res, 73, 1547-58; Lee et al., (2004) J Immunother, 27, 201-10). CD137 agonists have also been demonstrated to ameliorate autoimmunity in animal models of lupus, collagen induced arthritis, and experimental autoimmune encephalomyelitis (Vinay et al., (2006) J Immunol, 177, 5708-17).

Several CD137 antibodies are in clinical development. Urelumab (BMS-66513) is a fully human non-ligand-blocking IgG4 antibody developed by Bristol-Myers squibb. Several phase I and II studies in various indications are currently ongoing. Severe liver toxicity (grade IV hepatitis) has been observed in Phase I and II studies (NCT00309023, NCT00612664, NCT01471210) with Urelumab (Segal et al., (2017) Clin Cancer Res, 23, 1929-1936: Timmerman et al., (2020) Am J Hematol, 95, 510-520; Chester et al., Blood, 131, 49-57. Utomilumab is a ligand-blocking IgG2 antibody and shows reduced toxicity with fewer grade III-IV adverse effects and no dose-limiting toxicity reported for doses up to 10 mg/kg (Fisher et al., (2012) Cancer Immunol Immunother, 61, 1721-33; Segal et al., (2018) Clin Cancer Res, 24, 1816-1823; Gopal et al., (2020) Clin Cancer Res, 26, 2524-2534).

Interleukin-27 generation upon activation of CD137 on liver myeloid cells has been shown essential for liver toxicity (Bartkowiak et al., (2018) Clin Cancer Res, 24, 1138-1151). To avoid systemic toxicity by CD137 activation, while maintaining good anti-tumor efficacy in the tumor microenvironment, Tumor associated antigen (TAA)-directed CD137 multispecific antibodies can reduce toxicity. Without being held to any one mechanism of action, a CD137×TAA multispecific antibody will only cross-link CD137 receptor when a TAA is also present, thus leading to immune cell stimulation in the tumor microenvironment. Therefore, CD137×TAA multispecific antibodies can greatly reduce the likelihood of systemic toxicity.

There are no approved therapeutic antibodies against CD137, and there remains an unmet medical need for therapeutics targeting CD137. The disclosure contains specific antibodies and antibody fragments directed to human CD137. In addition, the CD137 VH domain fragments disclosed herein can be used to construct multispecific antibodies with other modalities such as TAA, immune checkpoints or immune stimulators. CD137 antibodies alone or in combination with other modalities antibodies could potentially be used for the treatment or prevention of cancer, autoimmune disease or infectious diseases.

### SUMMARY OF THE DISCLOSURE

The present disclosure is directed to anti-CD137 antibodies and antigen-binding antibody fragments thereof that specifically bind CD137.

In one embodiment, the disclosure provides for antibodies that bind to human CD137, or antigen-binding fragments thereof.

The present disclosure encompasses the following embodiments.

An antibody or antigen binding antibody fragment thereof which specifically binds human CD137.

An antibody antigen binding fragment which specifically binds human CD137, comprising:
(i) a heavy chain variable region that comprises (a) a HCDR1 (Heavy Chain Complementarity Determining Region 1) of SEQ ID NO: 14, (b) a HCDR2 of SEQ ID NO: 29, (c) a HCDR3 of SEQ ID NO: 30;
(ii) a heavy chain variable region that comprises (a) a HCDR1 of SEQ ID NO:14, (b) a HCDR2 of SEQ ID NO: 22, (c) a HCDR3 of SEQ ID NO: 16;
(iii) a heavy chain variable region that comprises (a) a HCDR1 of SEQ ID NO:14, (b) a HCDR2 of SEQ ID NO: 15, (c) a HCDR3 of SEQ ID NO: 16; or
(vi) a heavy chain variable region that comprises (a) a HCDR1 of SEQ ID NO:4, (b) a HCDR2 of SEQ ID NO: 5, (c) a HCDR3 of SEQ ID NO: 6.

The antibody antigen binding fragment that comprises:
(i) a heavy chain variable region (VH) comprising an amino acid sequence at least 90, 91, 92, 93, 94, 95, 96, 97, 98 or 99% identical to SEQ ID NO: 33;
(ii) a heavy chain variable region (VH) comprising an amino acid sequence at least 90, 91, 92, 93, 94, 95, 96, 97, 98 or 99% identical to SEQ ID NO: 24;
(iii) a heavy chain variable region (VH) comprising an amino acid sequence at least 90, 91, 92, 93, 94, 95, 96, 97, 98 or 99% identical to SEQ ID NO: 19;
(iv) a heavy chain variable region (VH) comprising an amino acid sequence at least 90, 91, 92, 93, 94, 95, 96, 97, 98 or 99% identical to SEQ ID NO: 9; or
(v) a heavy chain variable region (VH) comprising an amino acid sequence at least 90, 91, 92, 93, 94, 95, 96, 97, 98 or 99% identical to SEQ ID NO: 103.

The antibody antigen binding fragment, wherein one, two, three, four, five, six, seven, eight, nine, or ten amino acids within SEQ ID NO:33, 24, 19, 9 or 103 have been inserted, deleted or substituted.

The antibody antigen binding fragment, that comprises:
(i) a heavy chain variable region (VH) comprising SEQ ID NO: 33;
(ii) a heavy chain variable region (VH) comprising SEQ ID NO: 24;
(iii) a heavy chain variable region (VH) comprising SEQ ID NO: 19;
(iv) a heavy chain variable region (VH) comprising SEQ ID NO: 9
(iv) a heavy chain variable region (VH) comprising SEQ ID NO: 103.

The antibody antigen binding fragment, which is heavy chain (scFv), a heavy chain Fab fragment, a heavy chain Fab′ fragment, or a heavy chain F(ab′)₂ fragment.

A multispecific antibody comprising at least a first antigen binding domain that specifically binds human CD137, wherein the first antigen binding domain comprises: (i) a heavy chain variable region that comprises (a) a HCDR1 (Heavy Chain Complementarity Determining Region 1) of SEQ ID NO: 14, (b) a HCDR2 of SEQ ID NO: 29, (c) a HCDR3 of SEQ ID NO: 30;
(ii) a heavy chain variable region that comprises (a) a HCDR1 of SEQ ID NO:14, (b) a HCDR2 of SEQ ID NO: 22, (c) a HCDR3 of SEQ ID NO: 16;
(iii) a heavy chain variable region that comprises (a) a HCDR1 of SEQ ID NO:14, (b) a HCDR2 of SEQ ID NO: 15, (c) a HCDR3 of SEQ ID NO: 16; or
(iv) a heavy chain variable region that comprises (a) a HCDR1 of SEQ ID NO:4, (b) a HCDR2 of SEQ ID NO: 5, (c) a HCDR3 of SEQ ID NO: 6,
and at least a second antigen binding domain that specifically binds a human tumor-associated antigen (TAA).

The multispecific antibody, wherein the first antigen binding domain comprises:
(i) a heavy chain variable region (VH) comprising SEQ ID NO: 33;
(ii) a heavy chain variable region (VH) comprising SEQ ID NO: 24;
(iii) a heavy chain variable region (VH) comprising SEQ ID NO: 19;
(iv) a heavy chain variable region (VH) comprising SEQ ID NO: 9
(iv) a heavy chain variable region (VH) comprising SEQ ID NO: 103,
and at least a second antigen binding domain that specifically binds a human tumor associated antigen (TAA).

The multispecific antibody, wherein the multispecific antibody is a bispecific antibody.

The bispecific antibody, wherein the bispecific is in the 1+1 format.

The bispecific antibody, wherein the bispecific is in the 1+2 format.

The bispecific antibody, wherein the bispecific is in the 2+2 format.

The bispecific antibody, wherein the linker is any sequence of SEQ ID NO:239 to SEQ ID NO 280.

The bispecific antibody, wherein the linker is SEQ ID NO: 246.

The bispecific antibody, wherein the linker is SEQ ID NO: 251.

The antibody or antibody fragment, wherein the antibody or antibody fragment thereof has antibody dependent cellular cytotoxicity (ADCC) or complement dependent cytotoxicity (CDC).

The antibody or antibody fragment, wherein the antibody or antibody fragment has reduced glycosylation or no glycosylation or is hypofucosylated.

The antibody or antibody fragment, wherein the antibody or antibody fragment comprises increased bisecting GlcNac structures.

The antibody or antibody fragment, wherein the Fc domain is of an IgG1.

The antibody or antibody fragment, wherein the Fc domain is of an IgG4.

A pharmaceutical composition comprising the antibody or antibody fragment of any one of claims 1 to 15 further comprising a pharmaceutically acceptable carrier.

A method of treating cancer comprising administering to a patient in need an effective amount of the antibody or antibody fragment.

The method, wherein the cancer is gastric cancer, colon cancer, pancreatic cancer, breast cancer, head and neck cancer, kidney cancer, liver cancer, lung cancer, small cell lung cancer, non-small cell lung cancer, ovarian cancer, skin cancer, mesothelioma, lymphoma, leukemia, myeloma and sarcoma.

The method, wherein the antibody or antibody fragment is administered in combination with another therapeutic agent.

The method, wherein the therapeutic agent is paclitaxel or a paclitaxel agent, docetaxel, carboplatin, topotecan, cisplatin, irinotecan, doxorubicin, lenalidomide or 5-azacytidine.

The method, wherein the therapeutic agent is an anti-PD-1 antibody.

An isolated nucleic acid that encodes the antibody or antibody fragment.

A vector comprising the nucleic acid.

A host cell comprising the nucleic acid or the vector.

A process for producing an antibody or antibody fragment comprising cultivating the host cell and recovering the antibody or antibody fragment from the culture.

In one embodiment, the antibody or an antigen-binding fragment thereof comprises one or more complementarity determining regions (CDRs) comprising an amino acid sequence selected from a group consisting of SEQ ID NO: 4, SEQ ID NO: 5, SEQ ID NO: 6, SEQ ID NO: 14, SEQ ID NO: 15, SEQ ID NO: 16, SEQ ID NO: 22, SEQ ID NO: 29 and SEQ ID NO: 30.

In another embodiment, the antibody or an antigen-binding fragment thereof comprises: a heavy chain variable region comprising one or more heavy chain complementarity determining regions (HCDRs) comprising an amino acid sequence selected from the group consisting of SEQ ID NO: 4, SEQ ID NO: 5, SEQ ID NO: 6, SEQ ID NO: 14, SEQ ID NO: 15, SEQ ID NO: 16, SEQ ID NO: 22, SEQ ID NO: 29 and SEQ ID NO: 30.

In another embodiment, the antibody or an antigen-binding fragment thereof comprises: a heavy chain variable region comprising three heavy chain complementarity determining regions (HCDRs) which are HCDR1 comprising an amino acid sequence of SEQ ID NO: 4, or SEQ ID NO: 14; HCDR2 comprising an amino acid sequence of SEQ ID NO: 5 SEQ ID NO: 15, SEQ ID NO: 22 or SEQ ID NO: 29; and HCDR3 comprising an amino acid sequence of SEQ ID NO: 6; SEQ ID NO: 16 or SEQ ID NO: 30.

In another embodiment, the antibody or an antigen-binding fragment thereof comprises: a heavy chain variable region comprising three heavy chain complementarity determining regions (HCDRs) which are HCDR1 comprising an amino acid sequence of SEQ ID NO:4, HCDR2 comprising an amino acid sequence of SEQ ID NO: 5, and HCDR3 comprising an amino acid sequence of SEQ ID NO: 6; or HCDR1 comprising an amino acid sequence of SEQ ID NO: 14, HCDR2 comprising an amino acid sequence of SEQ ID NO: 15, and HCDR3 comprising an amino acid sequence of SEQ ID NO: 16, or HCDR1 comprising an amino acid sequence of SEQ ID NO: 14, HCDR2 comprising an amino acid sequence of SEQ ID NO: 22, and HCDR3 comprising an amino acid sequence of SEQ ID NO: 16, or HCDR1 comprising an amino acid sequence of SEQ ID NO: 14, HCDR2 comprising an amino acid sequence of SEQ ID NO: 29, and HCDR3 comprising an amino acid sequence of SEQ ID NO: 30.

In one embodiment, the antibody of the present disclosure or an antigen-binding fragment thereof comprises: (a) a heavy chain variable region comprising an amino acid sequence of SEQ ID NO: 33, SEQ ID NO: 19, SEQ ID NO: 24 or SEQ ID NO: 103, or an amino acid sequence at least 95%, 96%, 97%, 98% or 99% identical to any one of SEQ ID NO: 33, SEQ ID NO: 19, SEQ ID NO: 24 or SEQ ID NO: 103.

In another embodiment, the antibody of the present disclosure or an antigen-binding fragment thereof comprises: a heavy chain variable region comprising an amino acid sequence of SEQ ID NO: 9, SEQ ID NO: 19, SEQ ID NO: 24, SEQ ID NO: 33 or SEQ ID NO: 103, or an amino acid sequence with one, two, or three amino acid substitutions in the amino acid sequence of SEQ ID NO: 9, SEQ ID NO: 19, SEQ ID NO: 24, SEQ ID NO: 33 or SEQ ID NO: 103.

In one embodiment, the antibody of the present disclosure or an antigen-binding fragment thereof comprises:
(a) a heavy chain variable region comprising an amino acid sequence of SEQ ID NO: 9,
(b) a heavy chain variable region comprising an amino acid sequence of SEQ ID NO: 19,
(c) a heavy chain variable region comprising an amino acid sequence of SEQ ID NO: 24,
(c) a heavy chain variable region comprising an amino acid sequence of SEQ ID NO: 33,
(d) a heavy chain variable region comprising an amino acid sequence of SEQ ID NO: 103.

In one embodiment, the antibody of the present disclosure is of IgG1, IgG2, IgG3, or IgG4 isotype. In a more specific embodiment, the antibody of the present disclosure comprises Fc domain of wild-type human IgG1 (also referred as human IgG1wt or huIgG1) or IgG2. In another embodiment, the antibody of the present disclosure comprises Fc domain of human IgG4 with S228P and/or R409K substitutions (according to EU numbering system).

In one embodiment, the antibody of the present disclosure binds to CD137 with a binding affinity (K_{D}) of from 1×10⁻⁶ M to 1×10⁻¹⁰ M. In another embodiment, the antibody of the present disclosure binds to CD137 with a binding affinity (K_{D})) of about 1×10⁻⁶ M, about 1×10⁻⁷ M, about 1×10⁻⁸ M, about 1×10⁻⁹ M or about 1×10⁻¹⁰ M.

In another embodiment, the anti-human CD137 antibody of the present disclosure shows a cross-species binding activity to cynomolgus CD137.

In one embodiment, antibodies of the present disclosure have strong Fc-mediated effector functions. The antibodies mediate antibody-dependent cellular cytotoxicity (ADCC) against CD137 expressing target cells.

The present disclosure relates to isolated nucleic acids comprising nucleotide sequences encoding the amino acid sequence of the antibody or an antigen-binding fragment. In one embodiment, the isolated nucleic acid comprises a VH nucleotide sequence of SEQ ID NO: 10, SEQ ID NO: 20, SEQ ID NO: 25, SEQ ID NO: 34 or SEQ ID NO: 104, or a nucleotide sequence comprising at least 95%, 96%, 97%, 98% or 99% identity to SEQ ID NO: 10, SEQ ID NO: 20, SEQ ID NO: 25, SEQ ID NO: 34 or SEQ ID NO: 104, and encodes the VH region of the antibody or an antigen-binding fragment of the present disclosure.

In another aspect, the present disclosure relates to a pharmaceutical composition comprising the CD137 antibody or antigen-binding fragment thereof, and optionally a pharmaceutically acceptable excipient.

In yet another aspect, the present disclosure relates to a method of treating a disease in a subject, which comprises administering the CD137 antibody or antigen-binding antibody fragment thereof, or an CD137 antibody pharmaceutical composition in a therapeutically effective amount to a subject in need thereof. In another embodiment the disease to be treated by the antibody or the antigen-binding fragment is cancer.

The current disclosure relates to use of the antibody or the antigen-binding antibody fragment thereof, or an CD137 antibody pharmaceutical composition for treating a disease, such as cancer.

### BRIEF DESCRIPTION OF THE DRAWINGS

FIG. 1A is a summary of human anti-huCD137 VH domain antibodies identified from each sub-library. FIG. 1B is graphic phylogenetic trees of human anti-huCD137 VH domain antibodies from each sub-library. The VH sequences of candidate anti-huCD137 VH domain antibodies were aligned using DNASTAR's Megalign™ software. Sequence homology was displayed in phylogenetic trees.

FIG. 2A shows the schematic diagram of human Fc fusion VH antibody format (VH-Fc). VH domain antibodies were fused at the N terminal of an inert Fc (without FcγR-binding) with a G4S linker in between. FIG. 2B shows a representative screening result using supernatants containing VH-Fc proteins, and FIG. 2C shows one of the clones, BGA-4712 has been demonstrated to be capable to stimulate IL-2 production in Hut78/huCD137 cells in a dose dependent manner.

FIG. 3A-3C is the binding profiles of a representative anti-huCD137 VH domain antibody BGA-4712. FIG. 3A shows the affinity determination of purified human anti-huCD137 VH domain antibody BGA-4712 by surface plasmon resonance (SPR). FIG. 3B depicts the determination of human anti-huCD137 VH domain antibody BGA-4712 binding by flow cytometry. FIG. 3C shows the blocking of human anti-huCD137 VH domain antibody BGA-4712 by huCD137 ligand (human CD137 ligand-ECD-mIgG2a fusion protein) interaction. The binding of purified human anti-huCD137 VH domain antibody BGA-4712 to CD137-expressing Hut78/huCD137 cells (Hut78/huCD137) was determined by flow cytometry.

FIG. 4A-D is a schematic diagram of example CD137×CEA multispecific antibody formats.

FIG. 5A-5B is a comparison of cell binding of CD137×CEA multispecific antibodies by flow cytometry. FIG. 5A shows the binding to CEA-expressing cells CT26/CEA. FIG. 5B shows the binding to CD137-expressing cells Hut78/huCD137.

FIG. 6A-B demonstrates that A-CD137×CEA stimulates PBMCs to produce IFN-γ in the presence of CEA⁺ tumor cells. FIG. 6A shows one of CD137×CEA multispecific antibodies A-CD137×CEA induces CD137 expressing cell line Hut78/huCD137 to produce Il-2. FIG. 6B shows one of CD137×CEA multispecific antibodies A-CD137×CEA induces human peripheral blood mononuclear cells (PBMCs) to produce IFN-γ in a dose dependent manner.

FIG. 7 is the binding to CD137-expressing cells Hut78/huCD137 of optimized A-CD137×CEA BGA-4712 variants by flow cytometry.

FIG. 8 shows CD137 activation in a PBMC based cytokine release assay, demonstrating that functions of A-CD137×CEA-M3 is maintained after removal of PTM sites.

FIG. 9A is a schematic diagram of phage display CH3 fusion of anti-huCD137 VH domain antibody. The VH domain antibody is fused at the C terminal of CH3 to mimic the bispecific format A-CD137×CEA. FIG. 9B demonstrates the supernatants containing CH3-BGA-4712-M3 can bind CD137 (human CD137-ECD mIgG2a) by ELISA. BGA-4712(VH-Fc) is used as the positive control, whereas huIgG is used as the negative control. CH3-BGA-4712-M3 with W47G or W47F or W47Y mutations in VH region failed to bind pre-coated human CD137-ECD-mIgG2a.

FIG. 10 shows the seq logo of CDR regions of BGA-4712-M3 after four rounds of selections.

FIG. 11 is a binding assay of anti-huCD137 VH domain antibody BGA-5623 by flow cytometry, demonstrating that binding to CD137 is improved after affinity maturation.

FIG. 12A-B shows the epitope mapping of human anti-huCD137 VH domain antibody BGA-5623. FIG. 12A is a representative screening result in a cell based binding assay. Expression of huCD137 mutants was monitored by Urelumab analog. FIG. 12B shows BGA-5623 binding of purified huCD137 mutants.

FIG. 13 shows the molecular modeling of huCD137 monomer.

FIG. 14A demonstrates CD137 ligand competes with human anti-huCD137 VH domain antibody BGA-5623 via ELISA. FIG. 14B demonstrates an CD137×CEA multispecific antibody BGA-5623 could reduce CD137/CD137 ligand interaction in a cell-based ligand competition assay.

FIG. 15 demonstrates no off-target binding of BGA-5623 on other TNF Receptor family members by ELISA.

FIG. 16A-B shows the affinity determination of purified BGA-7556 variants by surface plasmon resonance (SPR).

FIG. 17 is a schematic diagram of CD137×CEA multispecific antibody formats for investigating other parameters, such as module ratio which might influence CD137 activation in vitro.

FIG. 18A-B demonstrates the bispecific antibody A-41A11/41A11 with a module ratio of 2:4 could activate CD137, no matter if CEA⁺ tumor cells are present or not.

FIG. 19 is a schematic diagram of CD137×CEA multispecific antibody formats for investigating other parameters, such as Fc functions and module orientation which might influence CD137 activation in vitro.

FIG. 20A-B demonstrates that studied CD137×CEA multispecific antibodies only stimulate PBMCs to produce IFN-γ in the presence of CEA⁺ tumor cells.

FIG. 21 demonstrates that the linker length has minimal influence on CD137 activation in vitro in the presence of CEA⁺ tumor cells.

FIG. 22A-D shows Format A-BGA-5623 induces significant inhibition of tumor growth in vivo, but not A-IgG1-BGA-5623.

FIG. 23 is a schematic diagram of designed tumor-targeted TAA×CD137 multispecific antibody format.

FIG. 24A-B shows the binding of BE-146 to MKN45 (FIG. 24A) and Hut78/huCD137 cells (FIG. 24B). FIG. 24C-D shows the binding of BE-830 to HepG2 (FIG. 24C) and Hut78/huCD137 cells (FIG. 24D).

FIG. 25A-C demonstrates CEA×CD137 multispecific antibody BE-146 induces the IL-2 and IFN-γ release from human PBMCs. FIG. 25A is a schematic diagram of CD137 activation via co-stimulating huPBMCs with BE-146 and HEK293/OS8 cells in the presence of MKN45 cells. FIG. 25B-C shows BE-146 could induce IL-2 (FIG. 25B) and IFN-γ (FIG. 25C) from human PBMCs. PBMCs from 2 donors were tested. Results were shown in mean±SD of duplicates.

FIG. 26A-B demonstrates CEA×CD137 multispecific antibody BE-146 induces the IL-2 and IFN-γ release from human T cells. FIG. 26A shows BE-146 could induce IL-2 and IFN-γ (FIG. 26B) from human PBMCs. PBMCs from 2 donors were tested. Results were shown in mean±SD of duplicates.

FIG. 27A-B demonstrates CEA×CD137 induced response is CEA dependent. FIG. 27A shows that BE-146 could induce significant IL-2 and IFN-γ release (FIG. 27B) from PBMCs against CEA over-expressing HEK293 cells, but not against HEK293 cells without CEA transduction. PBMCs from 3 donors were tested. Results were shown in mean±SD of duplicates.

FIG. 28A-B shows the CD137×CEA induced response is not significantly blocked by recombinant soluble CEA. The results show that BE-146 induced IL-2 (FIG. 28A) and IFN-γ (FIG. 28B) release from PBMCs were not significantly blocked by 50 ng/ml or 500 ng/ml soluble CEA. PBMCs from 2 donors were tested. Results were shown in mean±SD of duplicates.

FIG. 29 shows that BE-146 and Urelumab analog induce significant inhibition of tumor growth.

FIG. 30 shows the combination of BE-146 and anti-PD-1 induces significantly increased anti-tumor effects.

FIG. 31 shows that BE-146 does not have liver toxicity in vivo. High-dose Urelumab analog, but not BE-146, induced significantly increased alanine transaminase (ALT) and aspartate aminotransferase (AST) concentrations, and increased inflammatory cells infiltration in liver.

FIG. 32 shows that Claudin6×CD137 (BE-268) induces IFN-γ release from human PBMCs. Cancer cell lines with different Claudin6 expression level were used. PA-1 is with high expression of Claudin6, Bewo is with mid-range expression of Claudin6, and MKN45 is negative for Claudin6 expression. The IFN-γ release by BE-268 is correlated with the expression level of Claudin6.

FIG. 33 shows that Trop2×CD137 (BE-907) induces IFN-γ release from human PBMCs. PBMCs from 2 donors were tested. Results were shown in mean±SD of duplicates.

FIG. 34A-B demonstrates GPC3×CD137 multispecific antibody BE-830 induces significant cytokine release from human PBMCs in the presence of GPC3, such as IL-2 (FIG. 34A) and IFN-γ (FIG. 34B).

FIG. 35 shows that BE-830 and an Urelumab analog induce significant inhibition of tumor growth.

FIG. 36 shows partially competitive binding of VHH (BGA-5623) against CD137L for CD137. The crystal structure of VHH (BGA-5623)/CD137 was superposed with CD137L/CD137 complex (PDB: 6MGP) via CD137. The CD137, CD137L and VHH are colored in black, white and grey, respectively.

FIG. 37 indicates that the CDR3 of VHH (BGA-5623) undergoes dramatically conformation change upon CD137 binding. The CD137 bound VHH (BGA-5623) in black was superposed with apoVHH (BGA-5623) in white.

FIG. 38 shows the atomic interactions on the binding surface of VHH (BGA-5623)/CD137 complex. The binding interface between VHH (BGA-5623) and CD137 identifies certain key residues of BGA-5623 (paratope residues) and CD137 (epitope residues). The CRD1 and 2 domains of CD137 are shown in grey cartoon covered with white transparent surface. The paratope residues is colored in black.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “CD137” or “TNFRSF9,” “ILA” or “41BB” refers to the amino acid sequence of human CD137, (SEQ ID NO: 47) can also be found at accession number Q07011 (TNR9_HUMAN) or U03397. The nucleic acid sequence of CD137 is set forth in SEQ ID NO:48.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contains a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA and IgY), or subclass (e.g., IgG1, IgG2, IgG3, IgG4, IgA1 and IgA2).

In some embodiments, the anti-CD137 antibodies comprise at least one antigen-binding site, at least a variable region. In some embodiments, the anti-CD137 antibodies comprise an antigen-binding fragment from an CD137 antibody described herein. In some embodiments, the anti-CD137 antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies comprising different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair comprising one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGen Tics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203: 121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs are numbered 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)₂, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times greater when compared to the background level and do not specifically bind in a significant amount to other antigens present in the sample. In one aspect, under designated immunoassay conditions, the antibody or antigen-binding fragment thereof, specifically bind to a particular antigen at least ten (10) times greater when compared to the background level of binding and does not specifically bind in a significant amount to other antigens present in the sample.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to CD137. Common conservative substations of amino acids are well known in the art.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm which has been incorporated into the ALIGN program (version 2.0), using a PAM120 weight residue table, a gap length penalty of 12 and a gap penalty of 4 (E. Meyers and W. Miller, Comput. Appl. Biosci. 4: 11-17, (1988)). In addition, the percent identity between two amino acid sequences can be determined using the algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6 (Needleman and Wunsch, J. Mol. Biol. 48:444-453, (1970)).

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include at least an anti-CD137 binding antibody as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-CD137 antibody, antigen binding fragment or anti-CD137 containing multispecific antibody is administered to the subject at the same time as, just before, or just after administration of an additional therapeutic agent. In certain embodiments, an anti-CD137 antibody, antigen binding fragment or anti-CD137 containing multispecific antibody is administered as a co-formulation with an additional therapeutic agent.

### DETAILED DESCRIPTION

The present disclosure provides for antibodies, antigen-binding fragments or anti-CD137 containing multivalent antibodies that specifically bind human CD137. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies or antigen binding fragments and methods of making and using such pharmaceutical compositions for the prevention and treatment of cancer and associated disorders.

### Anti-CD137 Antibodies

The present disclosure provides for antibodies or antigen-binding fragments thereof that specifically bind to CD137. Antibodies or antigen-binding fragments of the present disclosure include, but are not limited to, the antibodies or antigen-binding fragments thereof, generated as described, below.

The present disclosure provides antibodies or antigen-binding fragments that specifically bind to CD137, wherein said antibodies or antibody fragments (e.g., antigen-binding fragments) comprise a VH domain comprising an amino acid sequence of SEQ ID NO: 9, SEQ ID NO: 19, SEQ ID NO: 24, SEQ ID NO: 33 or SEQ ID NO: 103 (Table 1). The present disclosure also provides antibodies or antigen-binding fragments that specifically bind CD137, wherein said antibodies or antigen-binding fragments comprise a HCDR (heavy chain complementarity determining region) comprising an amino acid sequence of any one of the HCDRs listed in Table 1. In one aspect, the present disclosure provides antibodies or antigen-binding fragments that specifically bind to CD137, wherein said antibodies comprise (or alternatively, consist of) one, two, three, or more HCDRs comprising an amino acid sequence of any of the HCDRs listed in Table 1.

Other antibodies or antigen-binding fragments thereof of the present disclosure include amino acids that have been changed, yet have at least 60%, 70%, 80%, 90%, 95% or 99% percent identity in the CDR regions with the CDR regions disclosed in Table 1. In some aspects, it includes amino acid changes wherein no more than 1, 2, 3, 4 or 5 amino acids have been changed in the CDR regions when compared with the CDR regions depicted in the sequence described in Table 1.

Other antibodies of the present disclosure include those where the amino acids or nucleic acids encoding the amino acids have been changed; yet have at least 60%, 70%, 80%, 90%, 95% or 99% percent identity to the sequences described in Table 1. In some aspects, it includes changes in the amino acid sequences wherein no more than 1, 2, 3, 4 or 5 amino acids have been changed in the variable regions when compared with the variable regions depicted in the sequence described in Table 1, while retaining substantially the same therapeutic activity.

The present disclosure also provides nucleic acid sequences that encode VH domain antibody and the full length heavy chain of the antibodies that specifically bind to CD137. Such nucleic acid sequences can be optimized for expression in mammalian cells.

TABLE 1

| Antibody | SEQ ID NO | | SEQUENCE |
|---|---|---|---|
| BGA-7207 | SEQ ID NO: 1 | HCDR1 (IMGT) | GFRLDTTE |
| (IMGT) | SEQ ID NO: 2 | HCDR2 (IMGT) | IMGISGST |
| | SEQ ID NO: 3 | HCDR3 (IMGT) | ARVVDSLFDDSAVFDY |
| BGA-7207 | SEQ ID NO: 4 | HCDR1 (Kabat) | TTEVG |
| (Kabat) | SEQ ID NO: 5 | HCDR2 (Kabat) | TIMGISGSTYYADSVKG |
| | SEQ ID NO: 6 | HCDR3 (Kabat) | VVDSLFDDSAVFDY |
| BGA-7207 | SEQ ID NO: 7 | HCDR1 (Chothia) | GFRLDTT |
| (Chothia) | SEQ ID NO: 8 | HCDR2 (Chothia) | MGISGS |
| | SEQ ID NO: 6 | HCDR3 (Chothia) | VVDSLFDDSAVFDY |
| BGA-7207 | SEQ ID NO: 9 | VH | EVQLLESGGGLVQPGGSLRLSCAASGFRLD |
| | | | TTEVGWVRQAPGKGLEWVSTIMGISGSTYY |
| | | | ADSVKGRFTISRDNSKNTLYLQMNSLRAED |
| | | | TAVYYCARVVDSLFDDSAVFDYWGQGTLVT |
| | | | VSS |
| | SEQ ID NO: 10 | VH DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCAGATTGG |
| | | | ACACCACCGAAGTAGGTTGGGTGCGTCAA |
| | | | GCGCCGGGGAAAGGACTGGAATGGGTCTC |
| | | | CACCATCATGGGTATTAGTGGTTCGACATA |
| | | | CTATGCGGACAGTGTCAAAGGGCGCTTTA |
| | | | CGATCTCGCGCGATAACTCAAAAAATACTC |
| | | | TTTACCTTCAAATGAATAGCCTTCGTGCTG |
| | | | AGGACACTGCGGTGTACTACTGCGCCCGC |
| | | | GTCGTCGATTCCCTCTTTGATGACAGCGCC |
| | | | GTTTTTGATTACTGGGGACAGGGCACCTTA |
| | | | GTTACAGTCTCATCG |
| BGA-4712 | SEQ ID NO: 11 | HCDR1 (IMGT) | GFMLSAED |
| (IMGT) | SEQ ID NO: 12 | HCDR2 (IMGT) | ILDFGGST |
| | SEQ ID NO: 13 | HCDR3 (IMGT) | ARVVYHAGGGVTFDY |
| BGA-4712 | SEQ ID NO: 14 | HCDR1 (Kabat) | AEDVG |
| (Kabat) | SEQ ID NO: 15 | HCDR2 (Kabat) | AILDFGGSTYYADSVKG |
| | SEQ ID NO: 16 | HCDR3 (Kabat) | VVYHAGGGVTFDY |
| BGA-4712 | SEQ ID NO: 17 | HCDR1 (Chothia) | GFMLSAE |
| (Chothia) | SEQ ID NO: 18 | HCDR2 (Chothia) | LDFGGS |
| | SEQ ID NO: 16 | HCDR3 (Chothia) | VVYHAGGGVTFDY |
| BGA-4712 | SEQ ID NO: 19 | VH | EVQLLESGGGLVQPGGSLRLSCAASGFMLS |
| | | | AEDVGWVRQAPGKGLEWVSAILDFGGSTY |
| | | | YADSVKGRFTISRDNSKNTLYLQMNSLRAE |
| | | | DTAVYYCARVVYHAGGGVTFDYWGQGTLV |
| | | | TVSS |
| | SEQ ID NO: 20 | VH DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCATGTTGT |
| | | | CCGCCGAAGACGTGGGTTGGGTGCGTCAA |
| | | | GCGCCGGGGAAAGGACTGGAATGGGTCTC |
| | | | CGCCATCTTGGATTTTGGTGGTTCGACATA |
| | | | CTATGCGGACAGTGTCAAAGGGCGCTTTA |
| | | | CGATCTCGCGCGATAACTCAAAAAATACTC |
| | | | TTTACCTTCAAATGAATAGCCTTCGTGCTG |
| | | | AGGACACTGCGGTGTACTACTGCGCCCGC |
| | | | GTCGTCTACCATGCTGGTGGTGGCGTCAC |
| | | | CTTTGATTACTGGGGACAGGGCACCTTAGT |
| | | | TACAGTCTCATCG |
| BGA-4712-M3 | SEQ ID NO: 21 | HCDR1 (IMGT) | GFTLSAED |
| (IMGT) | SEQ ID NO: 12 | HCDR2 (IMGT) | ILDFGGST |
| | SEQ ID NO: 13 | HCDR3 (IMGT) | ARVVYHAGGGVTFDY |
| BGA-4712-M3 | SEQ ID NO: 14 | HCDR1 (Kabat) | AEDVG |
| (Kabat) | SEQ ID NO: 22 | HCDR2 (Kabat) | AILDFGGSTYYAESVKG |
| | SEQ ID NO: 16 | HCDR3 (Kabat) | VVYHAGGGVTFDY |
| BGA-4712-M3 | SEQ ID NO: 23 | HCDR1 (Chothia) | GFTLSAE |
| (Chothia) | SEQ ID NO: 18 | HCDR2 (Chothia) | LDFGGS |
| | SEQ ID NO: 16 | HCDR3 (Chothia) | VVYHAGGGVTFDY |
| BGA-4712-M3 | SEQ ID NO: 24 | VH | EVQLLESGGGLVQPGGSLRLSCAASGFTLSA |
| | | | EDVGWVRQAPGKGLEWVSAILDFGGSTYY |
| | | | AESVKGRFTISRDNAKNTLYLQMSSLRAED |
| | | | TAVYYCARVVYHAGGGVTFDYWGQGTLVT |
| | | | VSS |
| | SEQ ID NO: 25 | VH DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCACGTTGT |
| | | | CCGCCGAAGACGTGGGTTGGGTGCGTCAA |
| | | | GCGCCGGGGAAAGGACTGGAATGGGTCTC |
| | | | CGCCATCTTGGATTTTGGTGGTTCGACATA |
| | | | CTATGCGGAAAGTGTCAAAGGGCGCTTTA |
| | | | CGATCTCGCGCGATAACGCAAAAAATACT |
| | | | CTTTACCTTCAAATGTCTAGCCTTCGTGCT |
| | | | GAGGACACTGCGGTGTACTACTGCGCCCG |
| | | | CGTCGTCTACCATGCTGGTGGTGGCGTCA |
| | | | CCTTTGATTACTGGGGACAGGGCACCTTA |
| | | | GTTACAGTCTCATCG |
| BGA-5623 | SEQ ID NO: 26 | HCDR1 (IMGT) | GFTVSAED |
| (IMGT) | SEQ ID NO: 27 | HCDR2 (IMGT) | ILDKGGST |
| | SEQ ID NO: 28 | HCDR3 (IMGT) | ARIVYHAGGGVTFDT |
| BGA-5623 | SEQ ID NO: 14 | HCDR1 (Kabat) | AEDVG |
| (Kabat) | SEQ ID NO: 29 | HCDR2 (Kabat) | AILDKGGSTYYAESVKG |
| | SEQ ID NO: 30 | HCDR3 (Kabat) | IVYHAGGGVTFDT |
| BGA-5623 | SEQ ID NO: 31 | HCDR1 (Chothia) | GFTVSAE |
| (Chothia) | SEQ ID NO: 32 | HCDR2 (Chothia) | LDKGGS |
| | SEQ ID NO: 30 | HCDR3 (Chothia) | IVYHAGGGVTFDT |
| BGA-5623 | SEQ ID NO: 33 | VH | EVQLLESGGGLVQPGGSLRLSCAASGFTVS |
| | | | AEDVGWVRQAPGKGLEWVSAILDKGGSTY |
| | | | YAESVKGRFTISRDNAKNTLYLQMSSLRAE |
| | | | DTAVYYCARIVYHAGGGVTFDTRGQGTQV |
| | | | TVSS |
| | SEQ ID NO: 34 | VH DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCACGGTTT |
| | | | CCGCCGAAGACGTGGGTTGGGTGCGTCAA |
| | | | GCGCCGGGGAAAGGACTGGAATGGGTCTC |
| | | | CGCCATCTTGGATAAGGGTGGTTCGACATA |
| | | | CTATGCGGAAAGTGTCAAAGGGCGCTTTA |
| | | | CGATCTCGCGCGATAACGCAAAAAATACT |
| | | | CTTTACCTTCAAATGTCTAGCCTTCGTGCT |
| | | | GAGGACACTGCGGTGTACTACTGCGCCCG |
| | | | CATTGTCTACCATGCTGGTGGTGGCGTCAC |
| | | | CTTTGATACTCGGGGACAGGGCACCCAAG |
| | | | TTACAGTCTCATCG |
| BGA-7556 | SEQ ID NO: 103 | VH | EVQLLESGGGLVQPGGSLRLSCAASGFTLSA |
| | | | EDVGWVRQAPGKGLEWVSAILDFGGSTYY |
| | | | AESVKGRFTISRDNAKNTLYLQMSSLRAED |
| | | | TAVYYCARVVYHAGGGVTFDYRGQGTQVT |
| | | | VSS |
| | SEQ ID NO: 104 | VH DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCACGTTGT |
| | | | CCGCCGAAGACGTGGGTTGGGTGCGTCAA |
| | | | GCGCCGGGGAAAGGACTGGAATGGGTCTC |
| | | | CGCCATCTTGGATTTTGGTGGTTCGACATA |
| | | | CTATGCGGAAAGTGTCAAAGGGCGCTTTA |
| | | | CGATCTCGCGCGATAACGCAAAAAATACT |
| | | | CTTTACCTTCAAATGTCTAGCCTTCGTGCT |
| | | | GAGGACACTGCGGTGTACTACTGCGCCCG |
| | | | CGTCGTCTACCATGCTGGTGGTGGCGTCA |
| | | | CCTTTGATTACCGGGGACAGGGCACCCAA |
| | | | GTTACAGTCTCATCG |

Identification of Epitopes and Antibodies that Bind to the Same Epitope

The present disclosure provides antibodies and antigen-binding fragments thereof that bind to an epitope of human CD137. In certain aspects the antibodies and antigen-binding fragments can bind to the same epitope of CD137.

The present disclosure also provides for antibodies and antigen-binding fragments thereof that bind to the same epitope as do the anti-CD137 antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CD137 demonstrates that the test antibody can compete with that antibody or antigen-binding fragments thereof for binding to CD137. Such an antibody can, without being bound to any one theory, bind to the same or a related (e.g., a structurally similar or spatially proximal) epitope on CD137 as the antibody or antigen-binding fragments thereof with which it competes. In a certain aspect, the antibody that binds to the same epitope on CD137 as the antibodies or antigen-binding fragments thereof of the present disclosure is a human or humanized monoclonal antibody. Such human or humanized monoclonal antibodies can be prepared and isolated as described herein.

### Alteration of the Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (Shields et al., J. Biol. Chem. 276:6591-6604, 2001).

In still another aspect, the glycosylation of the CD137 antibody or antigen binding fragment is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody comprising reduced amounts of fucosyl residues or an antibody comprising increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore et al., 2010 MAbs, 2:181-189). However, natural IgG4 was found less stable in stress conditions such as in acidic buffer or under increasing temperature (Angal, 1993 Mol Immunol, 30:105-108; Dall'Acqua et al, 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317: 1554-157). The mutation of serine to proline at position 228 (EU numbering system) appeared inhibitory to the IgG4 heavy chain separation (Angal, 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel et al., 1991 Proc. Natl. Acad. Sci. USA, 88:9036-9040; Mukherjee et al., 1995 FASEB J, 9:115-119; Armour et al., 1999 Eur J Immunol, 29:2613-2624; Clynes et al, 2000 Nature Medicine, 6:443-446; Arnold, 2007 Annu Rev immunol, 25:21-50). Furthermore, some rarely occurring IgG4 isoforms in human population can also elicit different physicochemical properties (Brusco et al., 1998 Eur J Immunogenet, 25:349-55; Aalberse et al., 2002 Immunol, 105:9-19). To generate CD137 antibodies with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

### CD137 Antibody Production

Anti-CD137 antibodies, antigen-binding fragments and multispecific antibodies can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO:10, SEQ ID NO: 20, SEQ ID NO: 25, SEQ ID NO: 33 or SEQ ID NO: 104.

The polynucleotides of the present disclosure can encode the variable region sequence of an anti-CD137 antibody. They can also encode both a variable region and a constant region of the antibody. Some of the polynucleotide sequences encode a polypeptide that comprises variable regions of both the heavy chain and the light chain of one of the exemplified anti-CD137 antibodies.

Also provided in the present disclosure are expression vectors and host cells for producing the anti-CD137 antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-CD137 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-CD137 antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-CD137 antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-CD137 polypeptides. Insect cells in combination with baculovirus vectors can also be used.

In other aspects, mammalian host cells are used to express and produce the anti-CD137 polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### CD137 Multispecific Antibodies

In one embodiment, the anti-CD137 antibodies as disclosed herein can be incorporated into an anti-CD137×TAA multispecific antibody, wherein TAA is an antibody or fragment thereof directed to any human tumor associated antigen (TAA). An antibody molecule is a multispecific antibody molecule, for example, it comprises a number of antigen binding domains, wherein at least one antigen binding domain sequence specifically binds CD137 and a second antigen binding domain sequence specifically binds a TAA. In one embodiment, the multispecific antibody comprises a third, fourth or fifth antigen binding domain. In one embodiment, the multispecific antibody is a bispecific antibody, a trispecific antibody, or tetraspecific antibody. In each example, the multispecific antibody comprises at least one anti-CD137 antigen binding domain and at least one anti-TAA antigen binding domain.

In one embodiment, the multispecific antibody is a bispecific antibody. As used herein, a bispecific antibody specifically binds only two antigens. The bispecific antibody comprises a first antigen binding domain which specifically binds CD137 and a second antigen binding domain that specifically binds a TAA. This includes a bispecific antibody comprising a heavy chain variable domain which specifically bind CD137 and a heavy chain variable domain and a light chain variable domain which specifically bind a TAA. In another embodiment, the bispecific antibody comprises an antigen binding fragment of an antibody that specifically binds CD137 and an antigen binding fragment that specially binds a TAA. The bispecific antibody that comprises antigen binding fragments, the antigen-binding fragment can be a Fab, F(ab′)₂, Fv, or a single chain Fv (ScFv) or a scFv.

Previous experimentation (Coloma and Morrison Nature Biotech. 15: 159-163 (1997)) described a tetravalent bispecific antibody which was engineered by fusing DNA encoding a single chain anti-dansyl antibody Fv (scFv) after the C terminus (CH3-scFv) or after the hinge (hinge-scFv) of an IgG3 anti-dansyl antibody. The present disclosure provides multivalent antibodies (e.g. tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains, which specifically bind at least two antigens.

The disclosure provides for a bispecific tetravalent antibody comprising VD1-CL-(X1)n-VD2-CH1-Fc or VD1-CH-(X1)n-VD2-CL-Fc, wherein VD1 is a first variable domain, VD2 is a second variable domain, Fc is one polypeptide chain of an Fc region, CH or CL is a constant heavy or constant light domain, and (X1)n is a linker of at least 2 amino acids.

In one embodiment the bispecific tetravalent antibody can be multimer of four polypeptide chains, two heavy chains each comprising a first VH domain (VH1), a first CH1 domain, a second VH domain (VH2) an Fc region comprising a second CH1, Hinge, CH2, a CH3 and two light chains, each light chain comprising a first VL domain (VL1), a first CL region, a second VL domain (VL2), and a second CL region. In another embodiment the bispecific tetravalent can comprise multiple antibody Fab fragments linked together to a single Fc domain. For example, a Fab1 can be linked via a polypeptide linker to a Fab2, which comprises the CH1 domain of one of the Fab, hinge region then CH2 and CH3 of the Fc domain. For example, an anti-TAA Fab can be linked via a linker from the CL domain of the anti-TAA Fab to a VH domain of anti-CD137 Fab and from the CH1 domain of the anti-CD137 Fab, the hinge region, CH2 and CH3 domains. In another example, an anti-CD137 Fab can be linked via a linker from the CL domain of the anti-CD137 Fab to a VH domain of anti-TAA Fab and from the CH1 domain of the anti-TAA Fab, the hinge region, CH2 and CH3 domains.

### Linkers

It is also understood that the domains and/or regions of the polypeptide chains of the multispecific antibody can be separated by linker regions of various lengths. In some embodiments, the epitope binding domains are separated from each other, a CL, CH1, hinge, CH2, CH3, or the entire Fc region by a linker region. For example, VL1-CL-(linker) VH2-CH1 Such linker region may comprise a random assortment of amino acids, or a restricted set of amino acids. Such linker regions can be flexible or rigid (see US2009/0155275).

Multispecific antibodies have been constructed by genetically fusing two single chain Fv (scFv) or Fab fragments with or without the use of flexible linkers (Mallender et al., J. Biol. Chem. 1994 269: 199-206; Macket et al., Proc. Natl. Acad. Sci. USA. 1995 92:7021-5; Zapata Protein Eng. 1995 8.1057-62), via a dimerization device such as leucine Zipper (Kostelny et al., J. Immunol. 1992148: 1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Packet al., Biochemistry 1992.31:1579-84; Packet al., Bio/Technology 1993 11:1271-7).

The multispecific antibodies as disclosed herein comprise a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its epitope binding domains, CL domains, CH1 domains, Hinge region, CH2 domains, CH3 domains, or Fc regions. In some embodiments, the amino acids glycine and serine comprise the amino acids within the linker region. In another embodiment, the linker can be GS (SEQ ID NO:239), GGS (SEQ ID NO: 240), GSG (SEQ ID NO: 241), SGG (SEQ ID NO: 242), GGG (SEQ ID NO: 243), GGGS (SEQ ID NO: 244), SGGG (SEQ ID NO: 245), GGGGS (SEQ ID NO: 246), GGGGSGS (SEQ ID NO: 247), GGGGSGS (SEQ ID NO: 248), GGGGSGGS (SEQ ID NO: 249), GGGGSGGGGS (SEQ ID NO: 250), GGGGSGGGGSGGGGS (SEQ ID NO: 251), AKTTPKLEEGEFSEAR (SEQ ID NO: 252), AKTTPKLEEGEFSEARV (SEQ ID NO: 253), AKTTPKLGG (SEQ ID NO: 254), SAKTTPKLGG (SEQ ID NO: 255), AKTTPKLEEGEFSEARV (SEQ ID NO: 256), SAKTTP (SEQ ID NO: 257), SAKTTPKLGG (SEQ ID NO: 258), RADAAP (SEQ ID NO: 259), RADAAPTVS (SEQ ID NO: 260), RADAAAAGGPGS (SEQ ID NO: 261), RADAAAA(G₄S)₄ (SEQ ID NO: 262), SAKTTP (SEQ ID NO: 263), SAKTTPKLGG (SEQ ID NO: 264), SAKTTPKLEEGEFSEARV (SEQ ID NO: 265), ADAAP (SEQ ID NO: 266), ADAAPTVSIFPP (SEQ ID NO: 267), TVAAP (SEQ ID NO: 268), TVAAPSVFIFPP (SEQ ID NO: 269), QPKAAP (SEQ ID NO: 270), QPKAAPSVTLFPP (SEQ ID NO: 271), AKTTPP (SEQ ID NO: 272), AKTTPPSVTPLAP (SEQ ID NO: 273), AKTTAP (SEQ ID NO: 274), AKTTAPSVYPLAP (SEQ ID NO: 275), ASTKGP (SEQ ID NO: 276), ASTKGPSVFPLAP (SEQ ID NO: 277), GENKVEYAPALMALS (SEQ ID NO: 278), GPAKELTPLKEAKVS (SEQ ID NO: 279), and GHEAAAVMQVQYPAS (SEQ ID NO: 280) or any combination thereof (see WO2007/024715).

### Dimerization Specific Amino Acids

In one embodiment, the multispecific antibody comprises at least one dimerization specific amino acid change. The dimerization specific amino acid changes result in “knobs into holes” interactions, and increases the assembly of correct multispecific antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 and WO2017059551. The dimerization specific amino acids can also be within the Fc domain and can be in combination with dimerization specific amino acids within the CH1 or CL domains.

### Methods of Detection and Diagnosis

The antibodies or antigen-binding fragments of the present disclosure are useful in a variety of applications including, but not limited to, methods for the detection of CD137. In one aspect, the antibodies or antigen-binding fragments are useful for detecting the presence of CD137 in a biological sample. The term “detecting” as used herein includes quantitative or qualitative detection. In certain aspects, a biological sample comprises a cell or tissue. In other aspects, such tissues include normal and/or cancerous tissues that express CD137 at higher levels relative to other tissues.

In one aspect, the present disclosure provides a method of detecting the presence of CD137 in a biological sample. In certain aspects, the method comprises contacting the biological sample with an anti-CD137 antibody or antigen binding fragment thereof under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of CD137. In certain aspects, the method comprises contacting a test cell with an anti-CD137 antibody or antigen binding fragment thereof; determining the level of expression (either quantitatively or qualitatively) of CD137 expressed by the test cell by detecting binding of the anti-CD137 antibody or antigen binding fragment thereof to the CD137 polypeptide; and comparing the level of expression by the test cell with the level of CD137 expression in a control cell (e.g., a normal cell of the same tissue origin as the test cell or a non-CD137 expressing cell), wherein a higher level of CD137 expression in the test cell as compared to the control cell indicates the presence of a disorder associated with expression of CD137.

### Methods of Treatment

The antibodies or antigen-binding fragments of the present disclosure are useful in a variety of applications including, but not limited to, methods for the treatment of a CD137-associated disorder or disease. In one aspect, the CD137-associated disorder or disease is a cancer. In the case of a CD137×TAA multispecific antibody, the cancer can be specific to the TAA, with CD137 acting to recruit immune cells to the TAA expressing tumor.

In one aspect, the present disclosure provides a method of treating cancer. In certain aspects, the method comprises administering to a patient in need an effective amount of an anti-CD137 antibody, antigen-binding fragment thereof or CD137 containing multispecific antibody. The cancer can include, without limitation, gastric cancer, colon cancer, pancreatic cancer, breast cancer, head and neck cancer, kidney cancer, liver cancer, lung cancer, small cell lung cancer, non-small cell lung cancer, ovarian cancer, skin cancer, mesothelioma, lymphoma, leukemia, myeloma and sarcoma.

The anti-CD137 antibody or antigen-binding fragment as disclosed herein can be administered by any suitable means, including parenteral, intrapulmonary, and intranasal, and, if desired for local treatment, intralesional or intratumoral administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of antibody, antigen-binding fragment thereof or multispecific antibody of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g. every week or every three weeks (e.g. such that the patient receives from about two to about twenty administrations). An initial high loading dose, followed by one or more lower doses can be administered. However, other dosage regimens can be useful and the progress of the therapy is easily monitored by conventional techniques and assays.

### Combination Therapy

In one aspect, CD137 antibodies, antigen binding fragments thereof or multispecific antibodies of the present disclosure can be used in combination with other therapeutic agents. Other therapeutic agents that can be used with the CD137 antibodies of the present disclosure include: but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

Anti-CD137×TAA antibodies of the present disclosure can be used in combination with other therapeutics, for example, other immune checkpoint antibodies. Such immune checkpoint antibodies can include anti-PD1 antibodies. Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), is disclosed in U.S. Pat. Nos. 8,354,509 and 8,900,587 and is a humanized IgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human IgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

### Pharmaceutical Compositions and Formulations

Also provided are compositions, including pharmaceutical formulations, comprising an anti-CD137 antibody, antigen binding fragment thereof, multispecific antibody, or polynucleotides comprising sequences encoding an anti-CD137 antibody, antigen binding fragment thereof or multispecific antibody. In certain embodiments, compositions comprise one or more CD137 antibodies or antigen binding fragments thereof that bind to CD137, or one or more polynucleotides comprising sequences encoding one or more CD137 antibodies or antigen binding fragments thereof that bind to CD137. These compositions can further comprise suitable carriers, such as pharmaceutically acceptable excipients including buffers, which are well known in the art.

Pharmaceutical formulations of an anti-CD137 antibody or antigen binding fragment thereof as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. Sustained-release preparations can be prepared. Suitable examples of sustained-release preparations include semipermeable matrices of solid hydrophobic polymers containing the antibody, which matrices are in the form of shaped articles, e.g. films, or microcapsules. The formulations to be used for in vivo administration are generally sterile. Sterility can be readily accomplished, e.g., by filtration through sterile filtration membranes.

### EXAMPLES

### Example 1. Construction of Synthetic Human VH Antibody Repertoires

Synthetic libraries were constructed essentially using the germline 3-23 (SEQ ID NO: 45 and 46). Randomization of heavy chain CDRs (HCDRs) was carried out by combinatorial mutagenesis using degenerate oligonucleotides (Table 2). Randomization of the HCDR1 and HCDR2 regions was carried out via multiple site-specific mutations by polymerase chain reaction as described by Meetei (Meetei et al., (1998) Anal Biochem, 264, 288-91; Meetei et al., (2002) Methods Mol Biol, 182, 95-102). For CDR3 regions, different lengths from 8 to 14 (Kabat definition) of degenerate oligonucleotides were synthesized (Invitrogen), and diversity was introduced by splice-overlap extension PCR. The PCR products after the mutagenesis steps, were double-digested by NcoI/NotI and ligated into the phagemid vector pCANTAB-5E. Repertoires were then transformed into Escherichia coli TG1 bacteria and validated by DNA Sanger sequencing of random clones (>96 clones analyzed). Phages were purified by two precipitations with PEG/NaCl directly from the culture supernatant after a rescue step using KM13 helper phage.

The libraries were designed to mimic amino-acid distribution commonly observed in the human repertoire, especially in HCDR1 and HCDR2 regions. It has been demonstrated that the introduction of negatively charged amino acids at CDR1 positions not only significantly improved the colloidal stability, but also expression and purification yields of human VH domains (Dudgeon et al., (2013) Protein Eng Des Sel, 26, 671-4; Dudgeon et al., (2012) Proc Natl Acad Sci USA, 109, 10879-84). Thus, two different degenerate oligonucleotides were designed for HCDR1 randomization with a high proportion of negatively charged amino acids. For the diversification of HCDR3, NNY and NNK were used to obtain a maximum degree of repertoire diversity (Table 2). In addition, individual sub libraries with the defined HCDR3 length (Kabat definition) were constructed (Table 3). A library with a total size of 1.38×10¹¹ was obtained after transformation into E. coli bacteria.

TABLE 2
Oligonucleotides used for library construction

| DESIGNATION | SEQ ID NO | SEQUENCE |
|---|---|---|
| HCDR1a | SEQ ID NO: 35 | CTGCGCCGCGTCTGGATTCABNTTBNMCNMCVAH |
| | | GACDTRGGTTGGGTGCGTCAAGCGC |
| HCDR1b | SEQ ID NO: 36 | CTGCGCCGCGTCTGGATTCABNTTBGACNMCNMC |
| | | VAHDTRGGTTGGGTGCGTCAAGCGC |
| HCDR2 | SEQ ID NO: 37 | GGACTGGAATGGGTCTCCRSCATCWBKRRHWBK |
| | | RRHGGTTCGACATACTATGCGG |
| HCDR3-8 | SEQ ID NO: 38 | GGTGTACTACTGCGCCCGCRNCNNKNNKNNKNN |
| | | KNTCGABWWCTGGGGACAGGGCACC |
| HCDR3-9 | SEQ ID NO: 39 | GTGTACTACTGCGCCCGCRNCNNKNNKNNKNNK |
| | | NNKNTCGABWWCTGGGGACAGGGCAC |
| HCDR3-10 | SEQ ID NO: 40 | GTGTACTACTGCGCCCGCRNCNNKNNKNNKNNK |
| | | NNKNNKTTBGATTACTGGGGACAGGG |
| HCDR3-11 | SEQ ID NO: 41 | GTGTACTACTGCGCCCGCRNCNNKNNKNNKNNK |
| | | NNKNNKNNYTTTGATTACTGGGGAC |
| HCDR3-12 | SEQ ID NO: 42 | GTACTACTGCGCCCGCRNCNNKNNKNNKNNKNN |
| | | KNNKNNKNNYTTTGATTACTGGGGAC |
| HCDR3-13 | SEQ ID NO: 43 | GTACTACTGCGCCCGCRNCNNYNNYNNYNNYNNY |
| | | NNYNNYNNYNNYTTTGATTACTGGG |
| HCDR3-14 | SEQ ID NO: 44 | GTACTACTGCGCCCGCRNCNNYNNYNNYNNYNNY |
| | | NNYNNYNNYNNYNNYTTTGATTACTGGGGAC |
| 3-23 germline AA | SEQ ID NO: 45 | EVQLLESGGGLVQPGGSLRLSCAASGFTFSSYAISW |
| | | VRQAPGKGLEWVSAISGSGGSTYYADSVKGRFTISR |
| | | DNSKNTLYLQMNSLRAEDTAVYYCAR |
| 3-23 germline DNA | SEQ ID NO: 46 | GAAGTGCAGCTGCTGGAAAGCGGCGGCGGCCTGG |
| | | TGCAGCCGGGCGGCAGCCTGCGCCTGAGCTGCGC |
| | | GGCGAGCGGCTTTACCTTTAGCAGCTATGCGATTA |
| | | GCTGGGTGCGCCAGGCGCCGGGCAAAGGCCTGGA |
| | | ATGGGTGAGCGCGATTAGCGGCAGCGGCGGTAGC |
| | | ACCTATTATGCGGATAGCGTGAAAGGCCGCTTTAC |
| | | CATTAGCCGCGATAACAGCAAAAACACCCTGTATC |
| | | TGCAGATGAACAGCCTGCGCGCGGAAGATACCGC |
| | | CGTGTATTATTGCGCGCGT |

Diversified positions are shown in boldface type (encoded amino acids (codons in parentheses)): ABN, 25% Ile, 16.67% Arg, Ser, 8.33% Met and 33.33% Thr, TTB, 66.67% Phe and 33.34% Leu; NMC 12.5% Ala, 12.5% Asp, 12.5% His, 12.5% Asn, 12.5% Pro, 12.5% Ser, 12.5% Thr, 12.5% Tyr; VAH, 11.11% Glu, Lys, Gin and 22.22% Asp, Asn, His; DTR, 16.67% Ile, 33.33% Leu, 16.67% Met and 33.33% Val; RSC, 25% Ala, 25% Gly, 25% Ser and 25% Thr; WBK, 8.33% Cys, Phe, Ile, Leu, Met, Arg, Trp, 25% Ser and 16.67% Thr; RRH, 8.33% Glu, Lys, Arg, 16.66% Asp, Gln, Ser and 25% Gly; RNC, 12.5% Ala, 12.5% Asp, 12.5% Gly, 12.5% Ile, 12.5% Gln, 12.5% Arg, 12.5% Ser and 12.5% Thr; NTC, 25% Phe, 25% Leu, 25% Val, 25% Ile; GAB, 66.67% Asp, 33.33% Glu; WWC, 25% Phe, 25% ILe, 25% Gln, 25% Tyr; NNY, 6.25% Ala, Cys, Asp, Phe, Gly, His, ILe, Leu, Gln, Phe, Arg, Thr, Val, Tyr and 12.5% Ser; NNK all 20 AAs.

TABLE 3
Construction of synthetic human VH libraries

| Libraries | Diversity | | | Actual |
|---|---|---|---|---|
| Num. | HCDR1 | HCDR2 | HCDR3 | library size |
| HC-8/9 | 7.68E+03 | 2.27E+03 | 8.19E+08 | 3.00E+10 |
| HC-10 | 7.68E+03 | 2.27E+03 | 1.02E+09 | 2.40E+10 |
| HC-11 | 7.68E+03 | 2.27E+03 | 7.68E+09 | 1.50E+10 |
| HC-12 | 7.68E+03 | 2.27E+03 | 1.54E+11 | 1.50E+10 |
| HC-13 | 7.68E+03 | 2.27E+03 | 3.08E+11 | 3.20E+10 |
| HC-14 | 7.68E+03 | 2.27E+03 | 4.61E+12 | 2.20E+10 |

### Example 2. Generation of Recombinant Proteins and Stable Cell Lines

### CD137 Recombinant Proteins for Phage Campaign and Binding Assays

To discover VH domain antibodies against CD137 with cross-binding of human and Macaca mulatta CD137, but without off-target binding with other human TNF receptor members, several recombinant proteins were designed and expressed for phage panning and screening (see Table 4). The cDNA coding regions for the full-length human CD137 (SEQ ID NO: 47) was ordered based on the CD137 GenBank sequence (Accession No: NM_001561.4, the gene is available from Sinobio, Cat.: HG10041-M). Human CD137 ligand (TNFSF9) (SEQ ID NO:57) was ordered based on (Accession No: NM_003811.3, the gene is available from Sinobio, Cat.: HG15693-G). Monkey (Macaca mulatta) CD137 (SEQ ID NO:63) was ordered based on (Accession No: NM_001266128.1, the gene is available from Genscript, Cat.: OMb00270). The full-length human CD40 (SEQ ID NO: 69) was ordered based on (Accession No: NM_001250.4, the gene is available from Sinobio, Cat.: HG10774-M). OX40 (SEQ ID NO: 75) was ordered based on (Accession No: NM_003327.2, the gene is available from Sinobio, Cat.: HG10481-UT). In brief, the coding region of extracellular domain (ECD) consisting of amino acid (AA) 24-183 of huCD137 (SEQ ID NO: 49), the coding region of ECD consisting of AA 71-254 of human CD137 ligand (SEQ ID NO: 59), the coding region of ECD consisting of AA 24-186 of cynoCD137 (SEQ ID NO: 65), and the coding region of ECD consisting of AA 1-194 of human CD40 (SEQ ID NO: 71) were PCR-amplified. The coding region of mIgG2a Fc (SEQ ID NO: 55) was PCR-amplified, and then conjugated with ECDs of human CD137, human CD137 ligand, monkey CD137 or human CD40 by overlap-PCR to make mIgG2a Fc-fusion proteins. PCR products were then cloned into a pcDNA3.1-based expression vector (Invitrogen, Carlsbad, CA, USA), which resulted in five recombinant mIgG2a Fc-fusion protein expression plasmids, human CD137 ECD-mIgG2a, human CD137 ligand-mIgG2a, cyno CD137 ECD-mIgG2a and human CD40 ECD-mIgG2a. Alternatively the coding regions of ECD consisting of AA 24-183 (SEQ ID NO: 49) of huCD137 (SEQ ID NO: 47) and the coding region of ECD consisting of AA 1-216 of human OX40 (SEQ ID NO: 77) were also cloned into a pcDNA3.1-based expression vector (Invitrogen, Carlsbad, CA, USA) with C-terminus fused with 6×His tags, which resulted in human CD137-his and human OX40-his, respectively. For the recombinant fusion protein production, plasmids were transiently transfected into a HEK293-based mammalian cell expression system (developed in house) and cultured for 5-7 days in a CO₂ incubator equipped with rotating shaker. The supernatants containing the recombinant proteins were collected and cleared by centrifugation. Recombinant proteins were purified using a Protein A column (Cat.: 17127901, GE Life Sciences) or a Ni-NTA agarose (Cat.: R90115, Invitrogen). All recombinant proteins were dialyzed against phosphate buffered saline (PBS) and stored in a −80° C. freezer in small aliquots.

### Stable Expression Cell Lines

To establish stable cell lines that express full-length human CD137 (huCD137), huCD137 sequences were cloned into a retroviral vector pFB-Neo (Cat.: 217561, Agilent, USA). Dual-tropic retroviral vectors were generated according to a previous protocol (Zhang, et al., (2005) Blood, 106, 1544-1551). Vectors containing huCD137 were transduced into Hut78 cells (ATCC, TIB-161) or NK92-mi cells (ATCC, CRL-2408), to generate the huCD137 expressing cell lines, Hut78/huCD137 or NK92-mi/huCD137. huCD137 expressing cell lines were selected by culture in medium containing 10% FBS with G418, and then verified via FACS.

TABLE 4
Sequences for recombinant CD137 proteins

| | SEQ ID | |
|---|---|---|
| CONSTRUCT | NO: | SEQUENCE |
| human CD137 | SEQ ID | MGNSCYNIVATLLLVLNFERTRSLQDPCSNCPAGTFCD |
| FL AA | NO: 47 | NNRNQICSPCPPNSFSSAGGQRTCDICRQCKGVFRTRK |
| | | ECSSTSNAECDCTPGFHCLGAGCSMCEQDCKQGQELT |
| | | KKGCKDCCFGTFNDQKRGICRPWTNCSLDGKSVLVN |
| | | GTKERDVVCGPSPADLSPGASSVTPPAPAREPGHSPQII |
| | | SFFLALTSTALLFLLFFLTLRFSVVKRGRKKLLYIFKQPF |
| | | MRPVQTTQEEDGCSCRFPEEEEGGCEL |
| human CD137 | SEQ ID | ATGGGAAACAGCTGTTACAACATAGTAGCCACTCTG |
| FL DNA | NO: 48 | TTGCTGGTCCTCAACTTTGAGAGGACAAGATCATTG |
| | | CAGGATCCTTGTAGTAACTGCCCAGCTGGTACATTCT |
| | | GTGATAATAACAGGAATCAGATTTGCAGTCCCTGTCC |
| | | TCCAAATAGTTTCTCCAGCGCAGGTGGACAAAGGAC |
| | | CTGTGACATATGCAGGCAGTGTAAAGGTGTTTTCAG |
| | | GACCAGGAAGGAGTGTTCCTCCACCAGCAATGCAG |
| | | AGTGTGACTGCACTCCAGGGTTTCACTGCCTGGGGG |
| | | CAGGATGCAGCATGTGTGAACAGGATTGTAAACAAG |
| | | GTCAAGAACTGACAAAAAAAGGTTGTAAAGACTGT |
| | | TGCTTTGGGACATTTAACGATCAGAAACGTGGCATC |
| | | TGTCGACCCTGGACAAACTGTTCTTTGGATGGAAAG |
| | | TCTGTGCTTGTGAATGGGACGAAGGAGAGGGACGT |
| | | GGTCTGTGGACCATCTCCAGCCGACCTCTCTCCGGG |
| | | AGCATCCTCTGTGACCCCGCCTGCCCCTGCGAGAGA |
| | | GCCAGGACACTCTCCGCAGATCATCTCCTTCTTTCTT |
| | | GCGCTGACGTCGACTGCGTTGCTCTTCCTGCTGTTC |
| | | TTCCTCACGCTCCGTTTCTCTGTTGTTAAACGGGGC |
| | | AGAAAGAAACTCCTGTATATATTCAAACAACCATTTA |
| | | TGAGACCAGTACAAACTACTCAAGAGGAAGATGGC |
| | | TGTAGCTGCCGATTTCCAGAAGAAGAAGAAGGAGG |
| | | ATGTGAACTG |
| human CD137 | SEQ ID | LQDPCSNCPAGTFCDNNRNQICSPCPPNSFSSAGGQRT |
| AA 24-183 | NO: 49 | CDICRQCKGVFRTRKECSSTSNAECDCTPGFHCLGAG |
| | | CSMCEQDCKQGQELTKKGCKDCCFGTFNDQKRGICR |
| | | PWTNCSLDGKSVLVNGTKERDVVCGPSPADLSPGASS |
| | | VTPPAPAREPGH |
| human CD137 | SEQ ID | CTGCAGGATCCTTGTAGTAACTGCCCAGCTGGTACA |
| 24-183 DNA | NO: 50 | TTCTGTGATAATAACAGGAATCAGATTTGCAGTCCCT |
| | | GTCCTCCAAATAGTTTCTCCAGCGCAGGTGGACAAA |
| | | GGACCTGTGACATATGCAGGCAGTGTAAAGGTGTTT |
| | | TCAGGACCAGGAAGGAGTGTTCCTCCACCAGCAAT |
| | | GCAGAGTGTGACTGCACTCCAGGGTTTCACTGCCTG |
| | | GGGGCAGGATGCAGCATGTGTGAACAGGATTGTAA |
| | | ACAAGGTCAAGAACTGACAAAAAAAGGTTGTAAAG |
| | | ACTGTTGCTTTGGGACATTTAACGATCAGAAACGTG |
| | | GCATCTGTCGACCCTGGACAAACTGTTCTTTGGATG |
| | | GAAAGTCTGTGCTTGTGAATGGGACGAAGGAGAGG |
| | | GACGTGGTCTGTGGACCATCTCCAGCCGACCTCTCT |
| | | CCGGGAGCATCCTCTGTGACCCCGCCTGCCCCTGCG |
| | | AGAGAGCCAGGACAC |
| human CD137- | SEQ ID | LQDPCSNCPAGTFCDNNRNQICSPCPPNSFSSAGGQRT |
| his | NO: 51 | CDICRQCKGVFRTRKECSSTSNAECDCTPGFHCLGAG |
| | | CSMCEQDCKQGQELTKKGCKDCCFGTFNDQKRGICR |
| | | PWTNCSLDGKSVLVNGTKERDVVCGPSPADLSPGASS |
| | | VTPPAPAREPGHHHHHHHGGGLNDIFEAQKIEWHE |
| human CD137- | SEQ ID | CTGCAGGATCCTTGTAGTAACTGCCCAGCTGGTACA |
| his DNA | NO: 52 | TTCTGTGATAATAACAGGAATCAGATTTGCAGTCCCT |
| | | GTCCTCCAAATAGTTTCTCCAGCGCAGGTGGACAAA |
| | | GGACCTGTGACATATGCAGGCAGTGTAAAGGTGTTT |
| | | TCAGGACCAGGAAGGAGTGTTCCTCCACCAGCAAT |
| | | GCAGAGTGTGACTGCACTCCAGGGTTTCACTGCCTG |
| | | GGGGCAGGATGCAGCATGTGTGAACAGGATTGTAA |
| | | ACAAGGTCAAGAACTGACAAAAAAAGGTTGTAAAG |
| | | ACTGTTGCTTTGGGACATTTAACGATCAGAAACGTG |
| | | GCATCTGTCGACCCTGGACAAACTGTTCTTTGGATG |
| | | GAAAGTCTGTGCTTGTGAATGGGACGAAGGAGAGG |
| | | GACGTGGTCTGTGGACCATCTCCAGCCGACCTCTCT |
| | | CCGGGAGCATCCTCTGTGACCCCGCCTGCCCCTGCG |
| | | AGAGAGCCAGGACACCATCACCATCACCATCACGG |
| | | AGGCGGTCTGAACGACATCTTCGAGGCTCAGAAAAT |
| | | CGAATGGCACGAA |
| human CD137 | SEQ ID | LQDPCSNCPAGTFCDNNRNQICSPCPPNSFSSAGGQRT |
| ECD-mIgG2a | NO: 53 | CDICRQCKGVFRTRKECSSTSNAECDCTPGFHCLGAG |
| AA | | CSMCEQDCKQGQELTKKGCKDCCFGTFNDQKRGICR |
| | | PWTNCSLDGKSVLVNGTKERDVVCGPSPADLSPGASS |
| | | VTPPAPAREPGHEPRGPTIKPCPPCKCPAPNLLGGPSVFI |
| | | FPPKIKDVLMISLSPIVTCVVVDVSEDDPDVQISWFVN |
| | | NVEVHTAQTQTHREDYNSTLRVVSALPIQHQDWMSG |
| | | KEFKCKVNNKDLPAPIERTISKPKGSVRAPQVYVLPPP |
| | | EEEMTKKQVTLTCMVTDFMPEDIYVEWTNNGKTELN |
| | | YKNTEPVLDSDGSYFMYSKLRVEKKNWVERNSYSCS |
| | | VVHEGLHNHHTTKSFSRTPGK |
| human CD137 | SEQ ID | CTGCAGGATCCTTGTAGTAACTGCCCAGCTGGTACA |
| ECD-mIgG2a | NO: 54 | TTCTGTGATAATAACAGGAATCAGATTTGCAGTCCCT |
| DNA | | GTCCTCCAAATAGTTTCTCCAGCGCAGGTGGACAAA |
| | | GGACCTGTGACATATGCAGGCAGTGTAAAGGTGTTT |
| | | TCAGGACCAGGAAGGAGTGTTCCTCCACCAGCAAT |
| | | GCAGAGTGTGACTGCACTCCAGGGTTTCACTGCCTG |
| | | GGGGCAGGATGCAGCATGTGTGAACAGGATTGTAA |
| | | ACAAGGTCAAGAACTGACAAAAAAAGGTTGTAAAG |
| | | ACTGTTGCTTTGGGACATTTAACGATCAGAAACGTG |
| | | GCATCTGTCGACCCTGGACAAACTGTTCTTTGGATG |
| | | GAAAGTCTGTGCTTGTGAATGGGACGAAGGAGAGG |
| | | GACGTGGTCTGTGGACCATCTCCAGCCGACCTCTCT |
| | | CCGGGAGCATCCTCTGTGACCCCGCCTGCCCCTGCG |
| | | AGAGAGCCAGGACACGAGCCCAGAGGGCCCACAAT |
| | | CAAGCCCTGTCCTCCATGCAAATGCCCAGCACCTAA |
| | | CCTCTTGGGTGGACCATCCGTCTTCATCTTCCCTCCA |
| | | AAGATCAAGGATGTACTCATGATCTCCCTGAGCCCC |
| | | ATAGTCACATGTGTGGTGGTGGATGTGAGCGAGGAT |
| | | GACCCAGATGTCCAGATCAGCTGGTTTGTGAACAAC |
| | | GTGGAAGTACACACAGCTCAGACACAAACCCATAG |
| | | AGAGGATTACAACAGTACTCTCCGGGTGGTCAGTGC |
| | | CCTCCCCATCCAGCACCAGGACTGGATGAGTGGCAA |
| | | GGAGTTCAAATGCAAGGTCAACAACAAAGACCTCC |
| | | CAGCGCCCATCGAGAGAACCATCTCAAAACCCAAA |
| | | GGGTCAGTAAGAGCTCCACAGGTATATGTCTTGCCT |
| | | CCACCAGAAGAAGAGATGACTAAGAAACAGGTCAC |
| | | TCTGACCTGCATGGTCACAGACTTCATGCCTGAAGA |
| | | CATTTACGTGGAGTGGACCAACAACGGGAAAACAG |
| | | AGCTAAACTACAAGAACACTGAACCAGTCCTGGAC |
| | | TCTGATGGTTCTTACTTCATGTACAGCAAGCTGAGA |
| | | GTGGAAAAGAAGAACTGGGTGGAAAGAAATAGCTA |
| | | CTCCTGTTCAGTGGTCCACGAGGGTCTGCACAATCA |
| | | CCACACGACTAAGAGCTTCTCCCGGACTCCGGGTAA |
| | | A |
| mIgG2a AA | SEQ ID | EPRGPTIKPCPPCKCPAPNLLGGPSVFIFPPKIKDVLMIS |
| | NO: 55 | LSPIVTCVVVDVSEDDPDVQISWFVNNVEVHTAQTQT |
| | | HREDYNSTLRVVSALPIQHQDWMSGKEFKCKVNNKD |
| | | LPAPIERTISKPKGSVRAPQVYVLPPPEEEMTKKQVTLT |
| | | CMVTDFMPEDIYVEWTNNGKTELNYKNTEPVLDSDG |
| | | SYFMYSKLRVEKKNWVERNSYSCSVVHEGLHNHHTT |
| | | KSFSRTPGK |
| mIgG2a DNA | SEQ ID | GAGCCCAGAGGGCCCACAATCAAGCCCTGTCCTCC |
| | NO: 56 | ATGCAAATGCCCAGCACCTAACCTCTTGGGTGGACC |
| | | ATCCGTCTTCATCTTCCCTCCAAAGATCAAGGATGTA |
| | | CTCATGATCTCCCTGAGCCCCATAGTCACATGTGTGG |
| | | TGGTGGATGTGAGCGAGGATGACCCAGATGTCCAGA |
| | | TCAGCTGGTTTGTGAACAACGTGGAAGTACACACA |
| | | GCTCAGACACAAACCCATAGAGAGGATTACAACAGT |
| | | ACTCTCCGGGTGGTCAGTGCCCTCCCCATCCAGCAC |
| | | CAGGACTGGATGAGTGGCAAGGAGTTCAAATGCAA |
| | | GGTCAACAACAAAGACCTCCCAGCGCCCATCGAGA |
| | | GAACCATCTCAAAACCCAAAGGGTCAGTAAGAGCT |
| | | CCACAGGTATATGTCTTGCCTCCACCAGAAGAAGAG |
| | | ATGACTAAGAAACAGGTCACTCTGACCTGCATGGTC |
| | | ACAGACTTCATGCCTGAAGACATTTACGTGGAGTGG |
| | | ACCAACAACGGGAAAACAGAGCTAAACTACAAGAA |
| | | CACTGAACCAGTCCTGGACTCTGATGGTTCTTACTT |
| | | CATGTACAGCAAGCTGAGAGTGGAAAAGAAGAACT |
| | | GGGTGGAAAGAAATAGCTACTCCTGTTCAGTGGTCC |
| | | ACGAGGGTCTGCACAATCACCACACGACTAAGAGC |
| | | TTCTCCCGGACTCCGGGTAAA |
| human CD137 | SEQ ID | MEYASDASLDPEAPWPPAPRARACRVLPWALVAGLLL |
| ligand AA | NO: 57 | LLLLAAACAVFLACPWAVSGARASPGSAASPRLREGP |
| | | ELSPDDPAGLLDLRQGMFAQLVAQNVLLIDGPLSWYS |
| | | DPGLAGVSLTGGLSYKEDTKELVVAKAGVYYVFFQLE |
| | | LRRVVAGEGSGSVSLALHLQPLRSAAGAAALALTVDL |
| | | PPASSEARNSAFGFQGRLLHLSAGQRLGVHLHTEARA |
| | | RHAWQLTQGATVLGLFRVTPEIPAGLPSPRSE |
| human CD137 | SEQ ID | ATGGAATACGCCTCTGACGCTTCACTGGACCCCGAA |
| ligand cDNA | NO: 58 | GCCCCGTGGCCTCCCGCGCCCCGCGCTCGCGCCTGC |
| | | CGCGTACTGCCTTGGGCCCTGGTCGCGGGGCTGCTG |
| | | CTGCTGCTGCTGCTCGCTGCCGCCTGCGCCGTCTTC |
| | | CTCGCCTGCCCCTGGGCCGTGTCCGGGGCTCGCGCC |
| | | TCGCCCGGCTCCGCGGCCAGCCCGAGACTCCGCGA |
| | | GGGTCCCGAGCTTTCGCCCGACGATCCCGCCGGCCT |
| | | CTTGGACCTGCGGCAGGGCATGTTTGCGCAGCTGGT |
| | | GGCCCAAAATGTTCTGCTGATCGATGGGCCCCTGAG |
| | | CTGGTACAGTGACCCAGGCCTGGCAGGCGTGTCCCT |
| | | GACGGGGGGCCTGAGCTACAAAGAGGACACGAAG |
| | | GAGCTGGTGGTGGCCAAGGCTGGAGTCTACTATGTC |
| | | TTCTTTCAACTAGAGCTGCGGCGCGTGGTGGCCGGC |
| | | GAGGGCTCAGGCTCCGTTTCACTTGCGCTGCACCTG |
| | | CAGCCACTGCGCTCTGCTGCTGGGGCCGCCGCCCTG |
| | | GCTTTGACCGTGGACCTGCCACCCGCCTCCTCCGAG |
| | | GCTCGGAACTCGGCCTTCGGTTTCCAGGGCCGCTTG |
| | | CTGCACCTGAGTGCCGGCCAGCGCCTGGGCGTCCAT |
| | | CTTCACACTGAGGCCAGGGCACGCCATGCCTGGCA |
| | | GCTTACCCAGGGCGCCACAGTCTTGGGACTCTTCCG |
| | | GGTGACCCCCGAAATCCCAGCCGGACTCCCTTCACC |
| | | GAGGTCGGAATAA |
| human CD137 | SEQ ID | REGPELSPDDPAGLLDLRQGMFAQLVAQNVLLIDGPLS |
| ligand 71-254 | NO: 59 | WYSDPGLAGVSLTGGLSYKEDTKELVVAKAGVYYVF |
| AA | | FQLELRRVVAGEGSGSVSLALHLQPLRSAAGAAALAL |
| | | TVDLPPASSEARNSAFGFQGRLLHLSAGQRLGVHLHT |
| | | EARARHAWQLTQGATVLGLFRVTPEIPAGLPSPRSE |
| human CD137 | SEQ ID | CGCGAGGGTCCCGAGCTTTCGCCCGACGATCCCGCC |
| ligand 71-254 | NO: 60 | GGCCTCTTGGACCTGCGGCAGGGCATGTTTGCGCAG |
| DNA | | CTGGTGGCCCAAAATGTTCTGCTGATCGATGGGCCC |
| | | CTGAGCTGGTACAGTGACCCAGGCCTGGCAGGCGT |
| | | GTCCCTGACGGGGGGCCTGAGCTACAAAGAGGACA |
| | | CGAAGGAGCTGGTGGTGGCCAAGGCTGGAGTCTAC |
| | | TATGTCTTCTTTCAACTAGAGCTGCGGCGCGTGGTG |
| | | GCCGGCGAGGGCTCAGGCTCCGTTTCACTTGCGCTG |
| | | CACCTGCAGCCACTGCGCTCTGCTGCTGGGGCCGCC |
| | | GCCCTGGCTTTGACCGTGGACCTGCCACCCGCCTCC |
| | | TCCGAGGCTCGGAACTCGGCCTTCGGTTTCCAGGGC |
| | | CGCTTGCTGCACCTGAGTGCCGGCCAGCGCCTGGGC |
| | | GTCCATCTTCACACTGAGGCCAGGGCACGCCATGCC |
| | | TGGCAGCTTACCCAGGGCGCCACAGTCTTGGGACTC |
| | | TTCCGGGTGACCCCCGAAATCCCAGCCGGACTCCCT |
| | | TCACCGAGGTCGGAA |
| human CD137 | SEQ ID | EPRGPTIKPCPPCKCPAPNLLGGPSVFIFPPKIKDVLMIS |
| ligand-mIgG2a | NO: 61 | LSPIVTCVVVDVSEDDPDVQISWFVNNVEVHTAQTQT |
| AA | | HREDYNSTLRVVSALPIQHQDWMSGKEFKCKVNNKD |
| | | LPAPIERTISKPKGSVRAPQVYVLPPPEEEMTKKQVTLT |
| | | CMVTDFMPEDIYVEWTNNGKTELNYKNTEPVLDSDG |
| | | SYFMYSKLRVEKKNWVERNSYSCSVVHEGLHNHHTT |
| | | KSFSRTPGKGGGGSREGPELSPDDPAGLLDLRQGMFA |
| | | QLVAQNVLLIDGPLSWYSDPGLAGVSLTGGLSYKEDT |
| | | KELVVAKAGVYYVFFQLELRRVVAGEGSGSVSLALHL |
| | | QPLRSAAGAAALALTVDLPPASSEARNSAFGFQGRLL |
| | | HLSAGQRLGVHLHTEARARHAWQLTQGATVLGLFRV |
| | | TPEIPAGLPSPRSE |
| human CD137 | SEQ ID | GAGCCCAGAGGGCCCACAATCAAGCCCTGTCCTCC |
| ligand-mIgG2a | NO: 62 | ATGCAAATGCCCAGCACCTAACCTCTTGGGTGGACC |
| DNA | | ATCCGTCTTCATCTTCCCTCCAAAGATCAAGGATGTA |
| | | CTCATGATCTCCCTGAGCCCCATAGTCACATGTGTGG |
| | | TGGTGGATGTGAGCGAGGATGACCCAGATGTCCAGA |
| | | TCAGCTGGTTTGTGAACAACGTGGAAGTACACACA |
| | | GCTCAGACACAAACCCATAGAGAGGATTACAACAGT |
| | | ACTCTCCGGGTGGTCAGTGCCCTCCCCATCCAGCAC |
| | | CAGGACTGGATGAGTGGCAAGGAGTTCAAATGCAA |
| | | GGTCAACAACAAAGACCTCCCAGCGCCCATCGAGA |
| | | GAACCATCTCAAAACCCAAAGGGTCAGTAAGAGCT |
| | | CCACAGGTATATGTCTTGCCTCCACCAGAAGAAGAG |
| | | ATGACTAAGAAACAGGTCACTCTGACCTGCATGGTC |
| | | ACAGACTTCATGCCTGAAGACATTTACGTGGAGTGG |
| | | ACCAACAACGGGAAAACAGAGCTAAACTACAAGAA |
| | | CACTGAACCAGTCCTGGACTCTGATGGTTCTTACTT |
| | | CATGTACAGCAAGCTGAGAGTGGAAAAGAAGAACT |
| | | GGGTGGAAAGAAATAGCTACTCCTGTTCAGTGGTCC |
| | | ACGAGGGTCTGCACAATCACCACACGACTAAGAGC |
| | | TTCTCCCGGACTCCGGGTAAAGGTGGAGGCGGTTCA |
| | | CGCGAGGGTCCCGAGCTTTCGCCCGACGATCCCGCC |
| | | GGCCTCTTGGACCTGCGGCAGGGCATGTTTGCGCAG |
| | | CTGGTGGCCCAAAATGTTCTGCTGATCGATGGGCCC |
| | | CTGAGCTGGTACAGTGACCCAGGCCTGGCAGGCGT |
| | | GTCCCTGACGGGGGGCCTGAGCTACAAAGAGGACA |
| | | CGAAGGAGCTGGTGGTGGCCAAGGCTGGAGTCTAC |
| | | TATGTCTTCTTTCAACTAGAGCTGCGGCGCGTGGTG |
| | | GCCGGCGAGGGCTCAGGCTCCGTTTCACTTGCGCTG |
| | | CACCTGCAGCCACTGCGCTCTGCTGCTGGGGCCGCC |
| | | GCCCTGGCTTTGACCGTGGACCTGCCACCCGCCTCC |
| | | TCCGAGGCTCGGAACTCGGCCTTCGGTTTCCAGGGC |
| | | CGCTTGCTGCACCTGAGTGCCGGCCAGCGCCTGGGC |
| | | GTCCATCTTCACACTGAGGCCAGGGCACGCCATGCC |
| | | TGGCAGCTTACCCAGGGCGCCACAGTCTTGGGACTC |
| | | TTCCGGGTGACCCCCGAAATCCCAGCCGGACTCCCT |
| | | TCACCGAGGTCGGAA |
| cyno CD137 FL | SEQ ID | MGNSCYNIVATLLLVLNFERTRSLQDLCSNCPAGTFCD |
| AA | NO: 63 | NNRSQICSPCPPNSFSSAGGQRTCDICRQCKGVFKTRK |
| | | ECSSTSNAECDCISGYHCLGAECSMCEQDCKQGQELT |
| | | KKGCKDCCFGTFNDQKRGICRPWTNCSLDGKSVLVN |
| | | GTKERDVVCGPSPADLSPGASSATPPAPAREPGHSPQII |
| | | FFLALTSTVVLFLLFFLVLRFSVVKRSRKKLLYIFKQPF |
| | | MRPVQTTQEEDGCSCRFPEEEEGGCEL |
| cyno CD137 FL | SEQ ID | ATGGGAAACAGCTGTTACAACATAGTAGCCACTCTG |
| DNA | NO: 64 | TTGCTGGTCCTGAACTTTGAGAGGACAAGATCCTTG |
| | | CAGGATCTGTGTAGTAACTGCCCAGCTGGTACATTCT |
| | | GTGATAATAACAGGAGTCAGATTTGCAGTCCCTGTC |
| | | CTCCAAATAGTTTCTCCAGCGCAGGTGGACAAAGGA |
| | | CCTGTGACATATGCAGGCAGTGTAAAGGTGTTTTCA |
| | | AGACCAGGAAGGAGTGTTCCTCCACCAGCAATGCA |
| | | GAGTGTGACTGCATTTCAGGATATCACTGCCTGGGG |
| | | GCAGAATGCAGCATGTGTGAACAGGATTGTAAACAA |
| | | GGTCAAGAATTGACAAAAAAAGGTTGTAAAGACTG |
| | | TTGCTTTGGGACATTTAATGACCAGAAACGTGGCAT |
| | | CTGTCGACCCTGGACAAACTGTTCTTTGGATGGAAA |
| | | GTCTGTGCTTGTGAATGGGACGAAGGAGAGGGACG |
| | | TGGTCTGCGGACCATCTCCAGCCGACCTCTCTCCAG |
| | | GAGCATCCTCTGCGACCCCGCCTGCCCCTGCGAGAG |
| | | AGCCAGGACACTCTCCGCAGATCATCTTCTTTCTTGC |
| | | GCTGACTTCGACTGTGGTACTCTTCCTGCTGTTCTTC |
| | | CTCGTGCTCCGTTTCTCTGTTGTTAAACGGAGCAGA |
| | | AAGAAACTCCTGTATATATTCAAACAACCATTTATGA |
| | | GACCGGTACAAACCACTCAAGAGGAAGATGGATGT |
| | | AGCTGCCGATTTCCAGAAGAAGAAGAAGGAGGATG |
| | | TGAACTGTGA |
| cyno CD137 | SEQ ID | LQDLCSNCPAGTFCDNNRSQICSPCPPNSFSSAGGQRT |
| ECD 24-186 | NO: 65 | CDICRQCKGVFKTRKECSSTSNAECDCISGYHCLGAE |
| AA | | CSMCEQDCKQGQELTKKGCKDCCFGTFNDQKRGICR |
| | | PWTNCSLDGKSVLVNGTKERDVVCGPSPADLSPGASS |
| | | ATPPAPAREPGHSPQ |
| cyno CD137 | SEQ ID | CTGCAGGATCTGTGTAGTAACTGCCCAGCTGGTACA |
| ECD 24-186 | NO: 66 | TTCTGTGATAATAACAGGAGTCAGATTTGCAGTCCCT |
| DNA | | GTCCTCCAAATAGTTTCTCCAGCGCAGGTGGACAAA |
| | | GGACCTGTGACATATGCAGGCAGTGTAAAGGTGTTT |
| | | TCAAGACCAGGAAGGAGTGTTCCTCCACCAGCAAT |
| | | GCAGAGTGTGACTGCATTTCAGGATATCACTGCCTG |
| | | GGGGCAGAATGCAGCATGTGTGAACAGGATTGTAA |
| | | ACAAGGTCAAGAATTGACAAAAAAAGGTTGTAAAG |
| | | ACTGTTGCTTTGGGACATTTAATGACCAGAAACGTG |
| | | GCATCTGTCGACCCTGGACAAACTGTTCTTTGGATG |
| | | GAAAGTCTGTGCTTGTGAATGGGACGAAGGAGAGG |
| | | GACGTGGTCTGCGGACCATCTCCAGCCGACCTCTCT |
| | | CCAGGAGCATCCTCTGCGACCCCGCCTGCCCCTGCG |
| | | AGAGAGCCAGGACACTCTCCGCAG |
| cyno CD137 | SEQ ID | LQDLCSNCPAGTFCDNNRSQICSPCPPNSFSSAGGQRT |
| ECD-mIgG2a | NO: 67 | CDICRQCKGVFKTRKECSSTSNAECDCISGYHCLGAE |
| AA | | CSMCEQDCKQGQELTKKGCKDCCFGTFNDQKRGICR |
| | | PWTNCSLDGKSVLVNGTKERDVVCGPSPADLSPGASS |
| | | ATPPAPAREPGHSPQEPRGPTIKPCPPCKCPAPNLLGGP |
| | | SVFIFPPKIKDVLMISLSPIVTCVVVDVSEDDPDVQISW |
| | | FVNNVEVHTAQTQTHREDYNSTLRVVSALPIQHQDW |
| | | MSGKEFKCKVNNKDLPAPIERTISKPKGSVRAPQVYV |
| | | LPPPEEEMTKKQVTLTCMVTDFMPEDIYVEWTNNGK |
| | | TELNYKNTEPVLDSDGSYFMYSKLRVEKKNWVERNS |
| | | YSCSVVHEGLHNHHTTKSFSRTPGK |
| cyno CD137 | SEQ ID | CTGCAGGATCTGTGTAGTAACTGCCCAGCTGGTACA |
| ECD-mIgG2a | NO: 68 | TTCTGTGATAATAACAGGAGTCAGATTTGCAGTCCCT |
| DNA | | GTCCTCCAAATAGTTTCTCCAGCGCAGGTGGACAAA |
| | | GGACCTGTGACATATGCAGGCAGTGTAAAGGTGTTT |
| | | TCAAGACCAGGAAGGAGTGTTCCTCCACCAGCAAT |
| | | GCAGAGTGTGACTGCATTTCAGGATATCACTGCCTG |
| | | GGGGCAGAATGCAGCATGTGTGAACAGGATTGTAA |
| | | ACAAGGTCAAGAATTGACAAAAAAAGGTTGTAAAG |
| | | ACTGTTGCTTTGGGACATTTAATGACCAGAAACGTG |
| | | GCATCTGTCGACCCTGGACAAACTGTTCTTTGGATG |
| | | GAAAGTCTGTGCTTGTGAATGGGACGAAGGAGAGG |
| | | GACGTGGTCTGCGGACCATCTCCAGCCGACCTCTCT |
| | | CCAGGAGCATCCTCTGCGACCCCGCCTGCCCCTGCG |
| | | AGAGAGCCAGGACACTCTCCGCAGGAGCCCAGAGG |
| | | GCCCACAATCAAGCCCTGTCCTCCATGCAAATGCCC |
| | | AGCACCTAACCTCTTGGGTGGACCATCCGTCTTCAT |
| | | CTTCCCTCCAAAGATCAAGGATGTACTCATGATCTCC |
| | | CTGAGCCCCATAGTCACATGTGTGGTGGTGGATGTG |
| | | AGCGAGGATGACCCAGATGTCCAGATCAGCTGGTTT |
| | | GTGAACAACGTGGAAGTACACACAGCTCAGACACA |
| | | AACCCATAGAGAGGATTACAACAGTACTCTCCGGGT |
| | | GGTCAGTGCCCTCCCCATCCAGCACCAGGACTGGAT |
| | | GAGTGGCAAGGAGTTCAAATGCAAGGTCAACAACA |
| | | AAGACCTCCCAGCGCCCATCGAGAGAACCATCTCAA |
| | | AACCCAAAGGGTCAGTAAGAGCTCCACAGGTATATG |
| | | TCTTGCCTCCACCAGAAGAAGAGATGACTAAGAAA |
| | | CAGGTCACTCTGACCTGCATGGTCACAGACTTCATG |
| | | CCTGAAGACATTTACGTGGAGTGGACCAACAACGG |
| | | GAAAACAGAGCTAAACTACAAGAACACTGAACCAG |
| | | TCCTGGACTCTGATGGTTCTTACTTCATGTACAGCAA |
| | | GCTGAGAGTGGAAAAGAAGAACTGGGTGGAAAGA |
| | | AATAGCTACTCCTGTTCAGTGGTCCACGAGGGTCTG |
| | | CACAATCACCACACGACTAAGAGCTTCTCCCGGACT |
| | | CCGGGTAAA |
| human CD40 | SEQ ID | MVRLPLQCVLWGCLLTAVHPEPPTACREKQYLINSQC |
| AA | NO: 69 | CSLCQPGQKLVSDCTEFTETECLPCGESEFLDTWNRET |
| | | HCHQHKYCDPNLGLRVQQKGTSETDTICTCEEGWHC |
| | | TSEACESCVLHRSCSPGFGVKQIATGVSDTICEPCPVGF |
| | | FSNVSSAFEKCHPWTSCETKDLVVQQAGTNKTDVVC |
| | | GPQDRLRALVVIPIIFGILFAILLVLVFIKKVAKKPTNKA |
| | | PHPKQEPQEINFPDDLPGSNTAAPVQETLHGCQPVTQE |
| | | DGKESRISVQERQ |
| human CD40 | SEQ ID | ATGGTTCGTCTGCCTCTGCAGTGCGTCCTCTGGGGC |
| DNA | NO: 70 | TGCTTGCTGACCGCTGTCCATCCAGAACCACCCACT |
| | | GCATGCAGAGAAAAACAGTACCTAATAAACAGTCA |
| | | GTGCTGTTCTTTGTGCCAGCCAGGACAGAAACTGGT |
| | | GAGTGACTGCACAGAGTTCACTGAAACGGAATGCC |
| | | TTCCTTGCGGTGAAAGCGAATTCCTAGACACCTGGA |
| | | ACAGAGAGACACACTGCCACCAGCACAAATACTGC |
| | | GACCCCAACCTAGGGCTTCGGGTCCAGCAGAAGGG |
| | | CACCTCAGAAACAGACACCATCTGCACCTGTGAAG |
| | | AAGGCTGGCACTGTACGAGTGAGGCCTGTGAGAGC |
| | | TGTGTCCTGCACCGCTCATGCTCGCCCGGCTTTGGG |
| | | GTCAAGCAGATTGCTACAGGGGTTTCTGATACCATCT |
| | | GCGAGCCCTGCCCAGTCGGCTTCTTCTCCAATGTGT |
| | | CATCTGCTTTCGAAAAATGTCACCCTTGGACAAGCT |
| | | GTGAGACCAAAGACCTGGTTGTGCAACAGGCAGGC |
| | | ACAAACAAGACTGATGTTGTCTGTGGTCCCCAGGAT |
| | | CGGCTGAGAGCCCTGGTGGTGATCCCCATCATCTTC |
| | | GGGATCCTGTTTGCCATCCTCTTGGTGCTGGTCTTTA |
| | | TCAAAAAGGTGGCCAAGAAGCCAACCAATAAGGCC |
| | | CCCCACCCCAAGCAGGAACCCCAGGAGATCAATTTT |
| | | CCCGACGATCTTCCTGGCTCCAACACTGCTGCTCCA |
| | | GTGCAGGAGACTTTACATGGATGCCAACCGGTCACC |
| | | CAGGAGGATGGCAAAGAGAGTCGCATCTCAGTGCA |
| | | GGAGAGACAGTGA |
| huCD40 ECD | SEQ ID | MVRLPLQCVLWGCLLTAVHPEPPTACREKQYLINSQC |
| AA (1-194) | NO: 71 | CSLCQPGQKLVSDCTEFTETECLPCGESEFLDTWNRET |
| | | HCHQHKYCDPNLGLRVQQKGTSETDTICTCEEGWHC |
| | | TSEACESCVLHRSCSPGFGVKQIATGVSDTICEPCPVGF |
| | | FSNVSSAFEKCHPWTSCETKDLVVQQAGTNKTDVVC |
| | | GPQDRLRA |
| huCD40 ECD | SEQ ID | ATGGTTCGTCTGCCTCTGCAGTGCGTCCTCTGGGGC |
| DNA (1-194) | NO: 72 | TGCTTGCTGACCGCTGTCCATCCAGAACCACCCACT |
| | | GCATGCAGAGAAAAACAGTACCTAATAAACAGTCA |
| | | GTGCTGTTCTTTGTGCCAGCCAGGACAGAAACTGGT |
| | | GAGTGACTGCACAGAGTTCACTGAAACGGAATGCC |
| | | TTCCTTGCGGTGAAAGCGAATTCCTAGACACCTGGA |
| | | ACAGAGAGACACACTGCCACCAGCACAAATACTGC |
| | | GACCCCAACCTAGGGCTTCGGGTCCAGCAGAAGGG |
| | | CACCTCAGAAACAGACACCATCTGCACCTGTGAAG |
| | | AAGGCTGGCACTGTACGAGTGAGGCCTGTGAGAGC |
| | | TGTGTCCTGCACCGCTCATGCTCGCCCGGCTTTGGG |
| | | GTCAAGCAGATTGCTACAGGGGTTTCTGATACCATCT |
| | | GCGAGCCCTGCCCAGTCGGCTTCTTCTCCAATGTGT |
| | | CATCTGCTTTCGAAAAATGTCACCCTTGGACAAGCT |
| | | GTGAGACCAAAGACCTGGTTGTGCAACAGGCAGGC |
| | | ACAAACAAGACTGATGTTGTCTGTGGTCCCCAGGAT |
| | | CGGCTGAGAGCC |
| human CD40 | SEQ ID | MVRLPLQCVLWGCLLTAVHPEPPTACREKQYLINSQC |
| ECD-mIgG2a | NO: 73 | CSLCQPGQKLVSDCTEFTETECLPCGESEFLDTWNRET |
| AA | | HCHQHKYCDPNLGLRVQQKGTSETDTICTCEEGWHC |
| | | TSEACESCVLHRSCSPGFGVKQIATGVSDTICEPCPVGF |
| | | FSNVSSAFEKCHPWTSCETKDLVVQQAGTNKTDVVC |
| | | GPQDRLRASEPRGPTIKPCPPCKCPAPNLLGGPSVFIFP |
| | | PKIKDVLMISLSPMVTCVVVDVSEDDPDVQISWFVNN |
| | | VEVLTAQTQTHREDYNSTLRVVSALPIQHQDWMSGK |
| | | EFKCKVNNKALPAPIERTISKPKGSVRAPQVYVLPPPE |
| | | EEMTKKQVTLTCMVTDFMPEDIYVEWTNNGKTELNY |
| | | KNTEPVLDSDGSYFMYSKLRVEKKNWVERNSYSCSV |
| | | VHEGLHNHHTTKSFSRTPGK |
| human CD40 | SEQ ID | ATGGTTCGTCTGCCTCTGCAGTGCGTCCTCTGGGGC |
| ECD-mIgG2a | NO: 74 | TGCTTGCTGACCGCTGTCCATCCAGAACCACCCACT |
| DNA | | GCATGCAGAGAAAAACAGTACCTAATAAACAGTCA |
| | | GTGCTGTTCTTTGTGCCAGCCAGGACAGAAACTGGT |
| | | GAGTGACTGCACAGAGTTCACTGAAACGGAATGCC |
| | | TTCCTTGCGGTGAAAGCGAGTTCCTAGACACCTGGA |
| | | ACAGAGAGACACACTGCCACCAGCACAAATACTGC |
| | | GACCCCAACCTAGGGCTTCGGGTCCAGCAGAAGGG |
| | | CACCTCAGAAACAGACACCATCTGCACCTGTGAAG |
| | | AAGGCTGGCACTGTACGAGTGAGGCCTGTGAGAGC |
| | | TGTGTCCTGCACCGCTCATGCTCGCCCGGCTTTGGG |
| | | GTCAAGCAGATTGCTACAGGGGTTTCTGATACCATCT |
| | | GCGAGCCCTGCCCAGTCGGCTTCTTCTCCAATGTGT |
| | | CATCTGCTTTCGAAAAATGTCACCCTTGGACAAGCT |
| | | GTGAGACCAAAGACCTGGTTGTGCAACAGGCAGGC |
| | | ACAAACAAGACTGATGTTGTCTGTGGTCCCCAGGAT |
| | | CGGCTGAGAGCTAGCGAGCCCAGAGGGCCCACAAT |
| | | CAAGCCCTGTCCTCCATGCAAATGCCCAGCACCTAA |
| | | CCTCTTGGGTGGACCATCCGTCTTCATCTTCCCTCCA |
| | | AAGATCAAGGATGTACTCATGATCTCCCTGAGCCCC |
| | | ATGGTCACATGTGTGGTGGTGGATGTGAGCGAGGAT |
| | | GACCCAGATGTCCAGATCAGCTGGTTCGTGAACAAC |
| | | GTGGAAGTACTCACAGCTCAGACACAAACCCATAG |
| | | AGAGGATTACAACAGTACTCTCCGGGTGGTCAGTGC |
| | | CCTCCCCATCCAGCACCAGGACTGGATGAGTGGCAA |
| | | GGAGTTCAAATGCAAGGTCAACAACAAAGCCCTCC |
| | | CAGCGCCCATCGAGAGAACCATCTCAAAACCCAAA |
| | | GGGTCAGTAAGAGCTCCACAGGTATATGTCTTGCCT |
| | | CCACCAGAAGAAGAGATGACTAAGAAACAGGTCAC |
| | | TCTGACCTGCATGGTCACAGACTTCATGCCTGAAGA |
| | | CATTTACGTGGAGTGGACCAACAACGGGAAAACAG |
| | | AGCTAAACTACAAGAACACTGAACCAGTCCTGGAC |
| | | TCTGATGGTTCTTACTTCATGTACAGCAAGCTGAGA |
| | | GTGGAAAAGAAGAACTGGGTGGAAAGAAATAGCTA |
| | | CTCCTGCTCAGTGGTCCACGAGGGTCTGCACAATCA |
| | | CCACACGACTAAGAGCTTCTCCCGGACTCCGGGTAA |
| | | A |
| human OX40 | SEQ ID | MCVGARRLGRGPCAALLLLGLGLSTVTGLHCVGDTY |
| AA | NO: 75 | PSNDRCCHECRPGNGMVSRCSRSQNTVCRPCGPGFYN |
| | | DVVSSKPCKPCTWCNLRSGSERKQLCTATQDTVCRCR |
| | | AGTQPLDSYKPGVDCAPCPPGHFSPGDNQACKPWTN |
| | | CTLAGKHTLQPASNSSDAICEDRDPPATQPQETQGPPA |
| | | RPITVQPTEAWPRTSQGPSTRPVEVPGGRAVAAILGLG |
| | | LVLGLLGPLAILLALYLLRRDQRLPPDAHKPPGGGSFR |
| | | TPIQEEQADAHSTLAKI |
| human OX40 | SEQ ID | ATGTGCGTGGGGGCTCGGCGGCTGGGCCGCGGGCC |
| DNA | NO: 76 | GTGTGCGGCTCTGCTCCTCCTGGGCCTGGGGCTGAG |
| | | CACCGTGACGGGGCTCCACTGTGTCGGGGACACCT |
| | | ACCCCAGCAACGACCGGTGCTGCCACGAGTGCAGG |
| | | CCAGGCAACGGGATGGTGAGCCGCTGCAGCCGCTC |
| | | CCAGAACACGGTGTGCCGTCCGTGCGGGCCGGGCT |
| | | TCTACAACGACGTGGTCAGCTCCAAGCCGTGCAAG |
| | | CCCTGCACGTGGTGTAACCTCAGAAGTGGGAGTGA |
| | | GCGGAAGCAGCTGTGCACGGCCACACAGGACACAG |
| | | TCTGCCGCTGCCGGGCGGGCACCCAGCCCCTGGAC |
| | | AGCTACAAGCCTGGAGTTGACTGTGCCCCCTGCCCT |
| | | CCAGGGCACTTCTCCCCAGGCGACAACCAGGCCTG |
| | | CAAGCCCTGGACCAACTGCACCTTGGCTGGGAAGC |
| | | ACACCCTGCAGCCGGCCAGCAATAGCTCGGACGCA |
| | | ATCTGTGAGGACAGGGACCCCCCAGCCACGCAGCC |
| | | CCAGGAGACCCAGGGCCCCCCGGCCAGGCCCATCA |
| | | CTGTCCAGCCCACTGAAGCCTGGCCCAGAACCTCAC |
| | | AGGGACCCTCCACCCGGCCCGTGGAGGTCCCCGGG |
| | | GGCCGTGCGGTTGCCGCCATCCTGGGCCTGGGCCTG |
| | | GTGCTGGGGCTGCTGGGCCCCCTGGCCATCCTGCTG |
| | | GCCCTGTACCTGCTCCGGAGGGACCAGAGGCTGCC |
| | | CCCCGATGCCCACAAGCCCCCTGGGGGAGGCAGTTT |
| | | CCGGACCCCCATCCAAGAGGAGCAGGCCGACGCCC |
| | | ACTCCACCCTGGCCAAGATCTGA |
| human OX40 | SEQ ID | MCVGARRLGRGPCAALLLLGLGLSTVTGLHCVGDTY |
| ECD (1-216) | NO: 77 | PSNDRCCHECRPGNGMVSRCSRSQNTVCRPCGPGFYN |
| AA | | DVVSSKPCKPCTWCNLRSGSERKQLCTATQDTVCRCR |
| | | AGTQPLDSYKPGVDCAPCPPGHFSPGDNQACKPWTN |
| | | CTLAGKHTLQPASNSSDAICEDRDPPATQPQETQGPPA |
| | | RPITVQPTEAWPRTSQGPSTRPVEVPGGRAVA |
| human OX40 | SEQ ID | ATGTGCGTGGGGGCTCGGCGGCTGGGCCGCGGGCC |
| ECD (1-216) | NO: 78 | GTGTGCGGCTCTGCTCCTCCTGGGCCTGGGGCTGAG |
| DNA | | CACCGTGACGGGGCTCCACTGTGTCGGGGACACCT |
| | | ACCCCAGCAACGACCGGTGCTGCCACGAGTGCAGG |
| | | CCAGGCAACGGGATGGTGAGCCGCTGCAGCCGCTC |
| | | CCAGAACACGGTGTGCCGTCCGTGCGGGCCGGGCT |
| | | TCTACAACGACGTGGTCAGCTCCAAGCCGTGCAAG |
| | | CCCTGCACGTGGTGTAACCTCAGAAGTGGGAGTGA |
| | | GCGGAAGCAGCTGTGCACGGCCACACAGGACACAG |
| | | TCTGCCGCTGCCGGGCGGGCACCCAGCCCCTGGAC |
| | | AGCTACAAGCCTGGAGTTGACTGTGCCCCCTGCCCT |
| | | CCAGGGCACTTCTCCCCAGGCGACAACCAGGCCTG |
| | | CAAGCCCTGGACCAACTGCACCTTGGCTGGGAAGC |
| | | ACACCCTGCAGCCGGCCAGCAATAGCTCGGACGCA |
| | | ATCTGTGAGGACAGGGACCCCCCAGCCACGCAGCC |
| | | CCAGGAGACCCAGGGCCCCCCGGCCAGGCCCATCA |
| | | CTGTCCAGCCCACTGAAGCCTGGCCCAGAACCTCAC |
| | | AGGGACCCTCCACCCGGCCCGTGGAGGTCCCCGGG |
| | | GGCCGTGCGGTTGCC |
| human OX40 | SEQ ID | MCVGARRLGRGPCAALLLLGLGLSTVTGLHCVGDTY |
| ECD-His AA | NO: 79 | PSNDRCCHECRPGNGMVSRCSRSQNTVCRPCGPGFYN |
| | | DVVSSKPCKPCTWCNLRSGSERKQLCTATQDTVCRCR |
| | | AGTQPLDSYKPGVDCAPCPPGHFSPGDNQACKPWTN |
| | | CTLAGKHTLQPASNSSDAICEDRDPPATQPQETQGPPA |
| | | RPITVQPTEAWPRTSQGPSTRPVEVPGGRAVAHHHHH |
| | | H |
| human OX40 | SEQ ID | ATGTGCGTGGGGGCTCGGCGGCTGGGCCGCGGGCC |
| ECD-His DNA | NO: 80 | GTGTGCGGCTCTGCTCCTCCTGGGCCTGGGGCTGAG |
| | | CACCGTGACGGGGCTCCACTGTGTCGGGGACACCT |
| | | ACCCCAGCAACGACCGGTGCTGCCACGAGTGCAGG |
| | | CCAGGCAACGGGATGGTGAGCCGCTGCAGCCGCTC |
| | | CCAGAACACGGTGTGCCGTCCGTGCGGGCCGGGCT |
| | | TCTACAACGACGTGGTCAGCTCCAAGCCGTGCAAG |
| | | CCCTGCACGTGGTGTAACCTCAGAAGTGGGAGTGA |
| | | GCGGAAGCAGCTGTGCACGGCCACACAGGACACAG |
| | | TCTGCCGCTGCCGGGCGGGCACCCAGCCCCTGGAC |
| | | AGCTACAAGCCTGGAGTTGACTGTGCCCCCTGCCCT |
| | | CCAGGGCACTTCTCCCCAGGCGACAACCAGGCCTG |
| | | CAAGCCCTGGACCAACTGCACCTTGGCTGGGAAGC |
| | | ACACCCTGCAGCCGGCCAGCAATAGCTCGGACGCA |
| | | ATCTGTGAGGACAGGGACCCCCCAGCCACGCAGCC |
| | | CCAGGAGACCCAGGGCCCCCCGGCCAGGCCCATCA |
| | | CTGTCCAGCCCACTGAAGCCTGGCCCAGAACCTCAC |
| | | AGGGACCCTCCACCCGGCCCGTGGAGGTCCCCGGG |
| | | GGCCGTGCGGTTGCCCATCACCATCACCATCAC |

### Example 3. Generation of Anti-huCD137 VH Domain Antibodies

### Phage Display Panning and Screening

Phage display selection was carried out by phage display using standard protocols (Silacci et al., (2005) Proteomics, 5, 2340-50; Zhao et al., (2014) PLOS One, 9, e111339). In brief, 10 μg/ml of immobilized human CD137 ECD-mIgG2a in immunotubes (Cat. 470319, ThermoFisher) was utilized in round 1 and 2. Hut78/huCD137 cells were used for selection in round 3 and 4. Immunotubes were blocked with 5% milk powder (w/v) in PBS supplemented with 1% Tween 20 (MPBST) for 1 hour. After washes with PBST (PBS buffer supplemented with 0.05% Tween 20), 5×10¹² (round 1) or 5×10¹¹ (rounds 2) phages from each sub library were depleted by human CD40 ECD-mIgG2a in MPBST for 1 hour and then incubated with the antigen for 1 hour. For the third and fourth rounds of selections, cell panning was carried out using Hut78/huCD137 cells (round 3) with HEK293(ATCC, CRL-1573) cells as depletion cells. After washes with PBST, bound phages were eluted with 100 mM triethylamine (Sigma-Aldrich). Eluted phages were used to infect mid-log phase E. coli TG1 bacteria and plated onto TYE-agar plates supplemented with 2% glucose and 100 μg/ml ampicillin. After four rounds of selections, individual clones were picked up and phage containing supernatants were prepared using standard protocols. Phage ELISA and FACS were used to screen anti-huCD137 VH domain antibodies.

For phage ELISA, a Maxisorp immunoplate was coated with antigens and blocked with 5% milk powder (w/v) in PBS buffer. Phage supernatant was blocked with MPBST for 30 min and added to wells of the ELISA plate for 1 hour. After washes with PBST, bound phage was detected using HRP-conjugated anti-M13 antibody (GE Healthcare) and 3,3′,5,5′-tetramethylbenzidine substrate (Cat.: 00-4201-56, eBioscience, USA). The ELISA-positive clones were further verified by flow cytometry using Hut78/huCD137 cells. CD137-expressing cells (10⁵ cells/well) were incubated with ELISA-positive phage supernatants, followed by binding with Alexa Fluro-647-labeled anti-M13 antibody (GE Healthcare). Cell fluorescence was quantified using a flow cytometer (Guava easyCyte™ 8HT, Merck-Millipore, USA).

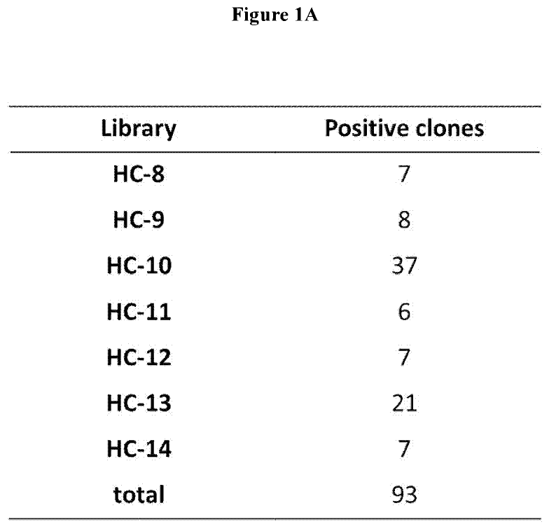

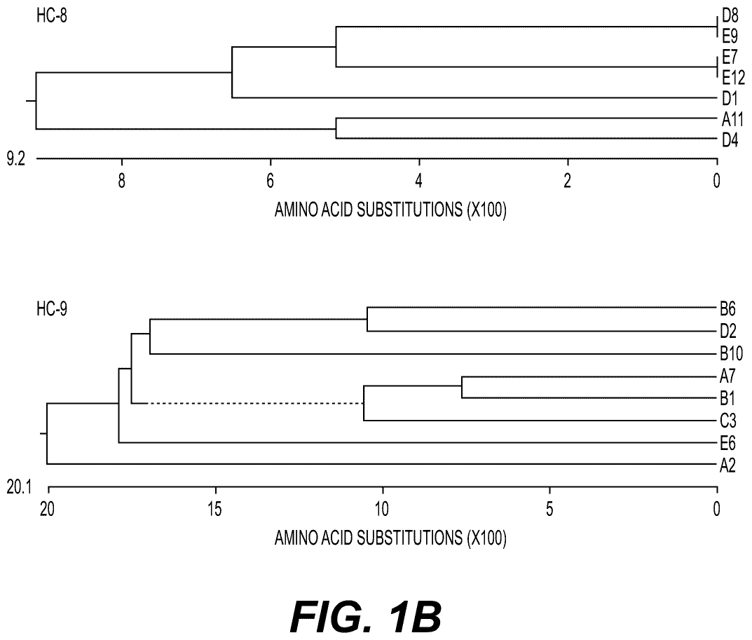

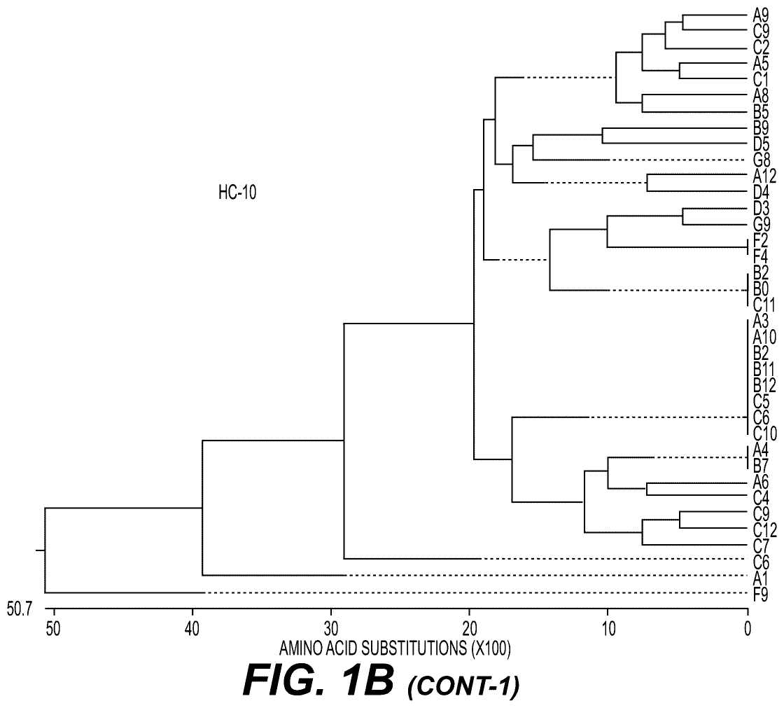

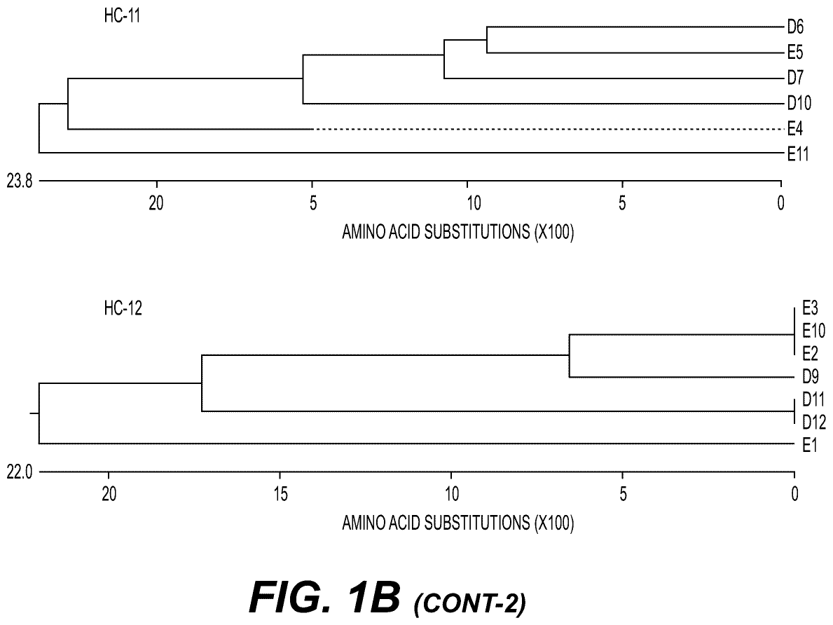

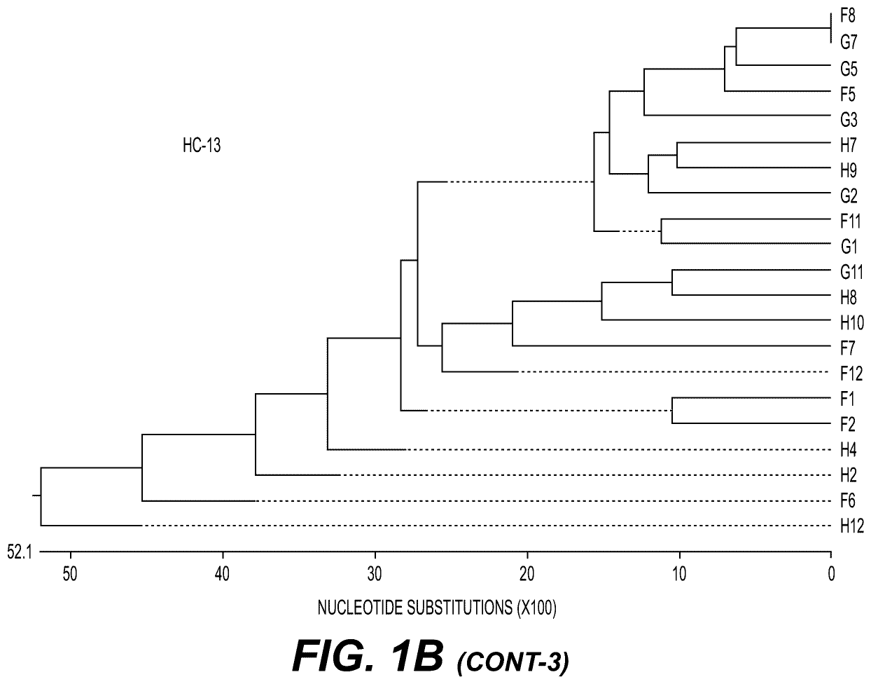

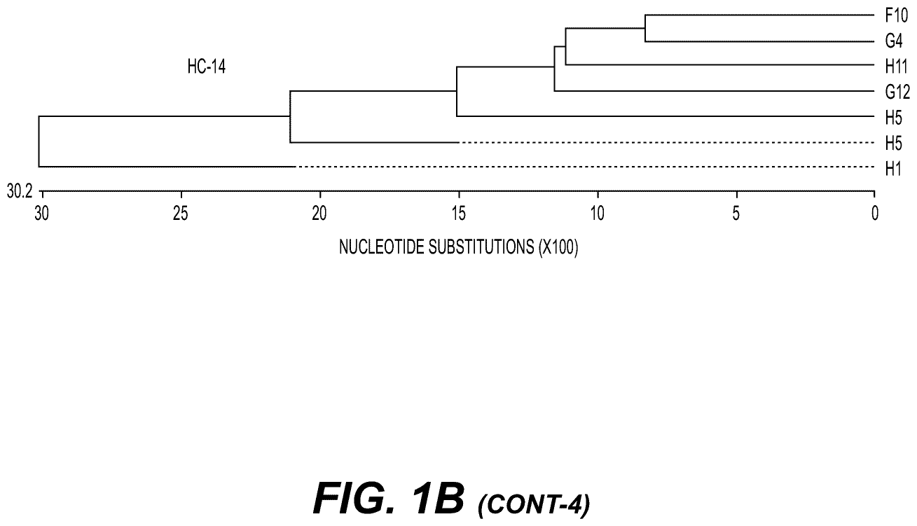

The clones that showed positive signals in FACS screening, and binding to both huCD137 and cynoCD137 but not huOX40 and huCD40, were picked up and sequenced. Approximately 76 unique sequences from 93 positive clones were identified, and a number of clones originated from two sub libraries, HC-10 and HC-13 (FIG. 1A-1B).

### Expression and Purification of Fc Fusion VH Antibodies

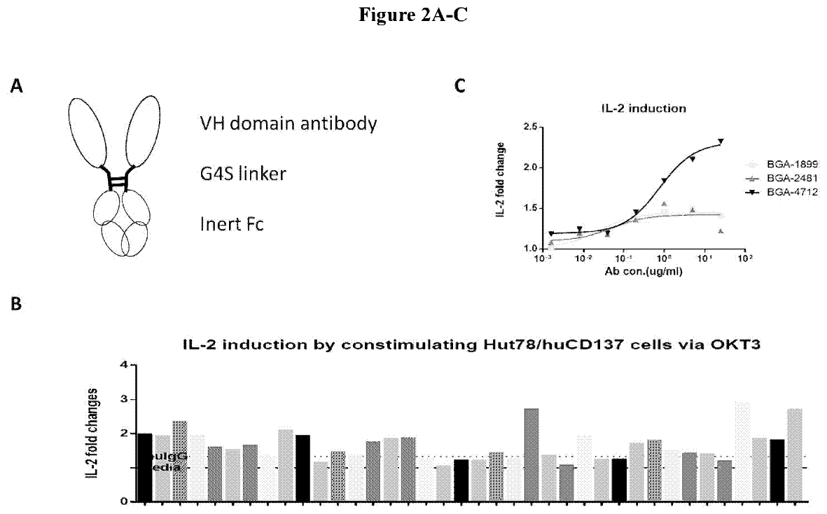

The VH sequences were analyzed by comparing sequence homology and grouped based on sequence similarity. Complementary determining regions (CDRs) were defined based on the Kabat (Wu and Kabat (1970) J. Exp. Med. 132:211-250) and IMGT (Lefranc (1999) Nucleic Acids Research 27:209-212) system by sequence annotation and by internet-based sequence analysis. The amino acid and DNA sequences of two representative top clones BGA-7207 and BGA-4712 are listed in Table 5 below. After the sequence checking and analysis of binding curve by SPR, anti-huCD137 VH domain antibodies were then constructed as human Fc fusion VH antibody format (VH-Fc) using in-house developed expression vectors. As shown in FIG. 2A, VH domain antibodies were fused at the N terminal of human Fc with a G4S (SEQ ID NO: 246) linker in between. A Fc-null version (an inert Fc without FcγR-binding) of human IgG1 (SEQ ID NO: 85) was used. Expression and preparation of Fc fusion VH antibodies were achieved by transfection into 293G cells and by purification using a protein A column (Cat. No. 17543802, GE Life Sciences). The purified antibodies were concentrated to 0.5-5 mg/mL in PBS and stored in aliquots in a −80° C. freezer.

TABLE 5
Amino acid and DNA sequences of two selected anti-huCD137 VH domain
antibodies

| Antibody | SEQ ID NO | | SEQUENCE |
|---|---|---|---|
| BGA-7207 | SEQ ID NO: 9 | VH | EVQLLESGGGLVQPGGSLRLSCAASGFRLDT |
| | | | TEVGWVRQAPGKGLEWVSTIMGISGSTYYA |
| | | | DSVKGRFTISRDNSKNTLYLQMNSLRAEDT |
| | | | AVYYCARVVDSLFDDSAVFDYWGQGTLVT |
| | | | VSS |
| | SEQ ID NO: 10 | VH DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCAGATTGG |
| | | | ACACCACCGAAGTAGGTTGGGTGCGTCAA |
| | | | GCGCCGGGGAAAGGACTGGAATGGGTCTC |
| | | | CACCATCATGGGTATTAGTGGTTCGACATA |
| | | | CTATGCGGACAGTGTCAAAGGGCGCTTTA |
| | | | CGATCTCGCGCGATAACTCAAAAAATACTC |
| | | | TTTACCTTCAAATGAATAGCCTTCGTGCTG |
| | | | AGGACACTGCGGTGTACTACTGCGCCCGC |
| | | | GTCGTCGATTCCCTCTTTGATGACAGCGCC |
| | | | GTTTTTGATTACTGGGGACAGGGCACCTTA |
| | | | GTTACAGTCTCATCG |
| | SEQ ID NO: 81 | VH-Fc | EVQLLESGGGLVQPGGSLRLSCAASGFRLDT |
| | | | TEVGWVRQAPGKGLEWVSTIMGISGSTYYA |
| | | | DSVKGRFTISRDNSKNTLYLQMNSLRAEDT |
| | | | AVYYCARVVDSLFDDSAVFDYWGQGTLVT |
| | | | VSSGGGGSDKTHTCPPCPAPELLGGPSVFLF |
| | | | PPKPKDTLMISRTPEVTCVVVDVSHEDPEVK |
| | | | FNWYVDGVEVHNAKTKPREEQYNSTYRVV |
| | | | SVLTVLHQDWLNGKEYKCKVSNKALPAPIE |
| | | | KTISKAKGQPREPQVYTLPPSRDELTKNQVS |
| | | | LTCLVKGFYPSDIAVEWESNGQPENNYKTTP |
| | | | PVLDSDGSFFLYSKLTVDKSRWQQGNVFSCS |
| | | | VMHEALHNHYTQKSLSLSPGK |
| | SEQ ID NO: 82 | VH-Fc DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCAGATTGG |
| | | | ACACCACCGAAGTAGGTTGGGTGCGTCAA |
| | | | GCGCCGGGGAAAGGACTGGAATGGGTCTC |
| | | | CACCATCATGGGTATTAGTGGTTCGACATA |
| | | | CTATGCGGACAGTGTCAAAGGGCGCTTTA |
| | | | CGATCTCGCGCGATAACTCAAAAAATACTC |
| | | | TTTACCTTCAAATGAATAGCCTTCGTGCTG |
| | | | AGGACACTGCGGTGTACTACTGCGCCCGC |
| | | | GTCGTCGATTCCCTCTTTGATGACAGCGCC |
| | | | GTTTTTGATTACTGGGGACAGGGCACCTTA |
| | | | GTTACAGTCTCATCGGGCGGCGGAGGGTC |
| | | | TGACAAAACTCACACATGCCCACCGTGCC |
| | | | CAGCACCTGAACTCCTGGGGGGACCGTCA |
| | | | GTCTTCCTCTTCCCCCCAAAACCCAAGGA |
| | | | CACCCTCATGATCTCCCGGACCCCTGAGGT |
| | | | CACATGCGTGGTGGTGGACGTGAGCCACG |
| | | | AAGACCCTGAGGTCAAGTTCAACTGGTAC |
| | | | GTGGACGGCGTGGAGGTGCATAATGCCAA |
| | | | GACAAAGCCGCGGGAGGAGCAGTACAAC |
| | | | AGCACGTACCGTGTGGTCAGCGTCCTCAC |
| | | | CGTCCTGCACCAGGACTGGCTGAATGGCA |
| | | | AGGAGTACAAGTGCAAGGTCTCCAACAAA |
| | | | GCCCTCCCAGCCCCCATCGAGAAAACCAT |
| | | | CTCCAAAGCCAAAGGGCAGCCCCGAGAA |
| | | | CCACAGGTGTACACCCTGCCCCCATCCCGG |
| | | | GATGAGCTGACCAAGAACCAGGTCAGCCT |
| | | | GACCTGCCTGGTCAAAGGCTTCTATCCCAG |
| | | | CGACATCGCCGTGGAGTGGGAGAGCAATG |
| | | | GGCAGCCGGAGAACAACTACAAGACCAC |
| | | | GCCTCCCGTGCTGGACTCCGACGGCTCCTT |
| | | | CTTCCTCTACAGCAAGCTCACCGTGGACA |
| | | | AGAGCAGGTGGCAGCAGGGGAACGTCTTC |
| | | | TCATGCTCCGTGATGCATGAGGCTCTGCAC |
| | | | AACCACTACACGCAGAAGAGCCTCTCCCT |
| | | | GTCTCCGGGTAAA |
| BGA-4712 | SEQ ID NO: 19 | VH | EVQLLESGGGLVQPGGSLRLSCAASGFMLS |
| | | | AEDVGWVRQAPGKGLEWVSAILDFGGSTY |
| | | | YADSVKGRFTISRDNSKNTLYLQMNSLRAE |
| | | | DTAVYYCARVVYHAGGGVTFDYWGQGTLV |
| | | | TVSS |
| | SEQ ID NO: 20 | VH DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCATGTTGT |
| | | | CCGCCGAAGACGTGGGTTGGGTGCGTCAA |
| | | | GCGCCGGGGAAAGGACTGGAATGGGTCTC |
| | | | CGCCATCTTGGATTTTGGTGGTTCGACATA |
| | | | CTATGCGGACAGTGTCAAAGGGCGCTTTA |
| | | | CGATCTCGCGCGATAACTCAAAAAATACTC |
| | | | TTTACCTTCAAATGAATAGCCTTCGTGCTG |
| | | | AGGACACTGCGGTGTACTACTGCGCCCGC |
| | | | GTCGTCTACCATGCTGGTGGTGGCGTCACC |
| | | | TTTGATTACTGGGGACAGGGCACCTTAGTT |
| | | | ACAGTCTCATCG |
| | SEQ ID NO: 83 | VH-Fc | EVQLLESGGGLVQPGGSLRLSCAASGFMLS |
| | | | AEDVGWVRQAPGKGLEWVSAILDFGGSTY |
| | | | YADSVKGRFTISRDNSKNTLYLQMNSLRAE |
| | | | DTAVYYCARVVYHAGGGVTFDYWGQGTLV |
| | | | TVSSGGGGSDKTHTCPPCPAPELLGGPSVFL |
| | | | FPPKPKDTLMISRTPEVTCVVVDVSHEDPEV |
| | | | KFNWYVDGVEVHNAKTKPREEQYNSTYRV |
| | | | VSVLTVLHQDWLNGKEYKCKVSNKALPAPI |
| | | | EKTISKAKGQPREPQVYTLPPSRDELTKNQV |
| | | | SLTCLVKGFYPSDIAVEWESNGQPENNYKTT |
| | | | PPVLDSDGSFFLYSKLTVDKSRWQQGNVFSC |
| | | | SVMHEALHNHYTQKSLSLSPGK |
| | SEQ ID NO: 84 | VH-Fc DNA | GAGGTCCAGTTACTTGAGAGTGGTGGAGG |
| | | | TCTGGTCCAACCAGGAGGTTCGCTGCGTT |
| | | | TATCCTGCGCCGCGTCTGGATTCATGTTGT |
| | | | CCGCCGAAGACGTGGGTTGGGTGCGTCAA |
| | | | GCGCCGGGGAAAGGACTGGAATGGGTCTC |
| | | | CGCCATCTTGGATTTTGGTGGTTCGACATA |
| | | | CTATGCGGACAGTGTCAAAGGGCGCTTTA |
| | | | CGATCTCGCGCGATAACTCAAAAAATACTC |
| | | | TTTACCTTCAAATGAATAGCCTTCGTGCTG |
| | | | AGGACACTGCGGTGTACTACTGCGCCCGC |
| | | | GTCGTCTACCATGCTGGTGGTGGCGTCACC |
| | | | TTTGATTACTGGGGACAGGGCACCTTAGTT |
| | | | ACAGTCTCATCGGGCGGCGGAGGGTCTGA |
| | | | CAAAACTCACACATGCCCACCGTGCCCAG |
| | | | CACCTGAACTCCTGGGGGGACCGTCAGTC |
| | | | TTCCTCTTCCCCCCAAAACCCAAGGACAC |
| | | | CCTCATGATCTCCCGGACCCCTGAGGTCAC |
| | | | ATGCGTGGTGGTGGACGTGAGCCACGAAG |
| | | | ACCCTGAGGTCAAGTTCAACTGGTACGTG |
| | | | GACGGCGTGGAGGTGCATAATGCCAAGAC |
| | | | AAAGCCGCGGGAGGAGCAGTACAACAGC |
| | | | ACGTACCGTGTGGTCAGCGTCCTCACCGTC |
| | | | CTGCACCAGGACTGGCTGAATGGCAAGGA |
| | | | GTACAAGTGCAAGGTCTCCAACAAAGCCC |
| | | | TCCCAGCCCCCATCGAGAAAACCATCTCC |
| | | | AAAGCCAAAGGGCAGCCCCGAGAACCAC |
| | | | AGGTGTACACCCTGCCCCCATCCCGGGATG |
| | | | AGCTGACCAAGAACCAGGTCAGCCTGACC |
| | | | TGCCTGGTCAAAGGCTTCTATCCCAGCGAC |
| | | | ATCGCCGTGGAGTGGGAGAGCAATGGGCA |
| | | | GCCGGAGAACAACTACAAGACCACGCCTC |
| | | | CCGTGCTGGACTCCGACGGCTCCTTCTTCC |
| | | | TCTACAGCAAGCTCACCGTGGACAAGAGC |
| | | | AGGTGGCAGCAGGGGAACGTCTTCTCATG |
| | | | CTCCGTGATGCATGAGGCTCTGCACAACC |
| | | | ACTACACGCAGAAGAGCCTCTCCCTGTCT |
| | | | CCGGGTAAA |
| Inert Fc | SEQ ID NO: 85 | A Fc-null | DKTHTCPPCPAPELLGGPSVFLFPPKPKDTL |
| | | version | MISRTPEVTCVVVDVSHEDPEVKFNWYVDG |
| | | IgG1 | VEVHNAKTKPREEQYNSTYRVVSVLTVLHQ |
| | | | DWLNGKEYKCKVSNKALPAPIEKTISKAKG |
| | | | QPREPQVYTLPPSRDELTKNQVSLTCLVKGF |
| | | | YPSDIAVEWESNGQPENNYKTTPPVLDSDGS |
| | | | FFLYSKLTVDKSRWQQGNVFSCSVMHEALH |
| | | | NHYTQKSLSLSPGK |
| | SEQ ID NO: 86 | A Fc-null | GACAAAACTCACACATGCCCACCGTGCCC |
| | | version | AGCACCTGAACTCCTGGGGGGACCGTCAG |
| | | IgG1 DNA | TCTTCCTCTTCCCCCCAAAACCCAAGGAC |
| | | | ACCCTCATGATCTCCCGGACCCCTGAGGTC |
| | | | ACATGCGTGGTGGTGGACGTGAGCCACGA |
| | | | AGACCCTGAGGTCAAGTTCAACTGGTACG |
| | | | TGGACGGCGTGGAGGTGCATAATGCCAAG |
| | | | ACAAAGCCGCGGGAGGAGCAGTACAACA |
| | | | GCACGTACCGTGTGGTCAGCGTCCTCACC |
| | | | GTCCTGCACCAGGACTGGCTGAATGGCAA |
| | | | GGAGTACAAGTGCAAGGTCTCCAACAAAG |
| | | | CCCTCCCAGCCCCCATCGAGAAAACCATCT |
| | | | CCAAAGCCAAAGGGCAGCCCCGAGAACC |
| | | | ACAGGTGTACACCCTGCCCCCATCCCGGG |
| | | | ATGAGCTGACCAAGAACCAGGTCAGCCTG |
| | | | ACCTGCCTGGTCAAAGGCTTCTATCCCAGC |
| | | | GACATCGCCGTGGAGTGGGAGAGCAATGG |
| | | | GCAGCCGGAGAACAACTACAAGACCACGC |
| | | | CTCCCGTGCTGGACTCCGACGGCTCCTTCT |
| | | | TCCTCTACAGCAAGCTCACCGTGGACAAG |
| | | | AGCAGGTGGCAGCAGGGGAACGTCTTCTC |
| | | | ATGCTCCGTGATGCATGAGGCTCTGCACAA |
| | | | CCACTACACGCAGAAGAGCCTCTCCCTGT |
| | | | CTCCGGGTAAA |

### Example 4. Functional Screening of Anti-huCD137 VH Domain Antibodies

Functional screening was first applied to selected anti-huCD137 VH domain antibodies with strong agonism using supernatant containing VH-Fc proteins. In brief, the 96-well white/clear bottom plates (Thermo Fisher) were pre-incubated with 3 μg/ml anti-hu CD3 (Invitrogen, Cat. No. 16-0037-85) at 50 μl/well for 5 min and then washed away by PBS buffer. Next, Hut78/huCD137 cells were resuspended at 5×10⁵ cells/ml, and directly plated into the pre-coated plates at 50 μl/well (25,000 well per well). Supernatants containing various VH-Fc proteins were mixed with the cells. Alternatively, for purified VH domain antibodies with Fc fusion, a dose titration of purified VH-Fc protein preparations was added in duplicate at 25, 5, 1, 0.2, 0.04, 0.008 or 0.0016 μg/ml at 50 μl/well. As a crosslinker, goat anti-hu IgG(H&L) polystyrene particles (6.46 um) (Cat. No. HUP-60-5, Spherotech) were added. Assay plates were incubated overnight at 37° C., and the concentrations of IL-2 were measured after 24 hours. Data was plotted as IL-2 fold increase compared with the concentration in the well with media only. FIG. 2B shows a representative screening result using supernatants containing VH-Fc proteins, and one of the clones, BGA-4712 has been shown to be capable to stimulate IL-2 production in Hut78/huCD137 cells in a dose dependent manner (FIG. 2C).

### Example 5. Characterization of Purified Anti-huCD137 VH Domain Antibodies

### Characterization of Purified Antibodies Via ELSIA

For antigen ELISA, a Maxisorp immunoplate was coated with antigens and blocked with 3% BSA (w/v) in PBS buffer (blocking buffer). Monoclonal VH domain antibodies were blocked with blocking buffer for 30 min and added to wells of the ELISA plate for 1 hour. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA). All selected clones were shown to cross-react with cynoCD137 and no binding to human OX40 ECD and human CD40 ECD.

### Characterization of Purified Antibodies Via SPR Analysis

Characterization of anti-huCD137 VH domain antibodies were made by SPR assays using BIAcore™ T-200 (GE Life Sciences). Briefly, anti-human IgG (Fc) antibody was immobilized on an activated CM5 biosensor chip (Cat.: BR100839, GE Life Sciences). Anti-huCD137 domain antibodies were flowed over the chip surface and captured by anti-human IgG (Fc) antibody. Then a serial dilution (6.0 nM to 2150 nM) of human CD137 ECD-mIgG2a was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ.

### Characterization of Purified Antibodies Via Flow Cytometry

For flow cytometry, human CD137 expressing cells (10⁵ cells/well) were incubated with various concentrations of purified VH domain antibodies, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). Ligand competition was also applied in a flow cytometry based assay. In brief, Hut78/huCD137 was incubated with Fc fusion VH domain antibodies (VH-Fc) in the presence of serially diluted human CD137 ligand-mIgG2a, followed by detection with Alexa Fluro-647-labeled anti-hu IgG Fc antibody (Cat.: 409320, BioLegend, USA).

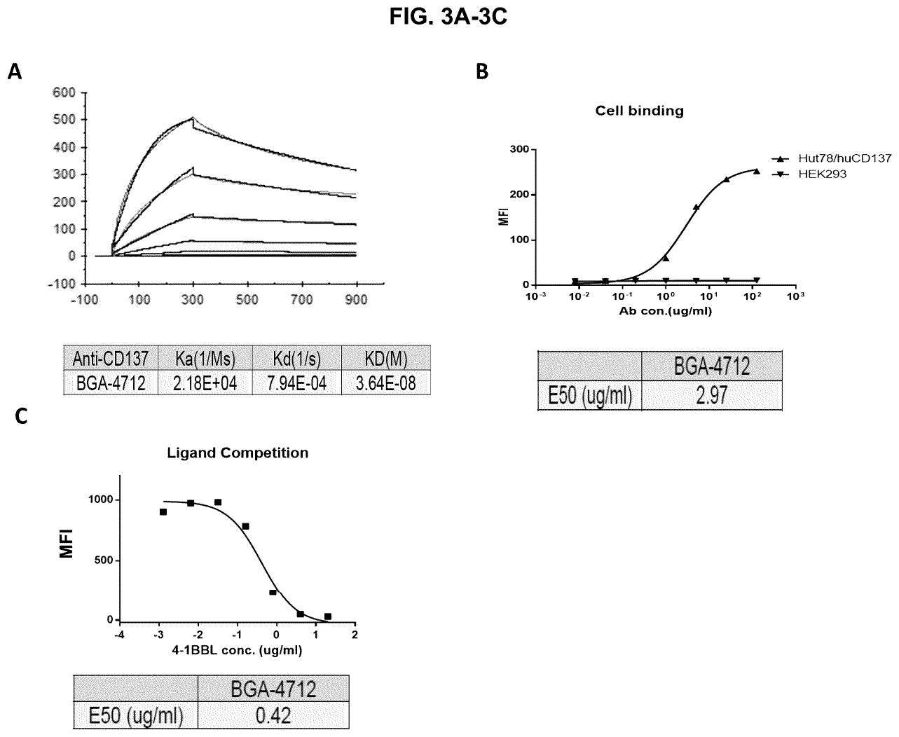

Selected VH domain antibodies were then characterized for affinity, cell binding and ligand competition. The SPR study, FACS analysis and the ligand competition result of one representative top clone BGA-4712 are shown in FIG. 3.

### Example 6. Construction of CD137×CEA Multispecific Antibodies Using Anti-CD137 VH Domain Antibody BGA-4712 and an Anti-CEA Antibody

To explore potential more effective CD137 based mechanisms of action (MOAs) than single antibody treatment, a number of multispecific formats utilizing the anti-huCD137 VH domain antibody have been constructed and tested. Here, multiple formats have been adopted to create CD137-based T cell-engagers (TCEs), in which a first antigen binding domain is directed against a tumor-associated antigen (TAA) and a second antigen binding domain targets a CD137 activating receptor. For example, a first antigen binding domain of an anti-CEA antibody (SEQ ID NO: 87 and 89) was used to pair with a second antigen binding domain of an anti-huCD137 VH domain antibody BGA-4712 (SEQ ID NO: 17) in specifically defined formats as shown below (Table 6). For this construct, an inert Fc was used (SEQ ID NO: 85). Expression and preparation of these multispecific antibodies were achieved by transfection into 293G cells and by purification using a protein A column (Cat. No. 17543802, GE Life Sciences). The purified antibodies were concentrated to 0.5-5 mg/mL in PBS and stored in aliquots in a −80° C. freezer.

### Format a (A-CD137×CEA)

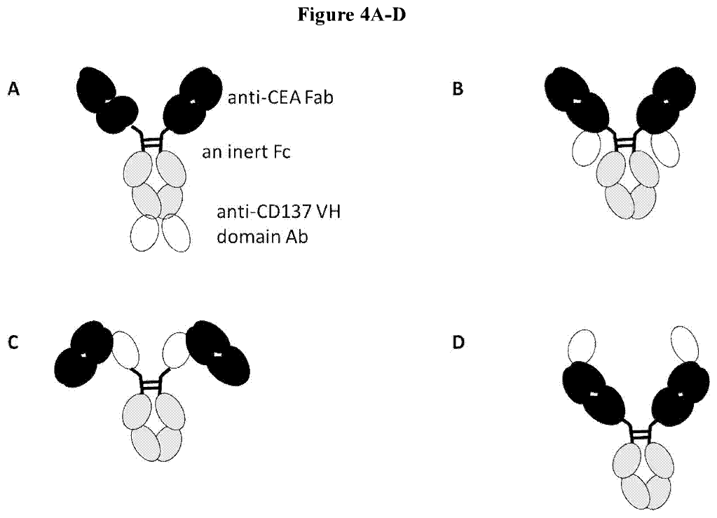

The format A provides a symmetric IgG-like multispecific molecule with Fab×VH configuration. Anti-huCD137 VH domain antibody BGA-4712 was fused to the c-termini of Fc (CH3 domain) of an anti-CEA antibody with one G4S linker in between (SEQ ID NOs: 89 and 91) as shown in FIG. 4A.

### Format B (B-CD137×CEA)

The format B also provides a symmetric IgG-like multispecific molecule with Fab×VH configuration. Anti-huCD137 VH domain antibody BGA-4712 was fused to the c-termini of light chain (CK) of an anti-CEA antibody with one G4S linker in between (SEQ ID NOs: 87 and 93) as shown in FIG. 4B.

### Format C (C-CD137×CEA)

The format C provides a symmetric VH antibody-like multispecific molecule with Fab×VH configuration. The Fab region of an anti-CEA antibody was fused to the N-termini of VH of anti-huCD137 VH domain antibody BGA-4712 with one G4S linker in between (SEQ ID NOs: 89 and 95) as shown in FIG. 4C.

### FORMAT D (D-CD137×CEA)

The format D also provides a symmetric IgG-like multispecific molecule with Fab×VH configuration. Anti-huCD137 VH domain antibody BGA-4712 was fused to the N-termini of heavy chain (Vh) of an anti-CEA antibody with one G4S linker in between (SEQ ID NOs: 89 and 97) as shown in FIG. 4D.

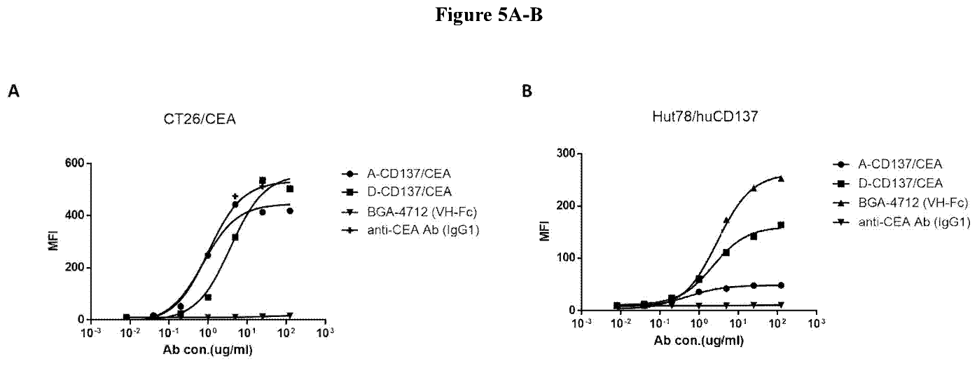

The yields and biochemical properties of various CD137×CEA multispecific antibodies were summarized in Table 7. For the two molecules A-CD137×CEA and D-CD137×CEA, the monomers are both above 95% based on the SEC-HPLC profiles (Table 7). By a flow cytometry-based assay, it is demonstrated that there is very low reduction of the affinity of the anti-CEA arm in the format A, whereas there is a significant affinity reduction of the anti-CEA arm in format D (FIG. 5A). It was also demonstrated that there is an affinity reduction of CD137 arm in the format A, whereas little or no influence in the format D (FIG. 5D).

TABLE 6
Amino acid and DNA sequences of CD137xCEA multispecific antibodies of various
formats

| | | SEQ ID | |
|---|---|---|---|
| CONSTRUCT | Chain | NO | SEQUENCE |
| A-CD137xCEA | A-BGA-4712/CEA | SEQ ID | QVQLVQSGAEVKKPGASVKVSCKASGYIFTSYY |
| | heavy chain AA | NO: 91 | LHWVRQAPGQGLEWIGYINPQTGKTSYAQKFQ |
| | | | GRVTMTRDTSTSTVYMELSSLRSEDTAVYYCAR |
| | | | EYGNYNYPLDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKKVEPKSCDKTHTCPP |
| | | | CPAPPAAGPSVFLFPPKPKDTLMISRTPEVTCVV |
| | | | VDVSHEDPEVKFNWYVDGVEVHNAKTKPREE |
| | | | QYNSTYRVVSVLTVLHQDWLNGKEYKCKVSN |
| | | | KALAAPIEKTISKAKGQPREPQVYTLPPSREEMT |
| | | | KNQVSLTCLVKGFYPSDIAVEWESNGQPENNYK |
| | | | TTPPVLDSDGSFFLYSKLTVDKSRWQQGNVFSC |
| | | | SVMHEALHNHYTQKSLSLSPGKGGGGSEVQLL |
| | | | ESGGGLVQPGGSLRLSCAASGFMLSAEDVGWV |
| | | | RQAPGKGLEWVSAILDFGGSTYYADSVKGRFTI |
| | | | SRDNSKNTLYLQMNSLRAEDTAVYYCARVVYH |
| | | | AGGGVTFDYWGQGTLVTVSS |
| | A-BGA-4712/CEA | SEQ ID | CAGGTGCAGCTGGTGCAGAGCGGCGCGGAAG |
| | heavy chain DNA | NO: 92 | TGAAAAAACCGGGCGCGAGCGTGAAAGTGAG |
| | | | CTGCAAAGCGAGCGGCTATATTTTTACCAGCTA |
| | | | TTACCTGCATTGGGTGCGCCAGGCGCCGGGCC |
| | | | AGGGCCTGGAATGGATTGGCTATATTAACCCG |
| | | | CAGACCGGCAAGACCAGCTATGCCCAGAAATT |
| | | | TCAGGGCCGCGTGACCATGACCCGCGATACCA |
| | | | GCACCAGCACCGTGTATATGGAACTGAGCAGC |
| | | | CTGCGCAGCGAAGATACCGCGGTGTATTATTG |
| | | | CGCGCGCGAATATGGCAACTATAACTATCCGCT |
| | | | GGATTATTGGGGCCAGGGCACCCTGGTGACCG |
| | | | TGAGCAGCGCTAGCACCAAGGGGCCCTCGGT |
| | | | CTTCCCCCTGGCACCCTCCTCCAAGAGTACTT |
| | | | CTGGGGGCACAGCGGCCCTGGGCTGCCTGGT |
| | | | CAAGGACTACTTCCCCGAACCGGTGACGGTGT |
| | | | CGTGGAACTCAGGCGCCCTGACCAGCGGCGT |
| | | | GCACACCTTCCCGGCTGTCCTACAGTCCTCAG |
| | | | GACTCTACTCCCTCAGCAGCGTGGTGACCGTG |
| | | | CCCTCCAGCAGCTTGGGCACCCAGACCTACAT |
| | | | CTGCAACGTGAATCACAAGCCCAGCAACACC |
| | | | AAGGTGGACAAGAAAGTTGAGCCAAAGTCCT |
| | | | GTGACAAGACCCACACATGCCCCCCTTGTCCT |
| | | | GCTCCACCAGCTGCAGGACCAAGCGTGTTCCT |
| | | | GTTTCCACCCAAGCCCAAGGATACCCTGATGA |
| | | | TCTCTCGGACCCCAGAGGTGACATGCGTGGTG |
| | | | GTGGATGTGAGCCACGAGGACCCCGAGGTGA |
| | | | AGTTCAACTGGTATGTGGACGGCGTGGAGGTG |
| | | | CACAATGCTAAGACCAAGCCCAGGGAGGAGC |
| | | | AGTACAACTCCACCTATAGAGTGGTGTCTGTG |
| | | | CTGACAGTGCTGCACCAGGATTGGCTGAACG |
| | | | GCAAGGAGTATAAGTGCAAGGTGTCCAATAAG |
| | | | GCCCTGGCCGCTCCTATCGAGAAGACCATCTC |
| | | | TAAGGCCAAGGGCCAGCCCAGAGAGCCTCAG |
| | | | GTGTACACACTGCCTCCATCCCGGGAAGAGAT |
| | | | GACCAAGAACCAGGTGTCTCTGACATGTCTGG |
| | | | TCAAGGGCTTCTATCCCTCTGACATCGCCGTG |
| | | | GAGTGGGAGAGCAATGGCCAGCCTGAGAACA |
| | | | ATTACAAGACCACACCCCCTGTGCTGGATTCC |
| | | | GACGGCTCTTTCTTTCTGTATAGCAAGCTGACC |
| | | | GTGGACAAGTCCCGGTGGCAGCAGGGCAACG |
| | | | TGTTCAGCTGTTCCGTGATGCACGAAGCTCTG |
| | | | CATAATCACTATACTCAGAAATCCCTGTCACTG |
| | | | TCACCTGGTAAAGGTGGAGGCGGTTCAGAGG |
| | | | TCCAGTTACTTGAGAGTGGTGGAGGTCTGGTC |
| | | | CAACCAGGAGGTTCGCTGCGTTTATCCTGCGC |
| | | | CGCGTCTGGATTCATGTTGTCCGCCGAAGACG |
| | | | TGGGTTGGGTGCGTCAAGCGCCGGGGAAAGG |
| | | | ACTGGAATGGGTCTCCGCCATCTTGGATTTTG |
| | | | GTGGTTCGACATACTATGCGGACAGTGTCAAA |
| | | | GGGCGCTTTACGATCTCGCGCGATAACTCAAA |
| | | | AAATACTCTTTACCTTCAAATGAATAGCCTTCG |
| | | | TGCTGAGGACACTGCGGTGTACTACTGCGCCC |
| | | | GCGTCGTCTACCATGCTGGTGGTGGCGTCACC |
| | | | TTTGATTACTGGGGACAGGGCACCTTAGTTAC |
| | | | AGTCTCATCG |
| | anti-CEA Ab light | SEQ ID | DIQMTQSPSSLSASVGDRVTITCRASENQYGYL |
| | chain AA | NO: 89 | AWYQQKPGKVPKLLIYNYKNLVEGVPSRFSGSG |
| | | | SGTDFTLTISSLQPEDVATYYCQHHLGTPYTFGQ |
| | | | GTKVEIKRTVAAPSVFIFPPSDEQLKSGTASVVCL |
| | | | LNNFYPREAKVQWKVDNALQSGNSQESVTEQD |
| | | | SKDSTYSLSSTLTLSKADYEKHKVYACEVTHQG |
| | | | LSSPVTKSFNRGEC |
| | anti-CEA Ab light | SEQ ID | GATATTCAGATGACCCAGAGCCCGAGCAGCCT |
| | chain DNA | NO: 90 | GAGCGCGAGCGTGGGCGATCGCGTGACCATTA |
| | | | CCTGCCGCGCGAGCGAAAACCAGTATGGCTAT |
| | | | CTGGCGTGGTATCAGCAGAAACCGGGCAAAG |
| | | | TGCCGAAACTGCTGATTTATAACTATAAAAACC |
| | | | TGGTGGAAGGCGTGCCGAGCCGCTTTAGCGG |
| | | | CAGCGGCAGCGGCACCGATTTTACCCTGACCA |
| | | | TTAGCAGCCTGCAGCCGGAAGATGTGGCGAC |
| | | | CTATTATTGCCAGCATCATCTGGGCACCCCGTA |
| | | | TACCTTTGGCCAGGGCACCAAAGTGGAAATTA |
| | | | AACGAACAGTGGCAGCCCCTTCCGTCTTCATT |
| | | | TTTCCCCCTTCTGACGAACAGCTGAAATCAGG |
| | | | AACTGCTAGCGTGGTCTGTCTGCTGAACAATT |
| | | | TCTACCCCAGAGAGGCCAAGGTGCAGTGGAA |
| | | | AGTCGATAACGCTCTGCAGTCCGGCAATTCTC |
| | | | AGGAGAGTGTGACCGAACAGGACTCAAAGGA |
| | | | TAGCACATATTCCCTGTCTAGTACTCTGACCCT |
| | | | GAGCAAAGCAGACTACGAGAAGCACAAAGTG |
| | | | TATGCCTGTGAAGTCACACACCAGGGGCTGAG |
| | | | TTCACCAGTCACCAAGAGTTTCAACAGAGGG |
| | | | GAATGC |
| B-CD137xCEA | anti-CEA Ab heavy | SEQ ID | QVQLVQSGAEVKKPGASVKVSCKASGYIFTSYY |
| | chain AA | NO: 87 | LHWVRQAPGQGLEWIGYINPQTGKTSYAQKFQ |
| | | | GRVTMTRDTSTSTVYMELSSLRSEDTAVYYCAR |
| | | | EYGNYNYPLDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKKVEPKSCDKTHTCPP |
| | | | CPAPPAAGPSVFLFPPKPKDTLMISRTPEVTCVV |
| | | | VDVSHEDPEVKFNWYVDGVEVHNAKTKPREE |
| | | | QYNSTYRVVSVLTVLHQDWLNGKEYKCKVSN |
| | | | KALAAPIEKTISKAKGQPREPQVYTLPPSREEMT |
| | | | KNQVSLTCLVKGFYPSDIAVEWESNGQPENNYK |
| | | | TTPPVLDSDGSFFLYSKLTVDKSRWQQGNVFSC |
| | | | SVMHEALHNHYTQKSLSLSPGK |
| | anti-CEA Ab heavy | SEQ ID | CAGGTGCAGCTGGTGCAGAGCGGCGCGGAAG |
| | chain DNA | NO: 88 | TGAAAAAACCGGGCGCGAGCGTGAAAGTGAG |
| | | | CTGCAAAGCGAGCGGCTATATTTTTACCAGCTA |
| | | | TTACCTGCATTGGGTGCGCCAGGCGCCGGGCC |
| | | | AGGGCCTGGAATGGATTGGCTATATTAACCCG |
| | | | CAGACCGGCAAGACCAGCTATGCCCAGAAATT |
| | | | TCAGGGCCGCGTGACCATGACCCGCGATACCA |
| | | | GCACCAGCACCGTGTATATGGAACTGAGCAGC |
| | | | CTGCGCAGCGAAGATACCGCGGTGTATTATTG |
| | | | CGCGCGCGAATATGGCAACTATAACTATCCGCT |
| | | | GGATTATTGGGGCCAGGGCACCCTGGTGACCG |
| | | | TGAGCAGCGCTAGCACCAAGGGGCCCTCGGT |
| | | | CTTCCCCCTGGCACCCTCCTCCAAGAGTACTT |
| | | | CTGGGGGCACAGCGGCCCTGGGCTGCCTGGT |
| | | | CAAGGACTACTTCCCCGAACCGGTGACGGTGT |
| | | | CGTGGAACTCAGGCGCCCTGACCAGCGGCGT |
| | | | GCACACCTTCCCGGCTGTCCTACAGTCCTCAG |
| | | | GACTCTACTCCCTCAGCAGCGTGGTGACCGTG |
| | | | CCCTCCAGCAGCTTGGGCACCCAGACCTACAT |
| | | | CTGCAACGTGAATCACAAGCCCAGCAACACC |
| | | | AAGGTGGACAAGAAAGTTGAGCCAAAGTCCT |
| | | | GTGACAAGACCCACACATGCCCCCCTTGTCCT |
| | | | GCTCCACCAGCTGCAGGACCAAGCGTGTTCCT |
| | | | GTTTCCACCCAAGCCCAAGGATACCCTGATGA |
| | | | TCTCTCGGACCCCAGAGGTGACATGCGTGGTG |
| | | | GTGGATGTGAGCCACGAGGACCCCGAGGTGA |
| | | | AGTTCAACTGGTATGTGGACGGCGTGGAGGTG |
| | | | CACAATGCTAAGACCAAGCCCAGGGAGGAGC |
| | | | AGTACAACTCCACCTATAGAGTGGTGTCTGTG |
| | | | CTGACAGTGCTGCACCAGGATTGGCTGAACG |
| | | | GCAAGGAGTATAAGTGCAAGGTGTCCAATAAG |
| | | | GCCCTGGCCGCTCCTATCGAGAAGACCATCTC |
| | | | TAAGGCCAAGGGCCAGCCCAGAGAGCCTCAG |
| | | | GTGTACACACTGCCTCCATCCCGGGAAGAGAT |
| | | | GACCAAGAACCAGGTGTCTCTGACATGTCTGG |
| | | | TCAAGGGCTTCTATCCCTCTGACATCGCCGTG |
| | | | GAGTGGGAGAGCAATGGCCAGCCTGAGAACA |
| | | | ATTACAAGACCACACCCCCTGTGCTGGATTCC |
| | | | GACGGCTCTTTCTTTCTGTATAGCAAGCTGACC |
| | | | GTGGACAAGTCCCGGTGGCAGCAGGGCAACG |
| | | | TGTTCAGCTGTTCCGTGATGCACGAAGCTCTG |
| | | | CATAATCACTATACTCAGAAATCCCTGTCACTG |
| | | | TCACCTGGTAAA |
| | B-BGA-4712/CEA | SEQ ID | DIQMTQSPSSLSASVGDRVTITCRASENQYGYL |
| | light chain AA | NO: 93 | AWYQQKPGKVPKLLIYNYKNLVEGVPSRFSGSG |
| | | | SGTDFTLTISSLQPEDVATYYCQHHLGTPYTFGQ |
| | | | GTKVEIKRTVAAPSVFIFPPSDEQLKSGTASVVCL |
| | | | LNNFYPREAKVQWKVDNALQSGNSQESVTEQD |
| | | | SKDSTYSLSSTLTLSKADYEKHKVYACEVTHQG |
| | | | LSSPVTKSFNRGECGGGGSEVQLLESGGGLVQP |
| | | | GGSLRLSCAASGFMLSAEDVGWVRQAPGKGLE |
| | | | WVSAILDFGGSTYYADSVKGRFTISRDNSKNTL |
| | | | YLQMNSLRAEDTAVYYCARVVYHAGGGVTFD |
| | | | YWGQGTLVTVSS |
| | B-BGA-4712/CEA | SEQ ID | GATATTCAGATGACCCAGAGCCCGAGCAGCCT |
| | light chain DNA | NO: 94 | GAGCGCGAGCGTGGGCGATCGCGTGACCATTA |
| | | | CCTGCCGCGCGAGCGAAAACCAGTATGGCTAT |
| | | | CTGGCGTGGTATCAGCAGAAACCGGGCAAAG |
| | | | TGCCGAAACTGCTGATTTATAACTATAAAAACC |
| | | | TGGTGGAAGGCGTGCCGAGCCGCTTTAGCGG |
| | | | CAGCGGCAGCGGCACCGATTTTACCCTGACCA |
| | | | TTAGCAGCCTGCAGCCGGAAGATGTGGCGAC |
| | | | CTATTATTGCCAGCATCATCTGGGCACCCCGTA |
| | | | TACCTTTGGCCAGGGCACCAAAGTGGAAATTA |
| | | | AACGAACAGTGGCAGCCCCTTCCGTCTTCATT |
| | | | TTTCCCCCTTCTGACGAACAGCTGAAATCAGG |
| | | | AACTGCTAGCGTGGTCTGTCTGCTGAACAATT |
| | | | TCTACCCCAGAGAGGCCAAGGTGCAGTGGAA |
| | | | AGTCGATAACGCTCTGCAGTCCGGCAATTCTC |
| | | | AGGAGAGTGTGACCGAACAGGACTCAAAGGA |
| | | | TAGCACATATTCCCTGTCTAGTACTCTGACCCT |
| | | | GAGCAAAGCAGACTACGAGAAGCACAAAGTG |
| | | | TATGCCTGTGAAGTCACACACCAGGGGCTGAG |
| | | | TTCACCAGTCACCAAGAGTTTCAACAGAGGG |
| | | | GAATGCGGGGGAGGCGGGTCCGAGGTCCAGT |
| | | | TACTTGAGAGTGGTGGAGGTCTGGTCCAACCA |
| | | | GGAGGTTCGCTGCGTTTATCCTGCGCCGCGTC |
| | | | TGGATTCATGTTGTCCGCCGAAGACGTGGGTT |
| | | | GGGTGCGTCAAGCGCCGGGGAAAGGACTGGA |
| | | | ATGGGTCTCCGCCATCTTGGATTTTGGTGGTTC |
| | | | GACATACTATGCGGACAGTGTCAAAGGGCGCT |
| | | | TTACGATCTCGCGCGATAACTCAAAAAATACT |
| | | | CTTTACCTTCAAATGAATAGCCTTCGTGCTGAG |
| | | | GACACTGCGGTGTACTACTGCGCCCGCGTCGT |
| | | | CTACCATGCTGGTGGTGGCGTCACCTTTGATTA |
| | | | CTGGGGACAGGGCACCTTAGTTACAGTCTCAT |
| | | | CG |
| | C-BGA-4712/CEA | SEQ ID | QVQLVQSGAEVKKPGASVKVSCKASGYIFTSYY |
| | heavy chain AA | NO: 95 | LHWVRQAPGQGLEWIGYINPQTGKTSYAQKFQ |
| | | | GRVTMTRDTSTSTVYMELSSLRSEDTAVYYCAR |
| | | | EYGNYNYPLDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKKVEPKSCDGGGGSE |
| | | | VQLLESGGGLVQPGGSLRLSCAASGFMLSAEDV |
| | | | GWVRQAPGKGLEWVSAILDFGGSTYYADSVKG |
| | | | RFTISRDNSKNTLYLQMNSLRAEDTAVYYCARV |
| | | | VYHAGGGVTFDYWGQGTLVTVSSSGGGGSEPK |
| | | | SCDKTHTCPPCPAPPAAGPSVFLFPPKPKDTLMIS |
| | | | RTPEVTCVVVDVSHEDPEVKFNWYVDGVEVHN |
| | | | AKTKPREEQYNSTYRVVSVLTVLHQDWLNGKE |
| | | | YKCKVSNKALAAPIEKTISKAKGQPREPQVYTL |
| | | | PPSREEMTKNQVSLTCLVKGFYPSDIAVEWESN |
| | | | GQPENNYKTTPPVLDSDGSFFLYSKLTVDKSRW |
| | | | QQGNVFSCSVMHEALHNHYTQKSLSLSPGK |
| C-CD137xCEA | C-BGA-4712/CEA | SEQ ID | CAGGTGCAGCTGGTGCAGAGCGGCGCGGAAG |
| | heavy chain DNA | NO: 96 | TGAAAAAACCGGGCGCGAGCGTGAAAGTGAG |
| | | | CTGCAAAGCGAGCGGCTATATTTTTACCAGCTA |
| | | | TTACCTGCATTGGGTGCGCCAGGCGCCGGGCC |
| | | | AGGGCCTGGAATGGATTGGCTATATTAACCCG |
| | | | CAGACCGGCAAGACCAGCTATGCCCAGAAATT |
| | | | TCAGGGCCGCGTGACCATGACCCGCGATACCA |
| | | | GCACCAGCACCGTGTATATGGAACTGAGCAGC |
| | | | CTGCGCAGCGAAGATACCGCGGTGTATTATTG |
| | | | CGCGCGCGAATATGGCAACTATAACTATCCGCT |
| | | | GGATTATTGGGGCCAGGGCACCCTGGTGACCG |
| | | | TGAGCAGCGCTAGCACCAAGGGGCCCTCGGT |
| | | | CTTCCCCCTGGCACCCTCCTCCAAGAGTACTT |
| | | | CTGGGGGCACAGCGGCCCTGGGCTGCCTGGT |
| | | | CAAGGACTACTTCCCCGAACCGGTGACGGTGT |
| | | | CGTGGAACTCAGGCGCCCTGACCAGCGGCGT |
| | | | GCACACCTTCCCGGCTGTCCTACAGTCCTCAG |
| | | | GACTCTACTCCCTCAGCAGCGTGGTGACCGTG |
| | | | CCCTCCAGCAGCTTGGGCACCCAGACCTACAT |
| | | | CTGCAACGTGAATCACAAGCCCAGCAACACC |
| | | | AAGGTGGACAAGAAAGTTGAGCCAAAGTCCT |
| | | | GTGACGGGGGAGGCGGGTCCGAGGTCCAGTT |
| | | | ACTTGAGAGTGGTGGAGGTCTGGTCCAACCA |
| | | | GGAGGTTCGCTGCGTTTATCCTGCGCCGCGTC |
| | | | TGGATTCATGTTGTCCGCCGAAGACGTGGGTT |
| | | | GGGTGCGTCAAGCGCCGGGGAAAGGACTGGA |
| | | | ATGGGTCTCCGCCATCTTGGATTTTGGTGGTTC |
| | | | GACATACTATGCGGACAGTGTCAAAGGGCGCT |
| | | | TTACGATCTCGCGCGATAACTCAAAAAATACT |
| | | | CTTTACCTTCAAATGAATAGCCTTCGTGCTGAG |
| | | | GACACTGCGGTGTACTACTGCGCCCGCGTCGT |
| | | | CTACCATGCTGGTGGTGGCGTCACCTTTGATTA |
| | | | CTGGGGACAGGGCACCTTAGTTACAGTCTCAT |
| | | | CGTCTGGTGGAGGCGGTTCAGAGCCCAAATCT |
| | | | TGTGACAAAACTCACACATGCCCACCGTGCCC |
| | | | AGCACCTCCTGCTGCCGGACCGTCAGTCTTCC |
| | | | TCTTCCCCCCAAAACCCAAGGACACCCTCATG |
| | | | ATCTCCCGGACCCCTGAGGTCACATGCGTGGT |
| | | | GGTGGACGTGAGCCACGAAGACCCTGAGGTC |
| | | | AAGTTCAACTGGTACGTGGACGGCGTGGAGG |
| | | | TGCATAATGCCAAGACAAAGCCGCGGGAGGA |
| | | | GCAGTACAACAGCACGTACCGTGTGGTCAGC |
| | | | GTCCTCACCGTCCTGCACCAGGACTGGCTGAA |
| | | | TGGCAAGGAGTACAAGTGCAAGGTCTCCAAC |
| | | | AAAGCCCTCGCAGCCCCCATCGAGAAAACCAT |
| | | | CTCCAAAGCCAAAGGGCAGCCCCGAGAACCA |
| | | | CAGGTCTACACCCTGCCCCCATCCCGGGAGGA |
| | | | GATGACCAAGAACCAGGTCAGCCTGACCTGC |
| | | | CTGGTCAAAGGCTTCTATCCCAGCGACATCGC |
| | | | CGTGGAGTGGGAGAGCAATGGGCAGCCGGAG |
| | | | AACAACTACAAGACCACGCCTCCCGTGCTGG |
| | | | ACTCCGACGGCTCCTTCTTCCTCTACAGCAAG |
| | | | CTCACCGTGGACAAGAGCAGGTGGCAGCAGG |
| | | | GGAACGTCTTCTCATGCTCCGTGATGCATGAG |
| | | | GCTCTGCACAACCACTACACGCAGAAGAGCC |
| | | | TCTCCCTGTCTCCGGGTAAA |
| | anti-CEA Ab light | SEQ ID | DIQMTQSPSSLSASVGDRVTITCRASENQYGYL |
| | chain AA | NO: 89 | AWYQQKPGKVPKLLIYNYKNLVEGVPSRFSGSG |
| | | | SGTDFTLTISSLQPEDVATYYCQHHLGTPYTFGQ |
| | | | GTKVEIKRTVAAPSVFIFPPSDEQLKSGTASVVCL |
| | | | LNNFYPREAKVQWKVDNALQSGNSQESVTEQD |
| | | | SKDSTYSLSSTLTLSKADYEKHKVYACEVTHQG |
| | | | LSSPVTKSFNRGEC |
| | anti-CEA Ab light | SEQ ID | GATATTCAGATGACCCAGAGCCCGAGCAGCCT |
| | chain DNA | NO: 90 | GAGCGCGAGCGTGGGCGATCGCGTGACCATTA |
| | | | CCTGCCGCGCGAGCGAAAACCAGTATGGCTAT |
| | | | CTGGCGTGGTATCAGCAGAAACCGGGCAAAG |
| | | | TGCCGAAACTGCTGATTTATAACTATAAAAACC |
| | | | TGGTGGAAGGCGTGCCGAGCCGCTTTAGCGG |
| | | | CAGCGGCAGCGGCACCGATTTTACCCTGACCA |
| | | | TTAGCAGCCTGCAGCCGGAAGATGTGGCGAC |
| | | | CTATTATTGCCAGCATCATCTGGGCACCCCGTA |
| | | | TACCTTTGGCCAGGGCACCAAAGTGGAAATTA |
| | | | AACGAACAGTGGCAGCCCCTTCCGTCTTCATT |
| | | | TTTCCCCCTTCTGACGAACAGCTGAAATCAGG |
| | | | AACTGCTAGCGTGGTCTGTCTGCTGAACAATT |
| | | | TCTACCCCAGAGAGGCCAAGGTGCAGTGGAA |
| | | | AGTCGATAACGCTCTGCAGTCCGGCAATTCTC |
| | | | AGGAGAGTGTGACCGAACAGGACTCAAAGGA |
| | | | TAGCACATATTCCCTGTCTAGTACTCTGACCCT |
| | | | GAGCAAAGCAGACTACGAGAAGCACAAAGTG |
| | | | TATGCCTGTGAAGTCACACACCAGGGGCTGAG |
| | | | TTCACCAGTCACCAAGAGTTTCAACAGAGGG |
| | | | GAATGC |
| D-CD137xCEA | D-BGA-4712/CEA | SEQ ID | EVQLLESGGGLVQPGGSLRLSCAASGFMLSAED |
| | heavy chain AA | NO: 97 | VGWVRQAPGKGLEWVSAILDFGGSTYYADSVK |
| | | | GRFTISRDNSKNTLYLQMNSLRAEDTAVYYCAR |
| | | | VVYHAGGGVTFDYWGQGTLVTVSSSGGGGSQ |
| | | | VQLVQSGAEVKKPGASVKVSCKASGYIFTSYYL |
| | | | HWVRQAPGQGLEWIGYINPQTGKTSYAQKFQG |
| | | | RVTMTRDTSTSTVYMELSSLRSEDTAVYYCARE |
| | | | YGNYNYPLDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKKVEPKSCDKTHTCPP |
| | | | CPAPPAAGPSVFLFPPKPKDTLMISRTPEVTCVV |
| | | | VDVSHEDPEVKFNWYVDGVEVHNAKTKPREE |
| | | | QYNSTYRVVSVLTVLHQDWLNGKEYKCKVSN |
| | | | KALAAPIEKTISKAKGQPREPQVYTLPPSREEMT |
| | | | KNQVSLTCLVKGFYPSDIAVEWESNGQPENNYK |
| | | | TTPPVLDSDGSFFLYSKLTVDKSRWQQGNVFSC |
| | | | SVMHEALHNHYTQKSLSLSPGK |
| | D-BGA-4712/CEA | SEQ ID | GAGGTCCAGTTACTTGAGAGTGGTGGAGGTCT |
| | heavy chain DNA | NO: 98 | GGTCCAACCAGGAGGTTCGCTGCGTTTATCCT |
| | | | GCGCCGCGTCTGGATTCATGTTGTCCGCCGAA |
| | | | GACGTGGGTTGGGTGCGTCAAGCGCCGGGGA |
| | | | AAGGACTGGAATGGGTCTCCGCCATCTTGGAT |
| | | | TTTGGTGGTTCGACATACTATGCGGACAGTGT |
| | | | CAAAGGGCGCTTTACGATCTCGCGCGATAACT |
| | | | CAAAAAATACTCTTTACCTTCAAATGAATAGCC |
| | | | TTCGTGCTGAGGACACTGCGGTGTACTACTGC |
| | | | GCCCGCGTCGTCTACCATGCTGGTGGTGGCGT |
| | | | CACCTTTGATTACTGGGGACAGGGCACCTTAG |
| | | | TTACAGTCTCATCGTCCGGTGGAGGCGGTTCA |
| | | | CAGGTGCAGCTGGTGCAGAGCGGCGCGGAAG |
| | | | TGAAAAAACCGGGCGCGAGCGTGAAAGTGAG |
| | | | CTGCAAAGCGAGCGGCTATATTTTTACCAGCTA |
| | | | TTACCTGCATTGGGTGCGCCAGGCGCCGGGCC |
| | | | AGGGCCTGGAATGGATTGGCTATATTAACCCG |
| | | | CAGACCGGCAAGACCAGCTATGCCCAGAAATT |
| | | | TCAGGGCCGCGTGACCATGACCCGCGATACCA |
| | | | GCACCAGCACCGTGTATATGGAACTGAGCAGC |
| | | | CTGCGCAGCGAAGATACCGCGGTGTATTATTG |
| | | | CGCGCGCGAATATGGCAACTATAACTATCCGCT |
| | | | GGATTATTGGGGCCAGGGCACCCTGGTGACCG |
| | | | TGAGCAGCGCTAGCACCAAGGGGCCCTCGGT |
| | | | CTTCCCCCTGGCACCCTCCTCCAAGAGTACTT |
| | | | CTGGGGGCACAGCGGCCCTGGGCTGCCTGGT |
| | | | CAAGGACTACTTCCCCGAACCGGTGACGGTGT |
| | | | CGTGGAACTCAGGCGCCCTGACCAGCGGCGT |
| | | | GCACACCTTCCCGGCTGTCCTACAGTCCTCAG |
| | | | GACTCTACTCCCTCAGCAGCGTGGTGACCGTG |
| | | | CCCTCCAGCAGCTTGGGCACCCAGACCTACAT |
| | | | CTGCAACGTGAATCACAAGCCCAGCAACACC |
| | | | AAGGTGGACAAGAAAGTTGAGCCAAAGTCCT |
| | | | GTGACAAGACCCACACATGCCCCCCTTGTCCT |
| | | | GCTCCACCAGCTGCAGGACCAAGCGTGTTCCT |
| | | | GTTTCCACCCAAGCCCAAGGATACCCTGATGA |
| | | | TCTCTCGGACCCCAGAGGTGACATGCGTGGTG |
| | | | GTGGATGTGAGCCACGAGGACCCCGAGGTGA |
| | | | AGTTCAACTGGTATGTGGACGGCGTGGAGGTG |
| | | | CACAATGCTAAGACCAAGCCCAGGGAGGAGC |
| | | | AGTACAACTCCACCTATAGAGTGGTGTCTGTG |
| | | | CTGACAGTGCTGCACCAGGATTGGCTGAACG |
| | | | GCAAGGAGTATAAGTGCAAGGTGTCCAATAAG |
| | | | GCCCTGGCCGCTCCTATCGAGAAGACCATCTC |
| | | | TAAGGCCAAGGGCCAGCCCAGAGAGCCTCAG |
| | | | GTGTACACACTGCCTCCATCCCGGGAAGAGAT |
| | | | GACCAAGAACCAGGTGTCTCTGACATGTCTGG |
| | | | TCAAGGGCTTCTATCCCTCTGACATCGCCGTG |
| | | | GAGTGGGAGAGCAATGGCCAGCCTGAGAACA |
| | | | ATTACAAGACCACACCCCCTGTGCTGGATTCC |
| | | | GACGGCTCTTTCTTTCTGTATAGCAAGCTGACC |
| | | | GTGGACAAGTCCCGGTGGCAGCAGGGCAACG |
| | | | TGTTCAGCTGTTCCGTGATGCACGAAGCTCTG |
| | | | CATAATCACTATACTCAGAAATCCCTGTCACTG |
| | | | TCACCTGGTAAA |
| | anti-CEA Ab light | SEQ ID | DIQMTQSPSSLSASVGDRVTITCRASENQYGYL |
| | chain AA | NO: 89 | AWYQQKPGKVPKLLIYNYKNLVEGVPSRFSGSG |
| | | | SGTDFTLTISSLQPEDVATYYCQHHLGTPYTFGQ |
| | | | GTKVEIKRTVAAPSVFIFPPSDEQLKSGTASVVCL |
| | | | LNNFYPREAKVQWKVDNALQSGNSQESVTEQD |
| | | | SKDSTYSLSSTLTLSKADYEKHKVYACEVTHQG |
| | | | LSSPVTKSFNRGEC |
| | anti-CEA Ab light | SEQ ID | GATATTCAGATGACCCAGAGCCCGAGCAGCCT |
| | chain DNA | NO: 90 | GAGCGCGAGCGTGGGCGATCGCGTGACCATTA |
| | | | CCTGCCGCGCGAGCGAAAACCAGTATGGCTAT |
| | | | CTGGCGTGGTATCAGCAGAAACCGGGCAAAG |
| | | | TGCCGAAACTGCTGATTTATAACTATAAAAACC |
| | | | TGGTGGAAGGCGTGCCGAGCCGCTTTAGCGG |
| | | | CAGCGGCAGCGGCACCGATTTTACCCTGACCA |
| | | | TTAGCAGCCTGCAGCCGGAAGATGTGGCGAC |
| | | | CTATTATTGCCAGCATCATCTGGGCACCCCGTA |
| | | | TACCTTTGGCCAGGGCACCAAAGTGGAAATTA |
| | | | AACGAACAGTGGCAGCCCCTTCCGTCTTCATT |
| | | | TTTCCCCCTTCTGACGAACAGCTGAAATCAGG |
| | | | AACTGCTAGCGTGGTCTGTCTGCTGAACAATT |
| | | | TCTACCCCAGAGAGGCCAAGGTGCAGTGGAA |
| | | | AGTCGATAACGCTCTGCAGTCCGGCAATTCTC |
| | | | AGGAGAGTGTGACCGAACAGGACTCAAAGGA |
| | | | TAGCACATATTCCCTGTCTAGTACTCTGACCCT |
| | | | GAGCAAAGCAGACTACGAGAAGCACAAAGTG |
| | | | TATGCCTGTGAAGTCACACACCAGGGGCTGAG |
| | | | TTCACCAGTCACCAAGAGTTTCAACAGAGGG |
| | | | GAATGC |

TABLE 7
Summary of yields and biochemical properties

| | | | | | human | | |
|---|---|---|---|---|---|---|---|
| | | | | | CD137 | CT26/ | Hut78/ |
| | | | | | ECD-mIgG2a | CEA | huCD137 |
| Antibody | Yield | HMW | Monomer | LMW | SPR KD | EC50 | EC50 |
| formats | (mg/L) | (%) | (%) | (%) | (M) | (ug/ml) | (ug/ml) |
| A-CD137 × CEA | 30.00 | 1.28 | 96.34 | 2.39 | 3.60E−07 | 0.75 | 0.57 |
| D-CD137 × CEA | 11.00 | 1.80 | 98.19% | 0.0 | 7.80E−08 | 3.78 | 2.28 |

### Example 7. CD137 Based Multispecific Antibody A-CD137×CEA Activates CD137 in a TAA (Tumor Associated Antigen) Dependent Way

### CD137 Based Multispecific Antibodies Induce CD137 Activation in CD137 Expressing Cells

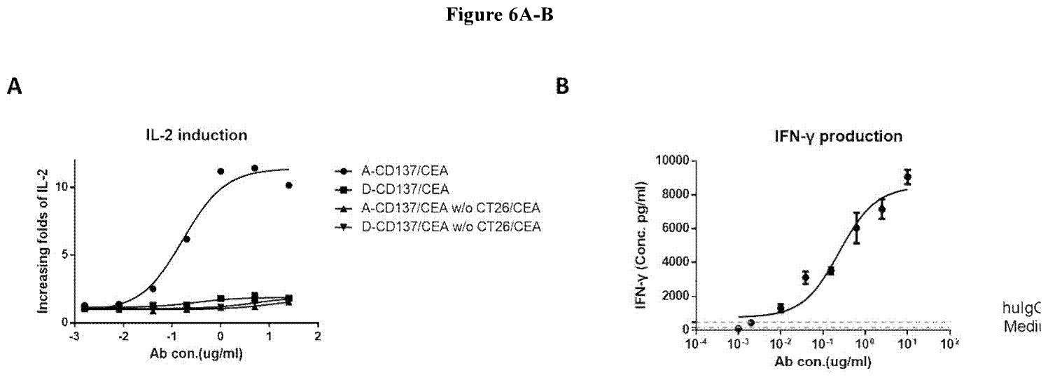

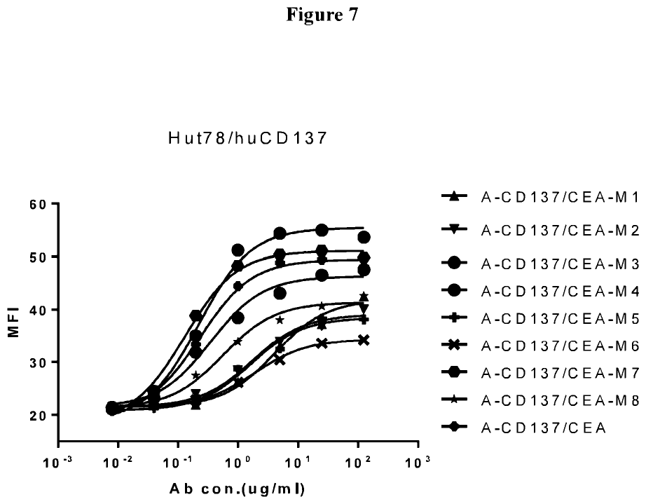

To test the ability of CD137 based multispecific antibodies to induce the response of CD137 cells to the stimuli by CEA⁺ tumor cells, the Hut78/huCD137 were used to test for CD137 activation. CEA expressing CT26 (CT26/CEA) cells were generated by retroviral transduction into CT26 cells (ATCC CRL-2638) according to the protocols described previously (Zhang et al., Blood. 2005 106(5): 1544-51). In OKT3 pre-coated 96-well plates, Hut78/huCD137 cells were co-cultured with CT26/CEA or CT26 (CEA-negative) cells overnight in the presence of CD137×CEA multispecific constructs and interleukin-2 (IL-2) was measured as an indicator of CD137 activation in Hut78/huCD137 cells. As shown in FIG. 6A, A-CD137×CEA induced Hut78/huCD137 cells to secrete IL-2 in a dose-dependent manner in the presence of the CEA⁺ CT26/CEA cells. Induction of IL-2 was not seen in the absence of CEA⁺ CT26/CEA cells.

### CD137 Based Multispecific Antibodies Induce CD137 Activation in Human Peripheral Blood Mononuclear Cells (PBMCs)

Human peripheral blood mononuclear cells (PBMCs) were isolated from whole blood of healthy donors by Ficoll (Histopaque-1077, Sigma-St. Louis MO) separation. OS8 expressing HEK293 (HEK293/OS8) cells was generated by retroviral transduction into HEK293 (ATCC CRL-1573) according to the protocols described previously (Zhang et al., Blood. 2005 106(5): 1544-51). To determine whether CD137×CEA multispecific antibodies could activate T cells in the presence of CEA⁺ tumor cells, PBMCs (2×10⁵/well) were co-cultured with HEK293/OS8 and CT26/CEA cells in the presence of CD137×CEA multispecific antibodies for 48 hours. Activation of CD137 by CD137×CEA multispecific antibodies was determined by measuring IFN-γ in PBMCs. The results showed that A-CD137×CEA could induce significant CD137 activation in PBMCs (FIG. 6B) in the presence of CEA expressing cells.

### Example 8. Removal of Post Translational Modifications

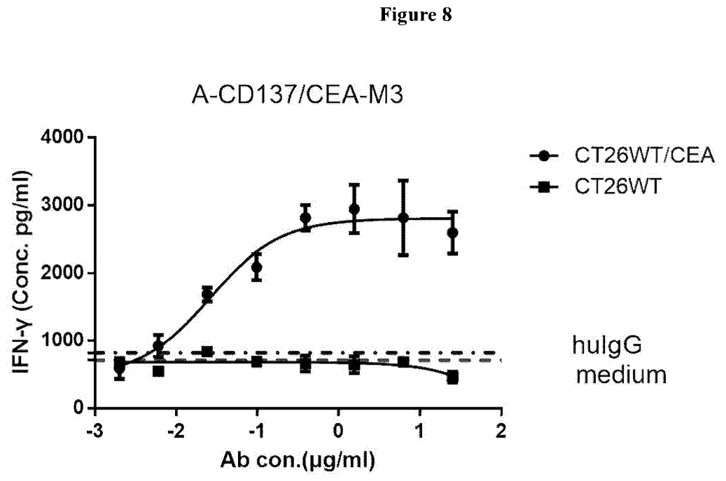

In order to remove post-translational modification (PTM) sites and to improve the expression, engineering was made by introducing mutations in HCDRs and framework regions based on the BGA-4712 sequence in the multispecific construct A-CD137×CEA. The substitutions included amino acid changes F28R, M29T, V35M, D62E, S75A and N84S in the BGA-4712 VH region. The engineering resulted in A-CD137×CEA-M1 (M28T, V34M, D62E, S75A and N84S), A-CD137×CEA-M2 (F27R, M28T, V35M, D62E, S75A and N84S), A-CD137×CEA-M3 (M28T, D62E, S75A and N84S), A-CD137×CEA-M4 (F27R, M28T, D62E, S75A and N84S), A-CD137×CEA-M5 (M28T, V35M, S75A and N84S), A-CD137×CEA-M6 (F27R, M28T, V35M, S75A and N84S), A-CD137×CEA-M7 (M28T, S75A and N84S) and A-CD137×CEA-M8 (F27R, M28T, S75A and N84S), and all of the antibodies had similar binding specificity to the parent A-CD137×CEA, and none of the changes abolished binding. While maintaining specificity, amino acid compositions and expression levels were also considered. All mutations were made using primers containing mutations at specific positions and a site directed mutagenesis kit (Cat. FM111-02, TransGen, Beijing, China). The desired mutations were verified by sequence analysis. These A-CD137×CEA variants were tested in binding (Table 8) and functional assays as described in Example 6. For affinity determination, A-CD137×CEA were captured by anti-human Fc surface, and used in the affinity assay based on surface plasmon resonance (SPR) technology. The results of SPR-determined binding profiles of variants were summarized in Table 8. The binding to Hut78/huCD137 cells of all the variants shown above were also confirmed (FIG. 8). The results showed BGA-4712-M3 (SEQ ID NO: 24-25) in A-CD137×CEA-M3 (SEQ ID NO: 101-102) was comparable with the parental antibody BGA-4712 (SEQ ID NO: 19-20). The sequence of BGA-4712-M3 is disclosed in Table 9. It is also demonstrated that A-CD137×CEA-M3 could induce CD137 activation in a PBMC based cytokine release assay as described above in the Example 7 (FIG. 8). The results further confirmed that A-CD137×CEA-M3 could induce IL-2 in a dose-dependent manner in the presence of CEA⁺ CT26/CEA cells. No induction of IL-2 and hence no activation of CD137 was seen in the absence of CEA expressing cells (FIG. 8).

TABLE 8
Comparison of A-CD137 × CEA binding affinities by SPR

| Anti-huCD137 | Ka(1/Ms) | Kd(1/s) | KD(M) |
|---|---|---|---|
| A-CD137 × CEA-M1 | 7.77E+03 | 4.55E−03 | 5.86E−07 |
| A-CD137 × CEA-M2 | 4.52E+04 | 1.05E−02 | 2.32E−07 |
| A-CD137 × CEA-M3 | 1.05E+04 | 2.67E−03 | 2.55E−07 |
| A-CD137 × CEA-M4 | 1.27E+05 | 2.47E−02 | 1.94E−07 |
| A-CD137 × CEA-M5 | 8.89E+03 | 5.74E−03 | 6.46E−07 |
| A-CD137 × CEA-M6 | 1.48E+04 | 1.14E−02 | 7.67E−07 |
| A-CD137 × CEA-M7 | 1.13E+04 | 3.00E−03 | 2.66E−07 |
| A-CD137 × CEA-M8 | 2.90E+05 | 5.34E−02 | 1.84E−07 |
| A-CD137 × CEA | 5.97E+03 | 2.15E−03 | 3.60E−07 |

TABLE 9
Sequence information of BGA-4712-M3

| Antibody | SEQ ID NO | | SEQUENCE |
|---|---|---|---|
| BGA-4712-M3 | SEQ ID NO: 24 | VH | EVQLLESGGGLVQPGGSLRLSCAASGFTLS |
| | | | AEDVGWVRQAPGKGLEWVSAILDFGGST |
| | | | YYAESVKGRFTISRDNAKNTLYLQMSSLRA |
| | | | EDTAVYYCARVVYHAGGGVTEDYWGQGT |
| | | | LVTVSS |
| | SEQ ID NO: 25 | VH DNA | GAGGTCCAGTTACTTGAGAGTGGTGGAG |
| | | | GTCTGGTCCAACCAGGAGGTTCGCTGCG |
| | | | TTTATCCTGCGCCGCGTCTGGATTCACGT |
| | | | TGTCCGCCGAAGACGTGGGTTGGGTGCG |
| | | | TCAAGCGCCGGGGAAAGGACTGGAATGG |
| | | | GTCTCCGCCATCTTGGATTTTGGTGGTTC |
| | | | GACATACTATGCGGAAAGTGTCAAAGGG |
| | | | CGCTTTACGATCTCGCGCGATAACGCAAA |
| | | | AAATACTCTTTACCTTCAAATGTCTAGCCT |
| | | | TCGTGCTGAGGACACTGCGGTGTACTACT |
| | | | GCGCCCGCGTCGTCTACCATGCTGGTGGT |
| | | | GGCGTCACCTTTGATTACTGGGGACAGG |
| | | | GCACCTTAGTTACAGTCTCATCG |
| BGA-4712-M3- | SEQ ID NO: 99 | AA | EVQLLESGGGLVQPGGSLRLSCAASGFTLS |
| mutFc | | | AEDVGWVRQAPGKGLEWVSAILDFGGST |
| | | | YYAESVKGRFTISRDNAKNTLYLQMSSLRA |
| | | | EDTAVYYCARVVYHAGGGVTFDYWGQGT |
| | | | LVTVSSGGGGSDKTHTCPPCPAPELLGGPS |
| | | | VFLFPPKPKDTLMISRTPEVTCVVVDVSHE |
| | | | DPEVKFNWYVDGVEVHNAKTKPREEQYN |
| | | | STYRVVSVLTVLHQDWLNGKEYKCKVSN |
| | | | KALPAPIEKTISKAKGQPREPQVYTLPPSRD |
| | | | ELTKNQVSLTCLVKGFYPSDIAVEWESNGQ |
| | | | PENNYKTTPPVLDSDGSFFLYSKLTVDKSR |
| | | | WQQGNVFSCSVMHEALHNHYTQKSLSLSP |
| | | | GK |
| | SEQ ID NO: 100 | DNA | GAGGTCCAGTTACTTGAGAGTGGTGGAG |
| | | | GTCTGGTCCAACCAGGAGGTTCGCTGCG |
| | | | TTTATCCTGCGCCGCGTCTGGATTCACGT |
| | | | TGTCCGCCGAAGACGTGGGTTGGGTGCG |
| | | | TCAAGCGCCGGGGAAAGGACTGGAATGG |
| | | | GTCTCCGCCATCTTGGATTTTGGTGGTTC |
| | | | GACATACTATGCGGAAAGTGTCAAAGGG |
| | | | CGCTTTACGATCTCGCGCGATAACGCAAA |
| | | | AAATACTCTTTACCTTCAAATGTCTAGCCT |
| | | | TCGTGCTGAGGACACTGCGGTGTACTACT |
| | | | GCGCCCGCGTCGTCTACCATGCTGGTGGT |
| | | | GGCGTCACCTTTGATTACTGGGGACAGG |
| | | | GCACCTTAGTTACAGTCTCATCGGGCGGC |
| | | | GGAGGGTCTGACAAAACTCACACATGCC |
| | | | CACCGTGCCCAGCACCTGAACTCCTGGG |
| | | | GGGACCGTCAGTCTTCCTCTTCCCCCCAA |
| | | | AACCCAAGGACACCCTCATGATCTCCCGG |
| | | | ACCCCTGAGGTCACATGCGTGGTGGTGG |
| | | | ACGTGAGCCACGAAGACCCTGAGGTCAA |
| | | | GTTCAACTGGTACGTGGACGGCGTGGAG |
| | | | GTGCATAATGCCAAGACAAAGCCGCGGG |
| | | | AGGAGCAGTACAACAGCACGTACCGTGT |
| | | | GGTCAGCGTCCTCACCGTCCTGCACCAG |
| | | | GACTGGCTGAATGGCAAGGAGTACAAGT |
| | | | GCAAGGTCTCCAACAAAGCCCTCCCAGC |
| | | | CCCCATCGAGAAAACCATCTCCAAAGCC |
| | | | AAAGGGCAGCCCCGAGAACCACAGGTGT |
| | | | ACACCCTGCCCCCATCCCGGGATGAGCTG |
| | | | ACCAAGAACCAGGTCAGCCTGACCTGCC |
| | | | TGGTCAAAGGCTTCTATCCCAGCGACATC |
| | | | GCCGTGGAGTGGGAGAGCAATGGGCAGC |
| | | | CGGAGAACAACTACAAGACCACGCCTCC |
| | | | CGTGCTGGACTCCGACGGCTCCTTCTTCC |
| | | | TCTACAGCAAGCTCACCGTGGACAAGAG |
| | | | CAGGTGGCAGCAGGGGAACGTCTTCTCA |
| | | | TGCTCCGTGATGCATGAGGCTCTGCACAA |
| | | | CCACTACACGCAGAAGAGCCTCTCCCTG |
| | | | TCTCCGGGTAAA |
| A-CD137xCEA- | SEQ ID NO: 101 | AA | GSATMDMRVPAQLLGLLLLWFPGSRSQVQ |
| M3 | | | LVQSGAEVKKPGASVKVSCKASGYIFTSY |
| | | | YLHWVRQAPGQGLEWIGYINPQTGKTSYA |
| | | | QKFQGRVTMTRDTSTSTVYMELSSLRSED |
| | | | TAVYYCAREYGNYNYPLDYWGQGTLVTV |
| | | | SSASTKGPSVFPLAPSSKSTSGGTAALGCLV |
| | | | KDYFPEPVTVSWNSGALTSGVHTFPAVLQS |
| | | | SGLYSLSSVVTVPSSSLGTQTYICNVNHKPS |
| | | | NTKVDKKVEPKSCDKTHTCPPCPAPPAAGP |
| | | | SVFLFPPKPKDTLMISRTPEVTCVVVDVSH |
| | | | EDPEVKFNWYVDGVEVHNAKTKPREEQY |
| | | | NSTYRVVSVLTVLHQDWLNGKEYKCKVS |
| | | | NKALAAPIEKTISKAKGQPREPQVYTLPPS |
| | | | REEMTKNQVSLTCLVKGFYPSDIAVEWESN |
| | | | GQPENNYKTTPPVLDSDGSFFLYSKLTVDK |
| | | | SRWQQGNVFSCSVMHEALHNHYTQKSLSL |
| | | | SPGKGGGGSEVQLLESGGGLVQPGGSLRLS |
| | | | CAASGFTLSAEDVGWVRQAPGKGLEWVS |
| | | | AILDFGGSTYYAESVKGRFTISRDNAKNTL |
| | | | YLQMSSLRAEDTAVYYCARVVYHAGGGV |
| | | | TFDYWGQGTLVTVSS |
| | SEQ ID NO: 102 | DNA | GGATCCGCCACCATGGATATGAGAGTTCC |
| | | | TGCTCAATTGCTGGGGTTGCTCTTGCTCT |
| | | | GGTTTCCTGGCTCGAGAAGCCAGGTGCA |
| | | | GCTGGTGCAGAGCGGCGCGGAAGTGAAA |
| | | | AAACCGGGCGCGAGCGTGAAAGTGAGCT |
| | | | GCAAAGCGAGCGGCTATATTTTTACCAGC |
| | | | TATTACCTGCATTGGGTGCGCCAGGCGCC |
| | | | GGGCCAGGGCCTGGAATGGATTGGCTATA |
| | | | TTAACCCGCAGACCGGCAAGACCAGCTA |
| | | | TGCCCAGAAATTTCAGGGCCGCGTGACC |
| | | | ATGACCCGCGATACCAGCACCAGCACCG |
| | | | TGTATATGGAACTGAGCAGCCTGCGCAGC |
| | | | GAAGATACCGCGGTGTATTATTGCGCGCG |
| | | | CGAATATGGCAACTATAACTATCCGCTGG |
| | | | ATTATTGGGGCCAGGGCACCCTGGTGACC |
| | | | GTGAGCAGCGCTAGCACCAAGGGGCCCT |
| | | | CGGTCTTCCCCCTGGCACCCTCCTCCAAG |
| | | | AGTACTTCTGGGGGCACAGCGGCCCTGG |
| | | | GCTGCCTGGTCAAGGACTACTTCCCCGA |
| | | | ACCGGTGACGGTGTCGTGGAACTCAGGC |
| | | | GCCCTGACCAGCGGCGTGCACACCTTCC |
| | | | CGGCTGTCCTACAGTCCTCAGGACTCTAC |
| | | | TCCCTCAGCAGCGTGGTGACCGTGCCCTC |
| | | | CAGCAGCTTGGGCACCCAGACCTACATCT |
| | | | GCAACGTGAATCACAAGCCCAGCAACAC |
| | | | CAAGGTGGACAAGAAAGTTGAGCCAAA |
| | | | GTCCTGTGACAAGACCCACACATGCCCC |
| | | | CCTTGTCCTGCTCCACCAGCTGCAGGACC |
| | | | AAGCGTGTTCCTGTTTCCACCCAAGCCCA |
| | | | AGGATACCCTGATGATCTCTCGGACCCCA |
| | | | GAGGTGACATGCGTGGTGGTGGATGTGA |
| | | | GCCACGAGGACCCCGAGGTGAAGTTCAA |
| | | | CTGGTATGTGGACGGCGTGGAGGTGCAC |
| | | | AATGCTAAGACCAAGCCCAGGGAGGAGC |
| | | | AGTACAACTCCACCTATAGAGTGGTGTCT |
| | | | GTGCTGACAGTGCTGCACCAGGATTGGC |
| | | | TGAACGGCAAGGAGTATAAGTGCAAGGT |
| | | | GTCCAATAAGGCCCTGGCCGCTCCTATCG |
| | | | AGAAGACCATCTCTAAGGCCAAGGGCCA |
| | | | GCCCAGAGAGCCTCAGGTGTACACACTG |
| | | | CCTCCATCCCGGGAAGAGATGACCAAGA |
| | | | ACCAGGTGTCTCTGACATGTCTGGTCAAG |
| | | | GGCTTCTATCCCTCTGACATCGCCGTGGA |
| | | | GTGGGAGAGCAATGGCCAGCCTGAGAAC |
| | | | AATTACAAGACCACACCCCCTGTGCTGG |
| | | | ATTCCGACGGCTCTTTCTTTCTGTATAGCA |
| | | | AGCTGACCGTGGACAAGTCCCGGTGGCA |
| | | | GCAGGGCAACGTGTTCAGCTGTTCCGTG |
| | | | ATGCACGAAGCTCTGCATAATCACTATAC |
| | | | TCAGAAATCCCTGTCACTGTCACCTGGTA |
| | | | AAGGTGGAGGCGGTTCAGAGGTCCAGTT |
| | | | ACTTGAGAGTGGTGGAGGTCTGGTCCAA |
| | | | CCAGGAGGTTCGCTGCGTTTATCCTGCGC |
| | | | CGCGTCTGGATTCACGTTGTCCGCCGAAG |
| | | | ACGTGGGTTGGGTGCGTCAAGCGCCGGG |
| | | | GAAAGGACTGGAATGGGTCTCCGCCATC |
| | | | TTGGATTTTGGTGGTTCGACATACTATGC |
| | | | GGAAAGTGTCAAAGGGCGCTTTACGATC |
| | | | TCGCGCGATAACGCAAAAAATACTCTTTA |
| | | | CCTTCAAATGTCTAGCCTTCGTGCTGAGG |
| | | | ACACTGCGGTGTACTACTGCGCCCGCGTC |
| | | | GTCTACCATGCTGGTGGTGGCGTCACCTT |
| | | | TGATTACTGGGGACAGGGCACCTTAGTTA |
| | | | CAGTCTCATCGTGAAAGCTTCGTCAG |

### Example 9. Camelization

Heavy-chain antibodies (VHH) is one type of single-domain antibody, and can be generated from camels and llamas as they lack the CH1 domain (Harmsen et al., (2007) Appl Microbiol Biotechnol, 77, 13-22; Kastelic et al., (2009) J Immunol Methods, 350, 54-62).

VHHs are the smallest (about 120 amino acids) antibody fragments capable of binding to antigens. Besides smaller size, VHH are usually more stable and soluble than conventional antibodies. Therefore, these single-domain antibodies can work as modular building units for bispecific and multispecific constructs (Els Conrath et al., (2001) J Biol Chem, 276, 7346-50). “Camelization” strategies have been developed to generate autonomous human VH domain antibodies (aVH) with the favorable properties on isolated human VH domains (Riechmann et al., (1999) J Immunol Methods, 231, 25-38). It is generally believed that a series of substitutions (Val37 to Phe/Tyr, Gly44 to Glu, Leu45 to Arg, and Trp47 to Gly/Leu, Trp103 to Arg/Gly) in germline contributed to those highly desirable properties (Vincke et al., (2009) J Biol Chem, 284, 3273-84; Nguyen et al., (2000) Embo J, 19, 921-30; Kunz et al., (2018) Sci Rep, 8, 7934). Other attempts to generate binders based on isolated human VH domains have also been tried and been successful (Jespers et al., (2004a) Nat Biotechnol, 22, 1161-5; Jespers et al., (2004b) J Mol Biol, 337, 893-903; Barthelemy et al., (2008) J Biol Chem, 283, 3639-54). It has been demonstrated that although consensus domains from many human families readily aggregated when expressed in isolation, human VH3 had more favorable properties Ewert et al., (2002) Biochemistry, 41, 3628-36; Ewert et al., (2003) BJ Mol Biol, 325, 531-53).

In order to improve biochemical and biophysical properties of the selected VH domain antibody, “camelization” strategies were applied based on BGA-4712-M3 (SEQ. NO. 24-25). The considerations include amino acid compositions, heat stability (Tm), surface hydrophobicity and isoelectronic points (pIs) while maintaining functional activities. Substitutions were made mainly in the framework 2 (FW2) and framework 4 (FW4), such as V37F or Y, G44/E, L45/R or G or Y, and W47/G or S or F or L or R or Y, W103/R (Kabat definition, Table 10). The variants were expressed in both Fc fusion VH and A-CD137×CEA multispecific antibody format as described previously in Example 6. The substitutions without significant affinity reduction were identified (Table 11). It was demonstrated that the change of W103R in BGA-4712-M3 showed improved solubility, non-specific binding and yield, and was given the designation BGA-7556. Taken together, the results showed BGA-7556 (SEQ. NO. 103-104) in format A-CD137×CEA (SEQ. NO. 107-108) was very similar in binding affinities as the parental antibody BGA-4712 (SEQ. NO. 19-20). The sequence information for BGA-7556 was listed in Table 12.

TABLE 10
Summary of residues for substitution

| Residue | AA substitution |
|---|---|
| V37 | F, Y |
| G44 | E |
| L45 | R, G, Y |
| W47 | G, S, F, L, R, Y |
| W103 | R |

TABLE 11
Comparison of binding affinities

| | | | | EC50 on |
|---|---|---|---|---|
| | | | | Hut78/ |
| | | | | huCD137 |
| | | SEC-HPLC | KD | cells |
| Antibody | Format | (%) | (M) | (ug/ml) |
| BGA-4712-M3 | Fc fusion VH | 97.98 | 1.69E−07 | 2.37 |
| BGA-4712-M3 | Format A- | 97.06 | 2.55E−07 | 0.52 |
| | CD137 × CEA | | | |
| BGA-7556 | Fc fusion VH | 98.36 | 2.00E−07 | 5.71 |
| BGA-7556 | Format A- | 92.30 | 6.69E−07 | 3.23 |
| | CD137 × CEA | | | |

TABLE 12
Sequence information of BGA-4712-M3 and BGA-7556 in Fc fusion VH and A-
CD137xCEA multispecific antibody format

| Antibody | SEQ ID NO | | SEQUENCE |
|---|---|---|---|
| BGA-7556 | SEQ ID NO: 103 | VH | EVQLLESGGGLVQPGGSLRLSCAASGFT |
| | | | LSAEDVGWVRQAPGKGLEWVSAILDFG |
| | | | GSTYYAESVKGRFTISRDNAKNTLYLQM |
| | | | SSLRAEDTAVYYCARVVYHAGGGVTFD |
| | | | YRGQGTQVTVSS |
| | SEQ ID NO: 104 | VH DNA | GAGGTCCAGTTACTTGAGAGTGGTGGA |
| | | | GGTCTGGTCCAACCAGGAGGTTCGCTG |
| | | | CGTTTATCCTGCGCCGCGTCTGGATTCA |
| | | | CGTTGTCCGCCGAAGACGTGGGTTGGG |
| | | | TGCGTCAAGCGCCGGGGAAAGGACTG |
| | | | GAATGGGTCTCCGCCATCTTGGATTTTG |
| | | | GTGGTTCGACATACTATGCGGAAAGTG |
| | | | TCAAAGGGCGCTTTACGATCTCGCGCG |
| | | | ATAACGCAAAAAATACTCTTTACCTTCA |
| | | | AATGTCTAGCCTTCGTGCTGAGGACAC |
| | | | TGCGGTGTACTACTGCGCCCGCGTCGT |
| | | | CTACCATGCTGGTGGTGGCGTCACCTTT |
| | | | GATTACCGGGGACAGGGCACCCAAGTT |
| | | | ACAGTCTCATCG |
| BGA-7556- | SEQ ID NO: 105 | AA | EVQLLESGGGLVQPGGSLRLSCAASGFT |
| mutFc | | | LSAEDVGWVRQAPGKGLEWVSAILDFG |
| | | | GSTYYAESVKGRFTISRDNAKNTLYLQM |
| | | | SSLRAEDTAVYYCARVVYHAGGGVTFD |
| | | | YRGQGTQVTVSSGGGGSDKTHTCPPCPA |
| | | | PELLGGPSVFLFPPKPKDTLMISRTPEVTC |
| | | | VVVDVSHEDPEVKFNWYVDGVEVHNA |
| | | | KTKPREEQYNSTYRVVSVLTVLHQDWL |
| | | | NGKEYKCKVSNKALPAPIEKTISKAKGQ |
| | | | PREPQVYTLPPSRDELTKNQVSLTCLVKG |
| | | | FYPSDIAVEWESNGQPENNYKTTPPVLDS |
| | | | DGSFFLYSKLTVDKSRWQQGNVFSCSVM |
| | | | HEALHNHYTQKSLSLSPGK |
| | SEQ ID NO: 106 | DNA | GAGGTCCAGTTACTTGAGAGTGGTGGA |
| | | | GGTCTGGTCCAACCAGGAGGTTCGCTG |
| | | | CGTTTATCCTGCGCCGCGTCTGGATTCA |
| | | | CGTTGTCCGCCGAAGACGTGGGTTGGG |
| | | | TGCGTCAAGCGCCGGGGAAAGGACTG |
| | | | GAATGGGTCTCCGCCATCTTGGATTTTG |
| | | | GTGGTTCGACATACTATGCGGAAAGTG |
| | | | TCAAAGGGCGCTTTACGATCTCGCGCG |
| | | | ATAACGCAAAAAATACTCTTTACCTTCA |
| | | | AATGTCTAGCCTTCGTGCTGAGGACAC |
| | | | TGCGGTGTACTACTGCGCCCGCGTCGT |
| | | | CTACCATGCTGGTGGTGGCGTCACCTTT |
| | | | GATTACCGGGGACAGGGCACCCAAGTT |
| | | | ACAGTCTCATCGGGCGGCGGAGGGTCT |
| | | | GACAAAACTCACACATGCCCACCGTGC |
| | | | CCAGCACCTGAACTCCTGGGGGGACCG |
| | | | TCAGTCTTCCTCTTCCCCCCAAAACCCA |
| | | | AGGACACCCTCATGATCTCCCGGACCC |
| | | | CTGAGGTCACATGCGTGGTGGTGGACG |
| | | | TGAGCCACGAAGACCCTGAGGTCAAGT |
| | | | TCAACTGGTACGTGGACGGCGTGGAGG |
| | | | TGCATAATGCCAAGACAAAGCCGCGGG |
| | | | AGGAGCAGTACAACAGCACGTACCGTG |
| | | | TGGTCAGCGTCCTCACCGTCCTGCACC |
| | | | AGGACTGGCTGAATGGCAAGGAGTACA |
| | | | AGTGCAAGGTCTCCAACAAAGCCCTCC |
| | | | CAGCCCCCATCGAGAAAACCATCTCCA |
| | | | AAGCCAAAGGGCAGCCCCGAGAACCA |
| | | | CAGGTGTACACCCTGCCCCCATCCCGG |
| | | | GATGAGCTGACCAAGAACCAGGTCAGC |
| | | | CTGACCTGCCTGGTCAAAGGCTTCTATC |
| | | | CCAGCGACATCGCCGTGGAGTGGGAGA |
| | | | GCAATGGGCAGCCGGAGAACAACTACA |
| | | | AGACCACGCCTCCCGTGCTGGACTCCG |
| | | | ACGGCTCCTTCTTCCTCTACAGCAAGCT |
| | | | CACCGTGGACAAGAGCAGGTGGCAGC |
| | | | AGGGGAACGTCTTCTCATGCTCCGTGA |
| | | | TGCATGAGGCTCTGCACAACCACTACA |
| | | | CGCAGAAGAGCCTCTCCCTGTCTCCGG |
| | | | GTAAA |
| Format A-BGA- | SEQ ID NO: 107 | AA | QVQLVQSGAEVKKPGASVKVSCKASGYI |
| 7556 | | | FTSYYLHWVRQAPGQGLEWIGYINPQTG |
| | | | KTSYAQKFQGRVTMTRDTSTSTVYMELS |
| | | | SLRSEDTAVYYCAREYGNYNYPLDYWG |
| | | | QGTLVTVSSASTKGPSVFPLAPSSKSTSG |
| | | | GTAALGCLVKDYFPEPVTVSWNSGALTS |
| | | | GVHTFPAVLQSSGLYSLSSVVTVPSSSLG |
| | | | TQTYICNVNHKPSNTKVDKKVEPKSCDK |
| | | | THTCPPCPAPPAAGPSVFLFPPKPKDTLMI |
| | | | SRTPEVTCVVVDVSHEDPEVKFNWYVD |
| | | | GVEVHNAKTKPREEQYNSTYRVVSVLTV |
| | | | LHQDWLNGKEYKCKVSNKALAAPIEKTI |
| | | | SKAKGQPREPQVYTLPPSREEMTKNQVS |
| | | | LTCLVKGFYPSDIAVEWESNGQPENNYK |
| | | | TTPPVLDSDGSFFLYSKLTVDKSRWQQG |
| | | | NVFSCSVMHEALHNHYTQKSLSLSPGKG |
| | | | GGGSEVQLLESGGGLVQPGGSLRLSCAA |
| | | | SGFTLSAEDVGWVRQAPGKGLEWVSAI |
| | | | LDFGGSTYYAESVKGRFTISRDNAKNTL |
| | | | YLQMSSLRAEDTAVYYCARVVYHAGGG |
| | | | VTFDYRGQGTQVTVSS |
| | SEQ ID NO: 108 | DNA | CAGGTGCAGCTGGTGCAGAGCGGCGC |
| | | | GGAAGTGAAAAAACCGGGCGCGAGCG |
| | | | TGAAAGTGAGCTGCAAAGCGAGCGGC |
| | | | TATATTTTTACCAGCTATTACCTGCATTG |
| | | | GGTGCGCCAGGCGCCGGGCCAGGGCC |
| | | | TGGAATGGATTGGCTATATTAACCCGCA |
| | | | GACCGGCAAGACCAGCTATGCCCAGAA |
| | | | ATTTCAGGGCCGCGTGACCATGACCCG |
| | | | CGATACCAGCACCAGCACCGTGTATATG |
| | | | GAACTGAGCAGCCTGCGCAGCGAAGAT |
| | | | ACCGCGGTGTATTATTGCGCGCGCGAAT |
| | | | ATGGCAACTATAACTATCCGCTGGATTA |
| | | | TTGGGGCCAGGGCACCCTGGTGACCGT |
| | | | GAGCAGCGCTAGCACCAAGGGGCCCTC |
| | | | GGTCTTCCCCCTGGCACCCTCCTCCAA |
| | | | GAGTACTTCTGGGGGCACAGCGGCCCT |
| | | | GGGCTGCCTGGTCAAGGACTACTTCCC |
| | | | CGAACCGGTGACGGTGTCGTGGAACTC |
| | | | AGGCGCCCTGACCAGCGGCGTGCACAC |
| | | | CTTCCCGGCTGTCCTACAGTCCTCAGG |
| | | | ACTCTACTCCCTCAGCAGCGTGGTGAC |
| | | | CGTGCCCTCCAGCAGCTTGGGCACCCA |
| | | | GACCTACATCTGCAACGTGAATCACAA |
| | | | GCCCAGCAACACCAAGGTGGACAAGA |
| | | | AAGTTGAGCCAAAGTCCTGTGACAAGA |
| | | | CCCACACATGCCCCCCTTGTCCTGCTCC |
| | | | ACCAGCTGCAGGACCAAGCGTGTTCCT |
| | | | GTTTCCACCCAAGCCCAAGGATACCCT |
| | | | GATGATCTCTCGGACCCCAGAGGTGAC |
| | | | ATGCGTGGTGGTGGATGTGAGCCACGA |
| | | | GGACCCCGAGGTGAAGTTCAACTGGTA |
| | | | TGTGGACGGCGTGGAGGTGCACAATGC |
| | | | TAAGACCAAGCCCAGGGAGGAGCAGT |
| | | | ACAACTCCACCTATAGAGTGGTGTCTGT |
| | | | GCTGACAGTGCTGCACCAGGATTGGCT |
| | | | GAACGGCAAGGAGTATAAGTGCAAGGT |
| | | | GTCCAATAAGGCCCTGGCCGCTCCTATC |
| | | | GAGAAGACCATCTCTAAGGCCAAGGGC |
| | | | CAGCCCAGAGAGCCTCAGGTGTACACA |
| | | | CTGCCTCCATCCCGGGAAGAGATGACC |
| | | | AAGAACCAGGTGTCTCTGACATGTCTG |
| | | | GTCAAGGGCTTCTATCCCTCTGACATCG |
| | | | CCGTGGAGTGGGAGAGCAATGGCCAG |
| | | | CCTGAGAACAATTACAAGACCACACCC |
| | | | CCTGTGCTGGATTCCGACGGCTCTTTCT |
| | | | TTCTGTATAGCAAGCTGACCGTGGACA |
| | | | AGTCCCGGTGGCAGCAGGGCAACGTGT |
| | | | TCAGCTGTTCCGTGATGCACGAAGCTC |
| | | | TGCATAATCACTATACTCAGAAATCCCT |
| | | | GTCACTGTCACCTGGTAAAGGTGGAGG |
| | | | CGGTTCAGAGGTCCAGTTACTTGAGAG |
| | | | TGGTGGAGGTCTGGTCCAACCAGGAGG |
| | | | TTCGCTGCGTTTATCCTGCGCCGCGTCT |
| | | | GGATTCACGTTGTCCGCCGAAGACGTG |
| | | | GGTTGGGTGCGTCAAGCGCCGGGGAA |
| | | | AGGACTGGAATGGGTCTCCGCCATCTT |
| | | | GGATTTTGGTGGTTCGACATACTATGCG |
| | | | GAAAGTGTCAAAGGGCGCTTTACGATC |
| | | | TCGCGCGATAACGCAAAAAATACTCTTT |
| | | | ACCTTCAAATGTCTAGCCTTCGTGCTGA |
| | | | GGACACTGCGGTGTACTACTGCGCCCG |
| | | | CGTCGTCTACCATGCTGGTGGTGGCGTC |
| | | | ACCTTTGATTACCGGGGACAGGGCACC |
| | | | CAAGTTACAGTCTCATCG |

### Example 10. Generation of Affinity Maturation Library

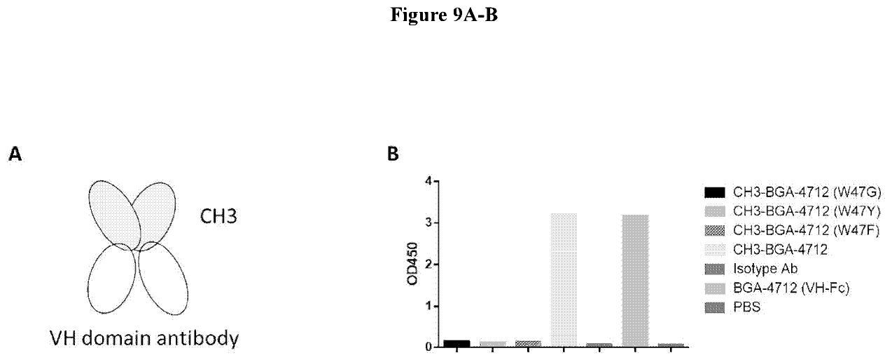

To further explore potential effective CD137 based mechanisms of action (MOAs), we aimed to generate affinity matured BGA-4712-M3 variants with improved drug-developability by phage display. In addition, we reasoned that affinity maturation library using the format of BGA-4712-M3 fusing to the c-termini of CH3 domain (SEQ ID NO: 109-110) could give us the highest possibility to gain the affinity-matured variants without CH3 interference (FIG. 9) as shown in Table 13. The library construction was described before. In brief, a phagemid vector pCANTAB 5E (GE Healthcare) was used by standard molecular biology techniques to construct a phagemid designed to display CH3-G4S (linker)-BGA-4712-M3 on the surface of M13 bacteriophage as a fusion with the N-terminus of a fragment of the gene-3 minor coat protein. There was an amber stop codon before the gene-3 sequence to allow expression of the dimer of CH3 fusion proteins directly from phagemid clones. The phagemid was used as the template to construct phage-displayed libraries containing 2.0×10⁸ unique members. All three CDRs were randomized but each CDR had a maximum of one mutation in each clone except HCDR3, which could have two simultaneous mutations. Each position was randomized with an NNK codon (IUPAC code) encoding any amino acid or an amber stop codon. The combined VH library design had a potential diversity of 5.0×10⁶ unique full-length clones and an expected distribution of about 0.02%, 1.1%, 17% and 82% of clones with 0, 1, 2, and 3 mutations, respectively. A minor fraction of heavy chain clones was expected to have 4 mutations due to primer design in the HCDR3 region. Randomization of the HCDR1, HCDR2 and HCDR3 regions was carried out via multiple site-specific mutations by polymerase chain reaction as described by Meetei et al., (1998) Anal Biochem, 264, 288-91; Meetei et al., (2002) Methods Mol Biol, 182, 95-102 and via splice-overlap extension PCR. The resulting fragments were then gel-purified and ligated with pCANTAB 5E after NcoI/NotI digestion. The purified ligations were transformed into TG1 bacteria by electroporation. Sequencing of 48 clones from each library confirmed the randomization of each position (not shown), although not all amino acid mutations could be observed in every position due to the limited sampling depth. Above 61% the library had full-length randomized clones, enough to cover all the potential diversity of the design with the 2.0×10⁸ independent clones generated even with moderate incorporation biases in oligonucleotide synthesis and library construction.

TABLE 13
Sequence information

| CONSTRUCT | SEQ ID NO | | SEQUENCE |
|---|---|---|---|
| CH3 | SEQ ID NO: | AA | GQPREPQVYTLPPSREEMTKNQVSLTCL |
| | 109 | | VKGFYPSDIAVEWESNGQPENNYKTTPP |
| | | | VLDSDGSFFLYSKLTVDKSRWQQGNVF |
| | | | SCSVMHEALHNHYTQKSLSLSPGK |
| | SEQ ID NO: | DNA | GGCCAGCCCAGAGAGCCTCAGGTGTA |
| | 110 | | CACACTGCCTCCATCCCGGGAAGAGA |
| | | | TGACCAAGAACCAGGTGTCTCTGACA |
| | | | TGTCTGGTCAAGGGCTTCTATCCCTCT |
| | | | GACATCGCCGTGGAGTGGGAGAGCAA |
| | | | TGGCCAGCCTGAGAACAATTACAAGA |
| | | | CCACACCCCCTGTGCTGGATTCCGACG |
| | | | GCTCTTTCTTTCTGTATAGCAAGCTGA |
| | | | CCGTGGACAAGTCCCGGTGGCAGCAG |
| | | | GGCAACGTGTTCAGCTGTTCCGTGATG |
| | | | CACGAAGCTCTGCATAATCACTATACT |
| | | | CAGAAATCCCTGTCACTGTCACCTGGT |
| | | | AAA |
| CH3- BGA-4712-M3 | SEQ ID NO: | AA | GQPREPQVYTLPPSREEMTKNQVSLTCL |
| | 111 | | VKGFYPSDIAVEWESNGQPENNYKTTPP |
| | | | VLDSDGSFFLYSKLTVDKSRWQQGNVF |
| | | | SCSVMHEALHNHYTQKSLSLSPGKGGG |
| | | | GSEVQLLESGGGLVQPGGSLRLSCAASG |
| | | | FTLSAEDVGWVRQAPGKGLEWVSAILD |
| | | | FGGSTYYAESVKGRFTISRDNAKNTLYL |
| | | | QMSSLRAEDTAVYYCARVVYHAGGGV |
| | | | TFDYWGQGTLVTVSS |
| | SEQ ID NO: | DNA | GGCCAGCCCAGAGAGCCTCAGGTGTA |
| | 112 | | CACACTGCCTCCATCCCGGGAAGAGA |
| | | | TGACCAAGAACCAGGTGTCTCTGACA |
| | | | TGTCTGGTCAAGGGCTTCTATCCCTCT |
| | | | GACATCGCCGTGGAGTGGGAGAGCAA |
| | | | TGGCCAGCCTGAGAACAATTACAAGA |
| | | | CCACACCCCCTGTGCTGGATTCCGACG |
| | | | GCTCTTTCTTTCTGTATAGCAAGCTGA |
| | | | CCGTGGACAAGTCCCGGTGGCAGCAG |
| | | | GGCAACGTGTTCAGCTGTTCCGTGATG |
| | | | CACGAAGCTCTGCATAATCACTATACT |
| | | | CAGAAATCCCTGTCACTGTCACCTGGT |
| | | | AAAGGTGGAGGCGGTTCAGAGGTCCA |
| | | | GTTACTTGAGAGTGGTGGAGGTCTGG |
| | | | TCCAACCAGGAGGTTCGCTGCGTTTAT |
| | | | CCTGCGCCGCGTCTGGATTCACGTTGT |
| | | | CCGCCGAAGACGTGGGTTGGGTGCGT |
| | | | CAAGCGCCGGGGAAAGGACTGGAATG |
| | | | GGTCTCCGCCATCTTGGATTTTGGTGG |
| | | | TTCGACATACTATGCGGAAAGTGTCAA |
| | | | AGGGCGCTTTACGATCTCGCGCGATAA |
| | | | CGCAAAAAATACTCTTTACCTTCAAAT |
| | | | GTCTAGCCTTCGTGCTGAGGACACTGC |
| | | | GGTGTACTACTGCGCCCGCGTCGTCTA |
| | | | CCATGCTGGTGGTGGCGTCACCTTTGA |
| | | | TTACTGGGGACAGGGCACCTTAGTTAC |
| | | | AGTCTCATCG |

### Example 11. Generation of Affinity Matured BGA-4712-M3 Variants

### Library Selection and Screening

Generation of affinity-matured BGA-4712-M3 variants was carried out by phage display using standard protocols (Silacci et al., (2005) Proteomics, 5, 2340-50; Zhao et al., (2014) PLOS One, 9, e111339). For the first and second rounds of selections, heat-denaturation selections (Jespers et al., (2004a) Nat Biotechnol, 22, 1161-5). were performed on immobilized human CD137 ECD-mIgG2a or human CD137 ECD-his in immunotubes (Cat. 470319, ThermoFisher). In brief, immunotubes were pre-coated with human CD137 ECD-mIgG2a or human CD137 ECD-HIS (10 μg/ml in PBS) overnight at 4° ° C. The affinity maturation library was heated to 70° ° C. for 10 min, then cooled down to 4ºC for 30 min. The heat-denatured phage library was incubated with the pre-coated immunotubes for 1 hour. For the third and fourth rounds of selections, cell panning was carried out using Hut78/huCD137 cells with HEK293 cells as depletion cells. After four rounds of selections, individual clones were picked up and phage containing supernatants were prepared as described in standard protocols. ELISA-positive clones were sequenced, and mutation sites were analyzed.

### Analysis of Mutation Frequency in CDRs

The frequency of mutations in each HCDR after four rounds of selection was relatively high. The mutation rates were 78.13% for HCDR1, 93.75% for HCDR2 and 96.85% for HCDR3. HCDR1, contain a more diverse array of changes. Residues 29 was mutated from Leu to Ile in 11.45% and Leu to Val in 30.21% of the clones. Positions 26, 28, 30, and 31 do not have a high mutation frequency, and with no obvious pattern, which included large, hydrophobic and polar residues, such as Tyr, Phe, Thr and Asn. For HCDR2, F55 had mutations occurring in 90.63% clones. The residue 55 was mutated form Phe to Asn (22.18%), Phe to Lys (22.18%), Phe to Ser (11.46%) and Phe to Gln (9.38%) of the clones. There were also a small number of clones that contained changes to other residues, such as Leu, Gly, Tyr, Thr and His. HCDR3 had changes occurring at three sites in at least 90% of the clones, and two of them had additional mutations in around 50% clones. Residue 109 was mutated from Phe to similar hydrophobic residues, such as Tyr and Trp. Residue 99 was mutated from Val to Tyr (15.63%) and Val to Ile (28.13%). Residue 110 was changed from Tyr to Thr (55.20%) and Tyr to Leu (7.29%) of the clones. FIG. 10 shows the sequences of HCDR regions after four rounds of selections.

### Expression of Selected Variants

All mutations were introduced in BGA-7556 (SEQ ID NO: 103-104) to make affinity-matured variants except for BGA-3386, of which the mutations were introduced in BGA-4712-M3 (SEQ ID NO: 24-25). All variants were expressed as both monoclonal antibodies (VH-Fc) and their corresponding multispecific antibodies in Format A (A-CD137×CEA) as described in Examples 5 and 6. The purified antibodies were concentrated to 0.5-10 mg/mL in PBS and stored in aliquots in a −80° C. freezer.

### Characterization of Selected Variants

Affinity comparison of and affinity matured clones was made by SPR assays using BIAcore™ T-200 (GE Life Sciences) and flow cytometry as described in Example 5. Briefly, anti-human IgG (Fc) antibody was immobilized on an activated CM5 biosensor chip (Cat.: BR100839, GE Life Sciences). Anti-huCD137 monoclonal antibodies or multispecific antibodies were flown through the chip surface and captured by anti-human IgG (Fc) antibody. Then a serial dilution of human CD137 ECD-mIgG2a (6.0 nM to 2150 nM) was flown over the chip surface and changes in surface plasmon resonance signals was analyzed to calculate the association rates (kₒₙ) and dissociation rates (k_{off}) by using the one-to-one Langmuir binding model (BIA Evaluation Software, GE Life Sciences). The equilibrium dissociation constant (K_{D})) was calculated as the ratio k_{off}/kₒₙ. For flow cytometry, CD137-expressing cells (10⁵ cells/well) were incubated with various concentrations of purified antibodies, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). Moreover, for multispecific antibodies, the affinity on human CEA were also measured by SPR assays using in-house made recombinant CEA proteins. The binding to CEA-expressing cells was also confirmed by flow cytometry. The sequence information is shown in Table 16 and the results of SPR-determined binding profiles of anti-huCD137 antibodies are summarized in Table 14 and 15. Three variants with different affinities towards human CD137 were selected for further characterization.

TABLE 14
Affinity comparison of affinity matured
BGA-4712 variants as Fc fusion

| Clone # | Format | SEC-HPLC (%) | KD (M) |
|---|---|---|---|
| BGA-5623 | Fc-fusion VH | 97.62 | 5.03E−08 |
| BGA-2690 | | 97.82 | 6.75E−05 |
| BGA-3849 | | 98.75 | 7.63E−08 |
| BGA-7916 | | 97.57 | 8.77E−08 |
| BGA-4988 | | 97.71 | 1.03E−06 |
| BGA-5164 | | 99.07 | 1.38E−05 |
| BGA-3953 | | 97.29 | 4.83E−08 |
| BGA-5385 | | 97.86 | 1.00E−07 |
| BGA-9468 | | 97.56 | 1.90E−07 |
| BGA-3285 | | 97.83 | 1.86E−07 |
| BGA-9442 | | 98.6 | 1.51E−07 |
| BGA-7746 | | 97.68 | 1.93E−07 |
| BGA-8425 | | 98.2 | 9.34E−08 |
| BGA-6468 | | 92.00 | 1.12E−08 |
| BGA-4712-M3 | | 97.98 | 1.69E−07 |
| BGA-7556 | | 98.36 | 2.00E−07 |

TABLE 15
Affinity Comparison of affinity matured BGA-4712 variants

| | | SEC- | |
|---|---|---|---|
| Clone # | Format | HPLC (%) | KD (M) |
| Format A -BGA-5623 | A-CD137 × CEA | 94.55 | 9.48E−08 |
| Format A - BGA-2690 | | — | — |
| Format A - BGA-3849 | | — | — |
| Format A- BGA-7916 | | 98.38 | 2.03E−07 |
| Format A- BGA-4988 | | 98.43 | — |
| Format A - BGA-5164 | | 98.36 | — |
| Format A - BGA-3953 | | 96.19 | 1.15E−07 |
| Format A - BGA-5385 | | 97.18 | 1.54E−07 |
| Format A - BGA-9468 | | 98.55 | 8.73E−07 |
| Format A - BGA-3285 | | 97.32 | 7.98E−07 |
| Format A - BGA-9442 | | 98.67 | 1.64E−07 |
| Format A - BGA-7746 | | 97.66 | 2.45E−07 |
| Format A - BGA-8425 | | 95.69 | 1.89E−07 |
| Format A - BGA-3386 | | 87.00 | 2.14E−08 |
| Format A - BGA-4712-M3 | | 97.06 | 2.89E−07 |
| Format A - BGA-7556 | | 92.3 | 6.49E−07 |

TABLE 16
Sequence information for affinity matured BGA-4712 variants

| | | |
|---|---|---|
| SEQ ID NO: 33 | BGA-5623 | EVQLLESGGGLVQPGGSLRLSCAASGFTVSAEDVGWVRQA |
| | AA | PGKGLEWVSAILDKGGSTYYAESVKGRFTISRDNAKNTLYL |
| | | QMSSLRAEDTAVYYCARIVYHAGGGVTFDTRGQGTQVTVS |
| | | S |
| SEQ ID NO: 34 | BGA-5623 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATAAGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCATTGTCTACCATGCTGGTGGT |
| | | GGCGTCACCTTTGATACTCGGGGACAGGGCACCCAAGTT |
| | | ACAGTCTCATCG |
| SEQ ID NO: 113 | BGA-2690 | EVQLLESGGGLVQPGGSLRLSCAASGFTLSREDVGWVRQAP |
| | AA | GKGLEWVSAILDFGSSTYYAESVKGRFTISRDNAKNTLYLQ |
| | | MSSLRAEDTAVYYCARVVYHAGGGVTYDYRGQGTQVTVS |
| | | S |
| SEQ ID NO: 114 | BGA-2690 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGCTTTCCAGAGAAGACGTGGGTTGGGTGCGTCAAG |
| | | CGCCGGGGAAAGGACTGGAATGGGTCTCCGCCATCTTGG |
| | | ATTTCGGTTCTTCGACATACTATGCGGAAAGTGTCAAAGG |
| | | GCGCTTTACGATCTCGCGCGATAACGCAAAAAATACTCTT |
| | | TACCTTCAAATGTCTAGCCTTCGTGCTGAGGACACTGCGG |
| | | TGTACTACTGCGCCCGCGTTGTCTACCATGCTGGTGGTGG |
| | | CGTCACCTATGATTATCGGGGACAGGGCACCCAAGTTAC |
| | | AGTCTCATCG |
| SEQ ID NO: 115 | BGA-3849 | EVQLLESGGGLVQPGGSLRLSCAASGFTLSHEDVGWVRQAP |
| | AA | GKGLEWVSAILDSGGSTYYAESVKGRFTISRDNAKNTLYLQ |
| | | MSSLRAEDTAVYYCARYVYHAGGGVTFDTRGQGTQVTVSS |
| SEQ ID NO: 116 | BGA-3849 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGCTTTCCCATGAAGACGTGGGTTGGGTGCGTCAAGC |
| | | GCCGGGGAAAGGACTGGAATGGGTCTCCGCCATCTTGGA |
| | | TTCCGGTGGTTCGACATACTATGCGGAAAGTGTCAAAGG |
| | | GCGCTTTACGATCTCGCGCGATAACGCAAAAAATACTCTT |
| | | TACCTTCAAATGTCTAGCCTTCGTGCTGAGGACACTGCGG |
| | | TGTACTACTGCGCCCGCTATGTCTACCATGCTGGTGGTGG |
| | | CGTCACCTTTGATACTCGGGGACAGGGCACCCAAGTTAC |
| | | AGTCTCATCG |
| SEQ ID NO: 117 | BGA-7916 | EVQLLESGGGLVQPGGSLRLSCAASGFTVSAEDVGWVRQA |
| | AA | PGKGLEWVSAILDSGGSTYYAESVKGRFTISRDNAKNTLYL |
| | | QMSSLRAEDTAVYYCARVVYHAGGGVTYDYRGQGTQVTV |
| | | SS |
| SEQ ID NO: 118 | BGA-7916 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTATGATACTCGGGGACAGGGCACCCAAGTT |
| | | ACAGTCTCATCG |
| SEQ ID NO: 119 | BGA-4988 | EVQLLESGGGLVQPGGSLRLSCAASGFTVSAEDVGWVRQA |
| | AA | PGKGLEWVSAILDRGGSTYYAESVKGRFTISRDNAKNTLYL |
| | | QMSSLRAEDTAVYYCARVVYHAGGGVTYDYRGQGTQVTV |
| | | SS |
| SEQ ID NO: 120 | BGA-4988 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATCGGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTATGATACTCGGGGACAGGGCACCCAAGTT |
| | | ACAGTCTCATCG |
| SEQ ID NO: 121 | BGA-5164 | EVQLLESGGGLVQPGGSLRLSCAASGFTLSAEDVGWVRQAP |
| | AA | GKGLEWVSAILDHGGSTYYAESVKGRFTISRDNAKNTLYLQ |
| | | MSSLRAEDTAVYYCARVVYHAGGGVTYDYRGQGTQVTVS |
| | | S |
| SEQ ID NO: 122 | BGA-5164 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGCTTTCCGCCGAAGACGTGGGTTGGGTGCGTCAAG |
| | | CGCCGGGGAAAGGACTGGAATGGGTCTCCGCCATCTTGG |
| | | ATCATGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTATGATTATCGGGGACAGGGCACCCAAGTT |
| | | ACAGTCTCATCG |
| SEQ ID NO: 123 | BGA-3953 | EVQLLESGGGLVQPGGSLRLSCAASGFTVSAEDVGWVRQA |
| | AA | PGKGLEWVSAILDSGGSTYYAESVKGRFTISRDNAKNTLYL |
| | | QMSSLRAEDTAVYYCARIVYHAGGGVTFDTRGQGTQVTVS |
| | | S |
| SEQ ID NO: 124 | BGA-3953 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCATTGTCTACCATGCTGGTGGT |
| | | GGCGTCACCTTTGATACTCGGGGACAGGGCACCCAAGTT |
| | | ACAGTCTCATCG |
| SEQ ID NO: 125 | BGA-5385 | EVQLLESGGGLVQPGGSLRLSCAASGFTLSAEDVGWVRQAP |
| | AA | GKGLEWVSAILDHGGSTYYAESVKGRFTISRDNAKNTLYLQ |
| | | MSSLRAEDTAVYYCARVIYHAGGGVTFDTRGQGTQVTVSS |
| SEQ ID NO: 126 | BGA-5385 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGCTTTCCGCCGAAGACGTGGGTTGGGTGCGTCAAG |
| | | CGCCGGGGAAAGGACTGGAATGGGTCTCCGCCATCTTGG |
| | | ATCATGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTATCTACCATGCTGGTGGT |
| | | GGCGTCACCTTTGATACTCGGGGACAGGGCACCCAAGTT |
| | | ACAGTCTCATCG |
| SEQ ID NO: 127 | BGA-9468 | EVQLLESGGGLVQPGGSLRLSCAASGFTVSAEDVGWVRQA |
| | AA | PGKGLEWVSAILDSGGSTYYAESVKGRFTISRDNAKNTLYL |
| | | QMSSLRAEDTAVYYCARVVYHSGGGVTFDLRGQGTQVTVS |
| | | S |
| SEQ ID NO: 128 | BGA-9468 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATTCTGGTGGT |
| | | GGCGTCACCTTTGATCTTCGGGGACAGGGCACCCAAGTT |
| | | ACAGTCTCATCG |
| SEQ ID NO: 129 | BGA-3285 | EVQLLESGGGLVQPGGSLRLSCAASGFTISAEDVGWVRQAP |
| | AA | GKGLEWVSAILDFGGSTYYAESVKGRFTISRDNAKNTLYLQ |
| | | MSSLRAEDTAVYYCARVVYHAGGGVTFDTRGQGTQVTVSS |
| SEQ ID NO: 130 | BGA-3285 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGATTTCCGCCGAAGACGTGGGTTGGGTGCGTCAAG |
| | | CGCCGGGGAAAGGACTGGAATGGGTCTCCGCCATCTTGG |
| | | ATTTTGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTTTGATACTCGGGGACAGGGCACCCAAGTT |
| | | ACAGTCTCATCG |
| SEQ ID NO: 131 | BGA-9442 | EVQLLESGGGLVQPGGSLRLSCAASGFTISAEDVGWVRQAP |
| | AA | GKGLEWVSAILDSGGSTYYAESVKGRFTISRDNAKNTLYLQ |
| | | MSSLRAEDTAVYYCARYVYHAGGGVTFDLRGQGTQVTVSS |
| SEQ ID NO: 132 | BGA-9442 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGATTTCCGCCGAAGACGTGGGTTGGGTGCGTCAAG |
| | | CGCCGGGGAAAGGACTGGAATGGGTCTCCGCCATCTTGG |
| | | ATTCTGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCTATGTCTACCATGCTGGTGGT |
| | | GGCGTCACCTTTGATCTTCGGGGACAGGGCACCCAAGTT |
| | | ACAGTCTCATCG |
| SEQ ID NO: 133 | BGA-7746 | EVQLLESGGGLVQPGGSLRLSCAASGFTVSAEDVGWVRQA |
| | AA | PGKGLEWVSAILDFGGSTYYAESVKGRFTISRDNAKNTLYL |
| | | QMSSLRAEDTAVYYCARVVYHAGGGVTFDTRGQGTQVTV |
| | | SS |
| SEQ ID NO: 134 | BGA-7746 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTTTGGTGGTTCGACATACTATGCAGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTCGTCTACCATGCTGGTGGT |
| | | GGCGTCACCTTTGATACGCGGGGACAGGGCACCCAAGTT |
| | | ACAGTCTCATCG |
| SEQ ID NO: 135 | BGA-8425 | EVQLLESGGGLVQPGGSLRLSCAASGFTLSAEDVGWVRQAP |
| | AA | GKGLEWVSAILDSGGSTYYAESVKGRFTISRDNAKNTLYLQ |
| | | MSSLRAEDTAVYYCARVVYHAGGGVTYDYRGQGTQVTVS |
| | | S |
| SEQ ID NO: 136 | BGA-8425 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGCTGTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTCGTCTACCATGCTGGTGGT |
| | | GGCGTCACCTATGATTACCGGGGACAGGGCACCCAAGTT |
| | | ACAGTCTCATCG |
| SEQ ID NO: 137 | BGA-3386 | EVQLLESGGGLVQPGGSLRLSCAASGFTVSAEDVGWVRQA |
| | AA | PGKGLEWVSAILDKGGSTYYAESVKGRFTISRDNAKNTLYL |
| | | QMSSLRAEDTAVYYCARIVYHAGGGVTFDTWGQGTLVTVS |
| | | S |
| SEQ ID NO: 138 | BGA-3386 | GAGGTCCAGTTACTTGAGAGTGGTGGAGGTCTGGTCCAA |
| | DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATAAGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCATTGTCTACCATGCTGGTGGT |
| | | GGCGTCACCTTTGATACTTGGGGACAGGGCACCTTAGTTA |
| | | CAGTCTCATCG |
| SEQ ID NO: 139 | BGA-5623- | EVQLLESGGGLVQPGGSLRLSCAASGFTVSAEDVGWVRQA |
| | mutFc-AA | PGKGLEWVSAILDKGGSTYYAESVKGRFTISRDNAKNTLYL |
| | | QMSSLRAEDTAVYYCARIVYHAGGGVTFDTRGQGTQVTVS |
| | | SGGGGSDKTHTCPPCPAPELLGGPSVFLFPPKPKDTLMISRTP |
| | | EVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 140 | BGA-5623- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATAAGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCATTGTCTACCATGCTGGTGGT |
| | | GGCGTCACCTTTGATACTCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 141 | BGA-2690- | EVQLLESGGGLVQPGGSLRLSCAASGFTLSREDVGWVRQAP |
| | mutFc AA | GKGLEWVSAILDFGSSTYYAESVKGRFTISRDNAKNTLYLQ |
| | | MSSLRAEDTAVYYCARVVYHAGGGVTYDYRGQGTQVTVS |
| | | SGGGGSDKTHTCPPCPAPELLGGPSVFLFPPKPKDTLMISRTP |
| | | EVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 142 | BGA-2690 | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGCTTTCCAGAGAAGACGTGGGTTGGGTGCGTCAAG |
| | | CGCCGGGGAAAGGACTGGAATGGGTCTCCGCCATCTTGG |
| | | ATTTCGGTTCTTCGACATACTATGCGGAAAGTGTCAAAGG |
| | | GCGCTTTACGATCTCGCGCGATAACGCAAAAAATACTCTT |
| | | TACCTTCAAATGTCTAGCCTTCGTGCTGAGGACACTGCGG |
| | | TGTACTACTGCGCCCGCGTTGTCTACCATGCTGGTGGTGG |
| | | CGTCACCTATGATTATCGGGGACAGGGCACCCAAGTTAC |
| | | AGTCTCATCGGGCGGCGGAGGGTCTGACAAAACTCACAC |
| | | ATGCCCACCGTGCCCAGCACCTGAACTCCTGGGGGGACC |
| | | GTCAGTCTTCCTCTTCCCCCCAAAACCCAAGGACACCCTC |
| | | ATGATCTCCCGGACCCCTGAGGTCACATGCGTGGTGGTG |
| | | GACGTGAGCCACGAAGACCCTGAGGTCAAGTTCAACTGG |
| | | TACGTGGACGGCGTGGAGGTGCATAATGCCAAGACAAAG |
| | | CCGCGGGAGGAGCAGTACAACAGCACGTACCGTGTGGTC |
| | | AGCGTCCTCACCGTCCTGCACCAGGACTGGCTGAATGGC |
| | | AAGGAGTACAAGTGCAAGGTCTCCAACAAAGCCCTCCCA |
| | | GCCCCCATCGAGAAAACCATCTCCAAAGCCAAAGGGCAG |
| | | CCCCGAGAACCACAGGTGTACACCCTGCCCCCATCCCGG |
| | | GATGAGCTGACCAAGAACCAGGTCAGCCTGACCTGCCTG |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCTAC |
| | | AGCAAGCTCACCGTGGACAAGAGCAGGTGGCAGCAGGG |
| | | GAACGTCTTCTCATGCTCCGTGATGCATGAGGCTCTGCAC |
| | | AACCACTACACGCAGAAGAGCCTCTCCCTGTCTCCGGGT |
| | | AAA |
| SEQ ID NO: 143 | BGA-3849- | EVQLLESGGGLVQPGGSLRLSCAASGFTLSHEDVGWVRQAP |
| | mutFc | GKGLEWVSAILDSGGSTYYAESVKGRFTISRDNAKNTLYLQ |
| | AA | MSSLRAEDTAVYYCARYVYHAGGGVTFDTRGQGTQVTVSS |
| | | GGGGSDKTHTCPPCPAPELLGGPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| SEQ ID NO: 144 | BGA-3849 | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGCTTTCCCATGAAGACGTGGGTTGGGTGCGTCAAGC |
| | | GCCGGGGAAAGGACTGGAATGGGTCTCCGCCATCTTGGA |
| | | TTCCGGTGGTTCGACATACTATGCGGAAAGTGTCAAAGG |
| | | GCGCTTTACGATCTCGCGCGATAACGCAAAAAATACTCTT |
| | | TACCTTCAAATGTCTAGCCTTCGTGCTGAGGACACTGCGG |
| | | TGTACTACTGCGCCCGCTATGTCTACCATGCTGGTGGTGG |
| | | CGTCACCTTTGATACTCGGGGACAGGGCACCCAAGTTAC |
| | | AGTCTCATCGGGCGGCGGAGGGTCTGACAAAACTCACAC |
| | | ATGCCCACCGTGCCCAGCACCTGAACTCCTGGGGGGACC |
| | | GTCAGTCTTCCTCTTCCCCCCAAAACCCAAGGACACCCTC |
| | | ATGATCTCCCGGACCCCTGAGGTCACATGCGTGGTGGTG |
| | | GACGTGAGCCACGAAGACCCTGAGGTCAAGTTCAACTGG |
| | | TACGTGGACGGCGTGGAGGTGCATAATGCCAAGACAAAG |
| | | CCGCGGGAGGAGCAGTACAACAGCACGTACCGTGTGGTC |
| | | AGCGTCCTCACCGTCCTGCACCAGGACTGGCTGAATGGC |
| | | AAGGAGTACAAGTGCAAGGTCTCCAACAAAGCCCTCCCA |
| | | GCCCCCATCGAGAAAACCATCTCCAAAGCCAAAGGGCAG |
| | | CCCCGAGAACCACAGGTGTACACCCTGCCCCCATCCCGG |
| | | GATGAGCTGACCAAGAACCAGGTCAGCCTGACCTGCCTG |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCTAC |
| | | AGCAAGCTCACCGTGGACAAGAGCAGGTGGCAGCAGGG |
| | | GAACGTCTTCTCATGCTCCGTGATGCATGAGGCTCTGCAC |
| | | AACCACTACACGCAGAAGAGCCTCTCCCTGTCTCCGGGT |
| | | AAA |
| SEQ ID NO: 145 | BGA-7916- | EVQLLESGGGLVQPGGSLRLSCAASGFTVSAEDVGWVRQA |
| | mutFc AA | PGKGLEWVSAILDSGGSTYYAESVKGRFTISRDNAKNTLYL |
| | | QMSSLRAEDTAVYYCARVVYHAGGGVTYDTRGQGTQVTV |
| | | SSGGGGSDKTHTCPPCPAPELLGGPSVFLFPPKPKDTLMISRT |
| | | PEVTCVVVDVSHEDPEVKFNWYVDGVEVHNAKTKPREEQ |
| | | YNSTYRVVSVLTVLHQDWLNGKEYKCKVSNKALPAPIEKTI |
| | | SKAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 146 | BGA-7916- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTATGATACTCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 147 | BGA-4988- | EVQLLESGGGLVQPGGSLRLSCAASGFTVSAEDVGWVRQA |
| | mutFc AA | PGKGLEWVSAILDRGGSTYYAESVKGRFTISRDNAKNTLYL |
| | | QMSSLRAEDTAVYYCARVVYHAGGGVTYDTRGQGTQVTV |
| | | SSGGGGSDKTHTCPPCPAPELLGGPSVFLFPPKPKDTLMISRT |
| | | PEVTCVVVDVSHEDPEVKFNWYVDGVEVHNAKTKPREEQ |
| | | YNSTYRVVSVLTVLHQDWLNGKEYKCKVSNKALPAPIEKTI |
| | | SKAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 148 | BGA-4988- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATCGGGGGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTATGATACTCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 149 | BGA-5164- | EVQLLESGGGLVQPGGSLRLSCAASGFTLSAEDVGWVRQAP |
| | mutFc AA | GKGLEWVSAILDHGGSTYYAESVKGRFTISRDNAKNTLYLQ |
| | | MSSLRAEDTAVYYCARVVYHAGGGVTYDYRGQGTQVTVS |
| | | SGGGGSDKTHTCPPCPAPELLGGPSVFLFPPKPKDTLMISRTP |
| | | EVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 150 | BGA-5164- | GAGGTCCAGTTACTTGAGAGTGGTGGAGGTCTGGTCCAA |
| | mutFc | CCAGGAGGTTCGCTGCGTTTATCCTGCGCCGCGTCTGGAT |
| | DNA | TCACGCTTTCCGCCGAAGACGTGGGTTGGGTGCGTCAAG |
| | | CGCCGGGGAAAGGACTGGAATGGGTCTCCGCCATCTTGG |
| | | ATCATGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTATGATTATCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 151 | BGA-3953- | EVQLLESGGGLVQPGGSLRLSCAASGFTVSAEDVGWVRQA |
| | mutFc AA | PGKGLEWVSAILDSGGSTYYAESVKGRFTISRDNAKNTLYL |
| | | QMSSLRAEDTAVYYCARIVYHAGGGVTFDTRGQGTQVTVS |
| | | SGGGGSDKTHTCPPCPAPELLGGPSVFLFPPKPKDTLMISRTP |
| | | EVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 152 | BGA-3953- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCATTGTCTACCATGCTGGTGGT |
| | | GGCGTCACCTTTGATACTCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 153 | BGA-5385- | EVQLLESGGGLVQPGGSLRLSCAASGFTLSAEDVGWVRQAP |
| | mutFc AA | GKGLEWVSAILDHGGSTYYAESVKGRFTISRDNAKNTLYLQ |
| | | MSSLRAEDTAVYYCARVIYHAGGGVTYDYRGQGTQVTVSS |
| | | GGGGSDKTHTCPPCPAPELLGGPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| SEQ ID NO: 154 | BGA-5385- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGcTTTCCGCCGAAGACGTGGGTTGGGTGCGTCAAGC |
| | | GCCGGGGAAAGGACTGGAATGGGTCTCCGCCATCTTGGA |
| | | TCATGGTGGTTCGACATACTATGCGGAAAGTGTCAAAGG |
| | | GCGCTTTACGATCTCGCGCGATAACGCAAAAAATACTCTT |
| | | TACCTTCAAATGTCTAGCCTTCGTGCTGAGGACACTGCGG |
| | | TGTACTACTGCGCCCGCGTTATCTACCATGCTGGTGGTGG |
| | | CGTCACCTATGATTATCGGGGACAGGGCACCCAAGTTAC |
| | | AGTCTCATCGGGCGGCGGAGGGTCTGACAAAACTCACAC |
| | | ATGCCCACCGTGCCCAGCACCTGAACTCCTGGGGGGACC |
| | | GTCAGTCTTCCTCTTCCCCCCAAAACCCAAGGACACCCTC |
| | | ATGATCTCCCGGACCCCTGAGGTCACATGCGTGGTGGTG |
| | | GACGTGAGCCACGAAGACCCTGAGGTCAAGTTCAACTGG |
| | | TACGTGGACGGCGTGGAGGTGCATAATGCCAAGACAAAG |
| | | CCGCGGGAGGAGCAGTACAACAGCACGTACCGTGTGGTC |
| | | AGCGTCCTCACCGTCCTGCACCAGGACTGGCTGAATGGC |
| | | AAGGAGTACAAGTGCAAGGTCTCCAACAAAGCCCTCCCA |
| | | GCCCCCATCGAGAAAACCATCTCCAAAGCCAAAGGGCAG |
| | | CCCCGAGAACCACAGGTGTACACCCTGCCCCCATCCCGG |
| | | GATGAGCTGACCAAGAACCAGGTCAGCCTGACCTGCCTG |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCTAC |
| | | AGCAAGCTCACCGTGGACAAGAGCAGGTGGCAGCAGGG |
| | | GAACGTCTTCTCATGCTCCGTGATGCATGAGGCTCTGCAC |
| | | AACCACTACACGCAGAAGAGCCTCTCCCTGTCTCCGGGT |
| | | AAA |
| SEQ ID NO: 155 | BGA-9468- | EVQLLESGGGLVQPGGSLRLSCAASGFTVSAEDVGWVRQA |
| | mutFc AA | PGKGLEWVSAILDSGGSTYYAESVKGRFTISRDNAKNTLYL |
| | | QMSSLRAEDTAVYYCARVVYHSGGGVTFDLRGQGTQVTVS |
| | | SGGGGSDKTHTCPPCPAPELLGGPSVFLFPPKPKDTLMISRTP |
| | | EVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 156 | BGA-9468- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATTCTGGTGGT |
| | | GGCGTCACCTTTGATCTTCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 157 | BGA-3285- | EVQLLESGGGLVQPGGSLRLSCAASGFTISAEDVGWVRQAP |
| | mutFc | GKGLEWVSAILDFGGSTYYAESVKGRFTISRDNAKNTLYLQ |
| | AA | MSSLRAEDTAVYYCARVVYHAGGGVTFDTRGQGTQVTVSS |
| | | GGGGSDKTHTCPPCPAPELLGGPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| SEQ ID NO: 158 | BGA-3285- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGATTTCCGCCGAAGACGTGGGTTGGGTGCGTCAAG |
| | | CGCCGGGGAAAGGACTGGAATGGGTCTCCGCCATCTTGG |
| | | ATTTTGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTTGTCTACCATGCTGGTGGT |
| | | GGCGTCACCTTTGATACTCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 159 | BGA-9442- | EVQLLESGGGLVQPGGSLRLSCAASGFTISAEDVGWVRQAP |
| | mutFc AA | GKGLEWVSAILDSGGSTYYAESVKGRFTISRDNAKNTLYLQ |
| | | MSSLRAEDTAVYYCARYVYHAGGGVTFDLRGQGTQVTVSS |
| | | GGGGSDKTHTCPPCPAPELLGGPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| SEQ ID NO: 160 | BGA-9442- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGATTTCCGCCGAAGACGTGGGTTGGGTGCGTCAAG |
| | | CGCCGGGGAAAGGACTGGAATGGGTCTCCGCCATCTTGG |
| | | ATTCTGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCTATGTCTACCATGCTGGTGGT |
| | | GGCGTCACCTTTGATCTTCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 161 | BGA-7746- | EVQLLESGGGLVQPGGSLRLSCAASGFTVSAEDVGWVRQA |
| | mutFc AA | PGKGLEWVSAILDFGGSTYYAESVKGRFTISRDNAKNTLYL |
| | | QMSSLRAEDTAVYYCARVVYHAGGGVTFDTRGQGTQVTV |
| | | SSGGGGSDKTHTCPPCPAPELLGGPSVFLFPPKPKDTLMISRT |
| | | PEVTCVVVDVSHEDPEVKFNWYVDGVEVHNAKTKPREEQ |
| | | YNSTYRVVSVLTVLHQDWLNGKEYKCKVSNKALPAPIEKTI |
| | | SKAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 162 | BGA-7746- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGGTTTCCGCCGAAGACGTGGGTTGGGTGCGTCAAG |
| | | CGCCGGGGAAAGGACTGGAATGGGTCTCCGCCATCTTGG |
| | | ATTTTGGTGGTTCGACATACTATGCAGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTCGTCTACCATGCTGGTGGT |
| | | GGCGTCACCTTTGATACGCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 163 | BGA-8425- | EVQLLESGGGLVQPGGSLRLSCAASGFTLSAEDVGWVRQAP |
| | mutFc AA | GKGLEWVSAILDSGGSTYYAESVKGRFTISRDNAKNTLYLQ |
| | | MSSLRAEDTAVYYCARVVYHAGGGVTYDYRGQGTQVTVS |
| | | SGGGGSDKTHTCPPCPAPELLGGPSVFLFPPKPKDTLMISRTP |
| | | EVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 164 | BGA-8425- | GAGGTCCAGTTACTTGAGAGTGGTGGAGGTCTGGTCCAA |
| | mutFc DNA | CCAGGAGGTTCGCTGCGTTTATCCTGCGCCGCGTCTGGAT |
| | | TCACGCTGTCCGCCGAAGACGTGGGTTGGGTGCGTCAAG |
| | | CGCCGGGGAAAGGACTGGAATGGGTCTCCGCCATCTTGG |
| | | ATTCGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCGTCGTCTACCATGCTGGTGGT |
| | | GGCGTCACCTATGATTACCGGGGACAGGGCACCCAAGTT |
| | | ACAGTCTCATCGGGCGGCGGAGGGTCTGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACCC |
| | | TCATGATCTCCCGGACCCCTGAGGTCACATGCGTGGTGGT |
| | | GGACGTGAGCCACGAAGACCCTGAGGTCAAGTTCAACTG |
| | | GTACGTGGACGGCGTGGAGGTGCATAATGCCAAGACAAA |
| | | GCCGCGGGAGGAGCAGTACAACAGCACGTACCGTGTGGT |
| | | CAGCGTCCTCACCGTCCTGCACCAGGACTGGCTGAATGG |
| | | CAAGGAGTACAAGTGCAAGGTCTCCAACAAAGCCCTCCC |
| | | AGCCCCCATCGAGAAAACCATCTCCAAAGCCAAAGGGCA |
| | | GCCCCGAGAACCACAGGTGTACACCCTGCCCCCATCCCG |
| | | GGATGAGCTGACCAAGAACCAGGTCAGCCTGACCTGCCT |
| | | GGTCAAAGGCTTCTATCCCAGCGACATCGCCGTGGAGTG |
| | | GGAGAGCAATGGGCAGCCGGAGAACAACTACAAGACCA |
| | | CGCCTCCCGTGCTGGACTCCGACGGCTCCTTCTTCCTCTA |
| | | CAGCAAGCTCACCGTGGACAAGAGCAGGTGGCAGCAGG |
| | | GGAACGTCTTCTCATGCTCCGTGATGCATGAGGCTCTGCA |
| | | CAACCACTACACGCAGAAGAGCCTCTCCCTGTCTCCGGGT |
| | | AAA |
| SEQ ID NO: 165 | BGA-3386- | EVQLLESGGGLVQPGGSLRLSCAASGFTVSAEDVGWVRQA |
| | mutFc AA | PGKGLEWVSAILDKGGSTYYAESVKGRFTISRDNAKNTLYL |
| | | QMSSLRAEDTAVYYCARIVYHAGGGVTFDTWGQGTLVTVS |
| | | SGGGGSDKTHTCPPCPAPELLGGPSVFLFPPKPKDTLMISRTP |
| | | EVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSRDELTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |
| SEQ ID NO: 166 | BGA-3386- | GAGGTCCAGTTACTTGAGAGTGGTGGAGGTCTGGTCCAA |
| | mutFc | CCAGGAGGTTCGCTGCGTTTATCCTGCGCCGCGTCTGGAT |
| | DNA | TCACGGTTTCCGCCGAAGACGTGGGTTGGGTGCGTCAAG |
| | | CGCCGGGGAAAGGACTGGAATGGGTCTCCGCCATCTTGG |
| | | ATAAGGGTGGTTCGACATACTATGCGGAAAGTGTCAAAG |
| | | GGCGCTTTACGATCTCGCGCGATAACGCAAAAAATACTC |
| | | TTTACCTTCAAATGTCTAGCCTTCGTGCTGAGGACACTGC |
| | | GGTGTACTACTGCGCCCGCATTGTCTACCATGCTGGTGGT |
| | | GGCGTCACCTTTGATACTTGGGGACAGGGCACCTTAGTTA |
| | | CAGTCTCATCGGGCGGCGGAGGGTCTGACAAAACTCACA |
| | | CATGCCCACCGTGCCCAGCACCTGAACTCCTGGGGGGAC |
| | | CGTCAGTCTTCCTCTTCCCCCCAAAACCCAAGGACACCCT |
| | | CATGATCTCCCGGACCCCTGAGGTCACATGCGTGGTGGTG |
| | | GACGTGAGCCACGAAGACCCTGAGGTCAAGTTCAACTGG |
| | | TACGTGGACGGCGTGGAGGTGCATAATGCCAAGACAAAG |
| | | CCGCGGGAGGAGCAGTACAACAGCACGTACCGTGTGGTC |
| | | AGCGTCCTCACCGTCCTGCACCAGGACTGGCTGAATGGC |
| | | AAGGAGTACAAGTGCAAGGTCTCCAACAAAGCCCTCCCA |
| | | GCCCCCATCGAGAAAACCATCTCCAAAGCCAAAGGGCAG |
| | | CCCCGAGAACCACAGGTGTACACCCTGCCCCCATCCCGG |
| | | GATGAGCTGACCAAGAACCAGGTCAGCCTGACCTGCCTG |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCTAC |
| | | AGCAAGCTCACCGTGGACAAGAGCAGGTGGCAGCAGGG |
| | | GAACGTCTTCTCATGCTCCGTGATGCATGAGGCTCTGCAC |
| | | AACCACTACACGCAGAAGAGCCTCTCCCTGTCTCCGGGT |
| | | AAA |
| SEQ ID NO: 167 | Format A | QVQLVQSGAEVKKPGASVKVSCKASGYIFTSYYLHWVRQA |
| | BGA- | PGQGLEWIGYINPQTGKTSYAQKFQGRVTMTRDTSTSTVY |
| | 5623AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTVSAEDVG |
| | | WVRQAPGKGLEWVSAILDKGGSTYYAESVKGRFTISRDNA |
| | | KNTLYLQMSSLRAEDTAVYYCARIVYHAGGGVTFDTRGQG |
| | | TQVTVSS |
| SEQ ID NO: 168 | Format A | CAGGTGCAGCTGGTGCAGAGCGGCGCGGAAGTGAAAAA |
| | BGA-5623 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGGTTTCC |
| | | GCCGAAGACGTGGGTTGGGTGCGTCAAGCGCCGGGGAAA |
| | | GGACTGGAATGGGTCTCCGCCATCTTGGATAAGGGTGGT |
| | | TCGACATACTATGCGGAAAGTGTCAAAGGGCGCTTTACG |
| | | ATCTCGCGCGATAACGCAAAAAATACTCTTTACCTTCAAA |
| | | TGTCTAGCCTTCGTGCTGAGGACACTGCGGTGTACTACTG |
| | | CGCCCGCATTGTCTACCATGCTGGTGGTGGCGTCACCTTT |
| | | GATACTCGGGGACAGGGCACCCAAGTTACAGTCTCATCG |
| SEQ ID NO: 169 | Format A | QVQLVQSGAEVKKPGASVKVSCKASGYIFTSYYLHWVRQA |
| | BGA-2690 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTLSREDVG |
| | | WVRQAPGKGLEWVSAILDFGSSTYYAESVKGRFTISRDNAK |
| | | NTLYLQMSSLRAEDTAVYYCARVVYHAGGGVTYDYRGQG |
| | | TQVTVSS |
| SEQ ID NO: 170 | Format A | CAGGTGCAGCTGGTGCAGAGCGGCGCGGAAGTGAAAAA |
| | BGA-2690 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGCTTTCC |
| | | AGAGAAGACGTGGGTTGGGTGCGTCAAGCGCCGGGGAA |
| | | AGGACTGGAATGGGTCTCCGCCATCTTGGATTTCGGTTCT |
| | | TCGACATACTATGCGGAAAGTGTCAAAGGGCGCTTTACG |
| | | ATCTCGCGCGATAACGCAAAAAATACTCTTTACCTTCAAA |
| | | TGTCTAGCCTTCGTGCTGAGGACACTGCGGTGTACTACTG |
| | | CGCCCGCGTTGTCTACCATGCTGGTGGTGGCGTCACCTAT |
| | | GATTATCGGGGACAGGGCACCCAAGTTACAGTCTCATCG |
| SEQ ID NO: 171 | Format A | QVQLVQSGAEVKKPGASVKVSCKASGYIFTSYYLHWVRQA |
| | BGA-3849 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTLSHEDVG |
| | | WVRQAPGKGLEWVSAILDSGGSTYYAESVKGRFTISRDNA |
| | | KNTLYLQMSSLRAEDTAVYYCARYVYHAGGGVTFDTRGQ |
| | | GTQVTVSS |
| SEQ ID NO: 172 | Format A | CAGGTGCAGCTGGTGCAGAGCGGCGCGGAAGTGAAAAA |
| | BGA-3849 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGCTTTCC |
| | | CATGAAGACGTGGGTTGGGTGCGTCAAGCGCCGGGGAAA |
| | | GGACTGGAATGGGTCTCCGCCATCTTGGATTCCGGTGGTT |
| | | CGACATACTATGCGGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCTATGTCTACCATGCTGGTGGTGGCGTCACCTTTG |
| | | ATACTCGGGGACAGGGCACCCAAGTTACAGTCTCATCG |
| SEQ ID NO: 173 | Format A | QVQLVQSGAEVKKPGASVKVSCKASGYIFTSYYLHWVRQA |
| | BGA-7916 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTVSAEDVG |
| | | WVRQAPGKGLEWVSAILDSGGSTYYAESVKGRFTISRDNA |
| | | KNTLYLQMSSLRAEDTAVYYCARVVYHAGGGVTYDYRGQ |
| | | GTQVTVSS |
| SEQ ID NO: 174 | Format A | CAGGTGCAGCTGGTGCAGAGCGGCGCGGAAGTGAAAAA |
| | BGA-7916 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGGTTTCC |
| | | GCCGAAGACGTGGGTTGGGTGCGTCAAGCGCCGGGGAAA |
| | | GGACTGGAATGGGTCTCCGCCATCTTGGATtcGGGTGGTTC |
| | | GACATACTATGCGGAAAGTGTCAAAGGGCGCTTTACGAT |
| | | CTCGCGCGATAACGCAAAAAATACTCTTTACCTTCAAATG |
| | | TCTAGCCTTCGTGCTGAGGACACTGCGGTGTACTACTGCG |
| | | CCCGCGTTGTCTACCATGCTGGTGGTGGCGTCACCTATGA |
| | | TACTCGGGGACAGGGCACCCAAGTTACAGTCTCATCG |
| SEQ ID NO: 175 | Format A | QVQLVQSGAEVKKPGASVKVSCKASGYIFTSYYLHWVRQA |
| | BGA-4988 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTVSAEDVG |
| | | WVRQAPGKGLEWVSAILDRGGSTYYAESVKGRFTISRDNA |
| | | KNTLYLQMSSLRAEDTAVYYCARVVYHAGGGVTYDYRGQ |
| | | GTQVTVSS |
| SEQ ID NO: 176 | Format A | CAGGTGCAGCTGGTGCAGAGCGGCGCGGAAGTGAAAAA |
| | BGA-4988 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGGTTTCC |
| | | GCCGAAGACGTGGGTTGGGTGCGTCAAGCGCCGGGGAAA |
| | | GGACTGGAATGGGTCTCCGCCATCTTGGATCGGGGTGGTT |
| | | CGACATACTATGCGGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCGTTGTCTACCATGCTGGTGGTGGCGTCACCTATG |
| | | ATACTCGGGGACAGGGCACCCAAGTTACAGTCTCATCG |
| SEQ ID NO: 177 | Format A | QVQLVQSGAEVKKPGASVKVSCKASGYIFTSYYLHWVRQA |
| | BGA-5164 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTLSAEDVG |
| | | WVRQAPGKGLEWVSAILDHGGSTYYAESVKGRFTISRDNA |
| | | KNTLYLQMSSLRAEDTAVYYCARVVYHAGGGVTYDYRGQ |
| | | GTQVTVSS |
| SEQ ID NO: 178 | Format A | CAGGTGCAGCTGGTGCAGAGCGGCGCGGAAGTGAAAAA |
| | BGA-5164 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGcTTTCCG |
| | | CCGAAGACGTGGGTTGGGTGCGTCAAGCGCCGGGGAAAG |
| | | GACTGGAATGGGTCTCCGCCATCTTGGATCATGGTGGTTC |
| | | GACATACTATGCGGAAAGTGTCAAAGGGCGCTTTACGAT |
| | | CTCGCGCGATAACGCAAAAAATACTCTTTACCTTCAAATG |
| | | TCTAGCCTTCGTGCTGAGGACACTGCGGTGTACTACTGCG |
| | | CCCGCGTTGTCTACCATGCTGGTGGTGGCGTCACCTATGA |
| | | TTATCGGGGACAGGGCACCCAAGTTACAGTCTCATCG |
| SEQ ID NO: 179 | Format A | QVQLVQSGAEVKKPGASVKVSCKASGYIFTSYYLHWVRQA |
| | BGA-3953 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTVSAEDVG |
| | | WVRQAPGKGLEWVSAILDSGGSTYYAESVKGRFTISRDNA |
| | | KNTLYLQMSSLRAEDTAVYYCARIVYHAGGGVTFDTRGQG |
| | | TQVTVSS |
| SEQ ID NO: 180 | Format A | CAGGTGCAGCTGGTGCAGAGCGGCGCGGAAGTGAAAAA |
| | BGA-3953 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGGTTTCC |
| | | GCCGAAGACGTGGGTTGGGTGCGTCAAGCGCCGGGGAAA |
| | | GGACTGGAATGGGTCTCCGCCATCTTGGATtcGGGTGGTTC |
| | | GACATACTATGCGGAAAGTGTCAAAGGGCGCTTTACGAT |
| | | CTCGCGCGATAACGCAAAAAATACTCTTTACCTTCAAATG |
| | | TCTAGCCTTCGTGCTGAGGACACTGCGGTGTACTACTGCG |
| | | CCCGCATTGTCTACCATGCTGGTGGTGGCGTCACCTTTGA |
| | | TACTCGGGGACAGGGCACCCAAGTTACAGTCTCATCG |
| SEQ ID NO: 181 | Format A | QVQLVQSGAEVKKPGASVKVSCKASGYIFTSYYLHWVRQA |
| | BGA-5385 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTLSAEDVG |
| | | WVRQAPGKGLEWVSAILDHGGSTYYAESVKGRFTISRDNA |
| | | KNTLYLQMSSLRAEDTAVYYCARVIYHAGGGVTFDTRGQG |
| | | TQVTVSS |
| SEQ ID NO: 182 | Format A | CAGGTGCAGCTGGTGCAGAGCGGCGCGGAAGTGAAAAA |
| | BGA-5385 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGCTTTCC |
| | | GCCGAAGACGTGGGTTGGGTGCGTCAAGCGCCGGGGAAA |
| | | GGACTGGAATGGGTCTCCGCCATCTTGGATCATGGTGGTT |
| | | CGACATACTATGCGGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCGTTATCTACCATGCTGGTGGTGGCGTCACCTTTG |
| | | ATACTCGGGGACAGGGCACCCAAGTTACAGTCTCATCG |
| SEQ ID NO: 183 | Format A | QVQLVQSGAEVKKPGASVKVSCKASGYIFTSYYLHWVRQA |
| | BGA-9468 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTVSAEDVG |
| | | WVRQAPGKGLEWVSAILDSGGSTYYAESVKGRFTISRDNA |
| | | KNTLYLQMSSLRAEDTAVYYCARVVYHSGGGVTFDLRGQ |
| | | GTQVTVSS |
| SEQ ID NO: 184 | Format A | CAGGTGCAGCTGGTGCAGAGCGGCGCGGAAGTGAAAAA |
| | BGA-9468 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGGTTTCC |
| | | GCCGAAGACGTGGGTTGGGTGCGTCAAGCGCCGGGGAAA |
| | | GGACTGGAATGGGTCTCCGCCATCTTGGATTCGGGTGGTT |
| | | CGACATACTATGCGGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCGTTGTCTACCATTCTGGTGGTGGCGTCACCTTTG |
| | | ATCTTCGGGGACAGGGCACCCAAGTTACAGTCTCATCG |
| SEQ ID NO: 185 | Format A | QVQLVQSGAEVKKPGASVKVSCKASGYIFTSYYLHWVRQA |
| | BGA-3285 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTISAEDVG |
| | | WVRQAPGKGLEWVSAILDFGGSTYYAESVKGRFTISRDNA |
| | | KNTLYLQMSSLRAEDTAVYYCARVVYHAGGGVTFDTRGQ |
| | | GTQVTVSS |
| SEQ ID NO: 186 | Format A | CAGGTGCAGCTGGTGCAGAGCGGCGCGGAAGTGAAAAA |
| | BGA-3285 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGATTTCC |
| | | GCCGAAGACGTGGGTTGGGTGCGTCAAGCGCCGGGGAAA |
| | | GGACTGGAATGGGTCTCCGCCATCTTGGATTTTGGTGGTT |
| | | CGACATACTATGCGGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCGTTGTCTACCATGCTGGTGGTGGCGTCACCTTTG |
| | | ATACTCGGGGACAGGGCACCCAAGTTACAGTCTCATCG |
| SEQ ID NO: 187 | Format A | QVQLVQSGAEVKKPGASVKVSCKASGYIFTSYYLHWVRQA |
| | BGA-9442 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTISAEDVG |
| | | WVRQAPGKGLEWVSAILDSGGSTYYAESVKGRFTISRDNA |
| | | KNTLYLQMSSLRAEDTAVYYCARYVYHAGGGVTFDLRGQ |
| | | GTQVTVSS |
| SEQ ID NO: 188 | Format A | CAGGTGCAGCTGGTGCAGAGCGGCGCGGAAGTGAAAAA |
| | BGA-9442 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGATTTCC |
| | | GCCGAAGACGTGGGTTGGGTGCGTCAAGCGCCGGGGAAA |
| | | GGACTGGAATGGGTCTCCGCCATCTTGGATTCTGGTGGTT |
| | | CGACATACTATGCGGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCTATGTCTACCATGCTGGTGGTGGCGTCACCTTTG |
| | | ATCTTCGGGGACAGGGCACCCAAGTTACAGTCTCATCG |
| SEQ ID NO: 189 | Format A | QVQLVQSGAEVKKPGASVKVSCKASGYIFTSYYLHWVRQA |
| | BGA-7746 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTVSAEDVG |
| | | WVRQAPGKGLEWVSAILDFGGSTYYAESVKGRFTISRDNA |
| | | KNTLYLQMSSLRAEDTAVYYCARVVYHAGGGVTFDTRGQ |
| | | GTQVTVSS |
| SEQ ID NO: 190 | Format A | CAGGTGCAGCTGGTGCAGAGCGGCGCGGAAGTGAAAAA |
| | BGA-7746 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGGTTTCC |
| | | GCCGAAGACGTGGGTTGGGTGCGTCAAGCGCCGGGGAAA |
| | | GGACTGGAATGGGTCTCCGCCATCTTGGATTTTGGTGGTT |
| | | CGACATACTATGCAGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCGTCGTCTACCATGCTGGTGGTGGCGTCACCTTTG |
| | | ATACGCGGGGACAGGGCACCCAAGTTACAGTCTCATCG |
| SEQ ID NO: 191 | Format A | QVQLVQSGAEVKKPGASVKVSCKASGYIFTSYYLHWVRQA |
| | BGA-8425 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTLSAEDVG |
| | | WVRQAPGKGLEWVSAILDSGGSTYYAESVKGRFTISRDNA |
| | | KNTLYLQMSSLRAEDTAVYYCARVVYHAGGGVTYDYRGQ |
| | | GTQVTVSS |
| SEQ ID NO: 192 | Format A | CAGGTGCAGCTGGTGCAGAGCGGCGCGGAAGTGAAAAA |
| | BGA-8425 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CCAAGGATACCCTGATGATCTCTCGGACCCCAGAGGTGA |
| | | CATGCGTGGTGGTGGATGTGAGCCACGAGGACCCCGAGG |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGCTGTCC |
| | | GCCGAAGACGTGGGTTGGGTGCGTCAAGCGCCGGGGAAA |
| | | GGACTGGAATGGGTCTCCGCCATCTTGGATTCGGGTGGTT |
| | | CGACATACTATGCGGAAAGTGTCAAAGGGCGCTTTACGA |
| | | TCTCGCGCGATAACGCAAAAAATACTCTTTACCTTCAAAT |
| | | GTCTAGCCTTCGTGCTGAGGACACTGCGGTGTACTACTGC |
| | | GCCCGCGTCGTCTACCATGCTGGTGGTGGCGTCACCTATG |
| | | ATTACCGGGGACAGGGCACCCAAGTTACAGTCTCATCG |
| SEQ ID NO: 193 | Format A | QVQLVQSGAEVKKPGASVKVSCKASGYIFTSYYLHWVRQA |
| | BGA-3386 | PGQGLEWIGYINPQTGKTSYAQKFQGRVTMTRDTSTSTVY |
| | AA | MELSSLRSEDTAVYYCAREYGNYNYPLDYWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPPAAGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALAAPIEKTISKAKGQPREPQVYTLPPSREEMTKNQVS |
| | | LTCLVKGFYPSDIAVEWESNGQPENNYKTTPPVLDSDGSFFL |
| | | YSKLTVDKSRWQQGNVFSCSVMHEALHNHYTQKSLSLSPG |
| | | KGGGGSEVQLLESGGGLVQPGGSLRLSCAASGFTVSAEDVG |
| | | WVRQAPGKGLEWVSAILDKGGSTYYAESVKGRFTISRDNA |
| | | KNTLYLQMSSLRAEDTAVYYCARIVYHAGGGVTFDTWGQ |
| | | GTLVTVSS |
| SEQ ID NO: 194 | Format A | CAGGTGCAGCTGGTGCAGAGCGGCGCGGAAGTGAAAAA |
| | BGA-3386 | ACCGGGCGCGAGCGTGAAAGTGAGCTGCAAAGCGAGCG |
| | DNA | GCTATATTTTTACCAGCTATTACCTGCATTGGGTGCGCCA |
| | | GGCGCCGGGCCAGGGCCTGGAATGGATTGGCTATATTAA |
| | | CCCGCAGACCGGCAAGACCAGCTATGCCCAGAAATTTCA |
| | | GGGCCGCGTGACCATGACCCGCGATACCAGCACCAGCAC |
| | | CGTGTATATGGAACTGAGCAGCCTGCGCAGCGAAGATAC |
| | | CGCGGTGTATTATTGCGCGCGCGAATATGGCAACTATAA |
| | | CTATCCGCTGGATTATTGGGGCCAGGGCACCCTGGTGACC |
| | | GTGAGCAGCGCTAGCACCAAGGGGCCCTCGGTCTTCCCC |
| | | CTGGCACCCTCCTCCAAGAGTACTTCTGGGGGCACAGCG |
| | | GCCCTGGGCTGCCTGGTCAAGGACTACTTCCCCGAACCG |
| | | GTGACGGTGTCGTGGAACTCAGGCGCCCTGACCAGCGGC |
| | | GTGCACACCTTCCCGGCTGTCCTACAGTCCTCAGGACTCT |
| | | ACTCCCTCAGCAGCGTGGTGACCGTGCCCTCCAGCAGCTT |
| | | GGGCACCCAGACCTACATCTGCAACGTGAATCACAAGCC |
| | | CAGCAACACCAAGGTGGACAAGAAAGTTGAGCCAAAGTC |
| | | CTGTGACAAGACCCACACATGCCCCCCTTGTCCTGCTCCA |
| | | CCAGCTGCAGGACCAAGCGTGTTCCTGTTTCCACCCAAGC |
| | | CATGCGTGGTGGTGGATGTGAGCCACGAGGACCCCGAGG |
| | | CCAAGGATACCCTGATGATCTCTCGGACCCCAGAGGTGA |
| | | TGAAGTTCAACTGGTATGTGGACGGCGTGGAGGTGCACA |
| | | ATGCTAAGACCAAGCCCAGGGAGGAGCAGTACAACTCCA |
| | | CCTATAGAGTGGTGTCTGTGCTGACAGTGCTGCACCAGG |
| | | ATTGGCTGAACGGCAAGGAGTATAAGTGCAAGGTGTCCA |
| | | ATAAGGCCCTGGCCGCTCCTATCGAGAAGACCATCTCTA |
| | | AGGCCAAGGGCCAGCCCAGAGAGCCTCAGGTGTACACAC |
| | | TGCCTCCATCCCGGGAAGAGATGACCAAGAACCAGGTGT |
| | | CTCTGACATGTCTGGTCAAGGGCTTCTATCCCTCTGACAT |
| | | CGCCGTGGAGTGGGAGAGCAATGGCCAGCCTGAGAACAA |
| | | TTACAAGACCACACCCCCTGTGCTGGATTCCGACGGCTCT |
| | | TTCTTTCTGTATAGCAAGCTGACCGTGGACAAGTCCCGGT |
| | | GGCAGCAGGGCAACGTGTTCAGCTGTTCCGTGATGCACG |
| | | AAGCTCTGCATAATCACTATACTCAGAAATCCCTGTCACT |
| | | GTCACCTGGTAAAGGTGGAGGCGGTTCAGAGGTCCAGTT |
| | | ACTTGAGAGTGGTGGAGGTCTGGTCCAACCAGGAGGTTC |
| | | GCTGCGTTTATCCTGCGCCGCGTCTGGATTCACGGTTTCC |
| | | GCCGAAGACGTGGGTTGGGTGCGTCAAGCGCCGGGGAAA |
| | | GGACTGGAATGGGTCTCCGCCATCTTGGATAAGGGTGGT |
| | | TCGACATACTATGCGGAAAGTGTCAAAGGGCGCTTTACG |
| | | ATCTCGCGCGATAACGCAAAAAATACTCTTTACCTTCAAA |
| | | TGTCTAGCCTTCGTGCTGAGGACACTGCGGTGTACTACTG |
| | | CGCCCGCATTGTCTACCATGCTGGTGGTGGCGTCACCTTT |
| | | GATACTTGGGGACAGGGCACCTTAGTTACAGTCTCATCG |

### Example 13. Binding Profiles of Anti-CD137 Antibody BGA-5623

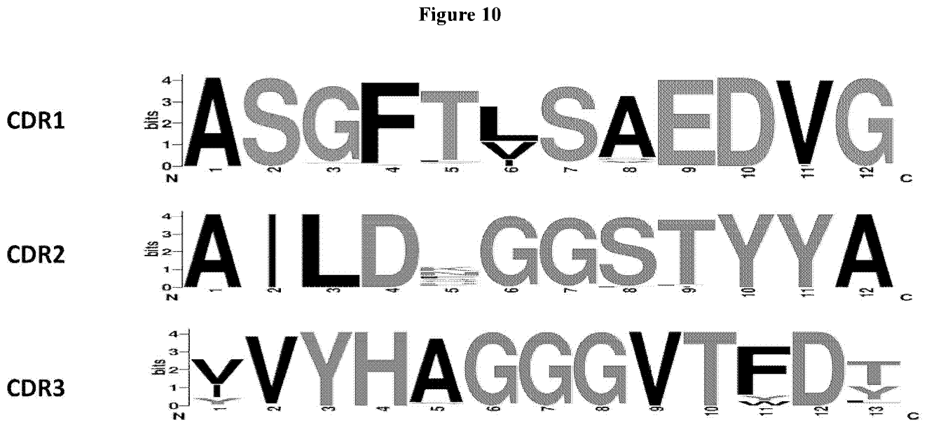

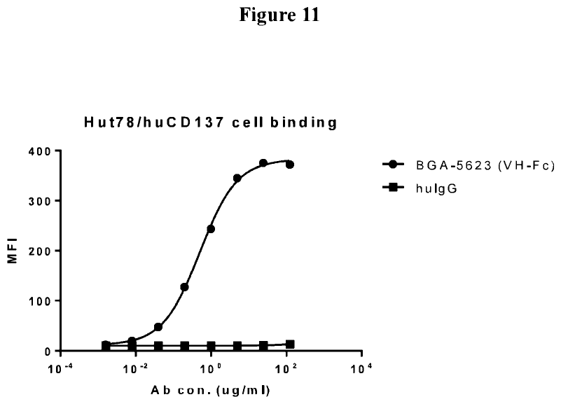

BGA-5623 was generated with human IgG1 Fc fusion and characterized for their binding kinetics by SPR assays using BIAcore™ T-200 (GE Life Sciences). Briefly, anti-human IgG (Fc) antibody was immobilized on an activated CM5 biosensor chip (Cat.: BR100839, GE Life Sciences). The anti-huCD137 domain antibody was flown through the chip surface and captured by anti-human IgG (Fc) antibody. Then a serial dilution (6.0 nM to 2150 nM) of human CD137 ECD-mIgG2a or cyno CD137 ECD-mIgG2a were flown over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The result demonstrated that BGA-5623 has higher affinity for cynoCD137 than huCD137, as shown in Table 17 below. To evaluate the binding activity of the anti-huCD137 VH domain antibody to native huCD137 on living cells, Hut78 cells were transfected to over-express human CD137. Live Hut78/huCD137 expressing cells were seeded in 96-well plates and were incubated with a serial dilution of anti-huCD137 VH domain antibodies. Goat anti-Human IgG was used as secondary antibody to detect antibody binding to the cell surface. EC₅₀ values for dose-dependent binding to human native CD137 were determined by fitting the dose-response data to the four-parameter logistic model with GraphPad Prism™. As shown in FIG. 11, BGA-5623 demonstrated high binding affinity to native CD137 on living cells.

TABLE 17
Affinity comparison of affinities by SPR

| Sample | Species | Ka(1/Ms) | Kd(1/s) | KD(M) |
|---|---|---|---|---|
| BGA-5623 | huCD137 | 4.05E+04 | 3.22E−03 | 7.94E−08 |
| (IgG1) | cynoCD137 | 1.62E+05 | 1.97E−03 | 1.22E−08 |

TABLE 18
Amino acid and DNA sequences

| | SEQ ID | | |
|---|---|---|---|
| Antibody | NO | | SEQUENCE |
| BGA-5623 | SEQ ID | VH | EVQLLESGGGLVQPGGSLRLSCAASGFTVSAEDVGWVRQAPG |
| | NO: 33 | | KGLEWVSAILDKGGSTYYAESVKGRFTISRDNAKNTLYLQMS |
| | | | SLRAEDTAVYYCARIVYHAGGGVTFDTRGQGTQVTVSS |
| | SEQ ID | VH DNA | GAGGTCCAGTTACTTGAGAGTGGTGGAGGTCTGGTCCAACC |
| | NO: 34 | | AGGAGGTTCGCTGCGTTTATCCTGCGCCGCGTCTGGATTCAC |
| | | | GGTTTCCGCCGAAGACGTGGGTTGGGTGCGTCAAGCGCCG |
| | | | GGGAAAGGACTGGAATGGGTCTCCGCCATCTTGGATAAGGG |
| | | | TGGTTCGACATACTATGCGGAAAGTGTCAAAGGGCGCTTTA |
| | | | CGATCTCGCGCGATAACGCAAAAAATACTCTTTACCTTCAAA |
| | | | TGTCTAGCCTTCGTGCTGAGGACACTGCGGTGTACTACTGCG |
| | | | CCCGCATTGTCTACCATGCTGGTGGTGGCGTCACCTTTGATA |
| | | | CTCGGGGACAGGGCACCCAAGTTACAGTCTCATCG |
| | SEQ ID | IgG1 AA | EVQLLESGGGLVQPGGSLRLSCAASGFTVSAEDVGWVRQAPG |
| | NO: 195 | | KGLEWVSAILDKGGSTYYAESVKGRFTISRDNAKNTLYLQMS |
| | | | SLRAEDTAVYYCARIVYHAGGGVTFDTRGQGTQVTVSSEPKS |
| | | | SDKTHTCPPCPAPELLGGPSVFLFPPKPKDTLMISRTPEVTCVV |
| | | | VDVSHEDPEVKFNWYVDGVEVHNAKTKPREEQYNSTYRVVS |
| | | | VLTVLHQDWLNGKEYKCKVSNKALPAPIEKTISKAKGQPREP |
| | | | QVYTLPPSRDELTKNQVSLTCLVKGFYPSDIAVEWESNGQPEN |
| | | | NYKTTPPVLDSDGSFFLYSKLTVDKSRWQQGNVFSCSVMHEA |
| | | | LHNHYTQKSLSLSPGK |
| | SEQ ID | IgG1 | GAGGTCCAGTTACTTGAGAGTGGTGGAGGTCTGGTCCAACC |
| | NO: 196 | DNA | AGGAGGTTCGCTGCGTTTATCCTGCGCCGCGTCTGGATTCAC |
| | | | GGTTTCCGCCGAAGACGTGGGTTGGGTGCGTCAAGCGCCG |
| | | | GGGAAAGGACTGGAATGGGTCTCCGCCATCTTGGATAAGGG |
| | | | TGGTTCGACATACTATGCGGAAAGTGTCAAAGGGCGCTTTA |
| | | | CGATCTCGCGCGATAACGCAAAAAATACTCTTTACCTTCAAA |
| | | | TGTCTAGCCTTCGTGCTGAGGACACTGCGGTGTACTACTGCG |
| | | | CCCGCATTGTCTACCATGCTGGTGGTGGCGTCACCTTTGATA |
| | | | CTCGGGGACAGGGCACCCAAGTTACAGTCTCATCGGAGCCC |
| | | | AAATCTTCTGACAAAACTCACACATGCCCACCGTGCCCAGC |
| | | | ACCTGAACTCCTGGGGGGACCGTCAGTCTTCCTCTTCCCCC |
| | | | CAAAACCCAAGGACACCCTCATGATCTCCCGGACCCCTGAG |
| | | | GTCACATGCGTGGTGGTGGACGTGAGCCACGAAGACCCTG |
| | | | AGGTCAAGTTCAACTGGTACGTGGACGGCGTGGAGGTGCAT |
| | | | AATGCCAAGACAAAGCCGCGGGAGGAGCAGTACAACAGCA |
| | | | CGTACCGTGTGGTCAGCGTCCTCACCGTCCTGCACCAGGAC |
| | | | TGGCTGAATGGCAAGGAGTACAAGTGCAAGGTCTCCAACA |
| | | | AAGCCCTCCCAGCCCCCATCGAGAAAACCATCTCCAAAGCC |
| | | | AAAGGGCAGCCCCGAGAACCACAGGTGTACACCCTGCCCC |
| | | | CATCCCGGGATGAGCTGACCAAGAACCAGGTCAGCCTGACC |
| | | | TGCCTGGTCAAAGGCTTCTATCCCAGCGACATCGCCGTGGA |
| | | | GTGGGAGAGCAATGGGCAGCCGGAGAACAACTACAAGACC |
| | | | ACGCCTCCCGTGCTGGACTCCGACGGCTCCTTCTTCCTCTAC |
| | | | AGCAAGCTCACCGTGGACAAGAGCAGGTGGCAGCAGGGGA |
| | | | ACGTCTTCTCATGCTCCGTGATGCATGAGGCTCTGCACAACC |
| | | | ACTACACGCAGAAGAGCCTCTCCCTGTCTCCGGGTAAA |
| | SEQ ID | IgG4 AA | EVQLLESGGGLVQPGGSLRLSCAASGFTVSAEDVGWVRQAPG |
| | NO: 197 | | KGLEWVSAILDKGGSTYYAESVKGRFTISRDNAKNTLYLQMS |
| | | | SLRAEDTAVYYCARIVYHAGGGVTFDTRGQGTQVTVSSESKY |
| | | | GPPCPPCPAPEFLGGPSVFLFPPKPKDTLMISRTPEVTCVVVDV |
| | | | SQEDPEVQFNWYVDGVEVHNAKTKPREEQFNSTYRWSVLTV |
| | | | LHQDWLNGKEYKCKVSNKGLPSSIEKTISKAKGQPREPQVYT |
| | | | LPPSQEEMTKNQVSLTCLVKGFYPSDIAVEWESNGQPENNYKT |
| | | | TPPVLDSDGSFFLYSRLTVDKSRWQEGNVFSCSVMHEALHNH |
| | | | YTQKSLSLSLGK |
| | SEQ ID | IgG4 | GAGGTCCAGTTACTTGAGAGTGGTGGAGGTCTGGTCCAACC |
| | NO: 198 | DNA | AGGAGGTTCGCTGCGTTTATCCTGCGCCGCGTCTGGATTCAC |
| | | | GGTTTCCGCCGAAGACGTGGGTTGGGTGCGTCAAGCGCCG |
| | | | GGGAAAGGACTGGAATGGGTCTCCGCCATCTTGGATAAGGG |
| | | | TGGTTCGACATACTATGCGGAAAGTGTCAAAGGGCGCTTTA |
| | | | CGATCTCGCGCGATAACGCAAAAAATACTCTTTACCTTCAAA |
| | | | TGTCTAGCCTTCGTGCTGAGGACACTGCGGTGTACTACTGCG |
| | | | CCCGCATTGTCTACCATGCTGGTGGTGGCGTCACCTTTGATA |
| | | | CTCGGGGACAGGGCACCCAAGTTACAGTCTCATCGGAATCC |
| | | | AAGTATGGTCCGCCATGTCCACCGTGCCCGGCACCCGAGTT |
| | | | TTTGGGAGGGCCTAGTGTGTTTTTGTTTCCTCCTAAGCCAAA |
| | | | GGACACGCTTATGATTAGTCGCACACCCGAGGTTACCTGTGT |
| | | | AGTGGTGGACGTATCACAAGAGGATCCGGAAGTCCAATTTA |
| | | | ATTGGTACGTCGACGGAGTGGAAGTCCACAATGCAAAGACC |
| | | | AAGCCTAGGGAAGAACAGTTCAATTCTACCTATCGCTGGTCT |
| | | | GTACTCACTGTACTCCACCAGGATTGGCTCAATGGAAAGGA |
| | | | ATACAAATGCAAGGTTTCCAACAAAGGGCTCCCCTCTTCCAT |
| | | | TGAGAAAACTATTAGCAAGGCTAAAGGCCAACCCAGAGAA |
| | | | CCACAGGTTTATACCCTTCCACCTTCCCAGGAGGAGATGAC |
| | | | GAAGAATCAGGTTAGTCTTACGTGCCTCGTTAAAGGATTCTA |
| | | | CCCTTCAGATATCGCAGTTGAATGGGAGTCTAACGGGCAAC |
| | | | CCGAGAACAACTACAAGACCACCCCGCCGGTGCTGGACTC |
| | | | AGATGGTAGTTTCTTTCTTTACAGTCGGCTTACCGTCGACAA |
| | | | GTCACGCTGGCAGGAGGGAAATGTATTCTCCTGCAGCGTAA |
| | | | TGCACGAAGCTCTGCACAATCACTATACCCAGAAGTCACTG |
| | | | AGCCTTTCTCTGGGTAAG |

### Example 14. Epitope Mapping of BGA-5623

To characterize the binding epitope of BGA-5623, 17 amino acid residues of human CD137 were mutated to alanine individually to generate 17 single-mutation huCD137 variants based upon the information from the crystal structure of CD137 reported previously (Bitra et al., (2018) J Biol Chem, 293, 9958-9969; Chin et al., (2018) Nat Commun, 9, 4679).

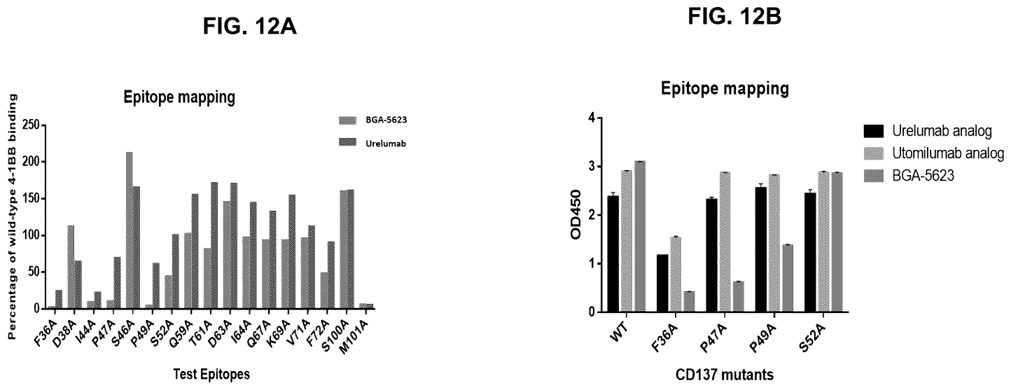

The CD137 mutants along with the wild-type CD137 were transiently expressed in HEK293 cells (ATCC CRL-1573). Their recognition and binding by BGA-5623 was analyzed by flow cytometry. An Urelumab analog (SEQ ID NO: 199-202) that was generated in house by using the publicly available sequences of Urelumab, was used in the same assay to monitor the expression of CD137 mutants. In this assay, human CD137 or CD137 mutants expressing cells (10⁵ cells/well) were incubated with 2 μg/ml of purified BGA-5623-mutFc (Fc fusion VH Ab) or Urelumab analog, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). All results were normalized using the mean values of the fluorescence reading of wild type CD137 binding signal as the standard. To simplify data analysis, if an antibody's FACS binding signal for a specific mutant CD137 dropped to or below 25%, then the amino acid at that site was considered critical to the epitope. As shown in the FIG. 12A, the epitope of BGA-5623 has important residues for binding at amino acids F36, I44, P47, P49 and S52 of CD137.

In order to further explore the BGA-5623 epitope, human CD137 ECD mutants with single-AA substitution were expressed and purified to prepare for ELISA. In addition, a Utomilumab analog antibody (SEQ ID NO: 203-206) was created in house by using the publicly available sequences of Utomilumab. The CD137 mutants along with the wild-type CD137 were analyzed for binding by BGA-5623 by direct ELISA. In brief, 50 ng each of wild-type or mutant CD137 was coated in an ELISA plate. After blocking, 100 μl of BGA-5623-mutFc, Urelumab analog or Utomilumab analog antibody at a concentration of 2 μg/ml was added to the plate and the binding signal of each antibody was detected by HRP-linked secondary antibody. In the ELISA binding assay using wild-type or mutant huCD137, amino acids F36A, P47A and P49A significantly impaired the binding of CD137 and BGA-5623

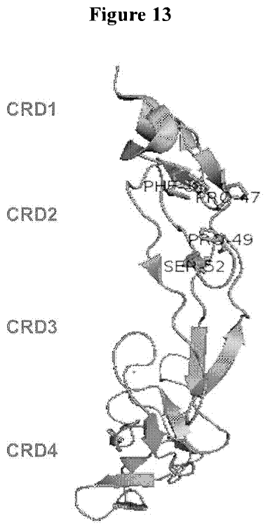

(FIGS. 12A-B). Changes at amino acid F36A only slightly reduced the binding of the Urelumab or Utomilumab analogs, which might indicate F36A plays a critical role in the conformation integrity of CD137. In contrast, neither changes at amino acids P47A or P49A disrupted the binding of the Urelumab or Utomilumab analog to CD137, indicating that BGA-5623, Urelumab analog or Utomilumab have different epitopes. This data indicated that amino acids F36A, P47A and P49A are critical residues in the epitope for antibody BGA-5623. The molecular modeling of CD137 shown in FIG. 13 shows that when CD137 is in its folded conformation, amino acids F36A, P47A and P49A are near each other, but located in two different domains CRD1 and CRD2 of CD137.

TABLE 19
Amino acid and DNA sequences

| | SEQ ID | | |
|---|---|---|---|
| Antibody | NO | | SEQUENCE |
| Urelumab | SEQ ID NO: | Heavy | QVQLQQWGAGLLKPSETLSLTCAVYGGSFSGYYWSWIR |
| analog | 199 | chain AA | QSPEKGLEWIGEINHGGYVTYNPSLESRVTISVDTSKNQF |
| | | | SLKLSSVTAADTAVYYCARDYGPGNYDWYFDLWGRGTL |
| | | | VTVSSASTKGPSVFPLAPCSRSTSESTAALGCLVKDYFPEP |
| | | | VTVSWNSGALTSGVHTFPAVLQSSGLYSLSSVVTVPSSSL |
| | | | GTKTYTCNVDHKPSNTKVDKRVESKYGPPCPPCPAPEFL |
| | | | GGPSVFLFPPKPKDTLMISRTPEVTCVVVDVSQEDPEVQF |
| | | | NWYVDGVEVHNAKTKPREEQFNSTYRWSVLTVLHQDW |
| | | | LNGKEYKCKVSNKGLPSSIEKTISKAKGQPREPQVYTLPP |
| | | | SQEEMTKNQVSLTCLVKGFYPSDIAVEWESNGQPENNYK |
| | | | TTPPVLDSDGSFFLYSRLTVDKSRWQEGNVFSCSVMHEA |
| | | | LHNHYTQKSLSLSLGK |
| | SEQ ID NO: | Heavy | CAAGTTCAACTTCAGCAGTGGGGAGCGGGTCTCTTGA |
| | 200 | chain DNA | AGCCTAGTGAGACGCTGAGCCTGACCTGTGCAGTCTAC |
| | | | GGGGGTTCATTTTCTGGGTACTACTGGTCCTGGATACGC |
| | | | CAAAGCCCAGAGAAAGGCCTGGAATGGATTGGGGAGA |
| | | | TAAATCACGGGGGTTATGTGACATATAATCCTTCTCTTG |
| | | | AGAGCCGGGTCACGATATCTGTTGACACAAGTAAAAAT |
| | | | CAGTTCTCCCTCAAACTGAGCTCCGTTACCGCAGCAGA |
| | | | CACTGCCGTTTACTACTGTGCGCGGGACTATGGGCCAG |
| | | | GTAATTACGATTGGTACTTCGACCTGTGGGGTAGAGGA |
| | | | ACGCTGGTCACTGTAAGCAGTGCATCTACAAAGGGCCC |
| | | | CAGCGTGTTTCCCCTTGCACCATGTTCACGGAGCACTA |
| | | | GTGAAAGCACTGCCGCGCTCGGGTGTCTCGTTAAGGAT |
| | | | TATTTTCCGGAGCCTGTCACTGTCTCTTGGAACTCTGGT |
| | | | GCACTGACATCAGGCGTTCACACGTTCCCTGCAGTACT |
| | | | CCAGAGCTCAGGATTGTACAGCCTGTCCAGTGTAGTTA |
| | | | CAGTGCCTTCCTCCTCCCTTGGAACAAAGACCTACACC |
| | | | TGCAACGTGGACCATAAGCCCAGCAATACGAAAGTAG |
| | | | ACAAACGAGTCGAATCCAAGTATGGTCCGCCATGTCCA |
| | | | CCGTGCCCGGCACCCGAGTTTTTGGGAGGGCCTAGTGT |
| | | | GTTTTTGTTTCCTCCTAAGCCAAAGGACACGCTTATGAT |
| | | | TAGTCGCACACCCGAGGTTACCTGTGTAGTGGTGGACG |
| | | | TATCACAAGAGGATCCGGAAGTCCAATTTAATTGGTAC |
| | | | GTCGACGGAGTGGAAGTCCACAATGCAAAGACCAAGC |
| | | | CTAGGGAAGAACAGTTCAATTCTACCTATCGCTGGTCT |
| | | | GTACTCACTGTACTCCACCAGGATTGGCTCAATGGAAA |
| | | | GGAATACAAATGCAAGGTTTCCAACAAAGGGCTCCCCT |
| | | | CTTCCATTGAGAAAACTATTAGCAAGGCTAAAGGCCAA |
| | | | CCCAGAGAACCACAGGTTTATACCCTTCCACCTTCCCA |
| | | | GGAGGAGATGACGAAGAATCAGGTTAGTCTTACGTGCC |
| | | | TCGTTAAAGGATTCTACCCTTCAGATATCGCAGTTGAAT |
| | | | GGGAGTCTAACGGGCAACCCGAGAACAACTACAAGAC |
| | | | CACCCCGCCGGTGCTGGACTCAGATGGTAGTTTCTTTC |
| | | | TTTACAGTCGGCTTACCGTCGACAAGTCACGCTGGCAG |
| | | | GAGGGAAATGTATTCTCCTGCAGCGTAATGCACGAAGC |
| | | | TCTGCACAATCACTATACCCAGAAGTCACTGAGCCTTT |
| | | | CTCTGGGTAAG |
| | SEQ ID NO: | Light chain | EIVLTQSPATLSLSPGERATLSCRASQSVSSYLAWYQQKPG |
| | 201 | AA | QAPRLLIYDASNRATGIPARFSGSGSGTDFTLTISSLEPEDF |
| | | | AVYYCQQRSNWPPALTFGGGTKVEIKRTVAAPSVFIFPPS |
| | | | DEQLKSGTASVVCLLNNFYPREAKVQWKVDNALQSGNS |
| | | | QESVTEQDSKDSTYSLSSTLTLSKADYEKHKVYACEVTH |
| | | | QGLSSPVTKSFNRGEC |
| | SEQ ID NO: | Light chain | GAAATCGTGCTGACCCAATCACCTGCGACCCTCTCCCT |
| | 202 | DNA | TAGCCCTGGAGAACGAGCAACTCTCAGTTGCAGAGCA |
| | | | AGCCAGAGCGTCAGCTCATATCTCGCATGGTATCAACA |
| | | | AAAACCAGGCCAAGCTCCTAGACTGTTGATTTATGATG |
| | | | CCTCTAACCGGGCGACTGGCATACCAGCCCGCTTTAGC |
| | | | GGGTCTGGGAGTGGCACTGATTTTACGCTCACGATTTC |
| | | | ATCTTTGGAGCCAGAAGATTTCGCTGTATACTACTGTCA |
| | | | ACAAAGGTCAAACTGGCCGCCTGCACTTACGTTCGGC |
| | | | GGGGGTACCAAGGTGGAGATTAAGAGGACAGTTGCGG |
| | | | CGCCATCAGTTTTTATATTCCCACCCTCCGATGAGCAGT |
| | | | TGAAAAGTGGCACGGCATCAGTTGTCTGTCTGCTGAAC |
| | | | AATTTCTATCCACGGGAGGCGAAAGTCCAATGGAAAGT |
| | | | GGACAATGCGCTTCAGAGCGGGAACTCTCAGGAGAGT |
| | | | GTCACTGAACAGGATTCAAAGGACTCCACGTACTCACT |
| | | | CTCATCCACTCTCACCCTTTCAAAGGCGGATTACGAAA |
| | | | AGCATAAGGTCTACGCTTGTGAGGTAACACATCAGGGG |
| | | | CTTTCCAGTCCTGTAACCAAGAGCTTCAATCGCGGCGA |
| | | | ATGC |
| Utomilumab | SEQ ID NO: | Heavy | EVQLVQSGAEVKKPGESLRISCKGSGYSFSTYWISWVRQ |
| analog | 203 | chain AA | MPGKGLEWMGKIYPGDSYTNYSPSFQGQVTISADKSIST |
| | | | AYLQWSSLKASDTAMYYCARGYGIFDYWGQGTLVTVSS |
| | | | ASTKGPSVFPLAPCSRSTSESTAALGCLVKDYFPEPVTVS |
| | | | WNSGALTSGVHTFPAVLQSSGLYSLSSVVTVPSSNFGTQT |
| | | | YTCNVDHKPSNTKVDKTVERKCCVECPPCPAPPVAGPSV |
| | | | FLFPPKPKDTLMISRTPEVTCVVVDVSHEDPEVQFNWYV |
| | | | DGVEVHNAKTKPREEQFNSTFRVVSVLTVVHQDWLNGK |
| | | | EYKCKVSNKGLPAPIEKTISKTKGQPREPQVYTLPPSREE |
| | | | MTKNQVSLTCLVKGFYPSDIAVEWESNGQPENNYKTTPP |
| | | | MLDSDGSFFLYSKLTVDKSRWQQGNVFSCSVMHEALHN |
| | | | HYTQKSLSLSPGK |
| | SEQ ID NO: | Heavy | GAAGTTCAACTGGTACAGTCCGGTGCTGAAGTTAAAA |
| | 204 | chain DNA | AACCCGGAGAGAGTCTGAGAATCTCTTGCAAGGGGTC |
| | | | TGGGTACTCTTTTAGTACATATTGGATATCTTGGGTGAG |
| | | | ACAAATGCCGGGGAAAGGCCTCGAATGGATGGGCAAG |
| | | | ATTTATCCCGGAGACAGCTATACAAATTACTCACCTAGT |
| | | | TTTCAAGGTCAGGTAACTATCAGCGCGGACAAATCCAT |
| | | | AAGTACGGCCTATCTGCAATGGTCTTCCTTGAAGGCATC |
| | | | CGACACGGCGATGTATTACTGCGCAAGAGGGTATGGTA |
| | | | TATTCGATTACTGGGGGCAGGGCACTCTCGTTACCGTG |
| | | | AGCAGCGCTTCAACAAAAGGCCCATCTGTCTTCCCTCT |
| | | | CGCGCCTTGCTCCCGCTCTACTTCAGAGTCAACAGCTG |
| | | | CTCTGGGGTGCTTGGTGAAGGATTACTTTCCTGAACCG |
| | | | GTTACTGTCAGCTGGAATTCTGGGGCTTTGACGAGCGG |
| | | | AGTTCACACTTTCCCAGCTGTACTGCAAAGTTCTGGAC |
| | | | TTTACAGCTTGAGCTCCGTGGTGACAGTGCCGAGCTCA |
| | | | AACTTCGGTACGCAGACATATACATGCAATGTGGATCAT |
| | | | AAACCCAGCAATACGAAAGTAGATAAGACAGTCGAAA |
| | | | GGAAGTGTTGTGTAGAGTGCCCACCGTGCCCCGCCCCT |
| | | | CCGGTTGCAGGCCCATCAGTGTTTCTCTTCCCGCCCAA |
| | | | ACCTAAGGACACGCTGATGATTTCACGAACGCCCGAGG |
| | | | TCACCTGTGTCGTAGTCGATGTGTCCCATGAGGACCCA |
| | | | GAAGTTCAGTTCAATTGGTATGTAGACGGCGTGGAGGT |
| | | | ACACAATGCAAAAACGAAACCACGCGAGGAACAGTTC |
| | | | AATAGTACGTTCCGGGTCGTGAGTGTGCTCACGGTGGT |
| | | | TCACCAAGACTGGCTGAACGGGAAAGAATATAAGTGC |
| | | | AAAGTATCCAATAAAGGCCTGCCCGCACCCATCGAAAA |
| | | | AACTATAAGTAAAACTAAAGGGCAACCGCGAGAGCCT |
| | | | CAGGTCTATACTTTGCCACCGTCACGCGAAGAGATGAC |
| | | | CAAGAACCAAGTAAGTCTTACTTGCTTGGTCAAGGGGT |
| | | | TCTACCCCTCAGATATAGCGGTGGAGTGGGAATCCAAT |
| | | | GGCCAACCCGAGAACAATTACAAGACAACACCACCAA |
| | | | TGCTGGACTCTGACGGGAGTTTCTTTCTGTATAGTAAAC |
| | | | TTACCGTCGATAAATCACGCTGGCAGCAAGGCAACGTA |
| | | | TTCTCATGCTCAGTAATGCATGAAGCCTTGCATAATCAC |
| | | | TACACTCAGAAAAGCCTGTCCCTCTCTCCAGGTAAG |
| | SEQ ID NO: | Light chain | SYELTQPPSVSVSPGQTASITCSGDNIGDQYAHWYQQKPG |
| | 205 | AA | QSPVLVIYQDKNRPSGIPERFSGSNSGNTATLTISGTQAMD |
| | | | EADYYCATYTGFGSLAVFGGGTKLTVLGQPKAAPSVTLF |
| | | | PPSSEELQANKATLVCLISDFYPGAVTVAWKADSSPVKAG |
| | | | VETTTPSKQSNNKYAASSYLSLTPEQWKSHRSYSCQVTH |
| | | | EGSTVEKTVAPTECS |
| | SEQ ID NO: | Light chain | AGCTACGAGTTGACTCAGCCACCATCCGTCAGTGTTAG |
| | 206 | DNA | CCCCGGACAGACGGCGTCAATTACTTGTAGTGGAGACA |
| | | | ACATTGGCGATCAGTACGCCCATTGGTATCAGCAAAAA |
| | | | CCGGGTCAATCACCAGTTCTGGTGATATATCAGGATAAG |
| | | | AATCGACCATCAGGGATTCCAGAGAGGTTCTCAGGTAG |
| | | | TAACTCAGGTAACACAGCTACTCTGACCATAAGTGGTA |
| | | | CCCAGGCTATGGACGAAGCAGACTACTACTGCGCCACC |
| | | | TACACAGGCTTCGGATCACTTGCGGTTTTCGGGGGCGG |
| | | | AACCAAACTAACCGTTCTTGGGCAACCTAAGGCTGCCC |
| | | | CGTCCGTGACTCTTTTTCCTCCGTCTTCCGAAGAGCTTC |
| | | | AAGCCAATAAAGCAACCTTGGTATGTTTGATTTCCGATT |
| | | | TTTACCCTGGAGCGGTGACGGTGGCATGGAAAGCTGAT |
| | | | TCAAGCCCTGTGAAAGCTGGGGTGGAAACTACTACGC |
| | | | CGTCCAAACAGAGCAATAATAAGTACGCGGCCTCCAGC |
| | | | TATTTGTCACTTACCCCGGAGCAATGGAAGTCACACCG |
| | | | AAGTTACAGTTGTCAAGTTACTCACGAAGGCAGCACTG |
| | | | TTGAAAAGACCGTAGCACCCACCGAATGCTCA |

### Example 15. Ligand Competition

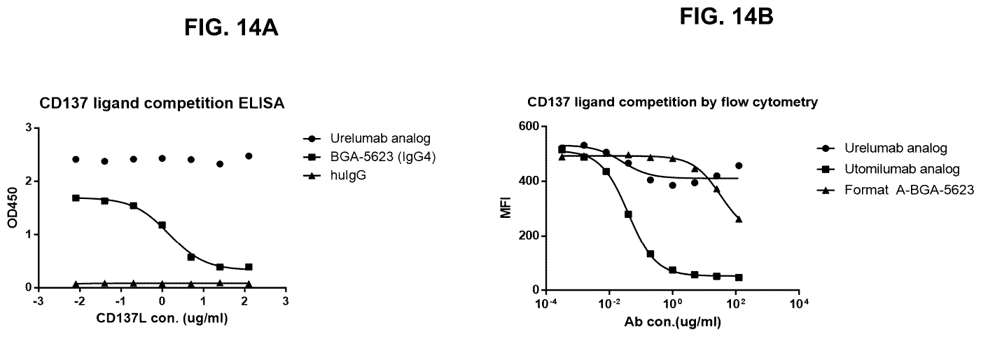

Human CD137 binds to its major ligand human CD137 ligand (CD137L) with weak affinity at an approximate Kd of three-digit M (Chin et al., (2018) Nat Commun, 9, 4679). The epitope mapping results in Example 14 above, shows that amino acid residues F36A, P47A and P49A of CD137 are critical amino acid residues that make up part of the epitope for the BGA-5623 antibody. In addition, the ligand binds CD137 along the entire length of receptor CRD-2 and the A2 motif of CRD-3, and the interface between the receptor and ligand is primarily mediated by hydrogen bonds and van der Waals interactions (Bitra et al., (2018) J Biol Chem, 293, 9958-9969. Based on this data, it was hypothesized that the BGA-5623 antibody can block CD137/CD137 ligand interaction. BGA-5623 was generated with a human IgG4 Fc fusion. For CD137 ligand competition ELISA, a Maxisorp immunoplate was coated with human CD137 ECD-mIgG2a and blocked with 3% BSA (w/v) in PBS buffer (blocking buffer). VH domain antibody BGA-5623 was blocked with blocking buffer for 30 minutes and added to wells of the ELISA plate for 1 hour in the presence of serially diluted human CD137 ligand ECD-mIgG2a. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA) (FIG. 14A). For the assay of CD137 ligand competition by flow cytometry, a CD137 stably transduced cell line Hut78/huCD137 was incubated with human CD137 ligand ECD-mIgG2a in the presence of serially diluted BGA-5623 (IgG4), followed by detection with goat-anti-murine IgG-APC (FIG. 14B). As shown in FIG. 14, BGA-5623 competes with CD137 ligand and reduces CD137/CD137 ligand interaction.

### Example 16. Evaluation of Off-Target Specificity

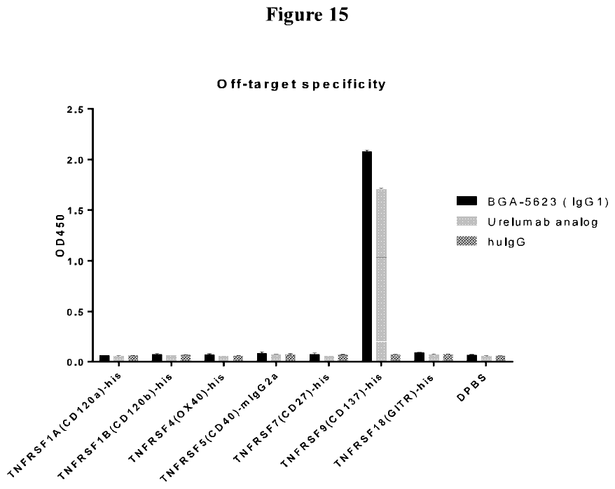

The off-target specificity of BGA-5623 was evaluated via ELISA. The antigen ELISA was performed as described above in Example 5. TNF receptor family members such as TNFRSF1A(CD120a) (Cat. No. 10872-H08H, Sino Biological, China), TNFRSF1B(CD120b) (Cat. No. 10417-H08H1, Sino Biological, China), TNFRSF4(OX40) (SEQ ID NO: 77), TNFRSF5(CD40) (SEQ ID NO: 71), TNFRSF7(CD27) (Cat. No. 10039-H08B1, Sino Biological, China), TNFRSF9(CD137) (SEQ ID NO: 49) and TNFRSF18(GITR) (Cat. No. 13643-H08H, Sino Biological, China) were coated in 96-well plates at a concentration of 10 μg/ml overnight at 4° C. BGA-5623 fused with wild type IgG1 Fc (SEQ ID NO: 195) was added. As shown in FIG. 15, no binding to other TNF receptor family members was observed.

### Example 17. Characterization of BGA-4712 Variants in the Multispecific Antibody Format A-CD137×CEA with Different Affinities

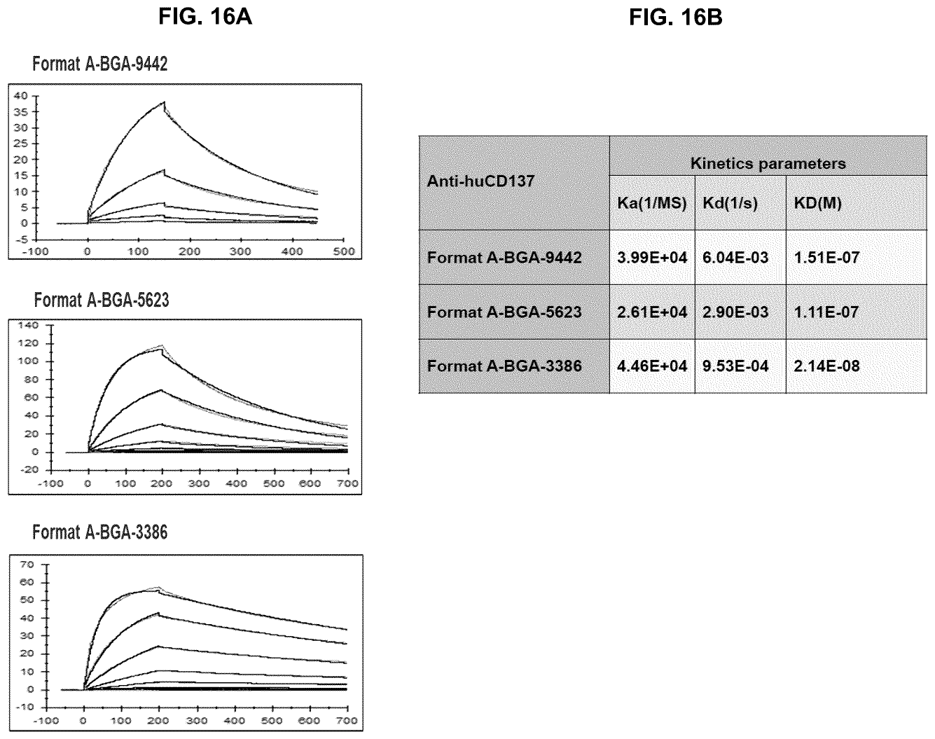

Three BGA-4712 variants (BGA-2164, BGA-6468 and BGA-9442) with high, intermediate and low affinities were selected for the potency comparison. SPR study and FACS analysis were performed as described above in Example 5 and shown in Table 20 and FIG. 16. For flow cytometry, human CD137⁺ or human CEA⁺ expressing cells (10⁵ cells/well) were incubated with various concentrations of purified VH domain antibodies, followed by binding with Alexa Fluro-647-labeled anti-hu IgG Fc antibody (Cat.: 409320, BioLegend, USA). Cell fluorescence was quantified using a flow cytometer (Guava easyCyte™ 8HT, Merck-Millipore, USA). There was no signification difference in CEA binding among three tested multispecific antibodies, whereas different binding affinities towards human CD137 were observed by flow cytometry as expected. Next, a PBMC based cytokine release assay as described above in Example 7 was applied to evaluate the potency of these BGA-4712 variants with different affinities in the multispecific antibody Format A-CD137×CEA. As shown in Table 20, the CD137 activation induced by these variants is proportionate to the increasing affinities of CD137 arm. When other biophysical properties such as non-specific binding and aggregates in SEC-HPLC were taken into consideration, BGA-5623 was selected for further characterization and investigation of parameters that could affect CD137 activation.

TABLE 20
Comparison of BGA-4712 variants in the multispecific antibody
format A-CD137 × CEA with different affinities

| | | Cell binding EC50 (ug/ml) | | Functions | |
|---|---|---|---|---|---|
| Format/clone | Format | CT26/CEA | Hut78/huCD137 | EC50(ug/ml) | Top |
| Format A-BGA-5623 | A-CD137 × CEA | 0.38 | 0.38 | 0.3412 | 2512 |
| Format A-BGA-9442 | | 0.5 | 0.5 | 0.2325 | 1722 |
| Format A BGA-3386 | | 0.21 | 0.21 | 0.07681 | 1953 |

### Example 18. Parameters which May Influence In Vitro CD137 Activation

The data above indicated that in addition to affinities, receptor density and epitope location in CD137 and molecular format, there are other key parameters such as module ratios, module orientation, linker length and Fc functions that could significantly affect cytokine release (Il-2 and IFN-γ). Therefore, to inform rational design of CD137 based multispecific antibodies, we took a systematic approach to interrogate how these parameters influence CD137 agonism. Expression and preparation of these multispecific antibodies were carried out as described above in Example 6.

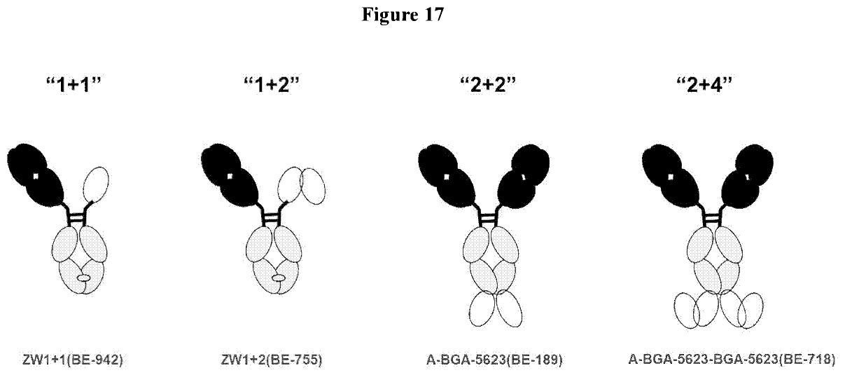

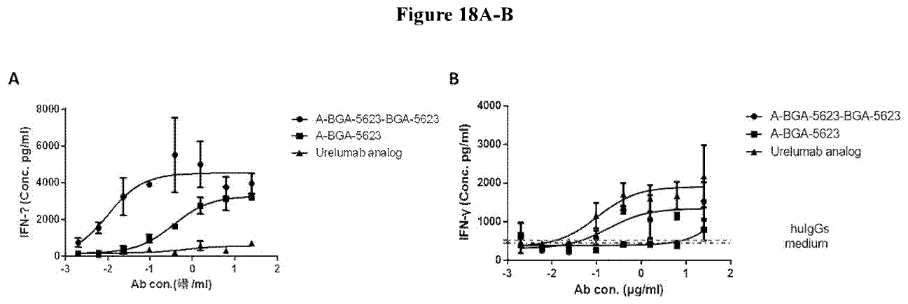

First, we constructed CD137×CEA multispecific antibody variants with different module ratios such as 2:4, 1:1 and 1:2, namely BE-718 (A-BGA-5623-BGA-5623) (SEQ ID NOs: 207 and 89), BE-942 (ZW 1+1) (SEQ ID NOs: 211, 213 and 215), which is BGA-5623 in the 1+1 configuration and BE-755 (ZW1+2) (SEQ ID NOs: 211, 213 and 217) which is BGA-5623 in the 1+2 configuration (FIG. 17). For antibody constructs with a “ZW” designation, an inert Fc was used for these multispecific antibodies and the Azymetric™ Platform from Zymeworks was utilized to assemble the Fab×VH configuration, in which ZW1 mutations (chain A: T350V/L351Y/F405A/Y407V; chain B: T350V/T366L/K392L/T394W) were introduced in the CH3 domain of heavy chain to allow efficient heterodimer formation (Von Kreudenstein et al., (2013) Mabs 5(5):646-54). For BE-189 (A-BGA-5623) (SEQ ID NOs: 167 and 89), which represents the multispecific antibody with a module ratio of 2:2, we were able to investigate how the module ratio influences cytokine release. As described above in Example 7, the high CEA expressing cell line, CT26/CEA, together with PBMCs (2× 10⁵/well) and HEK293/OS8 cells, which could trigger the first signal for T-cell activation were used for an in vitro CD137 activation assay. As shown in Table 21 and FIG. 18A-B, the multispecific antibody of a module ratio of 2:2 was demonstrated to be a potent CD137 agonist without CD137 intrinsic activation, which suggests BE-189 (Format A-BGA-5623) activates CD137 in a CEA dependent way. In contrast, the multispecific antibody BE-718 (A-BGA-5623-BGA-5623) with a module ratio of 2:4, was shown to activate CD137 even in the absence of CEA expressing cells.

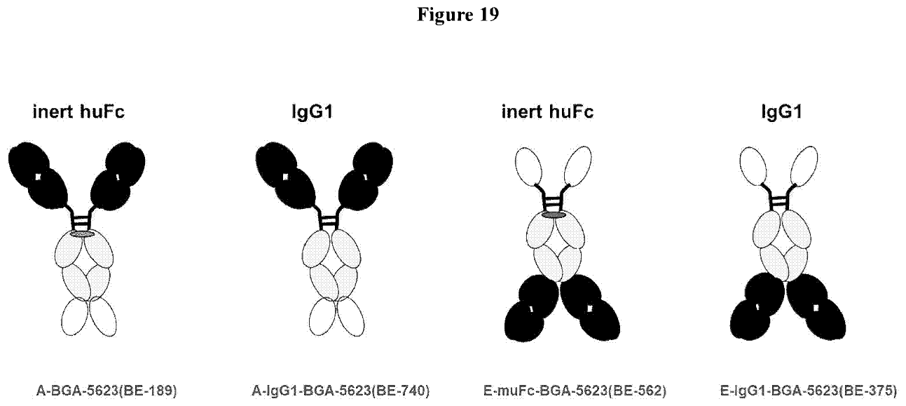

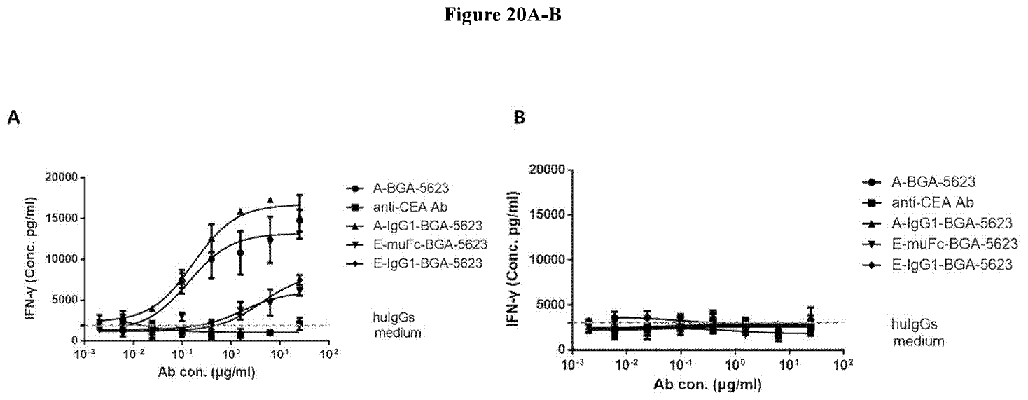

Next, we then investigated how module orientation and Fc functions influence CD137 activation. In this experiment, we constructed BE-740 (A-IgG1-BGA-5623) (SEQ ID NOS: 209 and 89), which was exactly the same as A-BGA-5623 in the format except for a wild-type IgG1 Fc was used to substitute the inert Fc. We also constructed BE-562 (E-muFc-BGA-5623) (SEQ ID NOs: 219 and 89) and BE-375 (E-IgG1-BGA-5623) (SEQ ID NOs: 221 and 89), respectively. As shown in FIG. 19, these two multispecific antibodies share the same pair of anti-CEA antibodies and anti-huCD137 VH domain (CEA and BGA-5623) as A-BGA-5623 and A-IgG1-BGA-5623, but with the opposite orientation. As described in Example 7, a PBMC based cytokine release assay was used to quantify the potency of CD137 activation. Based on the in vitro results, A-BGA-5623 and A-IgG1-BGA-5623 were demonstrated to be more potent in CD137 activation than E-muFc-BGA-5623 and E-IgG1-BGA-5623. In addition, based on this experiment, the Fc function seems to have minimal influence on CD137 activation (FIG. 20A-B).

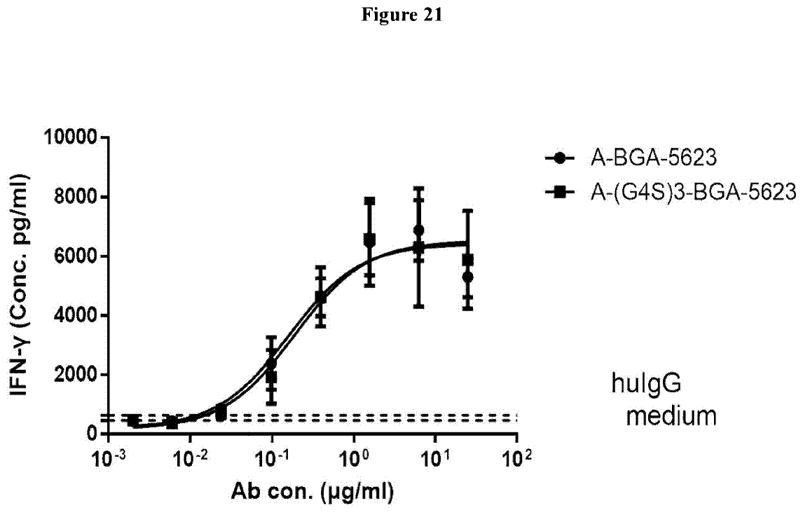

At last, the linker connecting Fc and VH domain antibody was evaluated for its influence on CD137 activation. A-(G4S)3-BGA-5623 (BE-244) (SEQ ID NOs: 223 and 89) was created with substitution of G4S with a 15 AA linker of (G4S)₃ (SEQ ID NO:251). Again, the PBMC based cytokine release assay was used to compare the potency. As shown in FIG. 21 and Table 21, the linker length has minimal influence in CD137 activation.

TABLE 21
Key parameters for in vitro CD137 activation

| | | | | | SEC-HPLC | Functional |
|---|---|---|---|---|---|---|
| | | | | | (%) after | assay |
| | Alternative | Pair of | Module | | Protein A | EC50 |
| Code | name | mAbs | ratio | Fc | purification | (ug/ml) |
| BE-189 | A-BGA- | BGA-5623/ | 2 + 2 | Inert Fc | 94.55 | 0.31 |
| | 5623 | CEA | | | | |
| BE-718 | A-BGA- | | 2 + 4 | Inert Fc | 89.47 | 0.01 |
| | 5623-BGA- | | | | | |
| | 5623 | | | | | |
| BE-740 | A-IgG1- | | 2 + 2 | wt-IgG1 | 98.71 | 0.17 |
| | BGA-5623 | | | | | |
| BE-942 | ZW1 + 1 | | 1 + 1 | LALADS | 92.65 | 25.06 |
| BE-755 | ZW1 + 2 | | 1 + 2 | LALADS | 83.19 | 1.16 |
| BE-562 | E-muFc- | | 2 + 2 | Inert Fc | 98.17 | 4.69 |
| | BGA-5623 | | | | | |
| BE-375 | E-IgG1- | | 2 + 2 | wt-IgG1 | 97.76 | 1.76 |
| | BGA-5623 | | | | | |
| BE-244 | A-(G4S)3- | | 2 + 2 | Inert Fc | 98.84 | 0.18 |
| | BGA-5623 | | | | | |

TABLE 22
Amino acid and DNA sequences of CD137xCEA multispecific antibodies

| | SEQ ID | |
|---|---|---|
| CONSTRUCT | NO: | SEQUENCE |
| BE718 | SEQ ID | QVQLVQSGAEVKKPGASVKVSCKASGYIFTSYYLHWVRQAPGQGLEWIGYI |
| A-BGA- | NO: 207 | NPQTGKTSYAQKFQGRVTMTRDTSTSTVYMELSSLRSEDTAVYYCAREYGN |
| 5623-BGA- | | YNYPLDYWGQGTLVTVSSASTKGPSVFPLAPSSKSTSGGTAALGCLVKDYFP |
| 5623 | | EPVTVSWNSGALTSGVHTFPAVLQSSGLYSLSSVVTVPSSSLGTQTYICNVNH |
| heavy | | KPSNTKVDKKVEPKSCDKTHTCPPCPAPPAAGPSVFLFPPKPKDTLMISRTPE |
| chain AA | | VTCVVVDVSHEDPEVKFNWYVDGVEVHNAKTKPREEQYNSTYRVVSVLTV |
| | | LHQDWLNGKEYKCKVSNKALAAPIEKTISKAKGQPREPQVYTLPPSREEMT |
| | | KNQVSLTCLVKGFYPSDIAVEWESNGQPENNYKTTPPVLDSDGSFFLYSKLT |
| | | VDKSRWQQGNVFSCSVMHEALHNHYTQKSLSLSPGKGGGGSEVQLLESGG |
| | | GLVQPGGSLRLSCAASGFTVSAEDVGWVRQAPGKGLEWVSAILDKGGSTY |
| | | YAESVKGRFTISRDNAKNTLYLQMSSLRAEDTAVYYCARIVYHAGGGVTFD |
| | | TRGQGTQVTVSSGGGGSEVQLLESGGGLVQPGGSLRLSCAASGFTVSAEDV |
| | | GWVRQAPGKGLEWVSAILDKGGSTYYAESVKGRFTISRDNAKNTLYLQMSS |
| | | LRAEDTAVYYCARIVYHAGGGVTFDTRGQGTQVTVSS |
| BE718 | SEQ ID | CAGGTGCAGCTGGTGCAGAGCGGCGCGGAAGTGAAAAAACCGGGCGCG |
| A-BGA- | NO: 208 | AGCGTGAAAGTGAGCTGCAAAGCGAGCGGCTATATTTTTACCAGCTATTA |
| 5623-BGA- | | CCTGCATTGGGTGCGCCAGGCGCCGGGCCAGGGCCTGGAATGGATTGGCT |
| 5623 | | ATATTAACCCGCAGACCGGCAAGACCAGCTATGCCCAGAAATTTCAGGGC |
| heavy | | CGCGTGACCATGACCCGCGATACCAGCACCAGCACCGTGTATATGGAACT |
| chain | | GAGCAGCCTGCGCAGCGAAGATACCGCGGTGTATTATTGCGCGCGCGAAT |
| DNA | | ATGGCAACTATAACTATCCGCTGGATTATTGGGGCCAGGGCACCCTGGTGA |
| | | CCGTGAGCAGCGCTAGCACCAAGGGGCCCTCGGTCTTCCCCCTGGCACC |
| | | CTCCTCCAAGAGTACTTCTGGGGGCACAGCGGCCCTGGGCTGCCTGGTCA |
| | | AGGACTACTTCCCCGAACCGGTGACGGTGTCGTGGAACTCAGGCGCCCT |
| | | GACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCA |
| | | GACCTACATCTGCAACGTGAATCACAAGCCCAGCAACACCAAGGTGGAC |
| | | AAGAAAGTTGAGCCAAAGTCCTGTGACAAGACCCACACATGCCCCCCTT |
| | | GTCCTGCTCCACCAGCTGCAGGACCAAGCGTGTTCCTGTTTCCACCCAAG |
| | | CCCAAGGATACCCTGATGATCTCTCGGACCCCAGAGGTGACATGCGTGGT |
| | | GGTGGATGTGAGCCACGAGGACCCCGAGGTGAAGTTCAACTGGTATGTG |
| | | GACGGCGTGGAGGTGCACAATGCTAAGACCAAGCCCAGGGAGGAGCAG |
| | | TACAACTCCACCTATAGAGTGGTGTCTGTGCTGACAGTGCTGCACCAGGA |
| | | TTGGCTGAACGGCAAGGAGTATAAGTGCAAGGTGTCCAATAAGGCCCTG |
| | | GCCGCTCCTATCGAGAAGACCATCTCTAAGGCCAAGGGCCAGCCCAGAG |
| | | AGCCTCAGGTGTACACACTGCCTCCATCCCGGGAAGAGATGACCAAGAA |
| | | CCAGGTGTCTCTGACATGTCTGGTCAAGGGCTTCTATCCCTCTGACATCGC |
| | | CGTGGAGTGGGAGAGCAATGGCCAGCCTGAGAACAATTACAAGACCACA |
| | | CCCCCTGTGCTGGATTCCGACGGCTCTTTCTTTCTGTATAGCAAGCTGACC |
| | | GTGGACAAGTCCCGGTGGCAGCAGGGCAACGTGTTCAGCTGTTCCGTGA |
| | | TGCACGAAGCTCTGCATAATCACTATACTCAGAAATCCCTGTCACTGTCAC |
| | | CTGGTAAAGGTGGAGGCGGTTCAGAGGTCCAGTTACTTGAGAGTGGTGG |
| | | AGGTCTGGTCCAACCAGGAGGTTCGCTGCGTTTATCCTGCGCCGCGTCTG |
| | | GATTCACGGTTTCCGCCGAAGACGTGGGTTGGGTGCGTCAAGCGCCGGG |
| | | GAAAGGACTGGAATGGGTCTCCGCCATCTTGGATAAGGGTGGTTCGACAT |
| | | ACTATGCGGAAAGTGTCAAAGGGCGCTTTACGATCTCGCGCGATAACGCA |
| | | AAAAATACTCTTTACCTTCAAATGTCTAGCCTTCGTGCTGAGGACACTGCG |
| | | GTGTACTACTGCGCCCGCATTGTCTACCATGCTGGTGGTGGCGTCACCTTT |
| | | GATACTCGGGGACAGGGCACCCAAGTTACAGTCTCATCGGGCGGCGGAG |
| | | GATCGGAGGTCCAGTTACTTGAGAGTGGTGGAGGTCTGGTCCAACCAGG |
| | | AGGTTCGCTGCGTTTATCCTGCGCCGCGTCTGGATTCACGGTTTCCGCCGA |
| | | AGACGTGGGTTGGGTGCGTCAAGCGCCGGGGAAAGGACTGGAATGGGTC |
| | | TCCGCCATCTTGGATAAGGGTGGTTCGACATACTATGCGGAAAGTGTCAA |
| | | AGGGCGCTTTACGATCTCGCGCGATAACGCAAAAAATACTCTTTACCTTCA |
| | | AATGTCTAGCCTTCGTGCTGAGGACACTGCGGTGTACTACTGCGCCCGCA |
| | | TTGTCTACCATGCTGGTGGTGGCGTCACCTTTGATACTCGGGGACAGGGC |
| | | ACCCAAGTTACAGTCTCATCG |
| BE-740 | SEQ ID | QVQLVQSGAEVKKPGASVKVSCKASGYIFTSYYLHWVRQAPGQGLEWIGYI |
| AA | NO: 209 | NPQTGKTSYAQKFQGRVTMTRDTSTSTVYMELSSLRSEDTAVYYCAREYGN |
| | | YNYPLDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLT |
| | | VDKSRWQQGNVFSCSVMHEALHNHYTQKSLSLSPGKGGGGSEVQLLESGG |
| | | GLVQPGGSLRLSCAASGFTVSAEDVGWVRQAPGKGLEWVSAILDKGGSTY |
| | | YAESVKGRFTISRDNAKNTLYLQMSSLRAEDTAVYYCARIVYHAGGGVTFD |
| | | TRGQGTQVTVSS |
| BE-740 | SEQ ID | CAGGTGCAGCTGGTGCAGAGCGGCGCGGAAGTGAAAAAACCGGGCGCG |
| DNA | NO: 210 | AGCGTGAAAGTGAGCTGCAAAGCGAGCGGCTATATTTTTACCAGCTATTA |
| | | CCTGCATTGGGTGCGCCAGGCGCCGGGCCAGGGCCTGGAATGGATTGGCT |
| | | ATATTAACCCGCAGACCGGCAAGACCAGCTATGCCCAGAAATTTCAGGGC |
| | | CGCGTGACCATGACCCGCGATACCAGCACCAGCACCGTGTATATGGAACT |
| | | GAGCAGCCTGCGCAGCGAAGATACCGCGGTGTATTATTGCGCGCGCGAAT |
| | | ATGGCAACTATAACTATCCGCTGGATTATTGGGGCCAGGGCACCCTGGTGA |
| | | CCGTGAGCAGCGCTAGCACCAAGGGGCCCTCGGTCTTCCCCCTGGCACC |
| | | CTCCTCCAAGAGTACTTCTGGGGGCACAGCGGCCCTGGGCTGCCTGGTCA |
| | | AGGACTACTTCCCCGAACCGGTGACGGTGTCGTGGAACTCAGGCGCCCT |
| | | GACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCA |
| | | GACCTACATCTGCAACGTGAATCACAAGCCCAGCAACACCAAGGTGGAC |
| | | AAGAAAGTTGAGCCCAAATCTTGTGACAAAACTCACACATGCCCACCGT |
| | | GCCCAGCACCTGAACTCCTGGGGGGACCGTCAGTCTTCCTCTTCCCCCCA |
| | | AAACCCAAGGACACCCTCATGATCTCCCGGACCCCTGAGGTCACATGCGT |
| | | GGTGGTGGACGTGAGCCACGAAGACCCTGAGGTCAAGTTCAACTGGTAC |
| | | GTGGACGGCGTGGAGGTGCATAATGCCAAGACAAAGCCGCGGGAGGAGC |
| | | AGTACAACAGCACGTACCGTGTGGTCAGCGTCCTCACCGTCCTGCACCAG |
| | | GACTGGCTGAATGGCAAGGAGTACAAGTGCAAGGTCTCCAACAAAGCCC |
| | | TCCCAGCCCCCATCGAGAAAACCATCTCCAAAGCCAAAGGGCAGCCCCG |
| | | AGAACCACAGGTGTACACCCTGCCCCCATCCCGGGATGAGCTGACCAAG |
| | | AACCAGGTCAGCCTGACCTGCCTGGTCAAAGGCTTCTATCCCAGCGACAT |
| | | CGCCGTGGAGTGGGAGAGCAATGGGCAGCCGGAGAACAACTACAAGAC |
| | | CACGCCTCCCGTGCTGGACTCCGACGGCTCCTTCTTCCTCTACAGCAAGC |
| | | TCACCGTGGACAAGAGCAGGTGGCAGCAGGGGAACGTCTTCTCATGCTC |
| | | CGTGATGCATGAGGCTCTGCACAACCACTACACGCAGAAGAGCCTCTCCC |
| | | TGTCTCCGGGTAAAGGTGGAGGCGGTTCAGAGGTCCAGTTACTTGAGAG |
| | | TGGTGGAGGTCTGGTCCAACCAGGAGGTTCGCTGCGTTTATCCTGCGCCG |
| | | CGTCTGGATTCACGGTTTCCGCCGAAGACGTGGGTTGGGTGCGTCAAGCG |
| | | CCGGGGAAAGGACTGGAATGGGTCTCCGCCATCTTGGATAAGGGTGGTTC |
| | | GACATACTATGCGGAAAGTGTCAAAGGGCGCTTTACGATCTCGCGCGATA |
| | | ACGCAAAAAATACTCTTTACCTTCAAATGTCTAGCCTTCGTGCTGAGGAC |
| | | ACTGCGGTGTACTACTGCGCCCGCATTGTCTACCATGCTGGTGGTGGCGTC |
| | | ACCTTTGATACTCGGGGACAGGGCACCCAAGTTACAGTCTCATCG |
| BE-942 | SEQ ID | QVQLVQSGAEVKKPGASVKVSCKASGYIFTSYYLHWVRQAPGQGLEWIGYI |
| (ZW1 + 1) | NO: 211 | NPQTGKTSYAQKFQGRVTMTRDTSTSTVYMELSSLRSEDTAVYYCAREYGN |
| anti-CEA | | YNYPLDYWGQGTLVTVSSASTKGPSVFPLAPSSKSTSGGTAWLGCEVTDYFP |
| heavy | | EPVTVSWNSGALTSGVHTFPAVLESSGLYSLSSVVTVPSSSLGTQTYICNVNH |
| chain AA | | KPSNTKVDKKVEPKSCDKTHTCPPCPAPEAAGGPSVFLFPPKPKDTLMISRTP |
| | | EVTCVVVSVSHEDPEVKFNWYVDGVEVHNAKTKPREEQYNSTYRVVSVLT |
| | | VLHQDWLNGKEYKCKVSNKALPAPIEKTISKAKGQPREPQVYVLPPSRDELT |
| | | KNQVSLLCLVKGFYPSDIAVEWESNGQPENNYLTWPPVLDSDGSFFLYSKLT |
| | | VDKSRWQQGNVFSCSVMHEALHNHYTQKSLSLSPGK |
| BE-942 | SEQ ID | CAGGTGCAGCTGGTGCAGAGCGGCGCGGAAGTGAAAAAACCGGGCGCG |
| (ZW1 + 1) | NO: 212 | AGCGTGAAAGTGAGCTGCAAAGCGAGCGGCTATATTTTTACCAGCTATTA |
| anti-CEA | | CCTGCATTGGGTGCGCCAGGCGCCGGGCCAGGGCCTGGAATGGATTGGCT |
| heavy | | ATATTAACCCGCAGACCGGCAAGACCAGCTATGCCCAGAAATTTCAGGGC |
| chain | | CGCGTGACCATGACCCGCGATACCAGCACCAGCACCGTGTATATGGAACT |
| DNA | | GAGCAGCCTGCGCAGCGAAGATACCGCGGTGTATTATTGCGCGCGCGAAT |
| | | ATGGCAACTATAACTATCCGCTGGATTATTGGGGCCAGGGCACCCTGGTGA |
| | | CCGTGAGCAGCGCTAGCACCAAGGGACCAAGCGTGTTCCCACTGGCACC |
| | | CAGCTCCAAGAGCACCTCCGGAGGAACAGCATGGCTGGGATGCGAGGTG |
| | | ACCGACTACTTCCCCGAGCCTGTGACAGTGTCTTGGAACAGCGGCGCCCT |
| | | GACCAGCGGAGTGCACACATTTCCTGCCGTGCTGGAGTCTAGCGGCCTGT |
| | | ACTCCCTGTCCTCTGTGGTGACCGTGCCTAGCTCCTCTCTGGGCACCCAG |
| | | ACATATATCTGTAACGTGAATCACAAGCCATCTAATACAAAGGTGGATAAG |
| | | AAGGTGGAGCCAAAGTCCTGCGACAAGACCCACACATGCCCACCTTGTC |
| | | CAGCACCAGAGGCAGCAGGAGGACCATCCGTGTTCCTGTTTCCACCCAA |
| | | GCCCAAGGATACCCTGATGATCTCTCGGACCCCCGAGGTGACATGCGTGG |
| | | TGGTGAGCGTGTCCCACGAGGACCCTGAGGTGAAGTTCAACTGGTACGT |
| | | GGATGGCGTGGAGGTGCACAATGCCAAGACAAAGCCCCGGGAGGAGCA |
| | | GTACAACAGCACCTATAGAGTGGTGTCCGTGCTGACAGTGCTGCACCAGG |
| | | ACTGGCTGAACGGCAAGGAGTATAAGTGCAAGGTGTCTAATAAGGCCCTG |
| | | CCTGCCCCAATCGAGAAGACCATCAGCAAGGCAAAGGGACAGCCCAGGG |
| | | AGCCTCAGGTGTACGTGCTGCCTCCAAGCCGCGACGAGCTGACAAAGAA |
| | | CCAGGTGTCCCTGCTGTGCCTGGTGAAGGGCTTCTATCCTTCCGATATCGC |
| | | CGTGGAGTGGGAGTCTAATGGCCAGCCAGAGAACAATTACCTGACCTGG |
| | | CCCCCTGTGCTGGACTCCGATGGCTCTTTCTTTCTGTATTCTAAGCTGACA |
| | | GTGGATAAGAGCAGGTGGCAGCAGGGCAACGTGTTTTCTTGCAGCGTGA |
| | | TGCACGAGGCCCTGCACAATCACTACACCCAGAAGTCCCTGTCTCTGAGC |
| | | CCTGGCAAG |
| BE-942 | SEQ ID | DIQMTQSPSSLSASVGDRVTITCRASENQYGYLAWYQQKPGKVPKLLIYNY |
| (ZW1 + 1) | NO: 213 | KNLVEGVPSRFSGSGSGTDFTLTISSLQPEDVATYYCQHHLGTPYTFGQGTKV |
| anti-CEA | | EIKRTVAAPSVAIFPPSDERLKSGTASVVCVLNNFYPREAKVQWKVDNALQS |
| light chain | | GNSQESVTEQDSKDSTYSLSSRLTLSKADYEKHKVYACEVTHQGLSSPVTKS |
| AA | | FNRGEC |
| BE-942 | SEQ ID | GATATTCAGATGACCCAGAGCCCGAGCAGCCTGAGCGCGAGCGTGGGCG |
| (ZW1 + 1) | NO: 214 | ATCGCGTGACCATTACCTGCCGCGCGAGCGAAAACCAGTATGGCTATCTG |
| anti-CEA | | GCGTGGTATCAGCAGAAACCGGGCAAAGTGCCGAAACTGCTGATTTATAA |
| light chain | | CTATAAAAACCTGGTGGAAGGCGTGCCGAGCCGCTTTAGCGGCAGCGGC |
| DNA | | AGCGGCACCGATTTTACCCTGACCATTAGCAGCCTGCAGCCGGAAGATGT |
| | | GGCGACCTATTATTGCCAGCATCATCTGGGCACCCCGTATACCTTTGGCCA |
| | | GGGCACCAAAGTGGAAATTAAAAGGACAGTGGCAGCACCAAGCGTGGC |
| | | AATCTTCCCACCTTCCGACGAGAGACTGAAGTCCGGCACCGCTAGCGTGG |
| | | TGTGCGTGCTGAACAACTTCTACCCCCGGGAGGCCAAGGTGCAGTGGAA |
| | | GGTGGATAACGCCCTGCAGTCCGGCAATTCTCAGGAGAGCGTGACCGAG |
| | | CAGGACTCCAAGGATTCTACATATAGCCTGAGCTCCAGGCTGACCCTGTC |
| | | TAAGGCCGACTACGAGAAGCACAAGGTGTATGCCTGCGAGGTGACACAC |
| | | CAGGGCCTGTCTAGCCCCGTGACCAAGTCCTTCAACCGCGGCGAGTGT |
| BE-942 | SEQ ID | EVQLLESGGGLVQPGGSLRLSCAASGFTVSAEDVGWVRQAPGKGLEWVSAI |
| (ZW1 + 1) | NO: 215 | LDKGGSTYYAESVKGRFTISRDNAKNTLYLQMSSLRAEDTAVYYCARIVYH |
| anti- | | AGGGVTFDTRGQGTQVTVSSEPKSSDKTHTCPPCPAPEAAGGPSVFLFPPKP |
| CD137 | | KDTLMISRTPEVTCVVVSVSHEDPEVKFNWYVDGVEVHNAKTKPREEQYN |
| heavy | | STYRVVSVLTVLHQDWLNGKEYKCKVSNKALPAPIEKTISKAKGQPREPQV |
| chain AA | | YVYPPSRDELTKNQVSLTCLVKGFYPSDIAVEWESNGQPENNYKTTPPVLDS |
| | | DGSFALVSKLTVDKSRWQQGNVFSCSVMHEALHNHYTQKSLSLSPGK |
| BE-942 | SEQ ID | GAGGTCCAGTTACTTGAGAGTGGTGGAGGTCTGGTCCAACCAGGAGGTT |
| (ZW1 + 1) | NO: 216 | CGCTGCGTTTATCCTGCGCCGCGTCTGGATTCACGGTTTCCGCCGAAGAC |
| anti- | | GTGGGTTGGGTGCGTCAAGCGCCGGGGAAAGGACTGGAATGGGTCTCCG |
| CD137 | | CCATCTTGGATAAGGGTGGTTCGACATACTATGCGGAAAGTGTCAAAGGG |
| heavy | | CGCTTTACGATCTCGCGCGATAACGCAAAAAATACTCTTTACCTTCAAATG |
| chain | | TCTAGCCTTCGTGCTGAGGACACTGCGGTGTACTACTGCGCCCGCATTGT |
| DNA | | CTACCATGCTGGTGGTGGCGTCACCTTTGATACTCGGGGACAGGGCACCC |
| | | AAGTTACAGTCTCATCGGAGCCAAAGTCCTCCGACAAGACCCACACATGC |
| | | CCACCTTGTCCAGCACCAGAGGCAGCAGGAGGACCATCCGTGTTCCTGTT |
| | | TCCACCCAAGCCCAAGGATACCCTGATGATCTCTCGGACCCCCGAGGTGA |
| | | CATGCGTGGTGGTGAGCGTGTCCCACGAGGACCCTGAGGTGAAGTTCAA |
| | | CTGGTACGTGGATGGCGTGGAGGTGCACAATGCCAAGACAAAGCCCCGG |
| | | GAGGAGCAGTACAACAGCACCTATAGAGTGGTGTCCGTGCTGACAGTGC |
| | | TGCACCAGGACTGGCTGAACGGCAAGGAGTATAAGTGCAAGGTGTCTAA |
| | | TAAGGCCCTGCCTGCCCCAATCGAGAAGACCATCAGCAAGGCAAAGGGA |
| | | CAGCCCAGGGAGCCTCAGGTGTACGTGTATCCTCCAAGCCGCGACGAGCT |
| | | GACCAAGAACCAGGTGTCCCTGACATGTCTGGTGAAGGGCTTTTACCCTT |
| | | CCGATATCGCCGTGGAGTGGGAGTCTAATGGCCAGCCAGAGAACAATTAT |
| | | AAGACCACACCCCCTGTGCTGGACTCCGATGGCTCTTTCGCCCTGGTGTC |
| | | TAAGCTGACCGTGGATAAGAGCAGGTGGCAGCAGGGCAACGTGTTTTCTT |
| | | GCAGCGTGATGCACGAGGCCCTGCACAATCACTACACACAGAAGTCCCT |
| | | GTCTCTGAGCCCTGGCAAG |
| BE-755 | SEQ ID | EVQLLESGGGLVQPGGSLRLSCAASGFTVSAEDVGWVRQAPGKGLEWVSAI |
| (ZW1 + 2) | NO: 217 | LDKGGSTYYAESVKGRFTISRDNAKNTLYLQMSSLRAEDTAVYYCARIVYH |
| anti- | | AGGGVTFDTRGQGTQVTVSSGGGGSEVQLLESGGGLVQPGGSLRLSCAASG |
| CD137 | | FTVSAEDVGWVRQAPGKGLEWVSAILDKGGSTYYAESVKGRFTISRDNAKN |
| heavy | | TLYLQMSSLRAEDTAVYYCARIVYHAGGGVTFDTRGQGTQVTVSSEPKSSD |
| chain AA | | KTHTCPPCPAPEAAGGPSVFLFPPKPKDTLMISRTPEVTCVVVSVSHEDPEVK |
| | | FNWYVDGVEVHNAKTKPREEQYNSTYRVVSVLTVLHQDWLNGKEYKCKV |
| | | SNKALPAPIEKTISKAKGQPREPQVYVYPPSRDELTKNQVSLTCLVKGFYPSD |
| | | IAVEWESNGQPENNYKTTPPVLDSDGSFALVSKLTVDKSRWQQGNVFSCSV |
| | | MHEALHNHYTQKSLSLSPGK |
| BE-755 | SEQ ID | GAGGTCCAGTTACTTGAGAGTGGTGGAGGTCTGGTCCAACCAGGAGGTT |
| (ZW1 + 2) | NO: 218 | CGCTGCGTTTATCCTGCGCCGCGTCTGGATTCACGGTTTCCGCCGAAGAC |
| anti- | | GTGGGTTGGGTGCGTCAAGCGCCGGGGAAAGGACTGGAATGGGTCTCCG |
| CD137 | | CCATCTTGGATAAGGGTGGTTCGACATACTATGCGGAAAGTGTCAAAGGG |
| heavy | | CGCTTTACGATCTCGCGCGATAACGCAAAAAATACTCTTTACCTTCAAATG |
| chain | | TCTAGCCTTCGTGCTGAGGACACTGCGGTGTACTACTGCGCCCGCATTGT |
| DNA | | CTACCATGCTGGTGGTGGCGTCACCTTTGATACTCGGGGACAGGGCACCC |
| | | AAGTTACAGTCTCATCGGGCGGCGGAGGGAGCGAGGTGCAGCTGCTTGA |
| | | GAGTGGTGGAGGTCTGGTCCAACCAGGAGGTTCGCTGCGTTTATCCTGCG |
| | | CCGCGTCTGGATTCACGGTTTCCGCCGAAGACGTGGGTTGGGTGCGTCAA |
| | | GCGCCGGGGAAAGGACTGGAATGGGTCTCCGCCATCTTGGATAAGGGTG |
| | | GTTCGACATACTATGCGGAAAGTGTCAAAGGGCGCTTTACGATCTCGCGC |
| | | GATAACGCAAAAAATACTCTTTACCTTCAAATGTCTAGCCTTCGTGCTGAG |
| | | GACACTGCGGTGTACTACTGCGCCCGCATTGTCTACCATGCTGGTGGTGG |
| | | CGTCACCTTTGATACTCGGGGACAGGGCACCCAAGTGACCGTGTCCAGC |
| | | GAGCCAAAGTCCTcCGACAAGACCCACACATGCCCACCTTGTCCAGCACC |
| | | AGAGGCAGCAGGAGGACCATCCGTGTTCCTGTTTCCACCCAAGCCCAAG |
| | | GATACCCTGATGATCTCTCGGACCCCCGAGGTGACATGCGTGGTGGTGAG |
| | | CGTGTCCCACGAGGACCCTGAGGTGAAGTTCAACTGGTACGTGGATGGC |
| | | GTGGAGGTGCACAATGCCAAGACAAAGCCCCGGGAGGAGCAGTACAAC |
| | | AGCACCTATAGAGTGGTGTCCGTGCTGACAGTGCTGCACCAGGACTGGCT |
| | | GAACGGCAAGGAGTATAAGTGCAAGGTGTCTAATAAGGCCCTGCCTGCCC |
| | | CAATCGAGAAGACCATCAGCAAGGCAAAGGGACAGCCCAGGGAGCCTC |
| | | AGGTGTACGTGTATCCTCCAAGCCGCGACGAGCTGACCAAGAACCAGGT |
| | | GTCCCTGACATGTCTGGTGAAGGGCTTTTACCCTTCCGATATCGCCGTGGA |
| | | GTGGGAGTCTAATGGCCAGCCAGAGAACAATTATAAGACCACACCCCCTG |
| | | TGCTGGACTCCGATGGCTCTTTCGCCCTGGTGTCTAAGCTGACCGTGGATA |
| | | AGAGCAGGTGGCAGCAGGGCAACGTGTTTTCTTGCAGCGTGATGCACGA |
| | | GGCCCTGCACAATCACTACACACAGAAGTCCCTGTCTCTGAGCCCTGGCA |
| | | AG |
| BE-562 | SEQ ID | EVQLLESGGGLVQPGGSLRLSCAASGFTVSAEDVGWVRQAPGKGLEWVSAI |
| (E-mutFc) | NO: 219 | LDKGGSTYYAESVKGRFTISRDNAKNTLYLQMSSLRAEDTAVYYCARIVYH |
| heavy | | AGGGVTFDTRGQGTQVTVSSEPKSSDKTHTCPPCPAPPAAGPSVFLFPPKPK |
| chain AA | | DTLMISRTPEVTCVVVDVSHEDPEVKFNWYVDGVEVHNAKTKPREEQYNS |
| | | TYRVVSVLTVLHQDWLNGKEYKCKVSNKALAAPIEKTISKAKGQPREPQVY |
| | | TLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGKGGGGS |
| | | QVQLVQSGAEVKKPGASVKVSCKASGYIFTSYYLHWVRQAPGQGLEWIGYI |
| | | NPQTGKTSYAQKFQGRVTMTRDTSTSTVYMELSSLRSEDTAVYYCAREYGN |
| | | YNYPLDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCD |
| BE-562 | SEQ ID | GAGGTCCAGTTACTTGAGAGTGGTGGAGGTCTGGTCCAACCAGGAGGTT |
| (E-mutFc) | NO: 220 | CGCTGCGTTTATCCTGCGCCGCGTCTGGATTCACGGTTTCCGCCGAAGAC |
| heavy | | GTGGGTTGGGTGCGTCAAGCGCCGGGGAAAGGACTGGAATGGGTCTCCG |
| chain | | CCATCTTGGATAAGGGTGGTTCGACATACTATGCGGAAAGTGTCAAAGGG |
| DNA | | CGCTTTACGATCTCGCGCGATAACGCAAAAAATACTCTTTACCTTCAAATG |
| | | TCTAGCCTTCGTGCTGAGGACACTGCGGTGTACTACTGCGCCCGCATTGT |
| | | CTACCATGCTGGTGGTGGCGTCACCTTTGATACTCGGGGACAGGGCACCC |
| | | AAGTTACAGTCTCATCGGAGCCAAAGTCCTCTGACAAGACCCACACATGC |
| | | CCCCCTTGTCCTGCTCCACCAGCTGCAGGACCAAGCGTGTTCCTGTTTCC |
| | | ACCCAAGCCCAAGGATACCCTGATGATCTCTCGGACCCCAGAGGTGACAT |
| | | GCGTGGTGGTGGATGTGAGCCACGAGGACCCCGAGGTGAAGTTCAACTG |
| | | GTATGTGGACGGCGTGGAGGTGCACAATGCTAAGACCAAGCCCAGGGAG |
| | | GAGCAGTACAACTCCACCTATAGAGTGGTGTCTGTGCTGACAGTGCTGCA |
| | | CCAGGATTGGCTGAACGGCAAGGAGTATAAGTGCAAGGTGTCCAATAAG |
| | | GCCCTGGCCGCTCCTATCGAGAAGACCATCTCTAAGGCCAAGGGCCAGCC |
| | | CAGAGAGCCTCAGGTGTACACACTGCCTCCATCCCGGGAAGAGATGACC |
| | | AAGAACCAGGTGTCTCTGACATGTCTGGTCAAGGGCTTCTATCCCTCTGA |
| | | CATCGCCGTGGAGTGGGAGAGCAATGGCCAGCCTGAGAACAATTACAAG |
| | | ACCACACCCCCTGTGCTGGATTCCGACGGCTCTTTCTTTCTGTATAGCAAG |
| | | CTGACCGTGGACAAGTCCCGGTGGCAGCAGGGCAACGTGTTCAGCTGTT |
| | | CCGTGATGCACGAAGCTCTGCATAATCACTATACTCAGAAATCCCTGTCAC |
| | | TGTCACCTGGTAAAGGTGGAGGCGGTTCACAGGTGCAGCTGGTGCAGAG |
| | | CGGCGCGGAAGTGAAAAAACCGGGCGCGAGCGTGAAAGTGAGCTGCAA |
| | | AGCGAGCGGCTATATTTTTACCAGCTATTACCTGCATTGGGTGCGCCAGGC |
| | | GCCGGGCCAGGGCCTGGAATGGATTGGCTATATTAACCCGCAGACCGGCA |
| | | AGACCAGCTATGCCCAGAAATTTCAGGGCCGCGTGACCATGACCCGCGAT |
| | | ACCAGCACCAGCACCGTGTATATGGAACTGAGCAGCCTGCGCAGCGAAG |
| | | ATACCGCGGTGTATTATTGCGCGCGCGAATATGGCAACTATAACTATCCGCT |
| | | GGATTATTGGGGCCAGGGCACCCTGGTGACCGTGAGCAGCGCTAGCACC |
| | | AAGGGGCCCTCGGTCTTCCCCCTGGCACCCTCCTCCAAGAGTACTTCTGG |
| | | GGGCACAGCGGCCCTGGGCTGCCTGGTCAAGGACTACTTCCCCGAACCG |
| | | GTGACGGTGTCGTGGAACTCAGGCGCCCTGACCAGCGGCGTGCACACCT |
| | | TCCCGGCTGTCCTACAGTCCTCAGGACTCTACTCCCTCAGCAGCGTGGTG |
| | | ACCGTGCCCTCCAGCAGCTTGGGCACCCAGACCTACATCTGCAACGTGAA |
| | | TCACAAGCCCAGCAACACCAAGGTGGACAAGAAAGTTGAGCCCAAATCT |
| | | TGTGAC |
| BE-375 | SEQ ID | EVQLLESGGGLVQPGGSLRLSCAASGFTVSAEDVGWVRQAPGKGLEWVSAI |
| (E- | NO: 221 | LDKGGSTYYAESVKGRFTISRDNAKNTLYLQMSSLRAEDTAVYYCARIVYH |
| wtIgG1 | | AGGGVTFDTRGQGTQVTVSSEPKSSDKTHTCPPCPAPELLGGPSVFLFPPKPK |
| Fc) heavy | | DTLMISRTPEVTCVVVDVSHEDPEVKFNWYVDGVEVHNAKTKPREEQYNS |
| chain AA | | TYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGKGGGGS |
| | | QVQLVQSGAEVKKPGASVKVSCKASGYIFTSYYLHWVRQAPGQGLEWIGYI |
| | | NPQTGKTSYAQKFQGRVTMTRDTSTSTVYMELSSLRSEDTAVYYCAREYGN |
| | | YNYPLDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCD |
| BE-375 | SEQ ID | GAGGTCCAGTTACTTGAGAGTGGTGGAGGTCTGGTCCAACCAGGAGGTT |
| (E- | NO: 222 | CGCTGCGTTTATCCTGCGCCGCGTCTGGATTCACGGTTTCCGCCGAAGAC |
| wtIgG1 | | GTGGGTTGGGTGCGTCAAGCGCCGGGGAAAGGACTGGAATGGGTCTCCG |
| Fc) heavy | | CCATCTTGGATAAGGGTGGTTCGACATACTATGCGGAAAGTGTCAAAGGG |
| chain | | CGCTTTACGATCTCGCGCGATAACGCAAAAAATACTCTTTACCTTCAAATG |
| DNA | | TCTAGCCTTCGTGCTGAGGACACTGCGGTGTACTACTGCGCCCGCATTGT |
| | | CTACCATGCTGGTGGTGGCGTCACCTTTGATACTCGGGGACAGGGCACCC |
| | | AAGTTACAGTCTCATCGGAGCCCAAATCTTCTGACAAAACTCACACATGC |
| | | CCACCGTGCCCAGCACCTGAACTCCTGGGGGGACCGTCAGTCTTCCTCTT |
| | | CCCCCCAAAACCCAAGGACACCCTCATGATCTCCCGGACCCCTGAGGTCA |
| | | CATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGG |
| | | GAGGAGCAGTACAACAGCACGTACCGTGTGGTCAGCGTCCTCACCGTCC |
| | | TGCACCAGGACTGGCTGAATGGCAAGGAGTACAAGTGCAAGGTCTCCAA |
| | | CAAAGCCCTCCCAGCCCCCATCGAGAAAACCATCTCCAAAGCCAAAGGG |
| | | CAGCCCCGAGAACCACAGGTGTACACCCTGCCCCCATCCCGGGATGAGCT |
| | | GACCAAGAACCAGGTCAGCCTGACCTGCCTGGTCAAAGGCTTCTATCCCA |
| | | GCGACATCGCCGTGGAGTGGGAGAGCAATGGGCAGCCGGAGAACAACTA |
| | | CAAGACCACGCCTCCCGTGCTGGACTCCGACGGCTCCTTCTTCCTCTACA |
| | | GCAAGCTCACCGTGGACAAGAGCAGGTGGCAGCAGGGGAACGTCTTCTC |
| | | ATGCTCCGTGATGCATGAGGCTCTGCACAACCACTACACGCAGAAGAGCC |
| | | TCTCCCTGTCTCCGGGTAAAGGTGGAGGCGGTTCACAGGTGCAGCTGGT |
| | | GCAGAGCGGCGCGGAAGTGAAAAAACCGGGCGCGAGCGTGAAAGTGAG |
| | | CTGCAAAGCGAGCGGCTATATTTTTACCAGCTATTACCTGCATTGGGTGCG |
| | | CCAGGCGCCGGGCCAGGGCCTGGAATGGATTGGCTATATTAACCCGCAGA |
| | | CCGGCAAGACCAGCTATGCCCAGAAATTTCAGGGCCGCGTGACCATGACC |
| | | CGCGATACCAGCACCAGCACCGTGTATATGGAACTGAGCAGCCTGCGCAG |
| | | CGAAGATACCGCGGTGTATTATTGCGCGCGCGAATATGGCAACTATAACTA |
| | | TCCGCTGGATTATTGGGGCCAGGGCACCCTGGTGACCGTGAGCAGCGCTA |
| | | GCACCAAGGGGCCCTCGGTCTTCCCCCTGGCACCCTCCTCCAAGAGTACT |
| | | TCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCA |
| | | CACCTTCCCGGCTGTCCTACAGTCCTCAGGACTCTACTCCCTCAGCAGCG |
| | | TGGTGACCGTGCCCTCCAGCAGCTTGGGCACCCAGACCTACATCTGCAAC |
| | | GTGAATCACAAGCCCAGCAACACCAAGGTGGACAAGAAAGTTGAGCCCA |
| | | AATCTTGTGAC |
| BE-244 | SEQ ID | QVQLVQSGAEVKKPGASVKVSCKASGYIFTSYYLHWVRQAPGQGLEWIGYI |
| (A- | NO: 223 | NPQTGKTSYAQKFQGRVTMTRDTSTSTVYMELSSLRSEDTAVYYCAREYGN |
| (G4S)3) | | YNYPLDYWGQGTLVTVSSASTKGPSVFPLAPSSKSTSGGTAALGCLVKDYFP |
| heavy | | EPVTVSWNSGALTSGVHTFPAVLQSSGLYSLSSVVTVPSSSLGTQTYICNVNH |
| chain AA | | KPSNTKVDKKVEPKSCDKTHTCPPCPAPPAAGPSVFLFPPKPKDTLMISRTPE |
| | | VTCVVVDVSHEDPEVKFNWYVDGVEVHNAKTKPREEQYNSTYRVVSVLTV |
| | | LHQDWLNGKEYKCKVSNKALAAPIEKTISKAKGQPREPQVYTLPPSREEMT |
| | | KNQVSLTCLVKGFYPSDIAVEWESNGQPENNYKTTPPVLDSDGSFFLYSKLT |
| | | VDKSRWQQGNVFSCSVMHEALHNHYTQKSLSLSPGKGGGGSGGGGSGGG |
| | | GSEVQLLESGGGLVQPGGSLRLSCAASGFTVSAEDVGWVRQAPGKGLEWV |
| | | SAILDKGGSTYYAESVKGRFTISRDNAKNTLYLQMSSLRAEDTAVYYCARIV |
| | | YHAGGGVTFDTRGQGTQVTVSS |
| BE-244 | SEQ ID | CAGGTGCAGCTGGTGCAGAGCGGCGCGGAAGTGAAAAAACCGGGCGCG |
| (A- | NO: 224 | AGCGTGAAAGTGAGCTGCAAAGCGAGCGGCTATATTTTTACCAGCTATTA |
| (G4S)3) | | CCTGCATTGGGTGCGCCAGGCGCCGGGCCAGGGCCTGGAATGGATTGGCT |
| heavy | | ATATTAACCCGCAGACCGGCAAGACCAGCTATGCCCAGAAATTTCAGGGC |
| chain | | CGCGTGACCATGACCCGCGATACCAGCACCAGCACCGTGTATATGGAACT |
| DNA | | GAGCAGCCTGCGCAGCGAAGATACCGCGGTGTATTATTGCGCGCGCGAAT |
| | | ATGGCAACTATAACTATCCGCTGGATTATTGGGGCCAGGGCACCCTGGTGA |
| | | CCGTGAGCAGCGCTAGCACCAAGGGGCCCTCGGTCTTCCCCCTGGCACC |
| | | CTCCTCCAAGAGTACTTCTGGGGGCACAGCGGCCCTGGGCTGCCTGGTCA |
| | | AGGACTACTTCCCCGAACCGGTGACGGTGTCGTGGAACTCAGGCGCCCT |
| | | GACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCA |
| | | GACCTACATCTGCAACGTGAATCACAAGCCCAGCAACACCAAGGTGGAC |
| | | AAGAAAGTTGAGCCAAAGTCCTGTGACAAGACCCACACATGCCCCCCTT |
| | | GTCCTGCTCCACCAGCTGCAGGACCAAGCGTGTTCCTGTTTCCACCCAAG |
| | | CCCAAGGATACCCTGATGATCTCTCGGACCCCAGAGGTGACATGCGTGGT |
| | | GGTGGATGTGAGCCACGAGGACCCCGAGGTGAAGTTCAACTGGTATGTG |
| | | GACGGCGTGGAGGTGCACAATGCTAAGACCAAGCCCAGGGAGGAGCAG |
| | | TACAACTCCACCTATAGAGTGGTGTCTGTGCTGACAGTGCTGCACCAGGA |
| | | TTGGCTGAACGGCAAGGAGTATAAGTGCAAGGTGTCCAATAAGGCCCTG |
| | | GCCGCTCCTATCGAGAAGACCATCTCTAAGGCCAAGGGCCAGCCCAGAG |
| | | AGCCTCAGGTGTACACACTGCCTCCATCCCGGGAAGAGATGACCAAGAA |
| | | CCAGGTGTCTCTGACATGTCTGGTCAAGGGCTTCTATCCCTCTGACATCGC |
| | | CGTGGAGTGGGAGAGCAATGGCCAGCCTGAGAACAATTACAAGACCACA |
| | | CCCCCTGTGCTGGATTCCGACGGCTCTTTCTTTCTGTATAGCAAGCTGACC |
| | | GTGGACAAGTCCCGGTGGCAGCAGGGCAACGTGTTCAGCTGTTCCGTGA |
| | | TGCACGAAGCTCTGCATAATCACTATACTCAGAAATCCCTGTCACTGTCAC |
| | | CTGGTAAAGGTGGAGGCGGTTCAGGCGGCGGAGGATCTGGTGGCGGTGG |
| | | GTCCGAGGTCCAGTTACTTGAGAGTGGTGGAGGTCTGGTCCAACCAGGA |
| | | GGTTCGCTGCGTTTATCCTGCGCCGCGTCTGGATTCACGGTTTCCGCCGAA |
| | | GACGTGGGTTGGGTGCGTCAAGCGCCGGGGAAAGGACTGGAATGGGTCT |
| | | CCGCCATCTTGGATAAGGGTGGTTCGACATACTATGCGGAAAGTGTCAAA |
| | | GGGCGCTTTACGATCTCGCGCGATAACGCAAAAAATACTCTTTACCTTCAA |
| | | ATGTCTAGCCTTCGTGCTGAGGACACTGCGGTGTACTACTGCGCCCGCATT |
| | | GTCTACCATGCTGGTGGTGGCGTCACCTTTGATACTCGGGGACAGGGCAC |
| | | CCAAGTTACAGTCTCATCG |

### Example 19. In Vivo Efficacy of Single-Agent CD137×CEA Multispecific Antibody

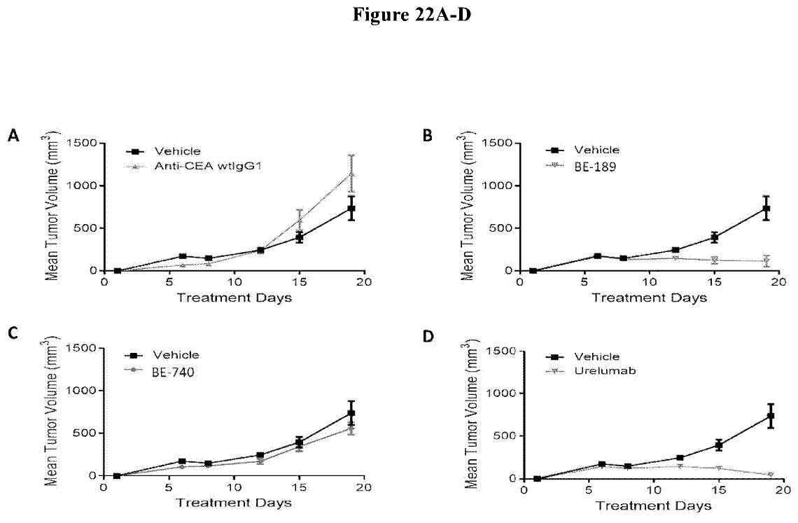

To determine the in vivo efficacy of CD137×CEA multispecific antibodies BE-189 and BE-740 against CEA⁺ tumor cells, CT26/CEA cells (1×10⁶) were injected subcutaneously into humanized CD137 mice of the BALB/c background. BE-189 (3 mg/kg), BE-740 (3 mg/kg), anti-CEA Ab (SEQ ID NO: 87 and 89) (3 mg/kg), Urelumab analog (3 mg/kg) or vehicle control were given twice per week starting on the day of tumor injection (6 mice per group). As compared to vehicle control, BE-189(A-BGA-5623) and Urelumab analog induced significant inhibition of tumor growth (P<0.001) as shown in FIG. 22A-D.

### Example 20. Design of Tumor-Targeted CD137 Agonists

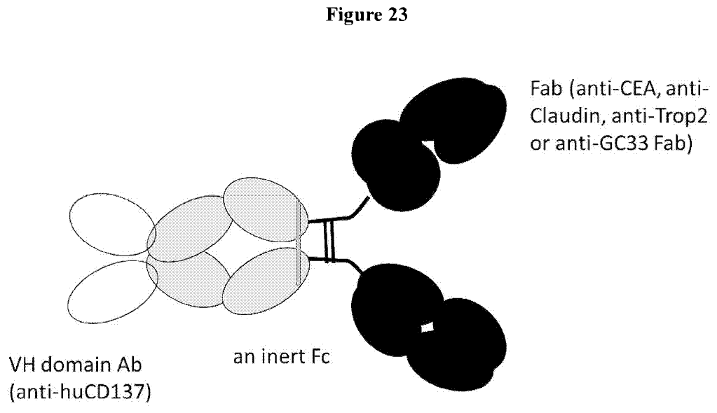

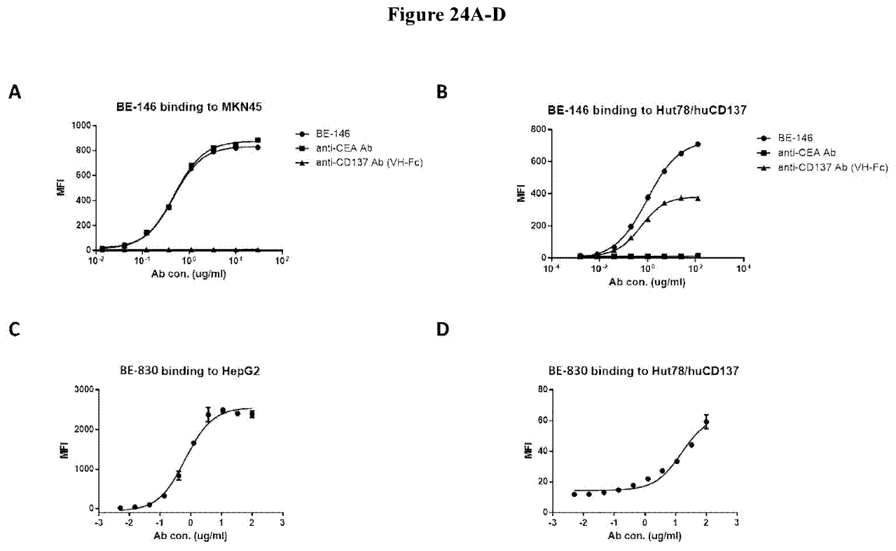

Agonistic anti-huCD137 antibodies have demonstrated toxicity in the clinical setting, which can indicate that systemic FcγR cross-linking is not ideal for CD137 activation. The aim was to achieve potent CD137 stimulation specifically at the tumor site without systemic CD137 activation for a broad range of cancers. To overcome the dependency of FcγR cross-linking, we generated TAA×CD137 multispecific antibodies with the following features as shown in FIG. 23. This specific construct included an IgG-fusion like multispecific antibody format with a module ratio of 2:2, a bivalent F(ab′)₂ fragment that binds to a TAA, for example, CEA, GPC3, Claudin6 or Trop2, VH domain fragments with a fusion at the C terminal of CH3, which bind huCD137, and a Fc null version of huIgG1, which has no FcγR binding but retain FcRn binding. The yields and biochemical properties of generated tumor-targeted CD137 agonists were summarized in Table 23 and the sequence information is shown in Table 24. A variant of the anti-CEA antibody with improved biophysical properties and a better yield was selected to construct CD137×CEA. FIG. 24A-D shows representative cell binding results of CD137×CEA (FIG. 24A-B) and Glypican 3 (GPC3)×CD137 (FIG. 24C-D).

TABLE 23
Summary of yields and biochemical properties

| | | Monomer | TAA KD | huCD137 |
|---|---|---|---|---|
| Construct | Code | (%) | (M) | (M) |
| CEA × CD137 | BE-146 | 99.09 | 6.50E−09 | 1.67E−07 |
| Claudin6 × CD137 | BE-268 | 99.76 | * | * |
| Trop2 × CD137 | BE-907 | 98.5 | * | * |
| GPC3 × CD137 | BE-830 | 99.03 | 1.47E−09 | 1.82E−07 |

*Not resolved

TABLE 24
Amino acid and DNA sequences of TAA x CD137

| Construct | SEQ ID NO | Sequence |
|---|---|---|
| BE-146 | SEQ ID NO: | QVQLVQSGAEVKKPGASVKVSCKASGYIFTSYYLHWVRQAPGQGLE |
| heavy chain | 225 | WIGYINPQTGKTSYAQKFQGRVTMTRDTSTSTVYMELSSLRSEDTAV |
| AA | | YYCAREYGNYNYPLDYWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAP |
| | | PAAGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSN |
| | | KALAAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQG |
| | | NVFSCSVMHEALHNHYTQKSLSLSPGKGGGGSEVQLLESGGGLVQPG |
| | | GSLRLSCAASGFTVSAEDVGWVRQAPGKGLEWVSAILDKGGSTYYA |
| | | ESVKGRFTISRDNAKNTLYLQMSSLRAEDTAVYYCARIVYHAGGGVT |
| | | FDTRGQGTQVTVSS |
| BE-146 | SEQ ID NO: | CAGGTGCAGCTGGTGCAGAGCGGCGCGGAAGTGAAAAAACCGGG |
| heavy chain | 226 | CGCGAGCGTGAAAGTGAGCTGCAAAGCGAGCGGCTATATTTTTAC |
| DNA | | CAGCTATTACCTGCATTGGGTGCGCCAGGCGCCGGGCCAGGGCCT |
| | | GGAATGGATTGGCTATATTAACCCGCAGACCGGCAAGACCAGCTA |
| | | TGCCCAGAAATTTCAGGGCCGCGTGACCATGACCCGCGATACCAG |
| | | CACCAGCACCGTGTATATGGAACTGAGCAGCCTGCGCAGCGAAGA |
| | | TACCGCGGTGTATTATTGCGCGCGCGAATATGGCAACTATAACTAT |
| | | CCGCTGGATTATTGGGGCCAGGGCACCCTGGTGACCGTGAGCAGC |
| | | GCTAGCACCAAGGGGCCCTCGGTCTTCCCCCTGGCACCCTCCTCCA |
| | | AGAGTACTTCTGGGGGCACAGCGGCCCTGGGCTGCCTGGTCAAGG |
| | | ACTACTTCCCCGAACCGGTGACGGTGTCGTGGAACTCAGGCGCCC |
| | | TGACCAGCGGCGTGCACACCTTCCCGGCTGTCCTACAGTCCTCAGG |
| | | ACTCTACTCCCTCAGCAGCGTGGTGACCGTGCCCTCCAGCAGCTTG |
| | | GGCACCCAGACCTACATCTGCAACGTGAATCACAAGCCCAGCAAC |
| | | ACCAAGGTGGACAAGAAAGTTGAGCCAAAGTCCTGTGACAAGACC |
| | | CACACATGCCCCCCTTGTCCTGCTCCACCAGCTGCAGGACCAAGCG |
| | | TGTTCCTGTTTCCACCCAAGCCCAAGGATACCCTGATGATCTCTCG |
| | | GACCCCAGAGGTGACATGCGTGGTGGTGGATGTGAGCCACGAGGA |
| | | CCCCGAGGTGAAGTTCAACTGGTATGTGGACGGCGTGGAGGTGCA |
| | | CAATGCTAAGACCAAGCCCAGGGAGGAGCAGTACAACTCCACCTA |
| | | TAGAGTGGTGTCTGTGCTGACAGTGCTGCACCAGGATTGGCTGAA |
| | | CGGCAAGGAGTATAAGTGCAAGGTGTCCAATAAGGCCCTGGCCGC |
| | | TCCTATCGAGAAGACCATCTCTAAGGCCAAGGGCCAGCCCAGAGA |
| | | GCCTCAGGTGTACACACTGCCTCCATCCCGGGAAGAGATGACCAA |
| | | GAACCAGGTGTCTCTGACATGTCTGGTCAAGGGCTTCTATCCCTCT |
| | | GACATCGCCGTGGAGTGGGAGAGCAATGGCCAGCCTGAGAACAAT |
| | | TACAAGACCACACCCCCTGTGCTGGATTCCGACGGCTCTTTCTTTC |
| | | TGTATAGCAAGCTGACCGTGGACAAGTCCCGGTGGCAGCAGGGCA |
| | | ACGTGTTCAGCTGTTCCGTGATGCACGAAGCTCTGCATAATCACTA |
| | | TACTCAGAAATCCCTGTCACTGTCACCTGGTAAAGGTGGAGGCGG |
| | | TTCAGAGGTCCAGTTACTTGAGAGTGGTGGAGGTCTGGTCCAACC |
| | | AGGAGGTTCGCTGCGTTTATCCTGCGCCGCGTCTGGATTCACGGTT |
| | | TCCGCCGAAGACGTGGGTTGGGTGCGTCAAGCGCCGGGGAAAGGA |
| | | CTGGAATGGGTCTCCGCCATCTTGGATAAGGGTGGTTCGACATACT |
| | | ATGCGGAAAGTGTCAAAGGGCGCTTTACGATCTCGCGCGATAACG |
| | | CAAAAAATACTCTTTACCTTCAAATGTCTAGCCTTCGTGCTGAGGA |
| | | CACTGCGGTGTACTACTGCGCCCGCATTGTCTACCATGCTGGTGGT |
| | | GGCGTCACCTTTGATACTCGGGGACAGGGCACCCAAGTTACAGTC |
| | | TCATCG |
| BE-146 | SEQ ID NO: | DIQMTQSPSSLSASVGDRVTITCRASENQYGYLAWYQQKPGKVPKLL |
| light chain | 89 | IYNYKNLVEGVPSRFSGSGSGTDFTLTISSLQPEDVATYYCQHHLGTP |
| AA | | YTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKH |
| | | KVYACEVTHQGLSSPVTKSFNRGEC |
| BE-146 light | SEQ ID NO: | GATATTCAGATGACCCAGAGCCCGAGCAGCCTGAGCGCGAGCGTG |
| chain DNA | 90 | GGCGATCGCGTGACCATTACCTGCCGCGCGAGCGAAAACCAGTAT |
| | | GGCTATCTGGCGTGGTATCAGCAGAAACCGGGCAAAGTGCCGAAA |
| | | CTGCTGATTTATAACTATAAAAACCTGGTGGAAGGCGTGCCGAGC |
| | | CGCTTTAGCGGCAGCGGCAGCGGCACCGATTTTACCCTGACCATTA |
| | | GCAGCCTGCAGCCGGAAGATGTGGCGACCTATTATTGCCAGCATC |
| | | ATCTGGGCACCCCGTATACCTTTGGCCAGGGCACCAAAGTGGAAA |
| | | TTAAACGAACAGTGGCAGCCCCTTCCGTCTTCATTTTTCCCCCTTCT |
| | | GACGAACAGCTGAAATCAGGAACTGCTAGCGTGGTCTGTCTGCTG |
| | | AACAATTTCTACCCCAGAGAGGCCAAGGTGCAGTGGAAAGTCGAT |
| | | AACGCTCTGCAGTCCGGCAATTCTCAGGAGAGTGTGACCGAACAG |
| | | GACTCAAAGGATAGCACATATTCCCTGTCTAGTACTCTGACCCTGA |
| | | GCAAAGCAGACTACGAGAAGCACAAAGTGTATGCCTGTGAAGTCA |
| | | CACACCAGGGGCTGAGTTCACCAGTCACCAAGAGTTTCAACAGAG |
| | | GGGAATGC |
| BE-268 | SEQ ID NO: | EVQLQQSGPELVKPGASMKISCKASGYSFTGYTMNWVKQSHGKNLE |
| heavy chain | 227 | WIGLINPYNGGTSYNQKFKGKATLTIDKSSSTAYMELLSLTSEDSAVYY |
| AA | | CARDYGYVLDYWGQGTTLTVSSASTKGPSVFPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPPAAGPSV |
| | | FLFPPKPKDTLMISRTPEVTCVVVDVSHEDPEVKFNWYVDGVEVHNA |
| | | KTKPREEQYNSTYRVVSVLTVLHQDWLNGKEYKCKVSNKALAAPIE |
| | | KTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVM |
| | | HEALHNHYTQKSLSLSPGKGGGGSEVQLLESGGGLVQPGGSLRLSCA |
| | | ASGFTVSAEDVGWVRQAPGKGLEWVSAILDKGGSTYYAESVKGRFTI |
| | | SRDNAKNTLYLQMSSLRAEDTAVYYCARIVYHAGGGVTFDTRGQGT |
| | | QVTVSS |
| BE-268 | SEQ ID NO: | GAAGTGCAACTTCAACAATCAGGACCTGAACTTGTGAAACCTGGA |
| heavy chain | 228 | GCATCAATGAAGATTTCGTGCAAAGCATCAGGATACTCATTTACAGG |
| DNA | | ATACACAATGAACTGGGTGAAACAATCACACGGAAAGAATTTGGA |
| | | ATGGATCGGACTTATCAACCCTTACAACGGAGGAACATCATACAAC |
| | | CAGAAGTTCAAGGGAAAGGCTACTCTTACAATCGATAAATCATCAT |
| | | CAACAGCATACATGGAACTTCTTTCACTTACATCAGAAGATTCAGC |
| | | AGTGTACTACTGCGCAAGAGATTACGGATACGTGCTTGATTACTGG |
| | | GGGCAGGGGACAACACTTACAGTGTCATCAGCTAGCACCAAGGGG |
| | | CCCTCGGTCTTCCCCCTGGCACCCTCCTCCAAGAGTACTTCTGGGG |
| | | GCACAGCGGCCCTGGGCTGCCTGGTCAAGGACTACTTCCCCGAAC |
| | | CGGTGACGGTGTCGTGGAACTCAGGCGCCCTGACCAGCGGCGTGC |
| | | ACACCTTCCCGGCTGTCCTACAGTCCTCAGGACTCTACTCCCTCAG |
| | | CAGCGTGGTGACCGTGCCCTCCAGCAGCTTGGGCACCCAGACCTA |
| | | CATCTGCAACGTGAATCACAAGCCCAGCAACACCAAGGTGGACAA |
| | | GAAAGTTGAGCCAAAGTCCTGTGACAAGACCCACACATGCCCCCC |
| | | TTGTCCTGCTCCACCAGCTGCAGGACCAAGCGTGTTCCTGTTTCCA |
| | | CCCAAGCCCAAGGATACCCTGATGATCTCTCGGACCCCAGAGGTGA |
| | | CATGCGTGGTGGTGGATGTGAGCCACGAGGACCCCGAGGTGAAGT |
| | | TCAACTGGTATGTGGACGGCGTGGAGGTGCACAATGCTAAGACCA |
| | | AGCCCAGGGAGGAGCAGTACAACTCCACCTATAGAGTGGTGTCTGT |
| | | GCTGACAGTGCTGCACCAGGATTGGCTGAACGGCAAGGAGTATAA |
| | | GTGCAAGGTGTCCAATAAGGCCCTGGCCGCTCCTATCGAGAAGACC |
| | | ATCTCTAAGGCCAAGGGCCAGCCCAGAGAGCCTCAGGTGTACACA |
| | | CTGCCTCCATCCCGGGAAGAGATGACCAAGAACCAGGTGTCTCTG |
| | | ACATGTCTGGTCAAGGGCTTCTATCCCTCTGACATCGCCGTGGAGT |
| | | GGGAGAGCAATGGCCAGCCTGAGAACAATTACAAGACCACACCCC |
| | | CTGTGCTGGATTCCGACGGCTCTTTCTTTCTGTATAGCAAGCTGACC |
| | | GTGGACAAGTCCCGGTGGCAGCAGGGCAACGTGTTCAGCTGTTCC |
| | | GTGATGCACGAAGCTCTGCATAATCACTATACTCAGAAATCCCTGTC |
| | | ACTGTCACCTGGTAAAGGTGGAGGCGGTTCAGAGGTCCAGTTACTT |
| | | GAGAGTGGTGGAGGTCTGGTCCAACCAGGAGGTTCGCTGCGTTTAT |
| | | CCTGCGCCGCGTCTGGATTCACGGTTTCCGCCGAAGACGTGGGTTG |
| | | GGTGCGTCAAGCGCCGGGGAAAGGACTGGAATGGGTCTCCGCCAT |
| | | CTTGGATAAGGGTGGTTCGACATACTATGCGGAAAGTGTCAAAGGG |
| | | CGCTTTACGATCTCGCGCGATAACGCAAAAAATACTCTTTACCTTCA |
| | | AATGTCTAGCCTTCGTGCTGAGGACACTGCGGTGTACTACTGCGCC |
| | | CGCATTGTCTACCATGCTGGTGGTGGCGTCACCTTTGATACTCGGGG |
| | | ACAGGGCACCCAAGTTACAGTCTCATCG |
| BE-268 | SEQ ID NO: | QIVLTQSPAIMSASPGEKVTITCSASSSVSYLHWFQQKPGTSPKLWVYS |
| light chain | 229 | TSNLPSGVPARFGGSGSGTSYSLTISRMEAEDAATYYCQQRSIYPPWTF |
| AA | | GGGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| BE-268 | SEQ ID NO: | CAAATCGTGCTTACACAATCACCTGCAATCATGTCAGCATCACCTGG |
| light chain | 230 | AGAGAAGGTTACTATCACATGCTCAGCATCATCATCAGTGTCATACC |
| DNA | | TTCACTGGTTTCAACAGAAGCCCGGGACATCACCTAAACTTTGGGT |
| | | GTACTCAACATCAAACCTTCCTTCAGGAGTGCCTGCAAGATTTGGA |
| | | GGATCAGGATCAGGAACATCATACTCACTTACAATCTCAAGAATGG |
| | | AAGCAGAAGATGCAGCAACATACTACTGCCAACAAAGATCAATCTA |
| | | CCCTCCTTGGACATTTGGAGGAGGAACAAAGTTGGAGATCAAACG |
| | | AACAGTGGCAGCCCCTTCCGTCTTCATTTTTCCCCCTTCTGACGAAC |
| | | AGCTGAAATCAGGAACTGCTAGCGTGGTCTGTCTGCTGAACAATTT |
| | | CTACCCCAGAGAGGCCAAGGTGCAGTGGAAAGTCGATAACGCTCT |
| | | GCAGTCCGGCAATTCTCAGGAGAGTGTGACCGAACAGGACTCAAA |
| | | GGATAGCACATATTCCCTGTCTAGTACTCTGACCCTGAGCAAAGCA |
| | | GACTACGAGAAGCACAAAGTGTATGCCTGTGAAGTCACACACCAG |
| | | GGGCTGAGTTCACCAGTCACCAAGAGTTTCAACAGAGGGGAATGC |
| BE-907 | SEQ ID NO: | QVQLQQSGSELKKPGASVKVSCKASGYTFTNYGMNWVKQAPGQGL |
| heavy chain | 231 | KWMGWINTYTGEPTYTDDFKGRFAFSLDTSVSTAYLQISSLKADDTA |
| AA | | VYFCARGGFGSSYWYFDVWGQGSLVTVSSASTKGPSVFPLAPSSKST |
| | | SGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPCP |
| | | APPAAGPSVFLFPPKPKDTLMISRTPEVTCVVVDVSHEDPEVKFNWY |
| | | VDGVEVHNAKTKPREEQYNSTYRVVSVLTVLHQDWLNGKEYKCKV |
| | | SNKALAAPIEKTISKAKGQPREPQVYTLPPSREEMTKNQVSLWCLVK |
| | | GFYPSDIAVEWESNGQPENNYKTTPPVLDSDGSFFLYSKLTVDKSRW |
| | | QQGNVFSCSVMHEALHNHYTQKSLSLSPGKGGGGSEVQLLESGGGL |
| | | VQPGGSLRLSCAASGFTVSAEDVGWVRQAPGKGLEWVSAILDKGGS |
| | | TYYAESVKGRFTISRDNAKNTLYLQMSSLRAEDTAVYYCARIVYHAG |
| | | GGVTFDTRGQGTQVTVSS |
| BE-907 | SEQ ID NO: | CAAGTGCAACTGCAACAATCTGGCTCTGAGCTGAAGAAGCCTGGG |
| heavy chain | 232 | GCCTCTGTGAAGGTGAGCTGCAAGGCCTCTGGCTACACCTTCACCA |
| DNA | | ACTATGGCATGAACTGGGTGAAGCAAGCCCCTGGCCAAGGCCTGA |
| | | AGTGGATGGGCTGGATCAACACCTACACTGGGGAGCCCACCTACA |
| | | CAGATGACTTCAAAGGTAGATTTGCCTTCAGCCTGGACACCTCTGT |
| | | GAGCACAGCCTACCTGCAGATCAGCAGCCTGAAGGCTGATGACAC |
| | | AGCTGTGTATTTCTGTGCTAGAGGGGGCTTTGGCAGCAGCTACTGG |
| | | TACTTTGATGTGTGGGGCCAAGGCAGCCTGGTGACTGTGAGCTCTG |
| | | CTAGCACCAAAGGCCCTTCTGTGTTCCCCCTGGCCCCCTCATCCAA |
| | | GAGCACCTCTGGGGGCACAGCTGCCTTAGGCTGTCTGGTGAAGGA |
| | | CTATTTCCCTGAGCCTGTGACAGTGAGCTGGAATTCTGGGGCCCTG |
| | | ACCTCTGGGGTGCATACCTTCCCTGCTGTGCTGCAGAGCTCTGGCC |
| | | TGTACAGCCTGAGCTCTGTGGTGACAGTGCCTAGCAGCAGCCTGG |
| | | GCACACAGACCTACATCTGCAATGTGAACCACAAGCCTAGCAACA |
| | | CCAAGGTGGACAAGAAGGTGGAGCCCAAGAGCTGTGACAAAACC |
| | | CACACCTGTCCCCCCTGCCCTGCCCCCCCTGCTGCTGGCCCCTCTG |
| | | TGTTCCTTTTCCCCCCCAAGCCCAAGGACACCCTGATGATCAGCAG |
| | | AACCCCTGAGGTGACCTGTGTGGTGGTGGATGTGAGCCATGAGGA |
| | | CCCTGAGGTGAAGTTCAACTGGTATGTGGATGGGGTGGAGGTGCA |
| | | CAATGCCAAGACCAAGCCTAGAGAGGAGCAGTACAACAGCACCT |
| | | ACAGAGTGGTGTCTGTGCTGACAGTGCTGCACCAAGACTGGCTGA |
| | | ATGGCAAGGAGTACAAGTGCAAGGTGAGCAACAAGGCCCTGGCT |
| | | GCCCCCATTGAGAAGACCATCAGCAAGGCCAAGGGGCAGCCTAGA |
| | | GAGCCCCAAGTGTACACCCTGCCCCCTAGCAGAGAGGAGATGACC |
| | | AAGAACCAAGTGAGCCTGTGGTGCCTGGTGAAAGGCTTCTACCCC |
| | | TCTGACATTGCTGTGGAGTGGGAGAGCAATGGGCAGCCTGAGAAC |
| | | AACTACAAGACCACCCCCCCTGTGCTGGACTCTGATGGCAGCTTCT |
| | | TCCTGTACAGCAAGCTGACAGTGGACAAGAGCAGATGGCAGCAAG |
| | | GCAATGTGTTCAGCTGCTCTGTGATGCATGAGGCCCTGCACAACCA |
| | | CTACACACAGAAGAGCCTGAGCCTGTCCCCTGGTAAGGGGGGTGG |
| | | GGGCTCTGAGGTGCAGCTGTTGGAGTCTGGGGGGGGCCTGGTGCA |
| | | GCCTGGGGGCAGTCTAAGACTGAGCTGTGCTGCCTCTGGCTTTACA |
| | | GTGTCTGCTGAGGATGTGGGCTGGGTAAGACAAGCCCCTGGCAAA |
| | | GGACTGGAGTGGGTGTCTGCCATCCTGGACAAGGGGGGCAGCACC |
| | | TACTATGCTGAGTCTGTGAAGGGCAGATTTACCATTAGCAGAGAT |
| | | AATGCCAAGAATACCCTGTACCTGCAGATGAGCAGCCTGAGAGCT |
| | | GAGGACACAGCTGTGTATTACTGTGCTAGAATTGTGTACCATGCTG |
| | | GGGGGGGGGTGACTTTTGACACAAGAGGCCAAGGCACCCAAGTG |
| | | ACAGTGAGCAGC |
| BE-907 | SEQ ID NO: | DIQLTQSPSSLSASVGDRVSITCKASQDVSIAVAWYQQKPGKAPKLLI |
| light chain | 233 | YSASYRYTGVPDRFSGSGSGTDFTLTISSLQPEDFAVYYCQQHYITPLT |
| AA | | FGAGTKVEIKRTVAAPSVFIFPPSDEQLKSGTASVVCLLNNFYPREAK |
| | | VQWKVDNALQSGNSQESVTEQDSKDSTYSLSSTLTLSKADYEKHKV |
| | | YACEVTHQGLSSPVTKSFNRGEC |
| BE-907 | SEQ ID NO: | GATATCCAACTTACACAATCACCTTCATCACTTTCGGCCTCGGTAG |
| light chain | 234 | GCGATAGGGTGTCAATCACATGCAAAGCATCACAAGATGTGTCAA |
| DNA | | TCGCAGTGGCATGGTACCAACAGAAGCCCGGTAAGGCTCCCAAAC |
| | | TTCTTATCTACTCAGCATCATACAGATACACAGGAGTGCCTGATAG |
| | | ATTCAGTGGCTCAGGATCAGGAACAGATTTCACTTTGACAATCTCA |
| | | TCACTTCAACCTGAAGATTTCGCTGTCTACTACTGCCAACAACACT |
| | | ACATCACACCTCTTACATTTGGAGCAGGAACAAAGGTAGAGATCA |
| | | AGCGAACCGTGGCAGCACCTTCAGTGTTTATCTTTCCTCCTTCAGA |
| | | TGAACAACTTAAATCAGGAACTGCGTCGGTAGTTTGTCTTCTTAAC |
| | | AACTTCTATCCCAGAGAAGCAAAGGTCCAGTGGAAAGTGGATAAC |
| | | GCACTTCAATCAGGAAACTCACAAGAATCAGTGACAGAACAAGAT |
| | | TCAAAGGACTCCACATACTCACTTTCATCAACACTTACACTTTCAA |
| | | AGGCCGACTACGAGAAGCATAAGGTGTACGCATGCGAAGTGACAC |
| | | ACCAAGGACTTTCATCACCTGTGACAAAGTCGTTCAACAGAGGAG |
| | | AATGC |
| BE-830 | SEQ ID NO: | QVQLVQSGAEVKKPGASVKVSCKASGYTFTDYEMHWVRQAPGQGL |
| heavy chain | 235 | EWMGALDPKTGDTAYSQKFKGRVTLTADKSTSTAYMELSSLTSEDTAV |
| AA | | YYCTRFYSYTYWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHE |
| | | ALHNHYTQKSLSLSPGKGGGGSEVQLLESGGGLVQPGGSLRLSCAAS |
| | | GFTVSAEDVGWVRQAPGKGLEWVSAILDKGGSTYYAESVKGRFTISR |
| | | DNAKNTLYLQMSSLRAEDTAVYYCARIVYHAGGGVTFDTRGQGTQV |
| | | TVSS |
| BE-830 | SEQ ID NO: | CAGGTGCAGCTGGTGCAGTCTGGAGCAGAGGTGAAGAAGCCAGG |
| heavy chain | 236 | AGCCAGCGTGAAGGTGTCCTGCAAGGCCTCTGGCTATACCTTCACA |
| DNA | | GACTACGAGATGCACTGGGTGCGGCAGGCACCAGGACAGGGACTG |
| | | GAGTGGATGGGCGCCCTGGACCCTAAGACCGGCGATACAGCCTATA |
| | | GCCAGAAGTTTAAGGGCAGAGTGACCCTGACAGCCGACAAGTCCA |
| | | CCTCTACAGCCTACATGGAGCTGAGCTCCCTGACCAGCGAGGATAC |
| | | AGCCGTGTACTATTGTACCCGGTTCTACTCCTATACATACTGGGGCC |
| | | AGGGCACCCTGGTGACAGTGTCTAGCGCTAGCACCAAGGGGCCCT |
| | | CGGTCTTCCCCCTGGCACCCTCCTCCAAGAGTACTTCTGGGGGCAC |
| | | AGCGGCCCTGGGCTGCCTGGTCAAGGACTACTTCCCCGAACCGGT |
| | | GACGGTGTCGTGGAACTCAGGCGCCCTGACCAGCGGCGTGCACAC |
| | | CTTCCCGGCTGTCCTACAGTCCTCAGGACTCTACTCCCTCAGCAGC |
| | | GTGGTGACCGTGCCCTCCAGCAGCTTGGGCACCCAGACCTACATCT |
| | | GCAACGTGAATCACAAGCCCAGCAACACCAAGGTGGACAAGAAA |
| | | GTTGAGCCCAAATCTTGTGACAAAACTCACACATGCCCACCGTGCC |
| | | CAGCACCTGAACTCCTGGGGGGACCGTCAGTCTTCCTCTTCCCCCC |
| | | AAAACCCAAGGACACCCTCATGATCTCCCGGACCCCTGAGGTCAC |
| | | ATGCGTGGTGGTGGACGTGAGCCACGAAGACCCTGAGGTCAAGTT |
| | | CAACTGGTACGTGGACGGCGTGGAGGTGCATAATGCCAAGACAAA |
| | | GCCGCGGGAGGAGCAGTACAACAGCACGTACCGTGTGGTCAGCGT |
| | | CCTCACCGTCCTGCACCAGGACTGGCTGAATGGCAAGGAGTACAA |
| | | GTGCAAGGTCTCCAACAAAGCCCTCCCAGCCCCCATCGAGAAAAC |
| | | CATCTCCAAAGCCAAAGGGCAGCCCCGAGAACCACAGGTGTACAC |
| | | CCTGCCCCCATCCCGGGATGAGCTGACCAAGAACCAGGTCAGCCT |
| | | GACCTGCCTGGTCAAAGGCTTCTATCCCAGCGACATCGCCGTGGAG |
| | | TGGGAGAGCAATGGGCAGCCGGAGAACAACTACAAGACCACGCCT |
| | | CCCGTGCTGGACTCCGACGGCTCCTTCTTCCTCTACAGCAAGCTCA |
| | | CCGTGGACAAGAGCAGGTGGCAGCAGGGGAACGTCTTCTCATGCT |
| | | CCGTGATGCATGAGGCTCTGCACAACCACTACACGCAGAAGAGCCT |
| | | CTCCCTGTCTCCGGGTAAAGGTGGAGGCGGTTCAGAGGTCCAGTTA |
| | | CTTGAGAGTGGTGGAGGTCTGGTCCAACCAGGAGGTTCGCTGCGT |
| | | TTATCCTGCGCCGCGTCTGGATTCACGGTTTCCGCCGAAGACGTGG |
| | | GTTGGGTGCGTCAAGCGCCGGGGAAAGGACTGGAATGGGTCTCCG |
| | | CCATCTTGGATAAGGGTGGTTCGACATACTATGCGGAAAGTGTCAA |
| | | AGGGCGCTTTACGATCTCGCGCGATAACGCAAAAAATACTCTTTAC |
| | | CTTCAAATGTCTAGCCTTCGTGCTGAGGACACTGCGGTGTACTACT |
| | | GCGCCCGCATTGTCTACCATGCTGGTGGTGGCGTCACCTTTGATACT |
| | | CGGGGACAGGGCACCCAAGTTACAGTCTCATCG |
| BE-830 light | SEQ ID NO: | DVVMTQSPLSLPVTPGEPASISCRSSQSLVHSNRNTYLHWYLQKPGQS |
| chain AA | 237 | PQLLIYKVSNRFSGVPDRFSGSGSGTDFTLKISRVEAEDVGVYYCSQN |
| | | THVPPTFGQGTKLEIKRTVAAPSVFIFPPSDEQLKSGTASVVCLLNNFY |
| | | PREAKVQWKVDNALQSGNSQESVTEQDSKDSTYSLSSTLTLSKADYE |
| | | KHKVYACEVTHQGLSSPVTKSFNRGEC |
| BE-830 light | SEQ ID NO: | GACGTGGTCATGACACAGTCCCCTCTGTCTCTGCCAGTGACCCCAG |
| chain DNA | 238 | GAGAGCCTGCCTCTATCAGCTGCAGGAGCTCCCAGAGCCTGGTGCA |
| | | CTCCAACCGCAATACATACCTGCACTGGTATCTGCAGAAGCCAGGC |
| | | CAGTCCCCCCAGCTGCTGATCTACAAGGTGTCTAACCGGTTCAGCG |
| | | GCGTGCCCGACAGATTTTCCGGCTCTGGCAGCGGCACCGATTTCAC |
| | | ACTGAAGATCTCCCGGGTGGAGGCAGAGGATGTGGGCGTGTACTAT |
| | | TGTTCTCAGAATACCCACGTGCCCCCTACATTTGGCCAGGGCACCA |
| | | AGCTGGAGATCAAGCGTACGGTGGCGGCGCCATCTGTCTTCATCTT |
| | | CCCGCCATCTGATGAGCAGTTGAAATCTGGTACCGCTAGCGTTGTG |
| | | TGCCTGCTGAATAACTTCTATCCCAGAGAGGCCAAAGTACAGTGGA |
| | | AGGTGGATAACGCCCTCCAATCGGGTAACTCCCAGGAGAGTGTCAC |
| | | AGAGCAGGACAGCAAGGACAGCACCTACAGCCTCAGCAGCACCCT |
| | | GACGCTGAGCAAAGCAGACTACGAGAAACACAAAGTCTACGCCTG |
| | | CGAAGTCACCCATCAGGGCCTGAGCTCGCCCGTCACAAAGAGCTT |
| | | CAACAGGGGAGAGTGT |

### Example 21. CD137×CEA Induces T Cell Activation in a CEA Dependent Manner

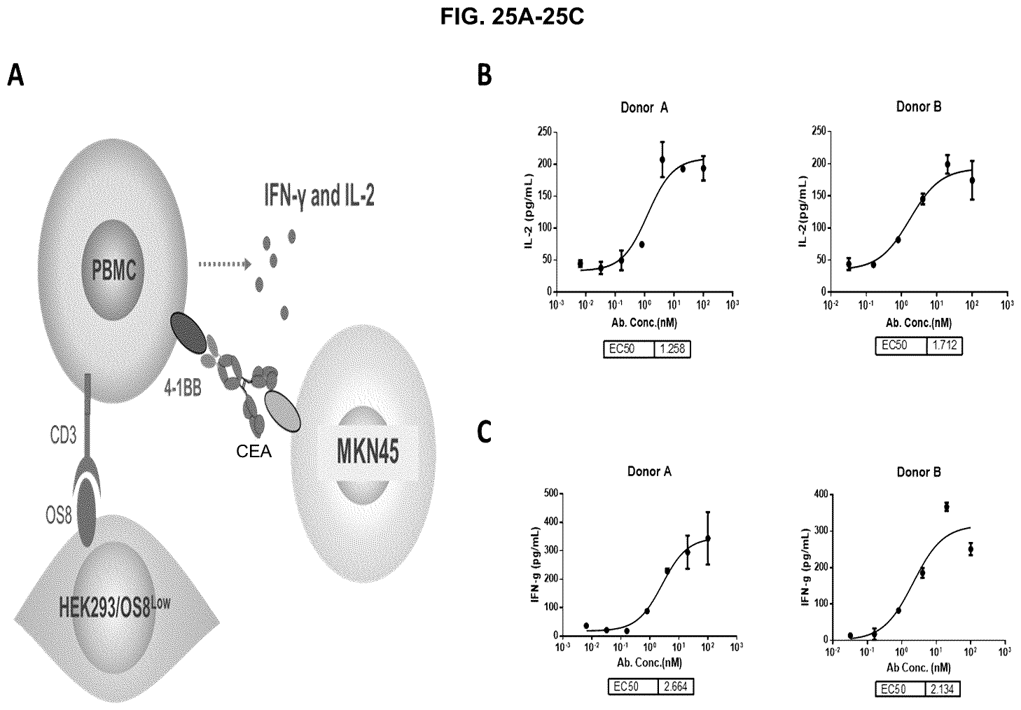

The functionality of CD137×CEA multispecific antibody BE-146 was assessed in different in vitro experiments. We first used human peripheral blood mononuclear cells (PBMCs) from healthy donors to activate human T cells with CD137×CEA and HEK293/OS8 providing the first stimulatory signal. PBMCs were isolated from whole blood of healthy donors by Ficoll (Histopaque-1077, Sigma-St. Louis MO) separation. To determine whether CD137×CEA could induce cytokine release from human PBMCs in the presence of CEA⁺ tumor cells, PBMCs (1×10⁵/well) were co-cultured with CEA⁺ MKN45 cells (2×10⁵/well) and HEK293/OS8 (1×10⁵/well) cell for 2 days in 96-well v-bottom plates. IL-2 and IFN-γ release from PBMCs were determined by ELISA. This is shown schematically in FIG. 25A. The results showed that CD137×CEA could induce significant cytokine release (FIGS. 25B-C). PBMCs from the 2 donors tested. Results are shown in mean±SD of duplicates.

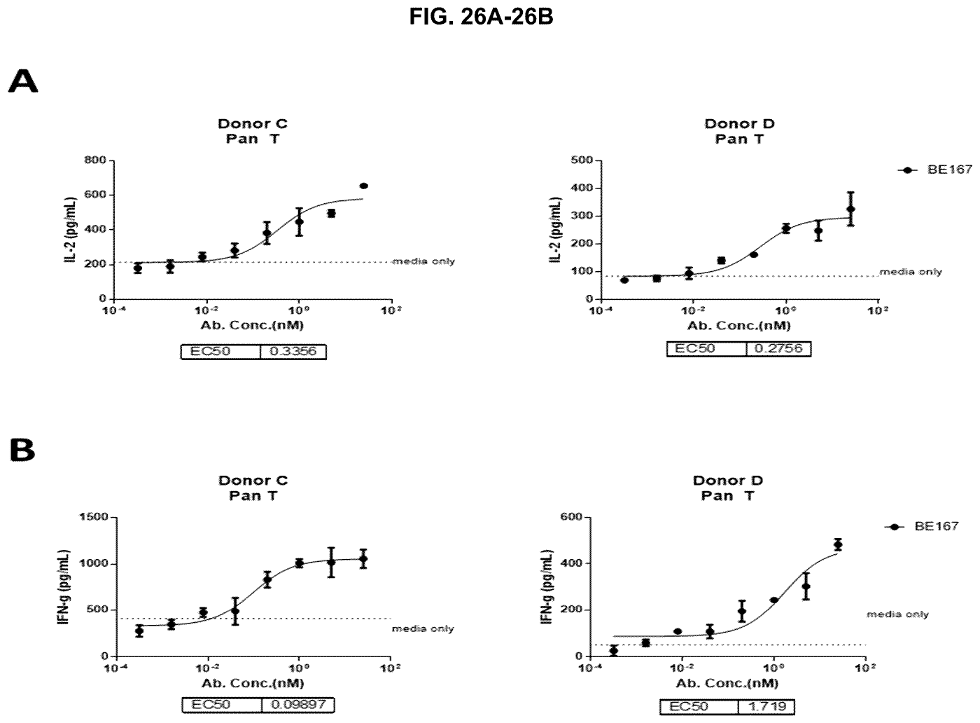

We next investigated whether CD137×CEA can enhance antigen-specific CD8⁺ T cell function. Human peripheral blood mononuclear cells (PBMCs) were isolated from whole blood of healthy donors by Ficoll (Histopaque-1077, Sigma-St. Louis MO) separation. T cells were isolated using the human Pan T cell isolation kit (Miltenyi, Cat. 130-096-535). To determine whether BE-146 could induce cytokine release from human T cells in the presence of CEA⁺ tumor cells, T cells (1×10⁵/well) were co-cultured with CEA+ MKN45 cells (2×10⁵/well) and HEK293/OS8 (1×10⁵/well) cell for 2 days (FIG. 26A) in 96-well v-bottom plates. IL-2 and IFN-γ release from T cells were determined by ELISA. The results showed that the multispecific antibody BE-146 could induce significant IL-2 (FIG. 26A and IFN-γ (FIG. 26B) release.

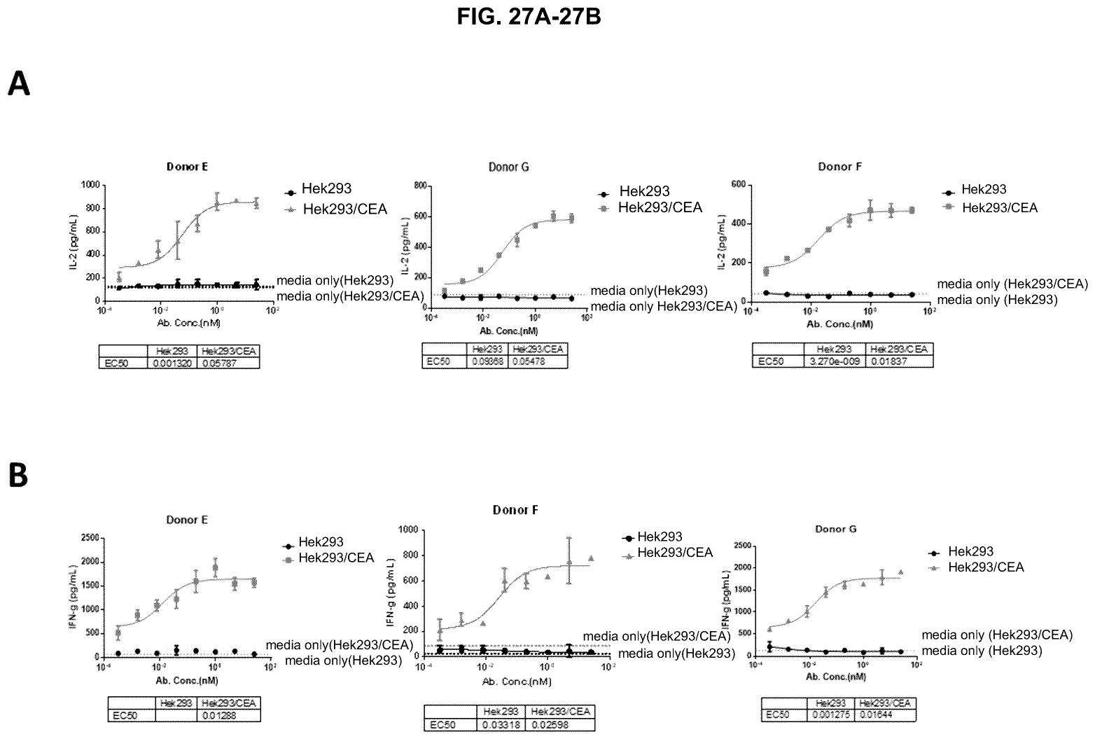

We then investigated whether CD137×CEA can induce a response that was CEA dependent. Human peripheral blood mononuclear cells (PBMCs) were isolated from whole blood of healthy donors by Ficoll (Histopaque-1077, Sigma-St. Louis MO) separation. To determine whether CD137×CEA induced cytokine release from human PBMCs is CEA dependent, PBMCs (1×10⁵/well) were co-cultured with HEK293 or CEA over-expressing HEK293 cells (HEK293/CEA) (1×10⁵/well) as target cells, and HEK293/OS8 (1×10⁵/well) cell for 2 days in 96-well v-bottom plates. IL-2 and IFN-γ release from PBMCs were determined by ELISA. The results showed that multispecific antibody BE-146 could induce significant IL-2 and IFN-γ release from PBMCs against CEA over-expressing HEK293 cells, but not against HEK293 cells without CEA transduction (FIGS. 27A-B).

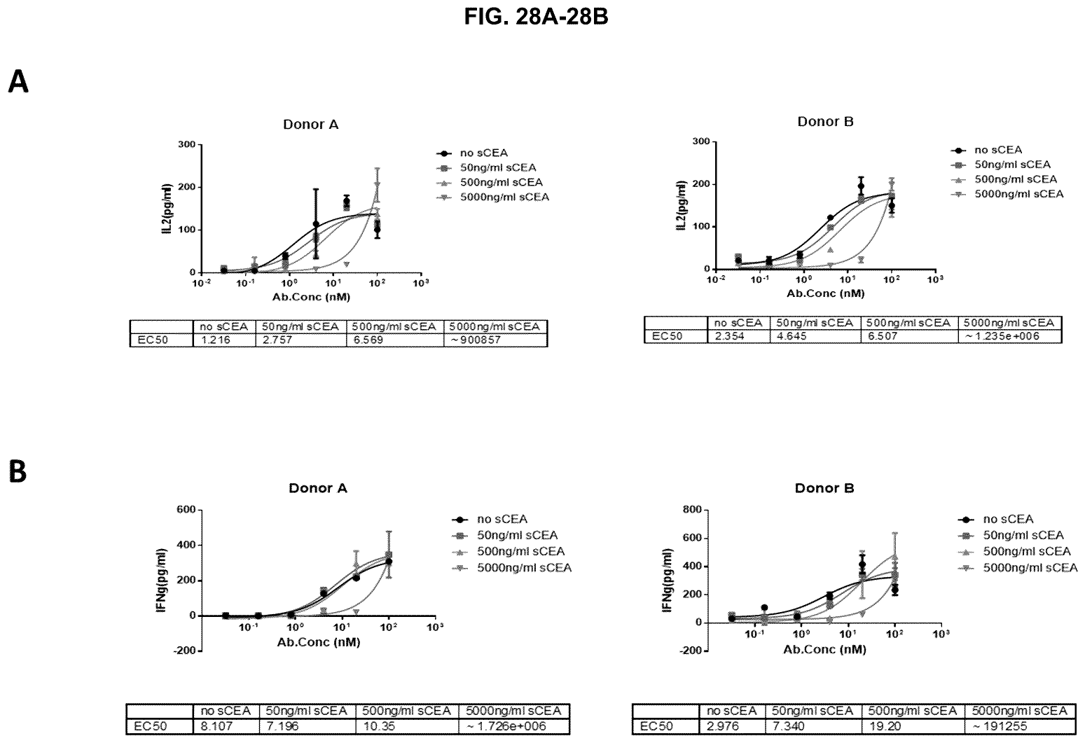

In addition, a series of experiments were conducted to determine whether the induced response from the CD137×CEA construct could be blocked by soluble CEA. Human peripheral blood mononuclear cells (PBMCs) were isolated from whole blood of healthy donors by Ficoll (Histopaque-1077, Sigma, St. Louis MO) separation. To determine whether BE-146 induced cytokine release from human PBMCs could be blocked by soluble CEA, PBMCs (1×10⁵/well) were co-cultured with MKN45 (1×10⁵/well), and HEK293/OS8 (1×10⁵/well) cell for 2 days in 96-well v-bottom plates, in the presence of different concentrations of recombinant soluble CEA. IL-2 and IFN-γ release from PBMCs were determined by ELISA. The results showed that the multispecific antibody BE-146 induced IL-2 (FIG. 28A) and IFN-γ (FIG. 28B) release from PBMCs and this release was not significantly blocked by 50 ng/ml or 500 ng/ml soluble CEA. Only extremely high concentrations of CEA (5000 ng/ml) led to a reduction.

These data show that CD137×CEA in the presence of CD38 or T cell receptor stimulation induces strong, CEA-dependent T cell activation.

### Example 22. CD137×CEA Reduces Tumors In Vivo

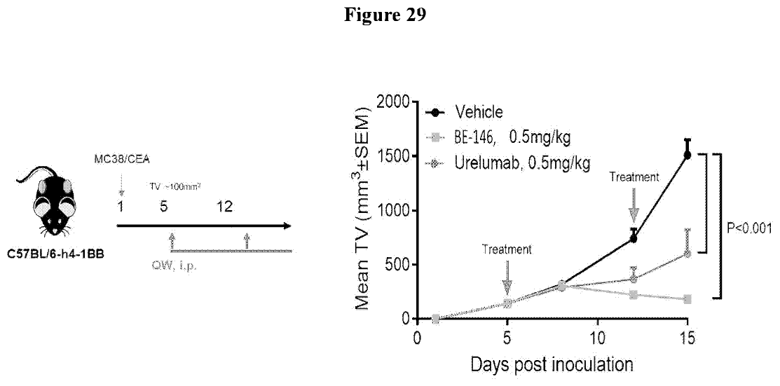

To determine the in vivo efficacy of CD137×CEA multispecific antibody BE-146 against CEA⁺ tumor cells, MC38/CEA cells (1×10⁶) were injected subcutaneously into humanized CD137 mice of the C57BL/6 background. Mice were randomized on day 5 post injection when the average tumor volume reached around 100 mm3. BE-146 (0.5 mg/kg), or Urelumab analog (0.5 mg/kg) or vehicle control was given once per week starting on day 5. As compared to vehicle control, both BE-146 and the Urelumab analog induced significant inhibition of tumor growth (P<0.001) (FIG. 29).

### Example 23. Combination Treatment of Anti-PD-1 Antibody and CD137×CEA Induces Increased Tumor Regression

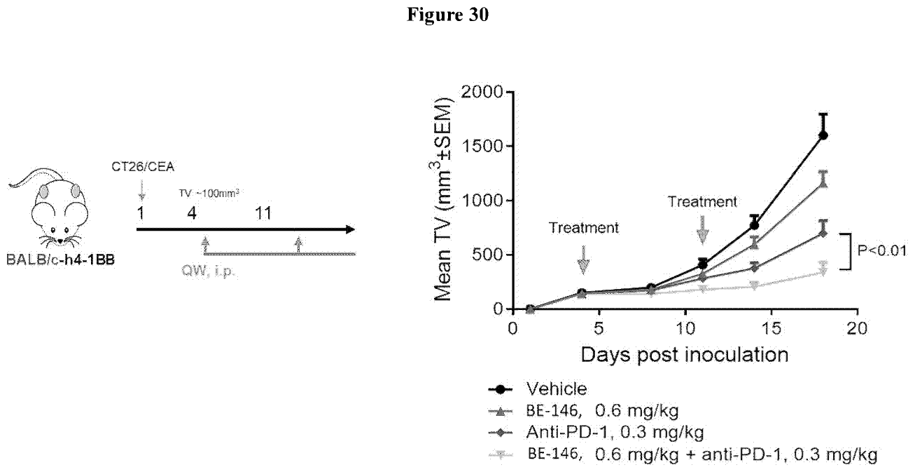

To investigate the combination treatment efficacy of CEA×CD137 multispecific antibody BE-146 and anti-PD-1 antibody against CEA⁺ tumor cells, CT26/CEA cells (1×10⁶) were injected subcutaneously into humanized CD137 mice of the BALB/c background. Mice were randomized on day 4 post injection when the average tumor volume reached around 100 mm3. BE-146 (0.6 mg/kg), anti-PD-1 antibody (0.3 mg/kg) or the combination of both was given once per week starting on day 4. As compared to vehicle control or single-agent treatments, the combination of BE-146 and anti-PD-1 induced significantly increased anti-tumor effects. (FIG. 30)

### Example 24. CD137×CEA does not Induce Liver Toxicity In Vivo

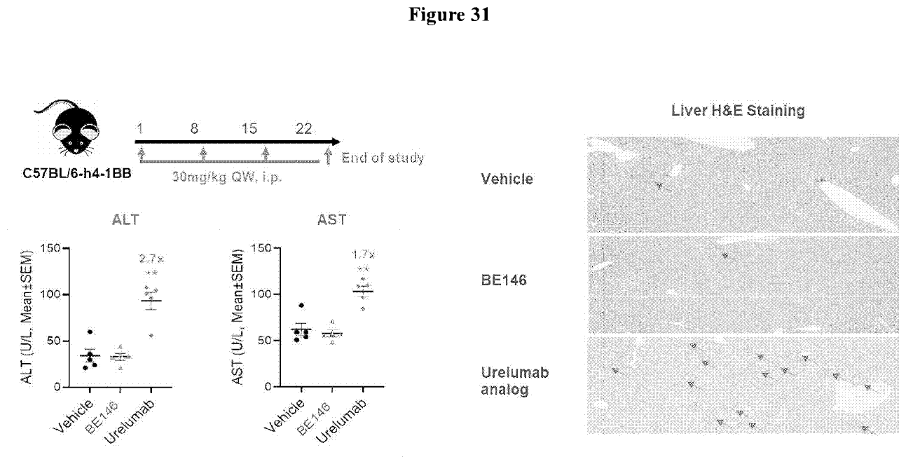

BE-146 or the Urelumab analog antibody (30 mg/kg) were injected into humanized CD137 mice of the C57BL/6 background, once per week for three doses. Blood was collected on day 22 and analyzed by blood biochemical tests. Compared with the vehicle control, high-dose of the Urelumab analog, but not BE-146, induced significantly increased alanine transaminase (ALT) and aspartate aminotransferase (AST) concentrations indicative of liver toxicity. In addition, microscopic changes of increased inflammatory cells were observed in hepatic tissues from the Urelumab analog-treated group while no significant microscopic changes were observed in the BE-146 treated group. (FIG. 31). Therefore, CD137×TAA is a promising combination partner for cancer immunotherapies without liver toxicity, including checkpoint inhibitors and T-cell engagers.

### Example 25. Other CD137×TAA Combinations Induce T Cell Activation in a TAA Target Dependent Manner

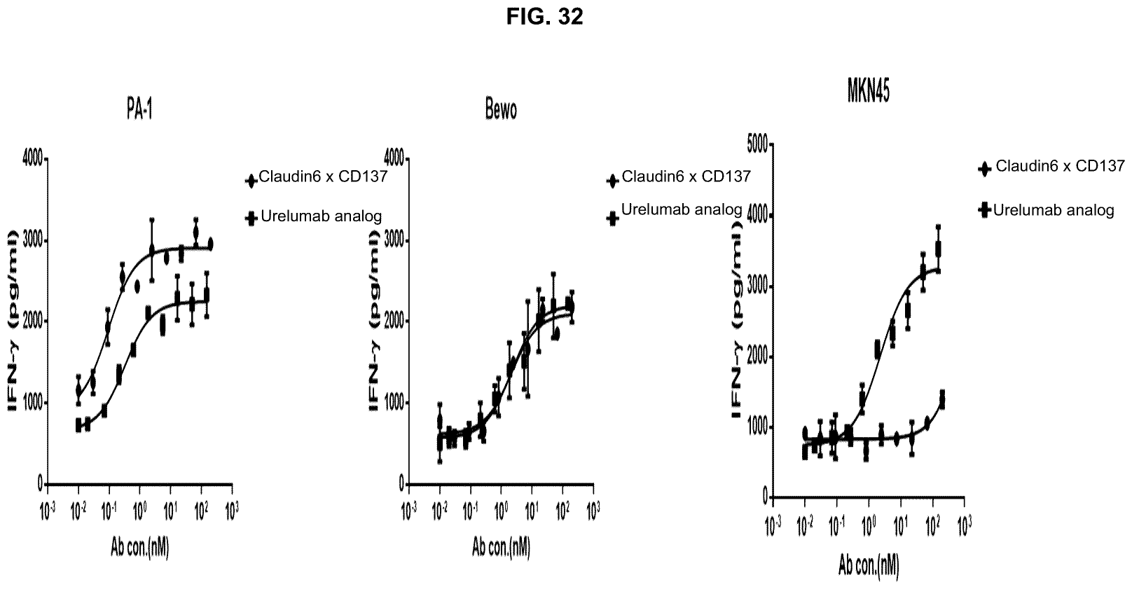

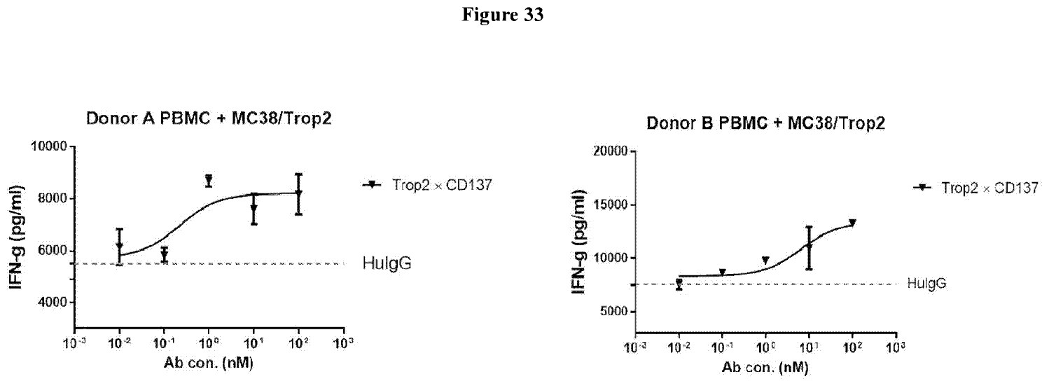

The same concept for Claudin6+ tumor cells and Trop2+ tumor cells was used to test other CD137×TAA combinations. PBMCs from healthy donors were used to activate human T cells with Claudin6×CD137 (BE-268) or Trop2×CD137 (BE-907) and with HEK293/OS8 providing readout. For BE-268, cancer cell lines with different Claudin6 expression level (1×10⁴ cells/well) were co-cultured in 96-well U bottom plates (Corning™ Costar™9018) for 2 days. PA-1 cells which have a high expression of Claudin6 and Bewo cells which have a mid-range expression of Claudin6 were purchased from ATCC. MKN45 cells that are negative for Claudin6 expression were purchased from JCRB cell bank. To assay for BE-907, the Trop2 expressing cell line Mc38/Trop2 was generated according to the protocols described previously (Zhang et al., Blood. 2005 106(5):1544-51). IFN-γ release from T cells were determined by ELISA. The results showed that both BE-268 and BE-907 could induce significant IFN-γ release (FIGS. 32 and 33). As expected, these results showed that BE-268 and BE-907 induced strong T cell activation in a Claudin6 and Trop2 dependent way.

### Example 26. CD137×GPC3 Induces T Cell Activation in a GPC3 Dependent Manner

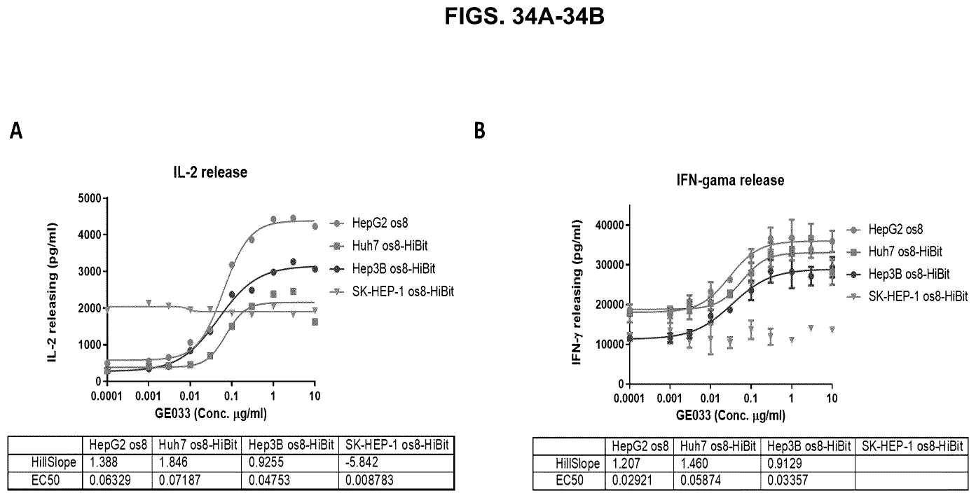

An additional TAA tested was Glypican 3 (GPC3). GPC3+ tumor cells were generated. As above, human peripheral blood mononuclear cells (PBMCs) were obtained from healthy donors to activate human T cells with CD137×GPC3 (BE-830) and OS8 to provide readout. To make the system very simple, OS8 expressing GPC3+ cell lines HepG2 OS8, Huh7 OS8-HiBit and Hep3B OS8-HiBit were generated by retroviral transduction into HepG2 (ATCC, HB8065), Huh7(JCRB, JCRB0403) and Hep3B (ATCC, HB8064) according to the protocols described previously (Zhang et al., Blood. 2005 106(5): 1544-51). SK-HEP-1 (ATCC, HTB-52) with OS8 (SK-HEP-1 OS8-HiBit) was generated in addition, which is negative for GPC3 expression and was used as a negative control. The co-culture was performed at an E:T ratio of 2:1 for 2 days in the presence of BE-830 at indicated concentrations (0.0001-10 μg/ml), and IFN-γ and IL-2 were determined by commercial ELISA kit. As shown in FIG. 34, in human HCC cell lines with GPC3 expression, regardless of the expression level of GPC3, BE-830 showed similar functional effect in inducing IFN-γ release in co-cultured PBMCs. BE-830 seems to stimulate higher IL-2 production in PBMCs cocultured with GPC3 high expressing HepG2 cells. These data showed that, similar to the CEA construct, BE-146, the multispecific antibody BE-830 is functionally active.

### Example 27. BE-830 Reduces Tumors In Vivo

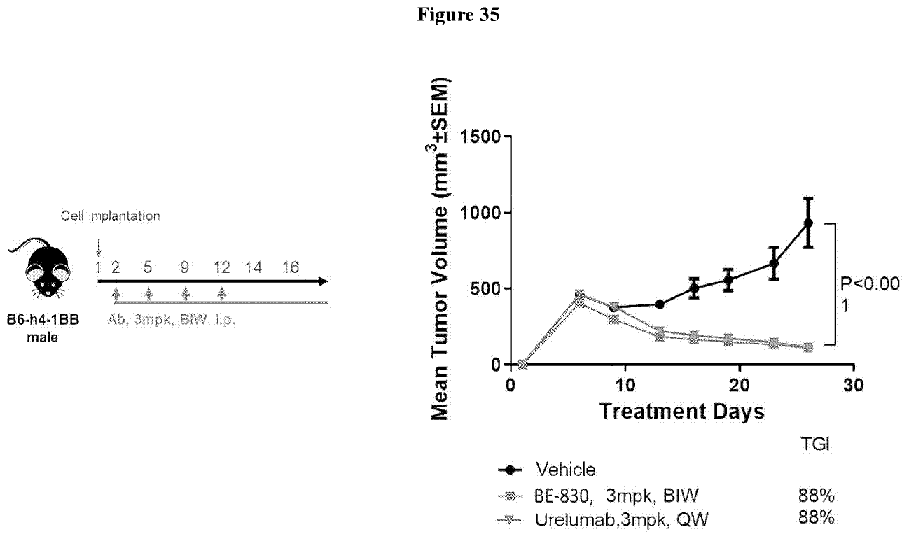

To determine the in vivo efficacy of BE-830 against GPC3+ tumor cells, Hepal-6T/hGPC3 cells (1×10⁷) were injected subcutaneously into humanized CD137 mice of the C57BL/6 background. BE-830 (0.5, 3 or 10 mpk) and vehicle control was given twice a week starting on the day of tumor injection (5 mice per group). As compared to vehicle, BE-830 at 0.5 mg/kg dosage showed tumor inhibition with statistically significant difference from vehicle control (P<0.01) (FIG. 35).

### Example 28. Crystal Structure Resolution

To better understand how the anti-CD137 single domain antibody arm is capable of high affinity for CD137, and robust agonist of CD137/CD137L interaction, the crystal structure of VHH (BGA-5623) in complex with CD137 was determined.

### CD137 and VHH (BGA-5623) Expression, Purification, and Crystallization

Human CD137 ectodomain containing four CRDs (1-4; amino acids 24-162) harboring C121S, N138D, and N149Q mutations was expressed in HEK293G cells. The cDNA coding CD137 was cloned into pMAX vector with an N-terminal secretion sequence and a C-terminal TEV cleavage site followed by an Fc tag. The culture supernatant containing the secreted CD137-Fc fusion protein was mixed with Mab Select Sure™ resin (GE Healthcare Life Sciences) for 3 hours at 4° C. The protein was washed with buffer containing 20 mM Tris-HCl pH 8.0, 150 mM NaCl, then eluted with 50 mM acetic acid (adjust pH value to 3.5 with 5 M NaOH), and finally neutralized with 1/10 CV 1.0M Tris-HCl pH8.0. The eluted protein was mixed with TEV proteases (10:1 molar ratio) and dialyzed against buffer (20 mM Tris-HCl, pH 8.0, 100 mM NaCl) at 4ºC overnight. The mixture was loaded onto a Ni-NTA column (Qiagen) and Mab Select Sure resin to remove the TEV proteases and Fc tag, and then the flow-through was further purified by size-exclusion chromatography in buffer (20 mM Tris pH 8.0, 100 mM NaCl) using a HiLoad 16/600 Superdex™ 75 pg column (GE Healthcare Life Sciences).

DNA sequence encoding VHH (BGA-5623) was cloned into a PET21a vector with N-terminal HIS-MBP tag followed by TEV protease site. Protein expression in Shuffle T7 was induced at OD600 of 0.6-1.0 with 1 mM IPTG at 18° C. for 16 hours. The cells were harvested by centrifugation at 7,000 g, 10 minutes. The cell pellets were re-suspended in lysis buffer (50 mM Na₃PO₄ pH 7.0, 300 mM NaCl) and lysed under sonication on ice. The lysate then was centrifuged at 48,000 g at 4° C. for 30 minutes. The supernatant was mixed with Talon resin and batched at 4° C. for 3 hours. The resin was washed with lysis buffer containing 5 mM imidazole, the protein was eluted in lysis buffer with additional 100 mM imidazole. The eluate was mixed with TEV proteases (10:1 molar ratio) and dialyzed against buffer (20 mM Tris-HCl, pH 8.0, 100 mM NaCl) at 4° C. overnight. The mixture was loaded onto a Talon column to remove the TEV proteases and HIS-MBP tag, and then the flow-through was further purified by size-exclusion chromatography in buffer (20 mM Tris pH 8.0, 100 mM NaCl) using a HiLoad 16/600 Superdex™ 75 pg column (GE Healthcare Life Sciences).

Purified CD137 was mixed with an excess of purified VHH (BGA-5623) (1:1.5 molar ratio) to generate the CD137/VHH (BGA-5623) complex. The complex was then further purified by gel filtration in buffer (20 mM Tris pH 8.0, 100 mM NaCl) using a HiLoad 16/600 Superdex™ 75 pg column (GE Healthcare Life Sciences). The CD137/VHH (BGA-5623) complex (10 mg/ml) was crystallized in 0.6 M Li₂SO4, 0.01 M NiCl₂,0.1 M Tris pH 9.0. Crystals cryoprotected with stepwise 5% D-(+)-Sucrose to a final 20% concentration were flash frozen in liquid nitrogen. Besides, the apoVHH (BGA-5623) was crystallized in 1.2 M (NH₄)₂SO₄, 0.1 M Citric Acid pH 5.0. Crystal was cryoprotected with 7% glycerol and flash frozen in liquid nitrogen. The X-ray diffraction data was collected at beamline BL45XU at Spring-8 synchrotron radiation facility (Hyogo, Japan).

### Data Collection and Structure Solution

The X-ray diffraction data was collected under cryo cooled conditions at 100 Kelvin at beamline BL45XU equipped with ZOO (Hirata, K., et al., Acta Crystallogr D Struct Biol, 2019. 75(Pt 2): 138-150) automated data collection system in Spring-8 synchrotron radiation facility (Hyogo, Japan). Diffraction images were processed with the integrated data processing software KAMO (Yamashita, et al., Acta Crystallogr D Struct Biol, 2018. 74 (Pt 5): 441-449) employing XDS (Kabsch W., Acta Crystallogr D Biol Crystallogr, 2010. 66 (Pt 2): 125-32). The structure of human CD137 (PDB: 6MGP) and VHH model (PDB:4U3X) were used as search models. The initial solution was found with molecular replacement program PHASER (McCoy et al., Phaser crystallographic software. J Appl Crystallogr, 2007. 40(Pt 4): 658-674). Then this model was iterative manually built with program COOT (Emsley et al., Acta Crystallogr D Biol Crystallogr, 2004. 60(Pt 12 Pt 1): 2126-32) and refinement using PHENIX (Adams et al., Acta Crystallogr D Biol Crystallogr, 2010. 66(Pt 2): 213-21). The final model was refined to acceptable R and R free values and Ramachandran statistics (calculated by Molprobity). Data processing and refinement statistics can be found in Table 25.

TABLE 25
Data collection and refinement statistics

| | apoVHH | | VHH (BGA-5623)/ | |
|---|---|---|---|---|
| Crystals | (BGA-5623) | | CD137 complex | |
| Data collection | | | | |
| X-ray source | BL45XU | | BL45XU | |
| Wavelength (Å) | 1.0 | | 1.0 | |
| Space group | P4₁2₁2 | | I4₁ | |
| Cell Dimensions | | | | |
| a (Å) | 68.342 | | 123.943 | |
| b (Å) | 68.342 | | 123.943 | |
| c (Å) | 102.952 | | 57.877 | |
| α (°) | 90 | | 90 | |
| β (°) | 90 | | 90 | |
| γ (°) | 90 | | 90 | |
| Resolution (Å) | 56.94-1.70 | (1.80-1.70)^{a} | 87.64-2.58 | (2.73-2.58) |
| Rsym (%) | 18.0 | (145.8) | 21.4 | (209.3) |
| <I/σ> | 14.3 | (1.6) | 12.3 | (1.2) |
| CC_{1/2} | 99.8 | (56.1) | 99.7 | (44.4) |
| Redundancy | 11.6 | (5.9) | 6.8 | (6.6) |
| Completeness (%) | 97.3 | (84.3) | 99.9 | (99.4) |

| | | |
|---|---|---|
| Refinement | | |
| Resolution (Å) | 48.33-1.70 | 43.82-2.58 |
| No. reflections | 26791 | 14038 |
| Rwork/Rfree ^{b}(%) | 19.84/23.46 | 22.8/28.9 |
| r.m.s.d. ^{c}bonds/ | 0.006/0.892 | 0.010/1.314 |
| angles | | |
| Number of atoms | | |
| Protein | 1787 | 1959 |
| Water | 204 | 5 |
| B factors | | |
| Protein | 27.37 | 83.97 |
| Water | 36.42 | 66.79 |
| Ramachandran | | |
| Favored (%) | 98.71 | 94.21 |
| Allowed (%) | 1.29 | 3.47 |
| Outlier (%) | 0 | 2.32 |

^{a}Values in parentheses are those of the highest resolution shell.
^{b}Calculated from about 5% of the reflection set aside during refinement
^{c}r.m.s.d., root mean square deviation

### The Structure of VHH (BGA-5623) Bound to Human CD137

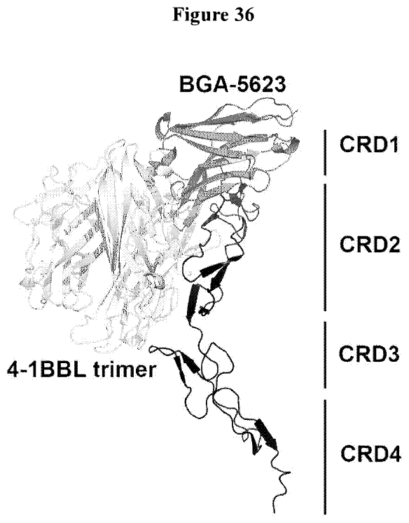

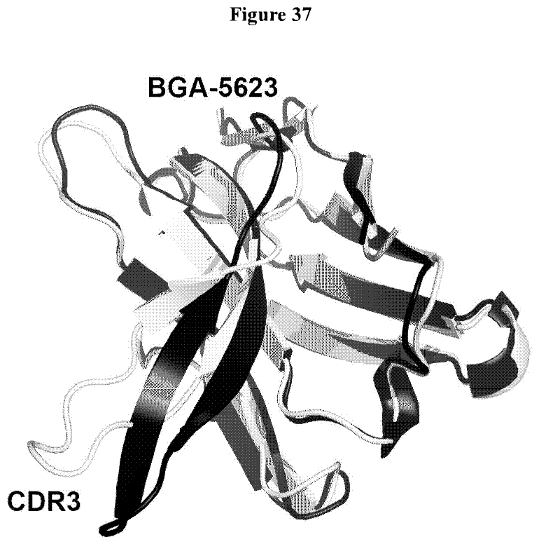

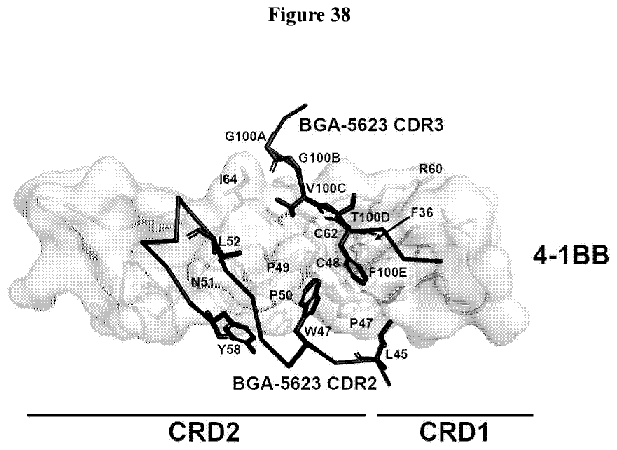

The VHH (BGA-5623) in complex with CD137 crystallized in the 141 space group, with one complex in the asymmetric unit, and diffracted to 2.58 Å. The structure of VHH (BGA-5623) bound to human CD137 shows that VHH (BGA-5623) partially sterically interfaces with CD137L binding (FIG. 36). The buried surface area between VHH (BGA-5623) and CD137 is approximately 571 Å². VHH (BGA-5623) interactions are clustered around CD137 CRD2 domain. These interactions are primarily mediated by VHH (BGA-5623) CDR2 and CDR3 and make more extensive contact with CD137. VHH (BGA-5623) CDR1 does not directly contact CD137 while CDR3 undergoes dramatically conformation change from unstructured loop to ß-sheet upon CD137 binding (FIG. 37). VHH (BGA-5623) CDR2 Leu52, Tyr58 contact CD137 residues Pro50, Asn51. VHH (BGA-5623) CDR3 residues Gly100A, Gly 100B, Val100C, Thr100D, Phe100E contact CD137 residues Phe36, Pro47, Pro49, Arg60, Cys62, Ile64. Besides, FR2 Leu45 and Trp47 contact CD137 residues Pro47, Cys48, Pro49, Pro50 which contribute significantly to CD137 binding. VHH (BGA-5623) interacts with CD137 using a combination of hydrogen bonds and hydrophobic interactions. For example, FR2 Trp47 forms strong hydrophobic contacts with CD137 residues Pro47, Cys48, Pro49 and Pro50. CDR3 residue Phe100E forms hydrophobic interactions with CD137 residues Phe36 and Pro47. FR2 residue Trp47 and CDR3 residue Gly 100A form one hydrogen bond with CD137 residues Pro47 and Ile64, respectively. CDR3 residue Val100C forms two hydrogen bonds with CD137 residue Cys62 (FIG. 38).

Based on the crystal structure of the VHH (BGA-5623)/CD137 complex, the residues of CD137 that are contacted by VHH (BGA-5623) (i.e., the epitopic residues of CD137 bound by VHH) and the residues of VHH (BGA-5623) that are contacted by CD137 (i.e. the paratopic residues of VHH contacted by CD137) were determined. Table 26, below, show the residues of CD137 and VHH (BGA-5623) to which they contact, as assessed using a contact distance stringency of 3.7 Å, a point at which van der Waals (non-polar) interaction forces are highest.

TABLE 26
Epitopic residues of CD137 and their corresponding
paratopic residues of VHH(BGA-5623)

| CD137 | | VHH (BGA-5623) | |
|---|---|---|---|
| Phe | 36 | Thr | 100D |
| | | Phe | 100E |
| Pro | 47 | Leu | 45 |
| | | Trp | 47 |
| | | Phe | 100E |
| Cys | 48 | Trp | 47 |
| Pro | 49 | Trp | 47 |
| | | Val | 100C |
| | | Thr | 100D |
| Pro | 50 | Trp | 47 |
| | | Tyr | 58 |
| Asn | 51 | Leu | 52 |
| Arg | 60 | Thr | 100D |
| Cys | 62 | Gly | 100B |
| | | Val | 100C |
| Ile | 64 | Gly | 100A |

The VHH (BGA-5623) residues are numbered in Kabat nomenclature.

1. An antibody or antigen-binding fragment thereof that specifically binds human CD137, the antibody or antigen-binding fragment thereof comprising:
(i) a heavy chain variable region that comprises (a) a HCDR1 of SEQ ID NO: 14, (b) a HCDR2 of SEQ ID NO: 29, and (c) a HCDR3 of SEQ ID NO: 30;
(ii) a heavy chain variable region that comprises (a) a HCDR1 of SEQ ID NO: 14, (b) a HCDR2 of SEQ ID NO: 22, and (c) a HCDR3 of SEQ ID NO: 16;
(iii) a heavy chain variable region that comprises (a) a HCDR1 of SEQ ID NO:14, (b) a HCDR2 of SEQ ID NO: 15, and (c) a HCDR3 of SEQ ID NO: 16; or
(iv) a heavy chain variable region that comprises (a) a HCDR1 of SEQ ID NO: 4, (b) a HCDR2 of SEQ ID NO: 5, and (c) a HCDR3 of SEQ ID NO: 6.

2. The antibody or antigen-binding fragment thereof of claim 1, wherein the antibody or antigen-binding fragment thereof comprises:
(i) a heavy chain variable region comprising an amino acid sequence at least 90% identical to SEQ ID NO: 33;
(ii) a heavy chain variable region comprising an amino acid sequence at least 90% identical to SEQ ID NO: 24;
(iii) a heavy chain variable region comprising an amino acid sequence at least 90% identical to SEQ ID NO: 19;
(iv) a heavy chain variable region comprising an amino acid sequence at least 90% identical to SEQ ID NO: 9; or
(v) a heavy chain variable region comprising an amino acid sequence at least 90 99% identical to SEQ ID NO: 103.

3. The antibody or antigen-binding fragment thereof of claim 2, wherein one, two, three, four, five, six, seven, eight, nine, or ten amino acids within SEQ ID NO:33, 24, 19, 9, or 103 have been inserted, deleted or substituted.

4. The antibody or antigen-binding fragment thereof of claim 1, wherein the antibody or antigen-binding fragment thereof comprises:
(i) a heavy chain variable region comprising SEQ ID NO: 33;
(ii) a heavy chain variable region comprising SEQ ID NO: 24;
(iii) a heavy chain variable region comprising SEQ ID NO: 19;
(iv) a heavy chain variable region comprising SEQ ID NO: 9; or
(v) a heavy chain variable region comprising SEQ ID NO: 103.

5. The antibody or antigen-binding fragment thereof of claim 1, wherein the antibody or antigen-binding fragment thereof is a heavy chain (scFv), a heavy chain Fab fragment, a heavy chain Fab′ fragment, or a heavy chain F(ab′)₂ fragment.

6. A multispecific antibody comprising at least a first antigen binding domain that specifically binds human CD137, wherein the first antigen binding domain comprises:
(i) a heavy chain variable region that comprises (a) a HCDR1 of SEQ ID NO: 14, (b) a HCDR2 of SEQ ID NO: 29, and (c) a HCDR3 of SEQ ID NO: 30;
(ii) a heavy chain variable region that comprises (a) a HCDR1 of SEQ ID NO: 14, (b) a HCDR2 of SEQ ID NO: 22, and (c) a HCDR3 of SEQ ID NO: 16;
(iii) a heavy chain variable region that comprises (a) a HCDR1 of SEQ ID NO:14, (b) a HCDR2 of SEQ ID NO: 15, and (c) a HCDR3 of SEQ ID NO: 16; or
(vi) a heavy chain variable region that comprises (a) a HCDR1 of SEQ ID NO:4, (b) a HCDR2 of SEQ ID NO: 5, and (c) a HCDR3 of SEQ ID NO: 6,
and at least a second antigen binding domain that specifically binds a human tumor-associated antigen (TAA).

7. The multispecific antibody of claim 6, wherein the first antigen binding domain comprises:
(i) a heavy chain variable region comprising SEQ ID NO: 33;
(ii) a heavy chain variable region comprising SEQ ID NO: 24;
(iii) a heavy chain variable region comprising SEQ ID NO: 19;
(iv) a heavy chain variable region comprising SEQ ID NO: 9; or
(iv) a heavy chain variable region comprising SEQ ID NO: 103.

8. The multispecific antibody of claim 7, wherein the multispecific antibody is a bispecific antibody.

9. The multispecific antibody of claim 8, wherein the bispecific antibody is in the 1+1 format, 1+2 format, or 2+2 format.

10.-12. (canceled)

13. The multispecific antibody of claim 8, further comprising a linker, wherein the linker is any sequence of SEQ ID NO:239 to SEQ ID NO:280.

14. The multispecific antibody of claim 13, wherein the linker is SEQ ID NO: 246 or SEQ ID NO: 251.

15. (canceled)

16. The antibody or antigen-binding fragment thereof of claim 1, wherein the antibody or antigen-binding fragment thereof has one or more of the following: antibody dependent cellular cytotoxicity (ADCC), complement dependent cytotoxicity (CDC), reduced glycosylation, no glycosylation, hypofucosylation, or increased bisecting GlcNac structures.

17.-18. (canceled)

19. The antibody or antigen-binding fragment thereof of claim 1, wherein the Fc domain is an IgG1.

20. The antibody or antigen-binding fragment thereof of claim 19, wherein the IgG1 has reduced ADCC.

21. A pharmaceutical composition comprising the antibody or antigen-binding fragment thereof of claim 1 and a pharmaceutically acceptable carrier.

22. A method of treating cancer comprising administering to a patient in need an effective amount of the antibody or antigen-binding fragment thereof of claim 1.

23.-26. (canceled)

27. An isolated nucleic acid that encodes the antibody or antigen-binding fragment thereof of claim 1.

28. A vector comprising the nucleic acid of claim 27.

29. A host cell comprising the nucleic acid of claim 27.

30. A process for producing an antibody or antigen-binding fragment thereof comprising cultivating the host cell of claim 29 and recovering the antibody or antigen-binding fragment thereof from the culture.
